SEQUENCE LISTING (4 sequences)

SEQ ID NO: 1 - DNA, 34 bp, Artificial
  PCR primer
ggggctcgaggcagggatggagaccaaaggctac

SEQ ID NO: 2 - DNA, 30 bp, Artificial
  PCR primer
ggatacccatcacttcttctagacgacagg

SEQ ID NO: 3 - DNA, 30 bp, Artificial
  PCR primer
agtgggtattaaacaagaaccagatgacgg

SEQ ID NO: 4 - DNA, 34 bp, Artificial
  PCR primer
gggaggtaccttctgggcagcgggcagtcacttc

(19) United States
(12) Patent Application Publication
(10) Pub. No.: US 2010/0094000 A1
(43) Pub. Date: Apr. 15, 2010

(54) PYRAZOLE COMPOUNDS

(75) Inventors: Shoji FUKUMOTO, Osaka-shi (JP); Norio OHYABU, Osaka-shi (JP); Taiichi OHRA, Osaka-shi (JP); Takahiro SUGIMOTO, Osaka-shi (JP); Tomoaki HASUI, Osaka-shi (JP); Koji FUJI, Osaka-shi (JP); Christopher S. SIEDEM, Boulder, CO (US); Cassandra GAUTHIER, Boulder, CO (US)

Correspondence Address: HAMRE, SCHUMANN, MUELLER & LARSON, P.C., P.O. BOX 2902, MINNEAPOLIS, MN, 55402-0902, US

(73) Assignee: TAKEDA PHARMACEUTICAL COMPANY LIMITED, Osaka (JP)

(21) Appl. No.: 12/552,701

(22) Filed: Sep. 2, 2009

Related U.S. Application Data
(60) Provisional application No. 61/093,893, filed on Sep. 3, 2008.

(51) Int. Cl. C07D 417/10

(52) U.S. Cl. 544/105

(57) ABSTRACT

The present invention relates to

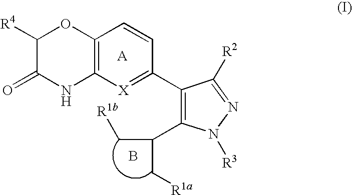

wherein each symbol is as defined in the specification. The compound has a superior mineralocorticoid receptor antagonistic action and is useful as an agent for the prophylaxis or treatment of a disease or condition mediated by the mineralocorticoid receptor activation.

### TECHNICAL FIELD

The present invention relates to a fused heterocyclic compound having a pyrazole ring, which is useful as an agent for the prophylaxis or treatment of hypertension, cardiac failure and the like; and the like.

### BACKGROUND OF THE INVENTION

Aldosterone is a final product of renin-angiotensin-aldosterone system (RAAS), which binds to a mineralocorticoid receptor (MR; aldosterone receptor). Since it expresses actions to adjust water and electrolyte, microvessel contraction, ischemia, induction of inflammation of blood vessel, promotion of tissue fibrosis and the like, it is suggested that excess production or secretion of aldosterone is involved in the diseases such as hypertension, congestive heart failure, arteriosclerosis, cerebral infarction, acute coronary diseases, nephropathy and the like. It has been reported that hypertension is developed in primary aldosteronism with increased secretion of aldosterone from the adrenal gland, and the complications in the cardiac or blood vessel system and kidney are observed at high frequency (see Journal of Clinical Endocrinology and Metabolism, 2003, vol. 88, p. 2364-2372). In addition, spironolactone and eplerenone having a steroid structure, which are used clinically, show a hypotensive action in patients with hypertension. In a large-scale clinical test, RALES (Randomized Aldactone Evaluation Study), it has been reported that spironolactone decreases the death rate of patients with severe cardiac failure (see New England Journal of Medicine, 1999, vol. 341, p. 709-717) and, in EPHESUS (Eplerenone Post-AMI Heart Failure Efficacy and Survival Study), it has been reported that eplerenone decreases the death rate and cardiovascular incidents in patients with cardiac infarction suffering from the complication of the decreased left ventricle function and cardiac failure (see New England Journal of Medicine, 2003, vol. 348, p. 1309-1321), and the usefulness of mineralocorticoid receptor antagonists in the treatment of hypertension and cardiac failure is being established.

As the mineralocorticoid receptor antagonist, compounds having a steroid structure such as canrenone and the like have been reported besides the above-mentioned spironolactone and eplerenone, and, as compounds having a non-steroidal skeleton, naphthalene derivative (see Biochemical Pharmacology, 1974, vol. 23, p. 1493), benzodiazepine derivative (see U.S. Pat. No. 4,251,443), indole derivative (see U.S. Pat. No. 4,179,503) and the like have been reported.

In addition, compounds having a non-steroidal skeleton, which interact with steroid hormone nuclear receptors including a mineralocorticoid receptor as a site of action, are disclosed in U.S. Pat. No. 6,964,973, WO03/078394, WO04/052847, WO05/066153, WO05/066161, WO05/087740, WO05/092854, WO05/097118, J. Comb. Chem., vol. 7, page 567-573 (2005), WO2006/015259, WO2007/077961 and the like. However, a compound having a structure as in the present invention is not disclosed.

### DISCLOSURE OF THE INVENTION

As a result of the intensive studies of the compounds having a mineralocorticoid receptor antagonistic action, the present inventors have surprisingly found compounds represented by the following formula (I), a salt thereof or a prodrug thereof has not only a superior mineralocorticoid receptor antagonistic action but also a better profile in toxicity, drug-drug interaction and the like, which resulted in the completion of the present invention.

Accordingly, the present invention provides the following.
[1] A compound represented by the formula (I):

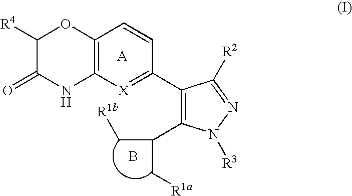

wherein

ring A is a benzene ring or a pyridine ring, each of which is optionally substituted;

ring B is a benzene ring or a 5- or 6-membered aromatic heterocycle, each of which is optionally substituted (wherein two substituents of said benzene ring or said 5- or 6-membered aromatic heterocycle can be bound and form a ring);

X is CX¹ or N;

X¹ is a hydrogen atom, a halogen atom or an optionally substituted C₁₋₆ alkyl;

each of R^{1a} and R^{1b} is independently a hydrogen atom, a halogen atom, a hydroxy, an optionally substituted C₁₋₆ alkyl or an optionally substituted C₁₋₆ alkoxy;

R² is a hydrogen atom, a halogen atom, an optionally substituted C₁₋₆ alkyl, an optionally substituted C₃₋₆ cycloalkyl, an optionally substituted C₁₋₆ alkoxy or an optionally substituted amino group;

R³ is a hydrogen atom or an optionally substituted hydrocarbon group; and

R⁴ is a hydrogen atom, a halogen atom, or an optionally substituted C₁₋₆ alkyl or an optionally substituted hydroxyl,

provided that, R³ is not a hydrogen atom when R² is a hydrogen atom, and R³ is not methyl when at least one of R^{1a} and R^{1b} is methyl.
[2] The compound of [1], wherein

each of R^{1a} and R^{1b} is independently a hydrogen atom or a halogen atom; and

R³ is an optionally substituted C₁₋₆ alkyl, an optionally substituted C₃₋₆ cycloalkyl, an optionally substituted C₃₋₆ alkenyl or an optionally substituted C₃₋₆ alkynyl.
[3] The compound of [1], wherein R² is an optionally substituted C₁₋₆ alkyl.
[4] The compound of [1], wherein R³ is a hydrocarbon group optionally substituted by halogen, cyano, nitro, optionally substituted hydroxy, optionally substituted alkylthio, acyl, optionally substituted carboxy, optionally substituted amino, optionally substituted cycloalkyl, optionally substituted aromatic hydrocarbon ring, optionally substituted aromatic heterocycle, optionally substituted non-aromatic heterocycle, or optionally substituted sulfonamido.
[5] The compound of [1] , wherein R³ is a C₁₋₆ alkyl optionally substituted by halogen, cyano, nitro, optionally substituted hydroxy, optionally substituted alkylthio, acyl, optionally substituted carboxy, optionally substituted amino, optionally substituted cycloalkyl, optionally substituted aromatic hydrocarbon ring, optionally substituted aromatic heterocycle, optionally substituted non-aromatic heterocycle, or optionally substituted sulfonamido.
[6] The compound of [1], wherein R⁴ is a hydrogen atom.
[7] The compound of [1], wherein ring B is an optionally substituted benzene ring.
[8] The compound of [1],
   wherein
   each of R^{1a} and R^{1b} is independently a hydrogen atom or a halogen atom;
   R² is an optionally substituted C₁₋₆ alkyl;
   R³ is an optionally substituted C₁₋₆ alkyl;
   R⁴ is a hydrogen atom; and
   ring B is an optionally substituted benzene ring.
[9] A prodrug of the compound of [1].
[10] A pharmaceutical composition comprising the compound of [1] or a prodrug thereof.
[11] The pharmaceutical composition of [10], wherein the composition is an aldosterone receptor antagonist.
[12] The pharmaceutical composition of [10], wherein the composition is an agent for preventing or treating hypertension.
[13] The pharmaceutical composition of [10], wherein the composition is an agent for preventing or treating organ is damage caused by hypertension.

### DETAILED DESCRIPTION OF THE INVENTION

The definition of each symbol in the formula (I) is described in detail in the following.

In the present specification, the “halogen atom” means, unless otherwise specified, fluorine, chlorine, bromine or iodine.

In the present specification, the “C₁₋₆ alkyl group” means, unless otherwise specified, methyl, ethyl, propyl, isopropyl, butyl, isobutyl, sec-butyl, tert-butyl, pentyl, isopentyl, neopentyl, 1-ethylpropyl, hexyl, isohexyl, 1,1-dimethylbutyl, 2,2-dimethylbutyl, 3,3-dimethylbutyl, 2-ethylbutyl or the like.

In the present specification, the “C₁₋₆ alkoxy group” means, unless otherwise specified, methoxy, ethoxy, propoxy, isopropoxy, butoxy, isobutoxy, sec-butoxy, tert-butoxy or the like.

In the present specification, the “C₁₋₆ alkoxy-carbonyl group” means, unless otherwise specified, methoxycarbonyl, ethoxycarbonyl, propoxycarbonyl, tert-butoxycarbonyl or the like.

In the present specification, the “C₁₋₆ alkyl-carbonyl group” means, unless otherwise specified, acetyl, propanoyl, butanoyl, isobutanoyl, pentanoyl, isopentanoyl, hexanoyl and the like.

In the present specification, the “C₂₋₆ alkenyl” means, unless otherwise specified, 1-propenyl, 2-propenyl, 2-methyl-1-propenyl, 1-butenyl, 2-butenyl, 3-butenyl, 3-methyl-2-butenyl, 1-pentenyl, 2-pentenyl, 3-pentenyl, 4-pentenyl, 4-methyl-3-pentenyl, 1-hexenyl, 3-hexenyl, 5-hexenyl and the like.

In the present specification, the “C₃₋₆ alkynyl” means, unless otherwise specified, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, 5-hexynyl and the like.

In the present specification, the “C₃₋₆ cycloalkyl group” means, unless otherwise specified, cyclopropyl, cyclobutyl, cyclopentyl, cyclohexyl and the like.

In the present specification, the “C₃₋₆ cycloalkenyl group” means, unless otherwise specified, 2-cyclopenten-1-yl, 3-cyclopenten-1-yl, 2-cyclohexen-1-yl, 3-cyclohexen-1-yl and the like.

In the present specification, the “C₆₋₁₄ aryl group” means, unless otherwise specified, phenyl, naphthyl, anthryl, phenanthryl, acenaphthylenyl, biphenylyl and the like.

In the present specification, the “C₇₋₁₃ aralkyl group” means, unless otherwise specified, benzyl, phenethyl, naphthylmethyl, biphenylylmethyl and the like.

In the present specification, the “C₈₋₁₃ arylalkenyl group” means, unless otherwise specified, styryl and the like.

In the present specification, the “C₁₋₃ alkylenedioxy group” means, unless otherwise specified, methylenedioxy, ethylenedioxy and the like.

R^{1a} and R^{1b} are the same or different and each is a hydrogen atom, a halogen atom, hydroxy optionally having substituent(s), C₁₋₆ alkyl optionally having substituent(s) or C₁₋₆ alkoxy optionally having substituent(s).

The aforementioned C₁₋₆ alkyl and C₁₋₆ alkoxy optionally have 1 to 3 substituents at substitutable position(s).

Examples of such substituent include
(1) a C₃₋₈ cycloalkyl group (e.g., cyclopropyl, cyclohexyl);
(2) a C₆₋₁₄ aryl group (e.g., phenyl, naphthyl) optionally substituted by 1 to 3 substituents selected from

(a) a C₁₋₆ alkyl group optionally substituted by 1 to 3 halogen atoms,

(b) a hydroxy group,

(c) a C₁₋₆ alkoxy group optionally substituted by 1 to 3 halogen atoms, and

(d) a halogen atom;
(3) an aromatic heterocyclic group (e.g., thienyl, furyl, pyridyl, pyrazolyl, imidazolyl, tetrazolyl, oxazolyl, thiazolyl, oxadiazolyl, thiadiazolyl) optionally substituted by 1 to 3 substituents selected from

(a) a C₁₋₆ alkyl group optionally substituted by 1 to 3 halogen atoms,

(b) a hydroxy group,

(c) a C₁₋₆ alkoxy group optionally substituted by 1 to 3 halogen atoms, and

(d) a halogen atom;
(4) a nonaromatic heterocyclic group (e.g., tetrahydrofuryl, morpholinyl, thiomorpholinyl, piperidinyl, pyrrolidinyl, piperazinyl) optionally substituted by 1 to 3 substituents selected from

(a) a C₁₋₆ alkyl group optionally substituted by 1 to 3 halogen atoms,

(b) a hydroxy group,

(c) a C₁₋₆ alkoxy group optionally substituted by 1 to 3 halogen atoms,

(d) a halogen atom, and

(e) an oxo group;
(5) an amino group optionally mono- or di-substituted by substituent(s) selected from

(a) a C₁₋₆ alkyl group optionally substituted by 1 to 3 halogen atoms,

(b) a C₁₋₆ alkyl-carbonyl group optionally substituted by 1 to 3 halogen atoms,

(c) a C₁₋₆ alkoxy-carbonyl group optionally substituted by 1 to 3 halogen atoms,

(d) a C₁₋₆ alkylsulfonyl group (e.g., methylsulfonyl) optionally substituted by 1 to 3 halogen atoms,

(e) a carbamoyl group optionally mono- or di-substituted by a C₁₋₆ alkyl group optionally substituted by 1 to 3 halogen atoms, and

(f) an aromatic heterocyclic group (e.g., thienyl, furyl, pyridyl, pyrazolyl, imidazolyl, tetrazolyl, oxazolyl, thiazolyl, oxadiazolyl, thiadiazolyl);
(6) a C₁₋₆ alkyl-carbonyl group optionally substituted by 1 to 3 halogen atoms;
(7) a C₁₋₆ alkoxy-carbonyl group optionally substituted by 1 to 3 substituents selected from

(a) a halogen atom,

(b) a C₁₋₆ alkoxy group, and

(c) a C₆₋₁₄ aryl group (e.g., phenyl);
(8) a C₁₋₆ alkylsulfonyl group optionally substituted by 1 to 3 halogen atoms (e.g., methylsulfonyl, ethylsulfonyl, isopropylsulfonyl);
(9) a C₁₋₆ alkylsulfinyl group optionally substituted by 1 to 3 halogen atoms (e.g., methylsulfinyl, ethylsulfinyl, isopropylsulfinyl);
(10) a carbamoyl group optionally mono- or di-substituted by a C₁₋₆ alkyl group optionally substituted by 1 to 3 halogen atoms;
(11) a thiocarbamoyl group optionally mono- or di-substituted by a C₁₋₆ alkyl group optionally substituted by 1 to 3 halogen atoms;
(12) a sulfamoyl group optionally mono- or di-substituted by a C₁₋₆ alkyl group optionally substituted by 1 to 3 halogen atoms;
(13) a carboxy group;
(14) a hydroxy group;
(15) a C₁₋₆ alkoxy group optionally substituted by 1 to 3 substituents selected from

(a) a halogen atom,

(b) a carboxy group,

(c) a C₁₋₆ alkoxy group,

(d) a C₁₋₆ alkoxy-carbonyl group optionally substituted by 1 to 3 C₆₋₁₄ aryl groups (e.g., phenyl), and

(e) an amino group optionally mono- or di-substituted by substituent(s) selected from a C₁₋₆ alkyl group and a C₁₋₆ alkoxy-carbonyl group;
(16) a C₂₋₆ alkenyloxy group optionally substituted by 1 to 3 halogen atoms (e.g., ethenyloxy);
(17) a C₇₋₁₃ aralkyloxy group (e.g., benzyloxy);
(18) a C₆₋₁₄ aryloxy group (e.g., phenyloxy, naphthyloxy);
(19) a C₁₋₆ alkyl-carbonyloxy group (e.g., acetyloxy, tert-butylcarbonyloxy);
(20) a C₆₋₁₄ aryl-carbonyl group (e.g., benzoyl) optionally substituted by 1 to 3 substituents selected from

(a) a halogen atom, and

(b) a C₁₋₆ alkyl group optionally substituted by 1 to 3 halogen atoms;
(21) a non-aromatic heterocyclyl-carbonyl group (e.g., pyrrolidinylcarbonyl, morpholinylcarbonyl) optionally substituted by 1 to 3 substituents selected from C₁₋₆ alkyl groups optionally substituted by 1 to 3 halogen atoms;
(22) a mercapto group;
(23) a C₁₋₆ alkylthio group (e.g., methylthio, ethylthio) optionally substituted by 1 to 3 substituents selected from

(a) a halogen atom, and

(b) a C₁₋₆ alkoxycarbonyl;
(24) a C₇₋₁₃ aralkylthio group (e.g., benzylthio);
(25) a C₆₋₁₄ arylthio group (e.g., phenylthio, naphthylthio);
(26) a cyano group;
(27) a nitro group;
(28) a halogen atom;
(29) a C₁₋₃ alkylenedioxy group;
(30) an aromatic heterocyclyl-carbonyl group (e.g., pyrazolylcarbonyl, pyrazinylcarbonyl, isoxazolylcarbonyl, pyridylcarbonyl, thiazolylcarbonyl) optionally substituted by 1 to 3 substituents selected from C₁₋₆ alkyl groups optionally substituted by 1 to 3 halogen atoms;
and the like. When the number of the substituents is two or more, the respective substituents may be the same or different.
R^{1a} and R^{1b} are each preferably
(1) a hydrogen atom; or
(2) a halogen atom.
R^{1a} and R^{1b} are each particularly preferably
(1) a hydrogen atom.

R² is a hydrogen atom, a halogen atom, C₁₋₆ alkyl optionally having substituent(s), C₃₋₆ cycloalkyl optionally having substituent(s), C₁₋₆ alkoxy optionally having substituent(s) or amino optionally having substituent(s).

The aforementioned C₁₋₆ alkyl, C₃₋₆ cycloalkyl and C₁₋₆ alkoxy optionally have 1 to 3 substituents at substitutable position(s).

Examples of such substituent include the following substituents shown as the substituents of C₁₋₆ alkyl for the aforementioned R^{1a} or R^{1b}.
(1) a C₃₋₈ cycloalkyl group (e.g., cyclopropyl, cyclohexyl);
(2) a C₆₋₁₄ aryl group (e.g., phenyl, naphthyl) optionally substituted by 1 to 3 substituents selected from

(a) a C₁₋₆ alkyl group optionally substituted by 1 to 3 halogen atoms,

(b) a hydroxy group,

(c) a C₁₋₆ alkoxy group optionally substituted by 1 to 3 halogen atoms, and

(d) a halogen atom;
(3) an aromatic heterocyclic group (e.g., thienyl, furyl, pyridyl, pyrazolyl, imidazolyl, tetrazolyl, oxazolyl, thiazolyl, oxadiazolyl, thiadiazolyl) optionally substituted by 1 to 3 substituents selected from

(a) a C₁₋₆ alkyl group optionally substituted by 1 to 3 halogen atoms,

(b) a hydroxy group,

(c) a C₁₋₆ alkoxy group optionally substituted by 1 to 3 halogen atoms, and

(d) a halogen atom;
(4) a nonaromatic heterocyclic group (e.g., tetrahydrofuryl, morpholinyl, thiomorpholinyl, piperidinyl, pyrrolidinyl, piperazinyl) optionally substituted by 1 to 3 substituents selected from

(a) a C₁₋₆ alkyl group optionally substituted by 1 to 3 halogen atoms,

(b) a hydroxy group,

(c) a C₁₋₆ alkoxy group optionally substituted by 1 to 3 halogen atoms,

(d) a halogen atom, and

(e) an oxo group;
(5) an amino group optionally mono- or di-substituted by substituent(s) selected from

(a) a C₁₋₆ alkyl group optionally substituted by 1 to 3 halogen atoms,

(b) a C₁₋₆ alkyl-carbonyl group optionally substituted by 1 to 3 halogen atoms,

(c) a C₁₋₆ alkoxy-carbonyl group optionally substituted by 1 to 3 halogen atoms,

(d) a C₁₋₆ alkylsulfonyl group optionally substituted by 1 to 3 halogen atoms (e.g., methylsulfonyl),

(e) a carbamoyl group optionally mono- or di-substituted by a C₁₋₆ alkyl group optionally substituted by 1 to 3 halogen atoms, and

(f) an aromatic heterocyclic group (e.g., thienyl, furyl, pyridyl, pyrazolyl, imidazolyl, tetrazolyl, oxazolyl, thiazolyl, oxadiazolyl, thiadiazolyl);
(6) a C₁₋₆ alkyl-carbonyl group optionally substituted by 1 to 3 halogen atoms;
(7) a C₁₋₆ alkoxy-carbonyl group optionally substituted by 1 to 3 substituents selected from

(a) a halogen atom,

(b) a C₁₋₆ alkoxy group, and

(c) a C₆₋₁₄ aryl group (e.g., phenyl);
(8) a C₁₋₆ alkylsulfonyl group optionally substituted by 1 to 3 halogen atoms (e.g., methylsulfonyl, ethylsulfonyl, isopropylsulfonyl);
(9) a C₁₋₆ alkylsulfinyl group optionally substituted by 1 to 3 halogen atoms (e.g., methylsulfinyl, ethylsulfinyl, isopropylsulfinyl);
(10) a carbamoyl group optionally mono- or di-substituted by a C₁₋₆ alkyl group optionally substituted by 1 to 3 halogen atoms;
(11) a thiocarbamoyl group optionally mono- or di-substituted by a C₁₋₆ alkyl group optionally substituted by 1 to 3 halogen atoms;
(12) a sulfamoyl group optionally mono- or di-substituted by a C₁₋₆ alkyl group optionally substituted by 1 to 3 halogen atoms;
(13) a carboxy group;
(14) a hydroxy group;
(15) a C₁₋₆ alkoxy group optionally substituted by 1 to 3 substituents selected from

(a) a halogen atom,

(b) a carboxy group,

(c) C₁₋₆ alkoxy group,

(d) a C₁₋₆ alkoxy-carbonyl group optionally substituted by 1 to 3 C₆₋₁₄ aryl groups (e.g., phenyl), and

(e) an amino group optionally mono- or di-substituted by substituent(s) selected from a C₁₋₆ alkyl group and a C₁₋₆ alkoxy-carbonyl group;
(16) a C₂₋₆ alkenyloxy group optionally substituted by 1 to 3 halogen atoms (e.g., ethenyloxy);
(17) a C₇₋₁₃ aralkyloxy group (e.g., benzyloxy);
(18) a C₆₋₁₄ aryloxy group (e.g., phenyloxy, naphthyloxy);
(19) a C₁₋₆ alkyl-carbonyloxy group (e.g., acetyloxy, tert-butylcarbonyloxy);
(20) a C₆₋₁₄ aryl-carbonyl group (e.g., benzoyl) optionally substituted by 1 to 3 substituents selected from

(a) a halogen atom, and

(b) a C₁₋₆ alkyl group optionally substituted by 1 to 3 halogen atoms;
(21) a non-aromatic heterocyclyl-carbonyl group (e.g., pyrrolidinylcarbonyl, morpholinylcarbonyl) optionally substituted by 1 to 3 substituents selected from C₁₋₆ alkyl groups optionally substituted by 1 to 3 halogen atoms;
(22) a mercapto group;
(23) a C₁₋₆ alkylthio group (e.g., methylthio, ethylthio) optionally substituted by 1 to 3 substituents selected from

(a) a halogen atom, and

(b) C₁₋₆ alkoxycarbonyl;
(24) a C₇₋₁₃ aralkylthio group (e.g., benzylthio);
(25) a C₆₋₁₄ arylthio group (e.g., phenylthio, naphthylthio);
(26) a cyano group;
(27) a nitro group;
(28) a halogen atom;
(29) a C₁₋₃ alkylenedioxy group;
(30) an aromatic heterocyclyl-carbonyl group (e.g., pyrazolylcarbonyl, pyrazinylcarbonyl, isoxazolylcarbonyl, pyridylcarbonyl, thiazolylcarbonyl) optionally substituted by 1 to 3 substituents selected from C₁₋₆ alkyl groups optionally substituted by 1 to 3 halogen atoms; and the like. When the number of the substituents is two or more, the respective substituents may be the same or different.

Examples of the “optionally substituted amino group” for R² include an amino group optionally mono- or di-substituted by substituent(s) selected from a C₁₋₆ alkyl group, a C₂₋₆ alkenyl group, a C₃₋₆ cycloalkyl group, a C₃₋₆ cycloalkenyl group, a C₆₋₁₄ aryl group, a C₇₋₁₃ aralkyl group, a C₈₋₁₃ arylalkenyl group, a heterocyclic group, an acyl group and the like, each of which is optionally substituted.

The C₁₋₆ alkyl group, C₂₋₆ alkenyl group, C₃₋₆ cycloalkyl group, C₃₋₆ cycloalkenyl group, C₆₋₁₄ aryl group, C₇₋₁₃ aralkyl group, C₈₋₁₃ arylalkenyl group and heterocyclic group each optionally have 1 to 3 substituents at substitutable position(s). When the number of the substituents is two or more, the respective substituents may be the same or different. As such substituent, those similar to the substituents that the C₁₋₆ alkyl group and the like for R^{1a} or R^{1b} may have can be mentioned.

R² is preferably a C₁₋₆ alkyl optionally having substituent(s).

R² is more preferably a C₁₋₆ alkyl optionally substituted by 1 to 3 halogen atoms, particularly preferably C₁₋₆ alkyl.

R³ is a hydrogen atom or a hydrocarbon group optionally having substituent(s).

Examples of the “hydrocarbon group” of the “hydrocarbon group optionally having substituent(s)” for R³ include C₁₋₁₀ alkyl group, C₂₋₁₀ alkenyl group, C₂₋₁₀ alkynyl group, C₃₋₁₀ cycloalkyl group, C₃₋₁₀ cycloalkenyl group, C₄₋₁₀ cycloalkadienyl group, C₆₋₁₄ aryl group, C₇₋₁₃ aralkyl group, C₈₋₁₃ arylalkenyl group and the like.

Examples of the C₁₋₁₀ alkyl group include methyl, ethyl, propyl, isopropyl, butyl, isobutyl, sec-butyl, tert-butyl, pentyl, isopentyl, neopentyl, 1-ethylpropyl, hexyl, isohexyl, 1,1-dimethylbutyl, 2,2-dimethylbutyl, 3,3-dimethylbutyl, 2-ethylbutyl, heptyl, octyl, nonyl, decyl and the like. It is preferably a C₁₋₆ alkyl group, particularly preferably a C₁₋₃ alkyl group.

Examples of the C₂₋₁₀ alkenyl group include ethenyl, 1-propenyl, 2-propenyl, 2-methyl-1-propenyl, 1-butenyl, 2-butenyl, 3-butenyl, 3-methyl-2-butenyl, 1-pentenyl, 2-pentenyl, 3-pentenyl, 4-pentenyl, 4-methyl-3-pentenyl, 1-hexenyl, 3-hexenyl, 5-hexenyl, 1-heptenyl, 1-octenyl and the like, with preference given to a C₃₋₆ alkenyl group.

Examples of the C₂₋₁₀ alkynyl group include ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, 5-hexynyl, 1-heptynyl, 1-octynyl and the like, with preference given to a C₃₋₆ alkynyl group.

Examples of the C₃₋₁₀ cycloalkyl group include cyclopropyl, cyclobutyl, cyclopentyl, cyclohexyl, cycloheptyl, cyclooctyl, bicyclo[2.2.1]heptyl, bicyclo[2.2.2]octyl, bicyclo[3.2.1]octyl, bicyclo[3.2.2]nonyl, bicyclo[3.3.1]nonyl, bicyclo[4.2.1]nonyl, bicyclo[4.3.1]decyl, adamantyl and the like, with preference given to a C₃₋₆ cycloalkyl group.

Examples of the C₃₋₁₀ cycloalkenyl group include 2-cyclopenten-1-yl, 3-cyclopenten-1-yl, 2-cyclohexen-1-yl, 3-cyclohexen-1-yl and the like, with preference given to a C₃₋₈ cycloalkenyl group.

Examples of the C₄₋₁₀ cycloalkadienyl group include 2,4-cyclopentadien-1-yl, 2,4-cyclohexadien-1-yl, 2,5-cyclohexadien-1-yl and the like, with preference given to a C₄₋₈ cycloalkadienyl group.

The above-mentioned C₃₋₁₀ cycloalkyl group, C₃₋₁₀ cycloalkenyl group and C₄₋₁₀ cycloalkadienyl group may be each condensed with 1 or 2 benzene rings to form a fused ring group. Examples of such fused ring group include indanyl, indenyl, dihydronaphthyl, tetrahydronaphthyl, fluorenyl and the like.

Examples of the C₆₋₁₄ aryl group include phenyl, naphthyl, anthryl, phenanthryl, acenaphthylenyl, biphenylyl and the like. It is preferably a C₆₋₁₀ aryl group, and particularly preferably phenyl.

Examples of the C₇₋₁₃ aralkyl group include benzyl, phenethyl, naphthylmethyl, biphenylylmethyl and the like.

Examples of the C₈₋₁₃ arylalkenyl group include styryl and the like.

The aforementioned “hydrocarbon group” optionally has 1 to 4 substituents at substitutable position(s).

Examples of such substituent include
(1) a halogen atom;
(2) a cyano group;
(3) a nitro group;
(4) a hydroxy group optionally having substituent(s);
(5) an alkylthio group optionally having substituent(s);
(6) an acyl group;
(7) an amino group optionally having substituent(s);
(8) a cycloalkyl group optionally having substituent(s);
(9) an aromatic hydrocarbon ring optionally having substituent(s);
(10) an aromatic hetero ring optionally having substituent(s);
(11) an aliphatic hetero ring optionally having substituent(s);
(12) a sulfonamido group optionally having substituent(s); and the like. When the number of the substituents is two or more, the respective substituents may be the same or different.

R³ is preferably a C₁₋₆ alkyl optionally substituted by 1 to 4 substituents selected from
(1) a C₃₋₈ cycloalkyl group (e.g., cyclopropyl, cyclohexyl) optionally substituted by 1 to 3 substituents selected from

(a) a C₁₋₆ alkyl group optionally substituted by 1 to 3 substituents selected from a halogen atom and a hydroxy group,

(b) a hydroxy group,

(c) a C₁₋₆ alkoxy group optionally substituted by 1 to 3 halogen atoms,

(d) a halogen atom, and

(e) an oxo group;
(2) a phenyl group optionally substituted by 1 to 3 substituents selected from

(a) a C₁₋₆ alkyl group optionally substituted by 1 to 3 halogen atoms,

(b) a hydroxy group,

(c) a C₁₋₆ alkoxy group optionally substituted by 1 to 3 halogen atoms, and

(d) a halogen atom, and

(e) a cyano group;
(3) an aromatic heterocyclic group (e.g., thienyl, furyl, pyridyl, pyrazolyl, imidazolyl, tetrazolyl, oxazolyl, thiazolyl, oxadiazolyl, thiadiazolyl, isoxazolyl) optionally substituted by 1 to 3 substituents selected from

(a) a C₁₋₆ alkyl group optionally substituted by 1 to 3 halogen atoms,

(b) a hydroxy group,

(c) a C₁₋₆ alkoxy group optionally substituted by 1 to 3 halogen atoms, and

(d) a halogen atom, and

(e) a cyano group;
(4) a nonaromatic heterocyclic group (e.g., tetrahydrofuryl, morpholinyl, thiomorpholinyl, piperidinyl, pyrrolidinyl, piperazinyl, tetrahydropyranyl, tetrahydrothiopyranyl) optionally substituted by 1 to 3 substituents selected from

(a) a C₁₋₆ alkyl group optionally substituted by 1 to 3 halogen atoms,

(b) a hydroxy group,

(c) a C₁₋₆ alkoxy group optionally substituted by 1 to 3 halogen atoms,

(d) a halogen atom, and

(e) an oxo group;
(5) an amino group optionally mono- or di-substituted by substituent(s) selected from

(a) a C₁₋₆ alkyl group optionally substituted by 1 to 3 halogen atoms,

(b) a C₁₋₆ alkyl-carbonyl group optionally substituted by 1 to 3 halogen atoms,

(c) a C₁₋₆ alkoxy-carbonyl group optionally substituted by 1 to 3 halogen atoms,

(d) a C₁₋₆ alkylsulfonyl group optionally substituted by 1 to 3 halogen atoms (e.g., methylsulfonyl),

(e) a carbamoyl group optionally mono- or di-substituted by a C₁₋₆ alkyl group optionally substituted by 1 to 3 halogen atoms, and

(f) an aromatic heterocyclic group (e.g., thienyl, furyl, pyridyl, pyrazolyl, imidazolyl, tetrazolyl, oxazolyl, thiazolyl, oxadiazolyl, thiadiazolyl);
(6) an acyl group;
(7) a hydroxy group;
(8) a C₁₋₆ alkoxy group optionally substituted by 1 to 3 substituents selected from

(a) a halogen atom,

(b) a carboxy group,

(c) a C₁₋₆ alkoxy group,

(d) a C₁₋₆ alkoxy-carbonyl group optionally substituted 1 to 3 C₆₋₁₄ aryl groups (e.g., phenyl),

(e) an amino group optionally mono- or di-substituted by substituent(s) selected from a C₁₋₆ alkyl group and a C₁₋₆ alkoxy-carbonyl group, and

(f) a carbamoyl group;
(9) a C₁₋₆ alkylthio group (e.g., methylthio, ethylthio) optionally substituted by 1 to 3 substituents selected from

(a) a halogen atom,

(b) C₁₋₆ alkoxycarbonyl, and

(c) a hydroxy group;
(10) a cyano group;
(11) a nitro group; and
(12) a halogen atom.

R³ is particularly preferably a C₁₋₆ alkyl optionally substituted by 1 to 4 substituents selected from
(1) a C₃₋₆ cycloalkyl group (e.g., cyclopropyl, cyclohexyl);
(2) an acyl group;
(3) a hydroxy group;
(4) a C₁₋₆ alkoxy group;
(5) a C₁₋₆ alkylthio group (e.g., methylthio, ethylthio); and
(6) a halogen atom.

R⁴ is a hydrogen atom, a halogen atom, C₁₋₆ alkyl optionally having substituent(s) or hydroxy optionally having substituent(s). The aforementioned C₁₋₆ alkyl optionally has 1 to 3 substituents at substitutable position(s).

Examples of such substituent include those shown as the substituents of C₁₋₆ alkyl for the aforementioned R^{1a} or R^{1b}.

R⁴ is preferably a hydrogen atom or C₁₋₆ alkyl optionally having substituent(s).

R⁴ is particularly preferably a hydrogen atom.

X is CX¹ or a nitrogen atom. Here, X¹ is a hydrogen atom, a halogen atom or C₁₋₆ alkyl optionally having substituent(s). Preferably, X is CX¹, particularly preferably CH.

Ring A is a benzene ring optionally having substituent(s) or a pyridine ring optionally having substituent(s). A benzene ring or pyridine ring for ring A optionally has 1 to 3 substituents at substitutable position(s). Examples of such substituent include those similar to the substituents that C₁₋₆ alkyl or C₁₋₆ alkoxy for R^{1a} or R^{1b} optionally has. When the number of the substituents is two or more, the respective substituents may be the same or different.

Ring A is preferably a benzene ring or pyridine ring optionally further substituted by 1 or 2 substituents selected from
(1) a halogen atom;
(2) a hydroxy group;
(3) a C₁₋₆ alkyl group (preferably, C₁₋₃ alkyl group) optionally substituted by 1 to 3 halogens;
(4) a C₃₋₈ cycloalkyl group;
(5) an amino group optionally mono- or di-substituted by a C₁₋₆ alkyl group (preferably C₁₋₃ alkyl group);
(6) a C₁₋₆ alkoxy group optionally substituted by 1 to 3 halogen atoms; and
(7) a C₁₋₆ alkyl (preferably C₁₋₃ alkyl)-carbonyl group.

Ring A is particularly preferably a benzene ring optionally further substituted by 1 or 2 substituents selected from
(1) a halogen atom; and
(2) a C₁₋₆ alkyl group (preferably, C₁₋₃ alkyl group).

Ring B is a benzene ring optionally having substituent(s) or a 5- or 6-membered aromatic heterocycle optionally having substituent(s). Here, two substituents of the benzene ring or aromatic heterocycle may be bonded to each other to form other ring.

As the “aromatic heterocycle”, 5- or 6-membered aromatic heterocycle containing 1 to 4 nitrogen atoms as ring-constituting atom(s) besides carbon atom can be mentioned.

Preferable examples of the “5- or 6-membered aromatic heterocycle” include pyridyl (e.g., 2-pyridyl, 3-pyridyl, 4-pyridyl), pyrimidinyl (e.g., 2-pyrimidinyl, 4-pyrimidinyl, 5-pyrimidinyl, 6-pyrimidinyl), pyridazinyl (e.g., 3-pyridazinyl, 4-pyridazinyl), pyrazinyl (e.g., 2-pyrazinyl), pyrrolyl (e.g., 1-pyrrolyl, 2-pyrrolyl, 3-pyrrolyl), imidazolyl (e.g., 1-imidazolyl, 2-imidazolyl, 4-imidazolyl, 5-imidazolyl), pyrazolyl (e.g., 1-pyrazolyl, 3-pyrazolyl, 4-pyrazolyl), triazolyl (e.g., 1,2,4-triazol-1-yl, 1,2,4-triazol-3-yl, 1,2,4-triazol-5-yl, 1,2,3-triazol-1-yl, 1,2,3-triazol-2-yl, 1,2,3-triazol-4-yl), tetrazolyl (e.g., tetrazol-1-yl, tetrazol-5-yl), thienyl (e.g., 2-thienyl, 3-thienyl) and the like.

The benzene ring optionally having substituent(s) or 5- or 6-membered aromatic heterocycle optionally having substituent(s) for ring B optionally has 1 to 3 substituents at substitutable position(s). Examples of such substituent include those similar to the substituents that C₁₋₆ alkyl or C₁₋₆ alkoxy for R^{1a} or R^{1b} optionally has. When the number of the substituents is two or more, the respective substituents may be the same or different.

Ring B is preferably a benzene ring optionally having substituent(s) or 5- or 6-membered aromatic heterocycle (preferably, thiophene).

More preferably, ring B is a benzene ring optionally substituted by 1 to 3 halogen atoms (preferably fluorine atom), particularly preferably, a benzene ring optionally having substituent(s).

Examples of the “acyl group” exemplified as the substituent of the “amino group optionally having substituent(s)” for R² and the “acyl group” exemplified as the substituent of the “hydrocarbon optionally having substituent(s)” for R³ include groups represented by the formulas: —COR^{A}, —CO—OR^{A}, —SO₃R^{A}, —SO₂R^{A}, —SOR^{A}, —CO—NR^{A}′R^{B}′, —CS—NR^{A}′R^{B}′ and —SO₂NR^{A}′R^{B}′, wherein R^{A} is a hydrogen atom, hydroxy, a lower aliphatic hydrocarbon group optionally having substituent(s) or heterocyclic group optionally having substituent(s). R^{A}′ and R^{B}′ are each independently a hydrogen atom, a lower aliphatic hydrocarbon group optionally having substituent(s) or a heterocyclic group optionally having substituent(s), or R^{A}′ and R^{B}′ form, together with the adjacent nitrogen atom, a nitrogen-containing heterocycle optionally having substituent(s) and the like.

Examples of the “lower aliphatic hydrocarbon group optionally having substituent(s)” for R^{A}, R^{A}′ or R^{B}′ include a C₁₋₁₀ alkyl group, a C₂₋₁₀ alkenyl group, a C₂₋₁₀ alkynyl group, a C₃₋₁₀ cycloalkyl group, a C₃₋₁₀ cycloalkenyl group, and a C₄₋₁₀ cycloalkadienyl group.

Examples of the “heterocyclic group” of the “heterocyclic group optionally having substituent(s)” for R^{A}, R^{A}′ or R^{B}′ include thienyl, furyl, pyridyl, pyrazolyl, imidazolyl, tetrazolyl, oxazolyl, thiazolyl, oxadiazolyl, thiadiazolyl, tetrahydrofuryl, morpholinyl, thiomorpholinyl, piperidinyl, pyrrolidinyl, piperazinyl and the like.

The “heterocyclic group” of the “optionally substituted heterocyclic group” for R^{A}, R^{A}′ or R^{B}′ optionally has 1 to 3 substituents at substitutable position(s). Examples of such substituent include those similar to the substituents that C₁₋₆ alkyl for R^{1a} or R^{1b} optionally has and the like. When the number of the substituents is two or more, the respective substituents may be the same or different.

Examples of the “nitrogen-containing heterocycle” of the “optionally substituted nitrogen-containing heterocycle” formed by R^{A}′ and R^{B}′ together with the adjacent nitrogen atom include a 5- to 7-membered nitrogen-containing heterocycle containing, as a ring-constituting atom besides carbon atoms, at least one nitrogen atom and optionally further containing one or two hetero atoms selected from an oxygen atom, a sulfur atom and a nitrogen atom. Preferable examples of the nitrogen-containing heterocycle include pyrrolidine, imidazolidine, pyrazolidine, piperidine, piperazine, morpholine, thiomorpholine and the like.

The nitrogen-containing heterocycle optionally has 1 to 3 substituents at substitutable position(s). Examples of such substituent include those similar to the substituents that C₁₋₆ alkyl for R^{1a} or R^{1b} optionally has and the like. When the number of the substituents is two or more, the respective substituents may be the same or different.

Preferable examples of the “acyl group” include
(1) a formyl group;
(2) a carboxy group;
(3) a C₁₋₆ alkyl-carbonyl group optionally substituted by 1 to 3 substituents selected from

(i) a halogen atom,

(ii) a C₁₋₆ alkoxy-carbonyl group,

(iii) a C₆₋₁₄ aryl group (e.g., phenyl), and

(iv) a C₁₋₆ alkoxy group;
(4) a C₁₋₆ alkoxy-carbonyl group optionally substituted by 1 to 3 substituents selected from

(i) a halogen atom,

(ii) a C₆₋₁₄ aryl group (e.g., phenyl), and

(iii) a C₁₋₆ alkoxy group;
(5) a C₃₋₁₀ cycloalkyl-carbonyl group (e.g., cyclopropylcarbonyl, cyclopentylcarbonyl, cyclohexylcarbonyl);
(6) a C₆₋₁₄ aryl-carbonyl group (e.g., benzoyl) optionally substituted by 1 to 3 halogen atoms;
(7) a carbamoyl group optionally mono- or di-substituted by substituent(s) selected from

(i) a C₁₋₆ alkyl group optionally substituted by 1 to 3 substituents selected from
(a) a halogen atom,
(b) a C₁₋₆ alkoxy-carbonyl group,
(c) a C₆₋₁₄ aryl group (e.g., phenyl),
(d) a C₁₋₆ alkoxy group, and
(e) an aromatic heterocyclic group (e.g., furyl),

(ii) a C₃₋₁₀ cycloalkyl group (e.g., cyclohexyl),

(iii) a C₆₋₁₄ aryl group (e.g., phenyl) optionally substituted by 1 to 3 substituents selected from
(a) a halogen atom,
(b) a C₁₋₆ alkyl group optionally substituted by 1 to 3 halogen atoms, and
(c) a C₁₋₆ alkoxy group, and

(iv) an aromatic heterocyclic group (e.g., pyridyl);
(8) a C₁₋₆ alkylsulfonyl group (e.g., methylsulfonyl, ethylsulfonyl, isopropylsulfonyl) optionally substituted by 1 to 3 substituents selected from

(i) a halogen atom,

(ii) a C₆₋₁₄ aryl group (e.g., phenyl), and

(iii) a hydroxy group;
(9) a C₆₋₁₄ arylsulfonyl group optionally substituted by 1 to 3 halogen atoms (e.g., benzenesulfonyl);
(10) a C₁₋₆ alkylsulfinyl group (e.g., methylsulfinyl, ethylsulfinyl, propylsulfinyl) optionally substituted by 1 to 3 substituents;
(11) a sulfamoyl group optionally mono- or di-substituted by substituent(s) selected from C₁₋₆ alkyl groups optionally substituted by 1 to 3 substituents selected from

(i) a halogen atom, and

(ii) nonaromatic heterocyclic group (e.g., pyrrolidinyl) optionally substituted by an oxo group;
(12) a thiocarbamoyl group optionally mono- or di-substituted by substituent(s) selected from C₁₋₆ alkyl groups optionally substituted by 1 to 3 halogen atoms;
(13) an aromatic heterocyclyl-carbonyl group (e.g., furylcarbonyl, thienylcarbonyl) optionally substituted by 1 to 3 substituents selected from C₁₋₆ alkyl groups optionally substituted by 1 to 3 halogen atoms;
(14) a non-aromatic heterocyclyl-carbonyl group (e.g., tetrahydrofurylcarbonyl) optionally substituted by 1 to 3 substituents selected from C₁₋₆ alkyl groups optionally substituted by 1 to 3 halogen atoms;
and the like.

Preferable examples of compound (I) include the following compounds.

### [Compound A1]

Compound (I) wherein

R^{1a} and R^{1b} are the same or different and each is a hydrogen atom or a halogen atom,

R² is C₁₋₆ alkyl optionally having substituent(s),

R³ is a C₁₋₁₀ alkyl group, C₂₋₁₀ alkenyl group, C₂₋₁₀ alkynyl group or C₃₋₁₀ cycloalkyl group optionally substituted by 1 to 4 substituents selected from
(1) a C₃₋₈ cycloalkyl group (e.g., cyclopropyl, cyclohexyl) optionally substituted by 1 to 3 substituents selected from

(a) a C₁₋₆ alkyl group optionally substituted by 1 to 3 substituents selected from a halogen atom and a hydroxy group,

(b) a hydroxy group,

(c) a C₁₋₆ alkoxy group optionally substituted by 1 to 3 halogen atoms,

(d) a halogen atom, and

(e) an oxo group;
(2) a C₆₋₁₄ aryl group (e.g., phenyl, naphthyl) optionally substituted by 1 to 3 substituents selected from

(a) a C₁₋₆ alkyl group optionally substituted by 1 to 3 halogen atoms,

(b) a hydroxy group,

(c) a C₁₋₆ alkoxy group optionally substituted by 1 to 3 is halogen atoms, and

(d) a halogen atom, and

(e) a cyano group;
(3) an aromatic heterocyclic group (e.g., thienyl, furyl, pyridyl, pyrazolyl, imidazolyl, tetrazolyl, oxazolyl, thiazolyl, oxadiazolyl, thiadiazolyl, isoxazolyl) optionally substituted by 1 to 3 substituents selected from

(a) a C₁₋₆ alkyl group optionally substituted by 1 to 3 halogen atoms,

(b) a hydroxy group,

(c) a C₁₋₆ alkoxy group optionally substituted by 1 to 3 halogen atoms,

(d) a halogen atom, and

(e) a cyano group;
(4) a nonaromatic heterocyclic group (e.g., tetrahydrofuryl, morpholinyl, thiomorpholinyl, piperidinyl, pyrrolidinyl, piperazinyl, tetrahydropyranyl, tetrahydrothiopyranyl) optionally substituted by 1 to 3 substituents selected from

(a) a C₁₋₆ alkyl group optionally substituted by 1 to 3 halogen atoms,

(b) a hydroxy group,

(c) a C₁₋₆ alkoxy group optionally substituted by 1 to 3 halogen atoms,

(d) a halogen atom, and

(e) an oxo group;
(5) an amino group optionally mono- or di-substituted by substituent(s) selected from

(a) a C₁₋₆ alkyl group optionally substituted by 1 to 3 halogen atoms,

(b) a C₁₋₆ alkyl-carbonyl group optionally substituted by 1 to 3 halogen atoms,

(c) a C₁₋₆ alkoxy-carbonyl group optionally substituted by 1 to 3 halogen atoms,

(d) a C₁₋₆ alkylsulfonyl group optionally substituted by 1 to 3 halogen atoms (e.g., methylsulfonyl),

(e) a carbamoyl group optionally mono- or di-substituted by a C₁₋₆ alkyl group optionally substituted by 1 to 3 halogen atoms, and

(f) an aromatic heterocyclic group (e.g., thienyl, furyl, pyridyl, pyrazolyl, imidazolyl, tetrazolyl, oxazolyl, thiazolyl, oxadiazolyl, thiadiazolyl);
(6) an acyl group;
(7) a hydroxy group;
(8) a C₁₋₆ alkoxy group optionally substituted by 1 to 3 substituents selected from

(a) a halogen atom,

(b) a carboxy group,

(c) a C₁₋₆ alkoxy group,

(d) a C₁₋₆ alkoxy-carbonyl group optionally substituted 1 to 3 C₆₋₁₄ aryl groups (e.g., phenyl),

(e) an amino group optionally mono- or di-substituted by substituent(s) selected from a C₁₋₆ alkyl group and a C₁₋₆ alkoxy-carbonyl group, and

(f) a carbamoyl group;
(9) a C₁₋₆ alkylthio group (e.g., methylthio, ethylthio) optionally substituted by 1 to 3 substituents selected from

(a) a halogen atom,

(b) C₁₋₆ alkoxycarbonyl, and

(c) a hydroxy group;
(10) a cyano group;
(11) a nitro group; and
(12) a halogen atom,

R⁴ is a hydrogen atom or C₁₋₆ alkyl optionally having substituent(s),

X is CX¹ wherein X¹ is as defined above,

ring A is a benzene ring optionally further substituted by 1 to 3 substituents selected from
(1) a halogen atom;
(2) a hydroxy group;
(3) a C₁₋₆ alkyl group (preferably, C₁₋₃ alkyl group) is optionally substituted by 1 to 3 halogen atoms;
(4) a C₃₋₆ cycloalkyl group;
(5) an amino group optionally mono- or di-substituted by a C₁₋₆ alkyl group (preferably C₁₋₃ alkyl group);
(6) a C₁₋₆ alkoxy group optionally substituted by 1 to 3 halogen atoms; and
(7) a C₁₋₆ alkyl (preferably C₁₋₃ alkyl)-carbonyl group, and

ring B is a benzene ring or 6-membered aromatic heterocycle (preferably, pyridine ring) optionally substituted by a halogen atom or C₁₋₆ alkyl.

### [Compound A2]

Compound (I) wherein

R^{1a} and R^{1b} are the same or different and each is a hydrogen atom or a halogen atom,

R² is C₁₋₆ alkyl optionally having substituent(s),

R³ is C₁₋₆ alkyl optionally substituted by
(1) a C₃₋₈ cycloalkyl group (e.g., cyclopropyl, cyclohexyl);
(2) a phenyl group optionally substituted by 1 to 3 substituents selected from

(a) a C₁₋₆ alkyl group optionally substituted by 1 to 3 halogen atoms,

(b) a hydroxy group,

(c) a C₁₋₆ alkoxy group optionally substituted by 1 to 3 halogen atoms, and

(d) a halogen atom;
(3) an aromatic heterocyclic group (e.g., thienyl, furyl, pyridyl, pyrazolyl, imidazolyl, tetrazolyl, oxazolyl, thiazolyl, oxadiazolyl, thiadiazolyl) optionally substituted by 1 to 3 substituents selected from

(a) a C₁₋₆ alkyl group optionally substituted by 1 to 3 halogen atoms,

(b) a hydroxy group,

(c) a C₁₋₆ alkoxy group optionally substituted by 1 to 3 halogen atoms, and

(d) a halogen atom;
(4) a nonaromatic heterocyclic group (e.g., tetrahydrofuryl, morpholinyl, thiomorpholinyl, piperidinyl, pyrrolidinyl, piperazinyl) optionally substituted by 1 to 3 substituents selected from

(a) a C₁₋₆ alkyl group optionally substituted by 1 to 3 halogen atoms,

(b) a hydroxy group,

(c) a C₁₋₆ alkoxy group optionally substituted by 1 to 3 halogen atoms,

(d) a halogen atom, and

(e) an oxo group;
(5) an amino group optionally mono- or di-substituted by substituent(s) selected from

(a) a C₁₋₆ alkyl group optionally substituted by 1 to 3 halogen atoms,

(b) a C₁₋₆ alkyl-carbonyl group optionally substituted by 1 to 3 halogen atoms,

(c) a C₁₋₆ alkoxy-carbonyl group optionally substituted by 1 to 3 halogen atoms,

(d) a C₁₋₆ alkylsulfonyl group (e.g., methylsulfonyl) optionally substituted by 1 to 3 halogen atoms,

(e) a carbamoyl group optionally mono- or di-substituted by a C₁₋₆ alkyl group optionally substituted by 1 to 3 halogen atoms, and

(f) an aromatic heterocyclic group (e.g., thienyl, furyl, pyridyl, pyrazolyl, imidazolyl, tetrazolyl, oxazolyl, thiazolyl, oxadiazolyl, thiadiazolyl);
(6) an acyl group;
(7) a hydroxy group;
(8) a C₁₋₆ alkoxy group optionally substituted by 1 to 3 substituents selected from

(a) a halogen atom,

(b) a carboxy group,

(c) a C₁₋₆ alkoxy group,

(d) a C₁₋₆ alkoxy-carbonyl group optionally substituted by 1 to 3 C₆₋₁₄ aryl groups (e.g., phenyl), and

(e) an amino group optionally mono- or di-substituted by substituent(s) selected from a C₁₋₆ alkyl group and a C₁₋₆ alkoxy-carbonyl group;
(9) a C₁₋₆ alkylthio group (e.g., methylthio, ethylthio) optionally substituted by 1 to 3 substituents selected from

(a) a halogen atom, and

(b) C₁₋₆ alkoxycarbonyl;
(10) a cyano group;
(11) a nitro group; or
(12) a halogen atom,

R⁴ is a hydrogen atom,

X is CH,

ring A is a benzene ring optionally further substituted by 1 to 3 substituents selected from
(1) a halogen atom; and
(2) a C₁₋₆ alkyl group (preferably, C₁₋₃ alkyl group), and

ring B is a benzene ring optionally substituted by a halogen atom or C₁₋₆ alkyl.

### [Compound A3]

Compound (I) wherein

R^{1a} and R^{1b} are each a hydrogen atom,

R² is methyl, ethyl or propyl,

R³ is C₁₋₆ alkyl optionally substituted by
(1) a C₃₋₈ cycloalkyl group (e.g., cyclopropyl, cyclohexyl);
(2) a phenyl group optionally substituted by 1 to 3 substituents selected from

(a) a C₁₋₆ alkyl group optionally substituted by 1 to 3 halogen atoms,

(b) a hydroxy group,

(c) a C₁₋₆ alkoxy group optionally substituted by 1 to 3 halogen atoms, and

(d) a halogen atom;
(3) an aromatic heterocyclic group (e.g., thienyl, furyl, pyridyl, pyrazolyl, imidazolyl, tetrazolyl, oxazolyl, thiazolyl, oxadiazolyl, thiadiazolyl) optionally substituted by 1 to 3 substituents selected from

(a) a C₁₋₆ alkyl group optionally substituted by 1 to 3 halogen atoms,

(b) a hydroxy group,

(c) a C₁₋₆ alkoxy group optionally substituted by 1 to 3 halogen atoms, and

(d) a halogen atom;
(4) a nonaromatic heterocyclic group (e.g., tetrahydrofuryl, morpholinyl, thiomorpholinyl, piperidinyl, pyrrolidinyl, piperazinyl) optionally substituted by 1 to 3 substituents selected from

(a) a C₁₋₆ alkyl group optionally substituted by 1 to 3 halogen atoms,

(b) a hydroxy group,

(c) a C₁₋₆ alkoxy group optionally substituted by 1 to 3 halogen atoms,

(d) a halogen atom, and

(e) an oxo group;
(5) an amino group optionally mono- or di-substituted by substituent(s) selected from

(a) a C₁₋₆ alkyl group optionally substituted by 1 to 3 halogen atoms,

(b) a C₁₋₆ alkyl-carbonyl group optionally substituted by 1 to 3 halogen atoms,

(c) a C₁₋₆ alkoxy-carbonyl group optionally substituted by 1 to 3 halogen atoms,

(d) a C₁₋₆ alkylsulfonyl group (e.g., methylsulfonyl) optionally substituted by 1 to 3 halogen atoms,

(e) a carbamoyl group optionally mono- or di-substituted by a C₁₋₆ alkyl group optionally substituted by 1 to 3 halogen atoms, and

(f) an aromatic heterocyclic group (e.g., thienyl, furyl, pyridyl, pyrazolyl, imidazolyl, tetrazolyl, oxazolyl, thiazolyl, oxadiazolyl, thiadiazolyl);
(6) an acyl group;
(7) a hydroxy group;
(8) a C₁₋₆ alkoxy group optionally substituted by 1 to 3 substituents selected from

(a) a halogen atom,

(b) a carboxy group,

(c) a C₁₋₆ alkoxy group,

(d) a C₁₋₆ alkoxy-carbonyl group optionally substituted 1 to 3 C₆₋₁₄ aryl groups (e.g., phenyl), and

(e) an amino group optionally mono- or di-substituted by substituent(s) selected from a C₁₋₆ alkyl group and a C₁₋₆ alkoxy-carbonyl group;
(9) a C₁₋₆ alkylthio group (e.g., methylthio, ethylthio) optionally substituted by 1 to 3 substituents selected from

(a) a halogen atom, and

(b) C₁₋₆ alkoxycarbonyl;
(10) a cyano group;
(11) a nitro group; or
(12) a halogen atom.

R⁴ is a hydrogen atom,

X is CH,

ring A is a benzene ring, and

ring B is a benzene ring optionally substituted by a halogen atom or C₁₋₆ alkyl.

### [Compound A4]

Compound (I) wherein

R^{1a} and R^{1b} are each a hydrogen atom,

R² is methyl, ethyl or propyl,

R³ is C₁₋₆ alkyl optionally substituted by
(1) a C₃₋₈ cycloalkyl group (e.g., cyclopropyl, cyclohexyl);
(2) an acyl group;
(3) a hydroxy group;
(4) a C₁₋₆ alkoxy group;
(5) a C₁₋₆ alkylthio group (e.g., methylthio, ethylthio); or
(6) a halogen atom,

R⁴ is a hydrogen atom,

X is CH,

ring A is a benzene ring, and

ring B is a benzene ring substituted by halogen at the 4-position.

Examples of the salt with a compound represented by the formula (I) include metal salt, ammonium salt, salt with organic base, salt with inorganic acid, salt with organic acid, basic or salt with acidic amino acid and the like.

Preferable examples of the metal salt include sodium salt, potassium salt and the like alkali metal salt; calcium salt, magnesium salt, barium salt and the like alkaline earth metal salt; aluminum salt and the like. Preferable examples of the salt with organic base include the salt with trimethylamine, triethylamine, pyridine, picoline, 2,6-lutidine, ethanolamine, diethanolamine, triethanolamine, tromethamine[tris(hydroxymethyl)methylamine], t-butylamine, cyclohexylamine, dicyclohexylamine, N,N′-dibenzylethylenediamine and the like.

Preferable examples of the salt with inorganic acid include the salt with hydrochloric acid, hydrobromic acid, nitric acid, sulfuric acid, phosphoric acid and the like. Preferable examples of the salt with organic acid include the salt with formic acid, acetic acid, trifluoroacetic acid, phthalic acid, fumaric acid, oxalic acid, tartaric acid, maleic acid, citric acid, succinic acid, malic acid, methanesulfonic acid, benzenesulfonic acid, p-toluenesulfonic acid and the like.

Preferable examples of the salt with basic amino acid include the salt with arginine, lysine, ornithine and the like.

Preferable examples of the salt with acidic amino acid include the salt with aspartic acid, glutamic acid and the like.

Of these, a pharmaceutically acceptable salt is preferable. For example, when an acidic functional group is contained in the compound, an inorganic salt such as an alkali metal salt (e.g., sodium salt, potassium salt etc.), an alkaline earth metal salt (e.g., calcium salt, magnesium salt, barium salt etc.), ammonium salt and the like can be mentioned. When a basic functional group is contained in the compound, for example, a salt with inorganic acid (e.g., hydrochloric acid, hydrobromic acid, nitric acid, sulfuric acid, phosphoric acid and the like), and a salt with organic acid (e.g., acetic acid, phthalic acid, fumaric acid, oxalic acid, tartaric acid, maleic acid, citric acid, succinic acid, methanesulfonic acid, p-toluenesulfonic acid and the like) can be mentioned.

The production methods of compound (I) are shown in the following.

Compounds (I) can be produced by a method known per se (e.g., the method described in Katritzky, A. R, COMPREHENSIVE HETEROCYCLIC CHEMISTRY, PERGAMON PRESS, 1984, vol. 3, pp. 1014-1037, vol. 5, p 273-291 and the like) or a method analogous thereto. In addition, compounds (I) can be produced, for example, by the method shown in the following. Each compound described in the following Reaction scheme may form a salt as long as it does not inhibit the reaction, and as such salt, salts similar to the salts of compound (I) can be mentioned.

When a specific production method is not described, a commercially available compound may be easily available as a starting material compound, or the starting material compound can be produced according to a method known per se or a method analogous thereto.

In addition, the compound obtained in each step can be used for the next reaction as a crude product (e.g., in the form of a reaction mixture), or isolated from a reaction mixture according to a conventional method, or further purified by a separation means such as recrystallization, distillation, chromatography and the like.

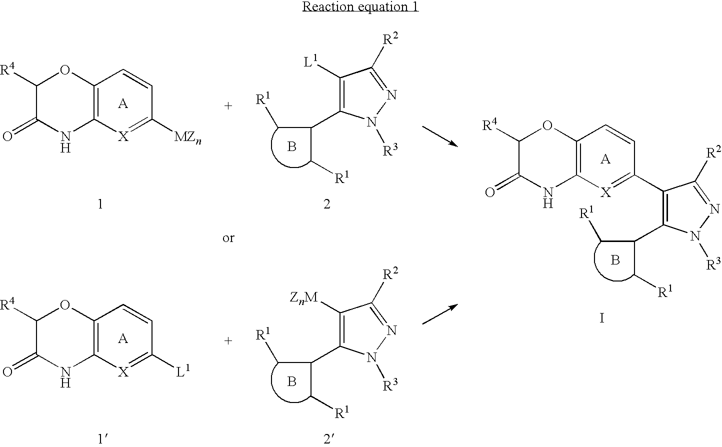

(In reaction equation 1, L₁ is a leaving group such as halogen or trifluoromethanesulfonyloxy, M includes boron, tin, silane, zinc, magnesium and the like, Z includes a halogen atom, hydroxy, alkoxy, alkyl and the like, n is an integer from 1 to 3, and each symbol has the same meaning as above.)

According to Reaction equation 1, compound (I) is obtained by reacting compound (1) and compound (2) or compound (1′) and compound (2′) in the presence of catalysts.

The amount of compound (1) used is generally about 0.1-about 10.0 moles with respect to 1 mole of compound (2).

As the catalyst, for example, palladiums such as for example, tetrakis(triphenylphosphine)palladium (0), bis(dibenzylideneacetone)palladium (0), tris(dibenzylideneacetone)dipalladium (0), bis(triphenylphosphine)palladium(II) dichloride, [1,1′-bis(diphenylphosphino)ferrocene]dichloropalladium(II)dichloromethane complex, Neolyst™ CX32, palladium (II) acetate and the like, and nickels such as for example, nickel(II) chloride, bis(triphenylphosphine)nickel(II) dichloride, [1,2-bis(diphenylphosphino)ethane]nickel(II) dichloride and the like can be mentioned.

The amount of catalyst used is generally about 0.001-about 10.0 moles with respect to 1 mole of compound (1).

This reaction is advantageously conducted using a solvent inert in the reaction. These types of solvents are not particularly limited so long as the reaction proceeds. However, as the solvent, for example, alcohols such as for example, methanol, ethanol, 1-propanol, 2-propanol, and the like, ethers such as for example, diethyl ether, tetrahydrofuran, dioxane, 1,2-dimethoxyethane and the like, hydrocarbons such as for example, benzene, toluene, cyclohexane, hexane and the like, amides such as for example, N,N-dimethylformamide, N,N-dimethylacetamide and the like, halogenated hydrocarbons such as for example, dichloromethane, chloroform, carbon tetrachloride, 1,2-dichloroethane and the like, nitriles such as for example, acetonitrile, propionitrile and the like, sulfoxides such as for example, dimethyl sulfoxide and the like, water, and the mixed solvent thereof can be mentioned.

The reaction temperature is generally about 0° C. to 200° C., and the reaction time is generally about 5 minutes to about 72 hours.

In addition, this reaction is advantageously conducted in the presence of ligand, additive and base as required.

As the ligand, for example, triphenylphosphine, tri-t-butylphosphine 1,1′-bis(diphenylphosphino)ferrocene, 2-(dicyclohexylphosphino)biphenyl, 2-(dicyclohexylphosphino)-2′,4′,6′-tri-i-propyl-1,1′-biphenyl and the like can be mentioned.

The amount of ligand used is generally about 0.001-about 20.0 moles with respect to 1 mole of compound (1).

As the additive, for example, lithium chloride, tetrabutylammonium chloride, potassium fluoride, cesium fluoride, and tetrabutylammonium fluoride and the like can be mentioned.

The amount of the additive used is generally about 0.001-about 20.0 moles with respect to 1 mole of compound (1).

As the base, for example, basic salts such as for example, sodium carbonate, potassium carbonate, cesium carbonate, sodium bicarbonate and the like, aromatic amines such as for example, pyridine, lutidine and the like, tertiary amines such as for example, triethylamine, tripropylamine, tributyl amine, cyclohexyl dimethylamine, 4-dimethylaminopyridine, N,N-dimethylaniline, N-methylpiperidine, N-methylpyrrolidine, N-methylmorpholine and the like, alkali metal hydroxides such as for example, sodium hydroxide, potassium hydroxide and the like, and metalalkoxides such as for example, sodium methoxide, sodium ethoxide, sodium t-butoxide, potassium t-butoxide and the like can be mentioned.

The amount of the base used is generally about 0.1-about 10.0 moles with respect to 1 mole of compound (1).

Where necessary, the ultrasonic and microwave irradiation reaction conditions are employed to carry out the reaction smoothly.

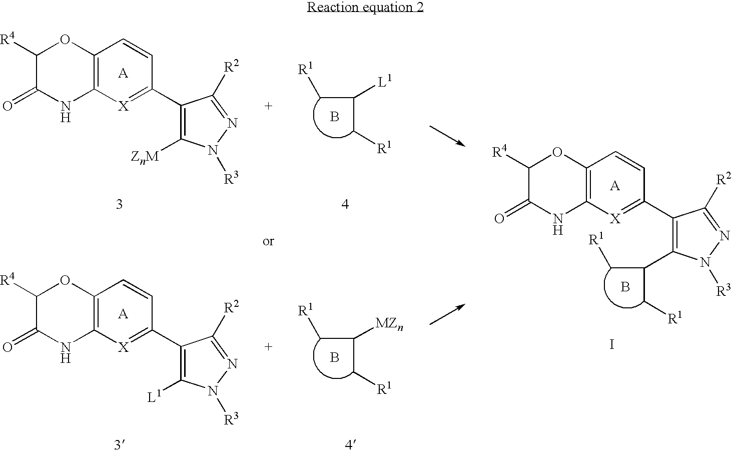

As described in Reaction equation 2, compound (I) is also obtained by reacting compound (3) and compound (4) or compound (3′) and compound (4′) according to the similar method described for Reaction equation 1.

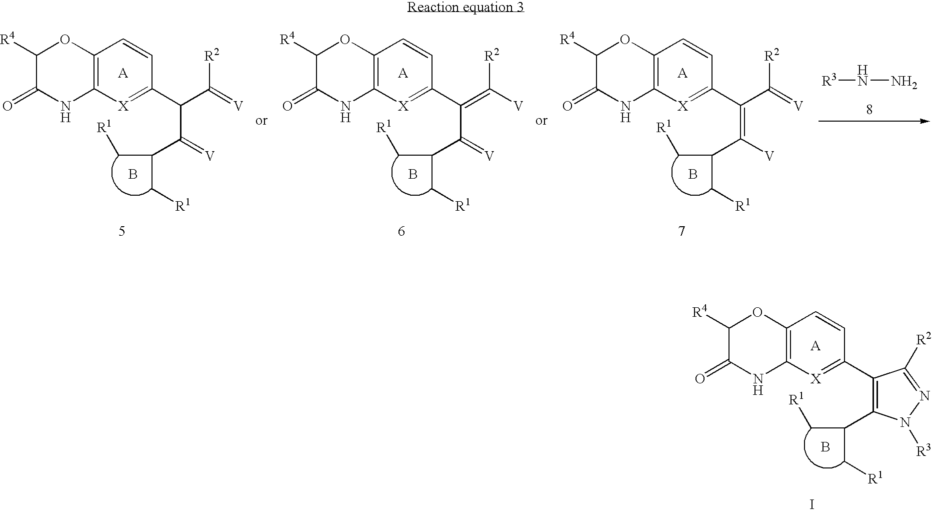

(In reaction equation 3, V is an oxygen atom, hydroxy, alkoxy, imino, optionally substituted imino, amino, optionally substituted amino and the like and each symbol has the same meaning as above.)

According to Reaction equation 3, compound (I) is produced by reacting compound (5) or compound (6) or compound (7) with compound (8).

The amount of compound (8) used is generally about 0.5-about 2.0 moles with respect to 1 mole of compound (5) or compound (6) or compound (7).

This reaction is advantageously conducted in the absence of solvent or using a solvent inert in the reaction. These types of solvents are not particularly limited so long as the reaction proceeds. However, as the solvent, for example, alcohols such as for example, methanol, ethanol, 1-propanol, 2-propanol and the like, ethers such as for example, diethyl ether, tetrahydrofuran, dioxane, 1,2-dimethoxyethane and the like, hydrocarbons such as for example, benzene, toluene, cyclohexane, hexane and the like, amides such as for example, N,N-dimethylformamide, N,N-dimethylacetamide and the like, halogenated hydrocarbons such as for example, dichloromethane, chloroform, carbon tetrachloride, 1,2-dichloroethane and the like, nitriles such as for example, acetonitrile, propionitrile and the like, sulfoxides such as for example, dimethyl sulfoxide and the like, water, and the mixed solvent thereof can be mentioned.

The reaction temperature is generally about −80° C. to about 200° C., and the reaction time is generally about 5 minutes to about 120 hours.

In addition, this reaction is advantageously conducted in the presence of acid catalyst and/or base catalyst, as required.

As the acid catalyst, for example, mineral acids such as for example, hydrochloric acid, hydrobromic acid, sulfuric acid and the like, organic acids such as for example, formic acid, acetic acid, trifluoroacetic acid, p-toluenesulfonic acid, camphor sulfonic acid and the like, and Lewis acids such as for example, aluminum chloride, boron trifluoride and the like can be mentioned.

As the base catalyst, for example, basic salts such as for example, sodium carbonate, potassium carbonate, cesium carbonate, sodium bicarbonate and the like, aromatic amines such as for example, pyridine, lutidine and the like, tertiary amines such as for example, triethylamine, tripropylamine, tributyl amine, cyclohexyl dimethylamine, 4-dimethylaminopyridine, N,N-dimethylaniline, N-methylpiperidine, N-methylpyrrolidine, N-methylmorpholine and the like, alkali metal hydroxides such as for example, sodium hydroxide, potassium hydroxide and the like and metalalkoxides such as for example, sodium methoxide, sodium ethoxide, sodium t-butoxide, potassium t-butoxide and the like can be mentioned.

The amount of the acid catalyst and/or the base catalyst used are generally about 0.1-about 2.0 moles with respect to 1 mole of compound (5) or compound (6) or compound (7).

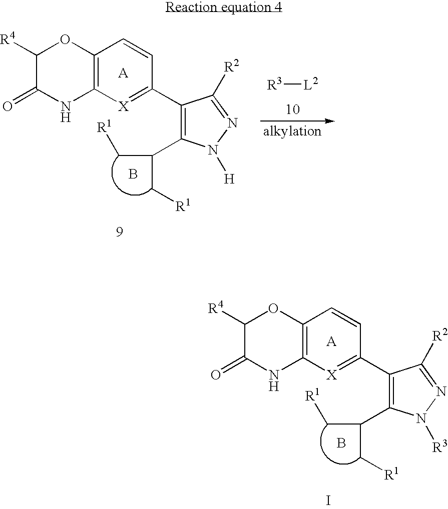

(In reaction equation 4, the leaving group represented by L² includes hydroxy, halogen atom (e.g., fluorine, chlorine, bromine, iodine and the like), C₁₋₆ alkylsulfonyloxy (e.g., methanesulfonyloxy, trifluoromethanesulfonyloxy, ethanesulfonyloxy and the like), optionally substituted C₆₋₁₀ arylsulfonyloxy and the like and each symbol has the same meaning as above.)

Compound (10) is readily available as a commercial product, and moreover it is produced by per se well-known processes, for example, the process disclosed in the 4th Edition of Jikken Kagaku Kouza 19 (edited by The Chemical Society of Japan), pp 363-482, published by Maruzen K K and processes corresponding to this.

According to Reaction equation 4, compound (I) is also produced by reacting compound (9) and compound (10) in the presence of base as required.

As the base, for example, basic salts such as for example, sodium carbonate, potassium carbonate, cesium carbonate, sodium bicarbonate and the like, aromatic amines such as for example, pyridine, lutidine and the like, tertiary amines such as for example, triethylamine, tripropylamine, tributyl amine, cyclohexyl dimethylamine, 4-dimethylaminopyridine, N,N-dimethylaniline, N-methylpiperidine, N-methylpyrrolidine, N-methylmorpholine and the like, alkali metal hydrides such as for example, sodium hydride, potassium hydride and the like, metallic amides such as for example, sodium amide, lithium diisopropylamide, lithium hexamethyl disilazide and the like, and metalalkoxides such as for example, sodium methoxide, sodium ethoxide, potassium t-butoxide and the like can be mentioned.

The amount of compound (10) used is generally about 0.8-about 5.0 moles with respect to 1 mole of compound (9).

The amount of the base used is generally about 0.8-about 5.0 moles with respect to 1 mole of compound (9). In addition, compound (I) can be produced in the presence of quaternary ammonium salt as required.

As the quaternary ammonium salt, for example, tetrabutyl ammonium iodide and the like can be mentioned.

The amount of the quaternary ammonium salt used is generally about 0.1-about 2.0 moles with respect to 1 mole of compound (9).

This reaction is advantageously conducted in the absence of solvent or using a solvent inert in the reaction, but this type of solvent is not particularly limited so long as the reaction proceeds. However, as the solvent, for example, ethers such as for example, diethyl ether, tetrahydrofuran, dioxane, 1,2-dimethoxyethane and the like, hydrocarbons such as for example, benzene, toluene, cyclohexane, hexane and the like, amides such as for example, N,N-dimethylformamide, N,N-dimethylacetamide and the like, halogenated hydrocarbons such as for example, dichloromethane, chloroform, carbon tetrachloride, 1,2-dichloroethane and the like, nitriles such as for example, acetonitrile, propionitrile and the like, sulfoxides such as for example, dimethyl sulfoxide and the like, water and the mixed solvent thereof can be mentioned.

The reaction time is usually about 30 minutes to about 96 hours and the reaction temperature is usually about —80° C.-about 120° C.

Instead of the aforementioned reaction, the Mitsunobu reaction (Synthesis, 1981, pp. 1-27) can be used.

In this reaction, compound (9) reacts with compound (10) in which L² is OH in the presence of azodicarboxylates (e.g., diethylazodicarboxylate, diisopropyl azodicarboxylate and the like) and phosphines (e.g., triphenylphosphine, tributylphosphine and the like).

The amounts of compound (10) used is generally about 0.8-about 5.0 moles with respect to 1 mole of compound (9).

The amounts of the azodicarboxylate and the phosphine used are respectively about 0.8-about 5.0 moles with respect to 1 mole of compound (9).

This reaction is advantageously conducted using a solvent inert in the reaction, but this type of solvent is not particularly limited so long as the reaction proceeds. However, ethers such as for example, diethyl ether, tetrahydrofuran, dioxane, 1,2-dimethoxyethane and the like, hydrocarbons such as for example, benzene, toluene, cyclohexane, hexane and the like, amides such as for example, N,N-dimethylformamide, N,N-dimethylacetamide and the like, halogenated hydrocarbons such as for example, dichloromethane, chloroform, carbon tetrachloride, 1,2-dichloroethane and the like, nitriles such as for example, acetonitrile, propionitrile and the like, sulfoxides such as for example, dimethyl sulfoxide and the like and the mixed solvents thereof can be mentioned.

The reaction time is usually about 5 minutes to about 48 hours, the reaction temperature is usually about −20° C.-about 200° C.

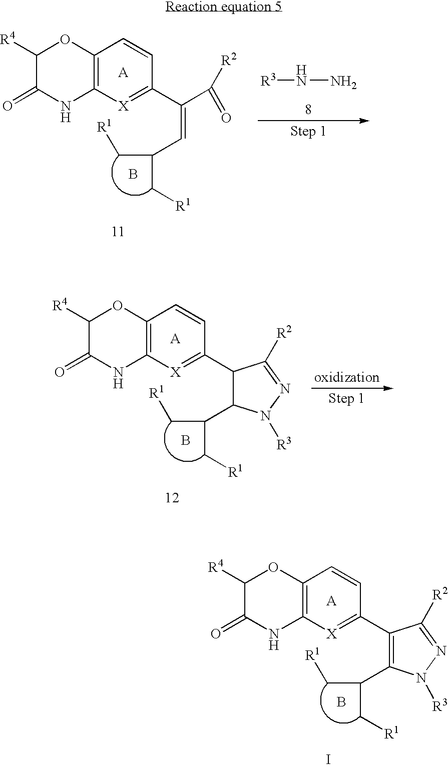

According to Reaction equation 4, compound (I) is also produced from compound (11) via compound (12).

### (Step 1)

The condensation can be carried out without solvent or in an inert solvent. As the solvent, for example, tetrahydrofuran, diethyl ether, dimethoxyethane, hexane, toluene, benzene, dichloromethane, chloroform, 1,2-dichloroethane, ethyl acetate, methanol, ethanol, dimethylformamide, dimethyl sulfoxide, pyridine and the like, water and the mixed solvent thereof can be mentioned.

The reaction temperature is generally about −80° C. to 200° C., and the reaction time is generally about 5 minutes to about 96 hours.

The amount of compound (8) used is generally about 0.8-5 mol with respect to 1 mol of compound (11).

Where necessary, a base such as pyridine, 4-dimethylaminopyridine, triethylamine, sodium hydride, potassium carbonate, sodium hydroxide and the like can be used to carry out the reaction smoothly.

### (Step 2)

Compound (I) can be produced by oxidizing compound (12).

The oxidation reaction can be carried out without solvent or in an inert solvent.

As the solvent, for example, tetrahydrofuran, diethyl ether, dimethoxyethane, acetone, water, methanol, ethanol, 1-propanol, 2-propanol, hexane, toluene, benzene, pyridine, dichloromethane, dimethylformamide, dimethyl sulfoxide and the like, and the mixed solvent thereof can be mentioned.

The reaction temperature is generally about −80° C. to 200° C., and the reaction time is generally about 5 minutes to about 96 hours.

Where necessary, an oxidant such as 2,3-dichloro-5,6-dicyano-1,4-benzoquinone, chloranil, manganese dioxide, oxygen, sulfur and the like, or dehydrogenation catalysts such as for example, palladium-carbon, platinum-carbon and the like can be used to carry out the reaction smoothly.

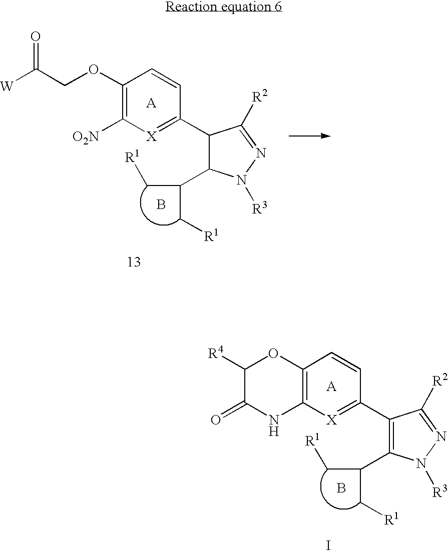

(In the reaction equation 6, W is an alkoxy, hydroxy, amino, optionally substituted amino, halogen atom (e.g., fluorine, chlorine, bromine, iodine and the like), C₁₋₆ alkylsulfonyloxy (e.g., methanesulfonyloxy, trifluoromethanesulfonyloxy, ethanesulfonyloxy and the like), optionally substituted C₆₋₁₀ arylsulfonyloxy and the like and each symbol has the same meaning as above.)

Compound (I) is also obtained by reducing compound (13).

The reducing agent which is used in the reduction is, for example, metal hydrides such as aluminum hydride, diisobutylaluminum hydride and the like, complex metal hydrides such as sodium borohydride, lithium aluminum hydride and the like, borane complexes such as borane tetrahydrofuran complex, borane dimethylsulfide and the like, alkylboranes such as thexylborane, diamylborane and the like, diborane, or metals such as zinc, aluminum, tin, iron and the like and alkali metals (sodium, lithium and the like)/liquid ammonia (batch reduction) and the like can be mentioned. Further, the hydrogenating catalyst is, for example, palladium carbon, platinum oxide, Raney nickel, Raney cobalt and the like can be mentioned. The hydrogen source is, for example, formic acid, ammonium formate, hydrazine and the like in addition to gas-phase hydrogen can be mentioned.

As the reducing agent is used in an amount of about 0.5 to about 20 moles relative to 1 mole of compound (13) when metal hydrides or complex metal hydride is used, about 0.5 to about 20 moles relative to 1 mole of compound (13) when borane complexes, alkylboranes or diborane is used, and about 0.5 to about 20 moles relative to 1 mole of compound (13) when metals or alkali metals are used. In case of hydrogenation, the catalyst such as for example, palladium-carbon, platinum oxide, Raney nickel and the like is used in an amount of about 1 to 1000% by weight relative to the amount of compound (13). When the hydrogen source other than gas-phase hydrogen is used, it is used in an amount of about 1.0 to about 20 moles relative to 1 mole of compound (13).

The present reaction is advantageously carried out using a solvent inert to the reaction. Such solvents are not particularly limited if the reaction proceeds. However, for example, alcohols such as for example, methanol, ethanol, 1-propanol, 2-propanol and the like, ethers such as for example, diethyl ether, diisopropyl ether, tetrahydrofuran, dioxane, 1,2-dimethoxyethane and the like, hydrocarbons such as for example, benzene, toluene, cyclohexane, hexane and the like, amides such as for example, N,N-dimethylformamide, N,N-dimethylacetamide and the like, organic acids such as for example, formic acid, acetic acid and the like, water, and the mixed solvent thereof can be mentioned.

The reaction time is varied depending on kinds or amount of reducing agent, or activity or amount of catalyst, but usually about 1 hour to about 100 hours, preferably about 1 hour to about 50 hours. The reaction temperature is usually about −80° C. to about 200° C. When a hydrogenation catalyst is used, hydrogen pressure is usually 1 to 100 atm.

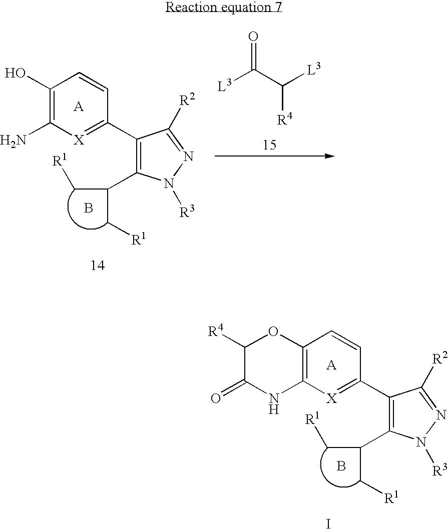

(In the reaction equation 7, the leaving group represented by L³ includes a halogen atom (e.g., fluorine, chlorine, bromine, iodine and the like), C₁₋₆ alkylsulfonyloxy (e.g., methanesulfonyloxy, trifluoromethanesulfonyloxy, ethanesulfonyloxy and the like), optionally substituted C₆₋₁₀ arylsulfonyloxy and the like and each symbol has the same meaning as above.)

According to Reaction equation 7, compound (I) is also produced by reacting compound (14) and compound (15) in the presence of base as required.

As the base, for example, basic salts such as for example, sodium carbonate, potassium carbonate, cesium carbonate, sodium bicarbonate and the like, aromatic amines such as for example, pyridine, lutidine and the like, tertiary amines such as for example, triethylamine, tripropylamine, tributyl amine, cyclohexyl dimethylamine, 4-dimethylaminopyridine, N,N-dimethylaniline, N-methylpiperidine, N-methylpyrrolidine, N-methylmorpholine and the like, alkali metal hydrides such as for example, sodium hydride, potassium hydride and the like, metallic amides such as for example, sodium amide, lithium diisopropylamide, lithium hexamethyl disilazide and the like, and metalalkoxides such as for example, sodium methoxide, sodium ethoxide, potassium t-butoxide and the like can be mentioned.

The amount of compound (15) used is generally about 0.8-about 5.0 moles with respect to 1 mole of compound (14).

The amount of the base used is generally about 0.8-about 5.0 moles with respect to 1 mole of compound (14).

This reaction is advantageously conducted in the absence of solvent or using a solvent inert in the reaction, but this type of solvent is not particularly limited so long as the reaction proceeds. However, as the solvent, ethers such as for example, diethyl ether, tetrahydrofuran, dioxane, 1,2-dimethoxyethane and the like, hydrocarbons such as for example, benzene, toluene, cyclohexane, hexane and the like, amides such as for example, N,N-dimethylformamide, N,N-dimethylacetamide and the like, halogenated hydrocarbons such as for example, dichloromethane, chloroform, carbon tetrachloride, 1,2-dichloroethane and the like, nitriles such as for example, acetonitrile, propionitrile and the like, sulfoxides such as for example, dimethyl sulfoxide and the like, water, and the mixed solvent thereof can be mentioned.

The reaction time is usually about 30 minutes to about 96 hours and the reaction temperature is usually about −80° C.-about 200° C.

In the thus-obtained compound (I), the functional group in a molecule can also be converted to an object functional group by a combination of chemical reactions known per se. Examples of such chemical reactions include oxidation reaction, reduction reaction, alkylation reaction, hydrolysis, amination reaction, amidation reaction, esterification reaction, aryl coupling reaction, deprotection and the like.

In each of the aforementioned reactions, when a starting material compound has an amino group, a carboxy group, a hydroxy group and/or a carbonyl group as a substituent, a protecting group generally used in the peptide chemistry may be introduced into these groups, and the object compound can be obtained by removing the protecting group as necessary after the reaction.

Examples of the amino-protecting group include a formyl group, a C₁₋₆ alkyl-carbonyl group, a C₁₋₆ alkoxy-carbonyl group, a benzoyl group, a C₇₋₁₀ aralkyl-carbonyl group (e.g., benzylcarbonyl), a C₇₋₁₄ aralkyloxy-carbonyl group (e.g., benzyloxycarbonyl, 9-fluorenylmethoxycarbonyl), a trityl group, a phthaloyl group, a N,N-dimethylaminomethylene group, a substituted silyl group (e.g., trimethylsilyl, triethylsilyl, dimethylphenylsilyl, tert-butyldimethylsilyl, tert-butyldiethylsilyl), a C₂₋₆ alkenyl group (e.g., 1-allyl) and the like. These groups are optionally substituted by 1 to 3 substituents selected from a halogen atom, a C₁₋₆ alkoxy group and a nitro group.

Examples of the carboxy-protecting group include a C₁₋₆ alkyl group, a C₇₋₁₁ aralkyl group (e.g., benzyl), a phenyl group, a trityl group, a substituted silyl group (e.g., trimethylsilyl, triethylsilyl, dimethylphenylsilyl, tert-butyldimethylsilyl, tert-butyldiethylsilyl), a C₂₋₆ alkenyl group (e.g., 1-allyl) and the like. These groups are optionally substituted by 1 to 3 substituents selected from a halogen atom, a C₁₋₆ alkoxy group and a nitro group.

Examples of the hydroxy-protecting group include a C₁₋₆ alkyl group, a phenyl group, a trityl group, a C₇₋₁₀ aralkyl group (e.g., benzyl), a formyl group, a C₁₋₆ alkyl-carbonyl group, a benzoyl group, a C₇₋₁₀ aralkyl-carbonyl group (e.g., benzylcarbonyl), a 2-tetrahydropyranyl group, a 2-tetrahydrofuranyl group, a substituted silyl group (e.g., trimethylsilyl, triethylsilyl, dimethylphenylsilyl, tert-butyldimethylsilyl, tert-butyldiethylsilyl), a C₂₋₆ alkenyl group (e.g., 1-allyl) and the like. These groups are optionally substituted by 1 to 3 substituents selected from a halogen atom, a C₁₋₆ alkyl group, a C₁₋₆ alkoxy group and a nitro group.

Examples of the carbonyl-protecting group include a cyclic acetal (e.g., 1,3-dioxane), a non-cyclic acetal (e.g., a di-C₁₋₆ alkylacetal) and the like.

The above-mentioned protecting groups can be removed by a method known per se, for example, based on the method described in Protective Groups in Organic Synthesis, John Wiley and Sons (1980) and the like. Specifically, methods using acid, base, ultraviolet rays, hydrazine, phenylhydrazine, sodium N-methyldithiocarbamate, tetrabutylammonium fluoride, palladium acetate, trialkylsilyl halide (e.g., trimethylsilyliodide, trimethylsilylbromide) and the like, reduction method and the like.

Compound (I) obtained by the above-mentioned production method can be isolated and purified by a known means such as concentration, concentration under reduced pressure, solvent extraction, crystallization, recrystallization, phase transfer, chromatography and the like. In addition, each starting material compound used in each of the above-mentioned production methods can be isolated and purified by a known means such as those mentioned above. Alternatively, these starting material compounds may be directly used in the form of a reaction mixture without isolation as the starting materials in the next step.

Compound (I) may be used as a prodrug. A prodrug of compound (I) means a compound which is converted to compound (I) with a reaction due to an enzyme, an gastric acid, etc. under the physiological condition in the living body, that is, a compound which is converted to compound (I) with oxidation, reduction, hydrolysis, etc. according to an enzyme; a compound which is converted to compound (I) by hydrolysis etc. due to gastric acid, etc.

A prodrug of compound (I) may be a compound obtained by subjecting an amino group in compound (I) to an acylation, alkylation or phosphorylation (e.g., a compound obtained by subjecting an amino group in compound (I) to an eicosanoylation, alanylation, pentylaminocarbonylation, (5-methyl-2-oxo-1,3-dioxolen-4-yl)methoxycarbonylation, tetrahydrofuranylation, pyrrolidylmethylation, pivaloyloxymethylation or tert-butylation, etc.); a compound obtained by subjecting a hydroxy group in compound (I) to an acylation, alkylation, phosphorylation or boration (e.g., a compound obtained by subjecting an hydroxy group in compound (I) to an acetylation, palmitoylation, propanoylation, pivaloylation, succinylation, fumarylation, alanylation or dimethylaminomethylcarbonylation); a compound obtained by subjecting a carboxyl group in compound (I) to an esterification or amidation (e.g., a compound obtained by subjecting a carboxyl group in compound (I) to an ethyl esterification, phenyl esterification, carboxymethyl esterification, dimethylaminomethyl esterification, pivaloyloxymethyl esterification, ethoxycarbonyloxyethyl esterification, phthalidyl esterification, (5-methyl-2-oxo-1,3-dioxolen-4-yl)methyl esterification, cyclohexyloxycarbonylethyl esterification or methylamidation) and the like. Any of these compounds can be produced from compound (I) by a method known per se.

A prodrug for compound (I) may also be one which is converted into compound (I) under a physiological condition, such as those described in IYAKUHIN no KAIHATSU (Development of Pharmaceuticals), Vol. 7, Design of Molecules, p. 163-198, Published by HIROKAWA SHOTEN (1990).

Preferable prodrug is a compound obtained by esterification, etherification, carbonation or carbamation of the hydroxyl group of compound (I).

When compound (I) has an isomer such as optical isomer, stereoisomer, positional isomer, rotational isomer and the like, any isomers and a mixture thereof are encompassed in compound (I). For example, when compound (I) has an optical isomer, an optical isomer resolved from a racemate is also encompassed in compound (I). Such isomer can be obtained as a single product by a synthesis method or a separation method (concentration, solvent extraction, column chromatography, recrystallization etc.) known per se.

Compound (I) may be a crystal, and both a single crystal and crystal mixtures are encompassed in compound (I). Crystals can be produced by crystallization according to crystallization methods known per se.

Compound (I) may be a solvate (e.g., hydrate etc.) or a non-solvate, both of which are encompassed in compound (I).

A compound labeled with an isotope (e.g., ³H, ¹⁴C, ³⁵S, ¹²⁵I and the like) and the like is also encompassed in compound (I).

The mineralocorticoid receptor antagonist of the present invention shows low toxicity (e.g., more superior than mineralocorticoid receptor antagonist as a pharmaceutical agent from the aspects of acute toxicity, chronic toxicity, genetic toxicity, reproductive toxicity, cardiotoxicity, drug interaction, carcinogenicity and the like), and is useful for the prophylaxis or treatment of disease or condition mediated by the activation of a mineralcorticoid receptor, for example, a disease or condition developed or whose onset is promoted by the presence of aldosterone, or a factor induced by the presence of aldosterone, and the like in an animal, particularly mammal (e.g., human, monkey, cat, swine, horse, bovine, mouse, rat, guinea pig, dog, rabbit etc.).

As such disease or condition, systemic disease, for example, essential hypertension, aldosteronism (including primary aldosteronism, secondary aldosteronism and pseudoaldosteronism) fluid accumulation type hypertension, low renin essential hypertension, malignant hypertension, renovascular hypertension, high renin hypertension, abnormal circadian variation of blood pressure, sleep apnea syndrome, cardiac failure, acute cardiac failure, chronic cardiac failure, cardiomyopathy, congestive heart failure, cardiac hypertrophy, angina pectoris, myocarditis, arrhythmia, fast pulse, cardiac infarction, asymptomatic cerebrovascular accident, transient cerebral ischemic attack, RIND, cerebral apoplexy, cerebrovascular dementia, hypertensive encephalopathy, cerebral infarction, brain edema, cerebral circulatory disturbance, recurrence and sequelae of cerebrovascular disorder (e.g., neural symptoms, mental symptoms, subjective symptoms, disorders of daily living activities etc.), ischemic peripheral circulation disorder, intermittent claudication, cardiac muscle ischemia, venous insufficiency, progress of cardiac failure after cardiac infraction, diabetic nephropathy, end stage renal failure, renal diseases (e.g., nephritis, glomerulonephritis, IgA nephropathy, progressive nephropathy, glomerulosclerosis, renal failure, thrombotic microangiopathy, complications of dialysis, organ damage including renal damage caused by irradiation etc.), arteriosclerosis including atherosclerosis (e.g., aneurysm, coronary arteriosclerosis, cerebral arteriosclerosis, peripheral arteriosclerosis etc.), vascular hypertrophy, vascular hypertrophy or occlusion and organ damage after intervention (e.g., percutaneous transluminal coronary angioplasty, stenting, coronary angioscopy, intravascular ultrasound, coronary infusion thrombolysis therapy etc.), blood vessel reocclusion or restenosis after bypass surgery, polycythemia, hypertension, organ or damage vascular hypertrophy after transplantation, rejection after transplantation, ophthalmic diseases (e.g., glaucoma, ocular hypertension disease etc.), thrombosis, multiple organ failure, endothelial dysfunction, hypertensive tinnitus, other circulatory diseases (e.g., deep-vein thrombosis, obstructive peripheral circulation disorder, obstructive arteriosclerosis, thromboangiitis obliterans, ischemic cerebral circulatory disturbance, Raynaud's disease, Buerger's disease etc.), metabolic syndrome, diabetes, diabetic complications (e.g., diabetic retinopathy, diabetic nephropathy, diabetic neuropathy etc.), metabolic or nutrient disturbance (e.g., obesity, diabetes, hyperlipidemia (including hypercholesterolemia, hyper-LDL-cholesterolemia, hypo-HDL-cholesterolemia, and hyperglyceridemia), hyperuricemia, hypokalemia, hypernatremia etc.), neurodegenerative disease (e.g., Alzheimer's disease, Parkinson's syndrome, amyotropic lateral sclerosis retinitis, AIDS encephalopathy etc.), central nerve disorders (e.g., disorder such as cerebral hemorrhage and cerebral infarction and the like and sequelae or complications thereof, head trauma, spinal injury, brain edema, disorders of sensory function, abnormality of sensory function, autonomic nervous system dysfunction, abnormality of autonomic nervous system function, multiple sclerosis etc.), dementia, memory disorders, disturbance of consciousness, amnesia, anxiety, tension, anxious mental state, mental diseases (e.g., depression, epilepsy, alcoholism etc.), inflammatory disease (e.g., arthritis such as chronic articular rheumatism, osteoarthritis, rheumatoid myelitis, periostitis and the like; inflammation after surgery or trauma; regression of puffiness; pharyngitis; cystitis; pneumonia; atopic dermatitis; inflammatory bowel disease such as Crohn's disease, ulcerative colitis and the like; meningitis; inflammatory ophthalmic diseases; inflammatory pulmonary disease such as pneumonia, silicosis, pulmonary sarcoidosis, pulmonary tuberculosis and the like), allergic disease (e.g., allergic rhinitis, conjunctivitis, gastrointestinal tract allergy, pollinosis, anaphylaxis etc.), chronic obliterative pulmonary diseases, interstitial pneumonia, carinii pneumonia, collagen disease (e.g., systemic lupus erythematosus, scleroderma, polyarteritis etc.), liver disease (e.g., hepatitis including chronic-, cirrhosis etc.), portal hypertension, gastrointestinal diseases (e.g., gastritis, gastric ulcer, gastric cancer, postgastrostomy disorder, dyspepsia, esophageal ulcer, pancreatitis, colonic polyp, cholelithiasis, hemorrhoids, variceal rupture of esophagus and stomach etc.), diseases of blood or hematopoietic organ (e.g., polycythemia, vascular purpura, autoimmune hemolytic anemia, disseminated intravascular coagulation syndrome, multiple myelopathy etc.), bone disease (e.g., bone fracture, bone refracture, osteoporosis, osteohalisteresis, Paget's disease of bone, rigid myelitis, chronic articular rheumatism, osteoarthrosis of knee and destruction of articular tissue of similar disease thereof etc.), solid tumor, tumor (e.g., malignant melanoma, malignant lymphoma, cancer of digestive organ (e.g., stomach, intestine etc.) etc.), cancer and cachexia therewith, metastasis of cancer, edema and ascites fluid associated with malignant tumor, endocrine diseases (e.g., Addison's disease, Cushing's syndrome, pheochromocytoma, primary aldosteronism etc.), Creutzfeldt-Jakob disease, diseases of urinary organ or male sex organ (e.g., cystitis, prostatomegaly, prostate cancer, sexually-transmitted diseases etc.), gynecologic diseases (e.g., climacteric disorder, gestational toxicosis, endometriosis, hysteromyoma, ovarian disease, mammary disease, sexually-transmitted diseases etc.), disease caused by environmental or occupational factor (e.g., radiation disorder, disorders caused by ultraviolet ray, infrared ray or laser beam, altitude sickness etc.), respiratory diseases (e.g., cold syndrome, pneumonia, asthma, pulmonary hypertension, pulmonary thrombosis or pulmonary embolus etc.), infections (e.g., virus infections such as cytomegalovirus, influenzavirus, herpesvirus and the like, rickettsial infections, bacterium infections etc.), toxemia (e.g., sepsis, septic shock, endotoxic shock, Gram-negative sepsis, toxic shock syndrome etc.), Otorhinolaryngological diseases (e.g., Meniere's syndrome, tinnitus, gustation disorder, dizziness, disequilibrium, dysphagia etc.), dermatic diseases (e.g., keloid, hemangioma, psoriasis etc.), dialysis hypotension, myasthenia gravis, chronic fatigue syndrome, renal edema, hepatic edema, idiopathic edema, trophedema and the like, can be mentioned.

The mineralocorticoid receptor antagonist of the present invention also shows a superior prophylactic and/or therapeutic effect on diseases for which a calcium antagonist fails to show sufficient efficacy.

As the mineralocorticoid receptor antagonist of the present invention, compound (I) or a prodrug thereof (hereinafter to be also referred to as the compound of the present invention) alone, or a pharmaceutical composition obtained by mixing with a pharmacologically acceptable carrier according to a conventional method (e.g., the method described in the Japan Pharmacopoeia etc.), such as oral preparations including tablets (including sugar-coated tablet, film-coated tablet, sublingual tablet, orally disintegrating tablet), powder, granule, capsule (including soft capsule, microcapsule), liquid, emulsion, suspension, film (e.g., orally disintegrable film) and the like; and parenteral agents including injection (e.g., subcutaneous injection, intravenous injection, intramuscular injection, intraperitoneal injection, drip infusion), external preparation (e.g., dermal preparation, ointment), suppository (e.g., rectal suppository, vaginal suppository), pellet, nasal preparations, pulmonary preparation (inhalant), eye drop and the like can be mentioned. Each of these can be safely administered orally or parenterally (e.g., topical, rectal, intravenous administrations).

The content of the compound of the present invention in the pharmaceutical composition is about 0.01 to 11 wt %, preferably about 2 to 85 wt %, of the whole composition.

While the dose of the compound of the present invention varies depending on the subject of administration, administration route, disease and the like, for example, for administration of an oral preparation to an adult (body weight about 60 kg) as a therapeutic agent for cardiac failure, it is about 1 to 1000 mg, preferably about 3 to 300 mg, more preferably about 10 to 200 mg, in the amount of the compound of the present invention as an active ingredient, which can be administered once a day or in several portions a day.

The mineralocorticoid receptor antagonist of the present invention can be used in combination with a pharmaceutical agent such as an antihypertensive agent, a therapeutic agent for diabetes, a therapeutic agent for diabetic complications, an antihyperlipidemic agent, an antiobesity agent, a diuretic agent, a chemotherapeutic agent, an immunotherapeutic agent and the like.

Examples of the antihypertensive agent include angiotensin converting enzyme inhibitors (e.g., captopril, enalapril, delapril etc.), angiotensin II antagonists (e.g., losartan, candesartan cilexetil, eprosartan, valsartan, telmisartan, irbesartan, tasosartan, olmesartan etc.), calcium antagonists (e.g., manidipine, nifedipine, amlodipine, efonidipine, nicardipine, azelnidipine, cilnidipine, phelodipine etc.), β-blockers (e.g., carvedilol, propranolol, metoprolol, atenolol, carteolol etc.), α-blockers (doxazosin) and the like.

Examples of the therapeutic agents for diabetes include insulin preparations (e.g., animal insulin preparations extracted from the pancreas of bovine, swine; human insulin preparations synthesized by genetic engineering techniques using Escherichia coli or yeast, etc.), α-glucosidase inhibitor (e.g., voglibose, acarbose, miglitol, emiglitate etc.), biguanides (e.g., phenformin, metformin, buformin etc.), insulin secretagogues [e.g., sulfonylurea (e.g., tolbutamide, glibenclamide, gliclazide, chlorpropamide, tolazamide, acetohexamide, glyclopyramide, glimepiride, glipizide, glybuzole etc.), repaglinide, senaglinide, nateglinide, mitiglinide or calcium salt hydrate thereof, GLP-1 etc.], amylin agonist (e.g., pramlintide etc.), phosphotyrosine phosphatase inhibitor (e.g., vanadic acid etc.) and the like.

Examples of the therapeutic agent for diabetic complications include aldose reductase inhibitors (e.g., tolrestat, epalrestat, zenarestat, zopolrestat, minalrestat, fidarestat, SNK-860, CT-112 etc.), neurotrophic factors (e.g., NGF, NT-3, BDNF etc.), neurotrophic factor-production promoter, PKC inhibitors (e.g., LY-333531 etc.), AGE inhibitors (e.g., ALT946, pimagedine, pyratoxanthine, N-phenacylthiazolium bromide (ALT766), EXO-226 etc.), active oxygen scavengers (e.g., thioctic acid etc.), cerebral vasodilators (e.g., tiapuride, mexiletine etc.) and the like.

Examples of the antihyperlipidemia agent include statin compounds which are cholesterol synthesis inhibitors (e.g., pravastatin, simvastatin, lovastatin, atorvastatin, fluvastatin, cerivastatin, itavastatin or a salt thereof (e.g., sodium salt etc.) etc.), squalene synthase inhibitor or fibrate compounds having a triglyceride lowering action (e.g., bezafibrate, clofibrate, simfibrate, clinofibrate etc.) and the like.

Examples of the antiobesity agents include antiobesity agents acting on the central nervous system (e.g., dexfenfluramine, fenfluramine, phentermine, sibutramine, amfepramone, dexamphetamine, mazindol, phenylpropanolamine, clobenzorex etc.), pancreatic lipase inhibitors (e.g., orlistat etc.), 133 agonists (e.g., CL-316243, SR-58611-A, UL-TG-307, SB-226552, AJ-9677, BMS-196085, AZ-40140 etc.), peptidic anorexiants (e.g., leptin, CNTF (Ciliary Neurotropic Factor) etc.), cholecystokinin agonists (e.g., lintitript, FPL-15849 etc.) and the like.

Examples of the diuretic agent include, for example, xanthine derivatives (e.g., theobromine sodium salicylate, theobromine calcium salicylate etc.), thiazide preparations (e.g., ethiazide, cyclopenthiazide, trichlormethiazide, hydrochlorothiazide, hydroflumethiazide, benzylhydrochlorothiazide, penflutizide, polythiazide, methyclothiazide etc.), carbonate dehydratase inhibitors (e.g., acetazolamide etc.), chlorobenzenesulfonamide preparations (e.g., chlorthalidone, mefruside, indapamide etc.), azosemide, isosorbide, ethacrynic acid, piretanide, bumetanide, furosemide and the like.

Examples of the chemotherapeutic agent include alkylating agents (e.g., cyclophosphamide, ifosfamide etc.), metabolic antagonists (e.g., methotrexate, 5-fluorouracil etc.), antitumor antibiotics (e.g., mitomycin, adriamycin etc.), plant-derived antitumor agents (e.g., vincristine, vindesine, Taxol etc.), cisplatin, carboplatin, etoposide and the like. Particularly, 5-fluorouracil derivatives (e.g., Furtulon, Neo-Furtulon and the like) are preferable.

Examples of the immunotherapeutic agent include microorganism or bacterium-derived components (e.g., muramyl dipeptide derivative, Picibanil etc.), polysaccharides having an immunity enhancing activity (e.g., lentinan, schizophyllan, krestin etc.), cytokine obtained by genetic engineering (e.g., interferon, interleukin (IL) etc.), colony stimulating agents (e.g., granulocyte colony stimulating factor, erythropoietin etc.) and the like. Particularly, IL-1, IL-2, IL-12 and the like are preferable. Moreover, pharmaceutical agents whose cachexia-improving effect is observed in animal models or clinically, that is, cyclooxygenase inhibitors (e.g., indomethacin etc.) (Cancer Research, vol. 49, p. 5935-5939, 1989), progesterone derivatives (e.g., megestrol acetate etc.) (Journal of Clinical Oncology, vol. 12, p. 213-225, 1994), glucocorticoids (e.g., dexamethasone etc.), metoclopramide pharmaceuticals, tetrahydrocannabinol pharmaceuticals (same as those mentioned above), fat metabolism ameliorating agents (e.g., eicosapentanoic acid etc.) (British Journal of Cancer, vol. 68, p. 314-318, 1993), growth hormone, IGF-1, or antibodies to TNF-α, LIF, IL-6 or oncostatin M, which are cachexia-inducing factors, and the like can also be used in combination with the pharmaceutical agent of the present invention.

Moreover, pharmaceutical agents generally used for the treatment of cardiac failure, such as digitalis, catecholamine (e.g., dobutamin, dopamine, denopamine, zamoterol etc.), nitrate drugs (e.g., nitroglycerol etc.), hydralazine, PDE inhibitors (e.g., milrinone etc.), Ca sensitivity increasing agents (e.g., pimobendan etc.), thrombolytic agents (e.g., t-PA etc.), anticoagulants (e.g., heparin, warfarin etc.), anti-platelet agents (e.g., aspirin etc.), antiarrhythmic agents (e.g., amiodarone etc.), α-blockers (e.g., prazosin etc.), atrial diuretic peptide, NEP inhibitors (e.g., fasidotril etc.), endothelin antagonists (e.g., bosentan etc.), vasopressin antagonists (e.g., conivaptan etc.), matrix metalloprotease inhibitors and the like can be mentioned.

The mineralocorticoid receptor antagonist of the present invention can also be used in combination with biological preparations (e.g., antibody, vaccine preparation etc.) when applying to the above-mentioned disease. In addition, it can also be applied for a combination therapy in combination with a gene therapy and the like. As the antibody and vaccine preparation, for example, vaccine preparations for angiotensin II, vaccine preparation for CETP, CETP antibody, TNF α-antibody, antibody to other cytokine, amyloid β vaccine preparation, diabetes type 1 vaccine (e.g., DIAPEP-277 of Peptor etc.) and the like, antibody to or vaccine preparation for cytokine, renin angiotensin enzyme and products thereof, antibody to or vaccine preparation for enzyme and protein involved in blood lipid metabolism, antibody to or vaccine relating to enzyme and protein involved in blood coagulation or fibrinolytic system, antibody to or vaccine preparation for protein involved in sugar metabolism and insulin resistance and the like can be mentioned. In addition, as methods for the gene therapy, for example, a treatment method using a gene relating to cytokine, rennin or angiotensin enzyme and a product thereof, a treatment method using a gene relating to the signal transduction system such as β receptor, adenyl cyclase and the like, a treatment method using a gene relating to GRK such as β ARKct, β arrestin and the like, a treatment method using a DNA decoy such as NFκB decoy and the like, a treatment method using antisense, a treatment method using a gene (e.g., gene relating to metabolism, excretion or absorption of cholesterol, triglyceride, HDL-cholesterol or blood phospholipid etc.) relating to enzyme or protein involved in blood lipid metabolism, a treatment method using a gene relating to enzyme or protein (e.g., growth factor such as HGF, VEGF and the like) involved in angiogenesis therapy for peripheral vessel obstruction and the like, a treatment method using a gene relating to protein involved in sugar metabolism or insulin resistance, antisense to cytokine such as TNF and the like, and the like can be mentioned. In addition, various organ regeneration methods such as cardiac regeneration, kidney regeneration, pancreas regeneration, revascularization and the like, a blood vessel and cardiac muscle neogenesis therapy utilizing transplantation of bone-marrow cell (e.g., myelomonocytic cells, myeloid stem cell), endothelial progenitor cells and other cells having a differentiation potential to muscle (e.g., embryonic stem cell, myoblast etc.) may be used in combination. When the agent of the present invention is used in combination with a combination drug, the agent of the present invention and the combination drug may be administered as separate pharmaceutical agents, or may be administered as a single pharmaceutical agent. For combined use as separate pharmaceutical agents, the time of administration of the agent of the present invention and that of the combination drug are not limited, and they may be administered simultaneously or in a staggered manner to the administration subject. Moreover, two or more kinds of combination drugs may be used in combination at an appropriate ratio.

The dose of the combination drug can be appropriately determined based on the dose of each drug employed clinically. In addition, the administration ratio of the agent of the present invention and the combination drug can be appropriately determined according to the administration subject, administration route, target disease, condition, combination, and the like.

The mineralocorticoid receptor antagonist of the present invention has a superior mineralocorticoid receptor antagonistic action, and is advantageously used for the prophylaxis or treatment of circulatory diseases such as hypertension, cardiac failure and the like.

### Examples

In the following Preparations and Examples, melting point, mass spectrum (MS) and nuclear magnetic resonance spectrum (NMR) were measured under the following conditions. melting point measurement tools: Yanagimoto micromelting point measuring apparatus, or Büchi melting point measuring apparatus type B-545 was used.

MS measurement tools: Waters Corporation ZMD, Waters Corporation ZQ2000 or Micromass Ltd., platform II, ionization method: Electron Spray Ionization (ESI) or Atmospheric Pressure Chemical Ionization (APCI). Unless specifically indicated, ESI was used.

NMR measurement tools: Varian Inc. Varian Mercury-300 (300 MHz), Varian INOVA-400 (400 MHz), Bruker BioSpin Corp. AVANCE 300. Chemical shifts are given in ppm with tetramethylsilane as the internal standard, and coupling constants (J) are given in hertz (Hz).

In Preparations and Examples, purification by preparative HPLC was performed under the following conditions.

Preparative HPLC tools: Waters Corporation, UV purification system

column: Develosil ODS-UG-10

solvent: Solution A; 0.1% trifluoroacetic acid-containing water, Solution B; 0.1% trifluoroacetic acid-containing acetonitrile

gradient: 10 min gradient, 5-100% gradient

Gradient cycle: 0.00 min (A/B=95/5), 1.00 min (A/B=95/5), 2.00 min (A/B=80/20), 5.00 min (A/B=5/95), 5.10 min (A/B=0/100), 7.00 min (A/B=100/0)

flow rate: 150 mL/min, detection method: UV 220 nm

The abbreviations in Reference Examples and Examples follow those generally used in the pertinent technical field and, for example, mean the following.

s: singlet

d: doublet

t: triplet

q: quartet

dd: double doublet

dt: double triplet

dq: double quartet

ddd: double double doublet

rt: room temperature

td: triple doublet

tt: triple triplet

m: multiplet

br: broad

brs: broad singlet

J: coupling constant

WSC: water-soluble carbodiimide

THF: tetrahydrofuran

DMF: dimethylformamide

DMSO: dimethyl sulfoxide

DBU: 1,8-diazabicyclo[5.4.0]undeca-7-en

EDCI: 1-ethyl-3-(3-dimethylaminopropyl)carbodiimide

HOBt: 1-hydroxybenzotriazole

IPE: diisopropyl ether

DMAP: 4-(dimethylamino)pyridine

DCM: dichloromethane

DCE: dichloroethane

IPA: isopropylalcohol

TFA: trifluoroacetic acid

TEA: triethylamine

RP-HPLC: reverse phase high performance liquid chromatography

EtOAc: ethyl acetate

### Preparation 1

### 1-(4-Fluorophenyl)butane-1,3-dione

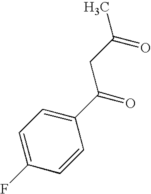

To the suspension of sodium hydride (60% in oil) (43.4 g, washed with hexane) in tetrahydrofuran (1500 ml) was added 4-fluoroacetophenone (50 g), and stirred at room temperature for 30 min. To the mixture was added ethyl acetate (127.6 g), and stirred at 40° C. for 3 h. The reaction mixture was poured into cooled (0° C.) 1N hydrochloric acid to acidify, and the tetrahydrofuran layer was removed. The aqueous solution was diluted with ethyl acetate. The organic layer was separated, washed with aqueous hydrochloric acid, and saturated brine, concentrated, and crystallized from cold hexane (0° C.) to give the title compound (48.3 g) as crude. The crude compound was used to the next reaction without further purification.

### Preparation 2

### 5-(4-Fluorophenyl)-1,3-dimethyl-1H-pyrazole

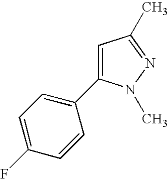

A mixture of 1-(4-fluorophenyl)butane-1,3-dione (12.6 g), methyl hydrazine (6.4 g), trifluoroacetic acid (10.7 ml), and triethylamine (19.4 ml) in isopropanol (350 ml) was stirred at 80° C. for 1 h. The solvent was removed under reduced pressure. Then, the residue was diluted with ethyl acetate, and washed with aqueous sodium bicarbonate, and saturated brine. The organic layer was separated, washed with water, dried over anhydrous magnesium sulfate and concentrated. The residue was chromatographed on silica gel using hexane/ethyl acetate as an eluent to give the title compound (10.02 g).

¹H-NMR (DMSO-d₆) δ: 2.16 (3H, s), 3.74 (3H, s), 6.16 (1H, s), 7.27-7.40 (2H, m), 7.54 (2H, dd, J=8.7, 5.5 Hz).

### Preparation 3

### 4-Bromo-5-(4-fluorophenyl)-1,3-dimethyl-1H-pyrazole

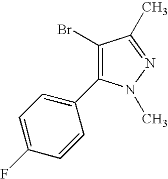

A mixture of 5-(4-fluorophenyl)-1,3-dimethyl-1H-pyrazole (5 g) and N-bromosuccinimide (4.68 g) in acetonitrile (40 ml) was stirred at room temperature for 10 min. The mixture was diluted with ethyl acetate, and washed with aqueous sodium bicarbonate, and saturated brine. The organic layer was separated, dried over anhydrous magnesium sulfate and concentrated. The residue was chromatographed on basic silica gel using hexane/ethyl acetate as an eluent to give the title compound (2.11 g).

¹H-NMR (DMSO-d₆) δ: 2.17 (3H, s), 3.69 (3H, s), 7.39 (2H, dd), 7.54 (2H, dd, J=8.7, 5.7 Hz).

### Preparation 4

### (5-Methyl-2H-1,4-benzoxazin-3(4H)-one

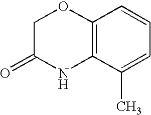

To a mixture of 3-methyl-2-nitrophenol (10 g) and potassium carbonate (13.54 g) in dimethyl sulfoxide (60 ml) was added methyl bromoacetate (19.98 g), and stirred at room temperature for 3 h. The reaction mixture was diluted with ethyl acetate, and washed with water, and saturated brine. The organic layer was separated, dried over anhydrous magnesium sulfate and concentrated. To the residue solution in acetic acid/tetrahydrofuran (210 ml/420 ml) was added zinc powder, refluxed for 18 h, and filtered. The filtrate was concentrated, then the residue was diluted with ethyl acetate, and washed with saturated aqueous. sodium bicarbonate, and saturated brine. The organic layer was separated, dried over anhydrous magnesium sulfate and concentrated. The residue was crystallized from hexane/ethyl acetate to give the product (4.86 g).

¹H-NMR (DMSO-d₆) δ: 2.22 (3H, s), 4.50 (2H, s), 6.71-6.91 (3H, m), 10.21 (1H, br. s)

### Preparation 5

### 6-Bromo-5-methyl-2H-1,4-benzoxazin-3(4H)-one

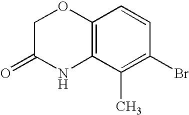

A mixture of (5-methyl-2H-1,4-benzoxazin-3(4H)-one (100 mg) and N-bromosuccinimide (109 mg) in N,N-dimethylformamide (2 ml) was stirred at room temperature for 20 h. The mixture was diluted with ethyl acetate, and washed with aqueous sodium bicarbonate, and saturated brine. The organic layer was separated, dried over anhydrous magnesium sulfate and concentrated. The residue was chromatographed on basic silica gel using hexane/ethyl acetate as an eluent to give the title compound (91 mg).

¹H-NMR (DMSO-d₆) δ: 2.30 (3H, s), 4.53 (2H, s), 6.81 (1H, d, J=8.3 Hz), 7.18 (1H, d, J=8.7 Hz), 10.40 (1H, br. s).

### Preparation 6

### 5-Methyl-6-(4,4,5,5-tetramethyl-1,3,2-dioxaborolan-2-yl)-2H-1,4-benzoxazin-3(4H)-one

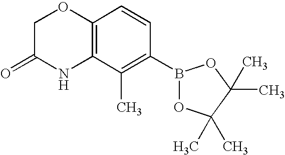

A mixture of 6-bromo-5-methyl-2H-1,4-benzoxazin-3(4H)-one (10 g), 4,4,4′,4′,5,5,5′,5′-octamethyl-2,2′-bi-1,3,2-dioxaborolane (20.98 g), potassium acetate (12.16 g), and [1,1′-bis(diphenylphosphino)ferrocene]dichloropalladium(II) dichloromethane adduct (3.37 g) in 1,4-dioxane (200 ml) was well evacuated, and refluxed for 16 h. The reaction mixture was diluted with ethyl acetate, and washed with water, and saturated brine. The organic layer was separated, dried over anhydrous magnesium sulfate and concentrated. The residue was crystallized from ethyl acetate/hexane to give the title compound (4.82 g).

¹H-NMR (DMSO-d₆) δ: 1.28 (12H, s), 2.41 (3H, s), 4.53 (2H, s), 6.80 (1H, d, J=8.0 Hz), 7.25 (1H, d, J=8.0 Hz), 10.13 (1H, s).

### Preparation 7

### 2-[5-(4-Fluorophenyl)-3-methyl-1H-pyrazol-1-yl]ethanol

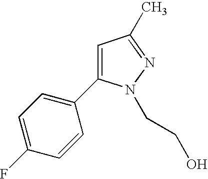

The title compound was obtained from 2-hydrazinylethanol (10 g) according to the similar procedure described for Preparation 2 (10.32 g).

¹H-NMR (DMSO-d₆) δ: 2.18 (3H, s), 3.75 (2H, q, J=5.5 Hz), 3.98 (2H, t, J=5.7 Hz), 4.93 (1H, t, J=5.2 Hz), 6.12 (1H, s), 7.31 (2H, t, J=8.9 Hz), 7.59 (2H, dd, J=8.9, 5.5 Hz).

### Preparation 8

### 2-[5-(4-Fluorophenyl)-3-methyl-1H-pyrazol-1-yl]ethyl4-methylbenzenesulfonate

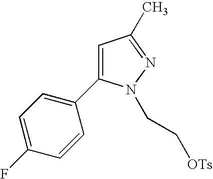

To a solution of 2-[5-(4-fluorophenyl)-3-methyl-1H-pyrazol-1-yl]ethanol (10 g) in pyridine (60 ml) was added 4-methylbenzenesulfonyl chloride (11.25 g), and stirred at room temperature for 3.5 h. The reaction mixture was diluted with ethyl acetate, and washed with 6N hydrochloric acid, and saturated brine. The organic layer was separated, dried over anhydrous magnesium sulfate and concentrated to give the title compound (16 g).

¹H-NMR (DMSO-d₆) δ: 2.10 (3H, s), 2.42 (3H, s), 4.18 (2H, t, J=5.0 Hz), 4.33 (2H, t, J=5.0 Hz), 6.09 (1H, s), 7.31 (2H, t, J=8.9 Hz), 7.36-7.48 (4H, m), 7.51-7.62 (2H, m).

### Preparation 9

### 5-(4-Fluorophenyl)-3-methyl-1-[2-(methylsulfanyl)ethyl]-1H-pyrazole

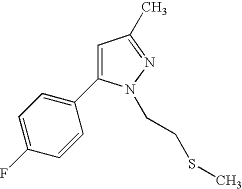

A mixture of 2-[5-(4-fluorophenyl)-3-methyl-1H-pyrazol-1-yl]ethyl-4-methylbenzenesulfonate (10 g) and sodium methanethiolate (2.06 g) in ethanol (35 ml) was stirred at room temperature for 2 h. The solvent was removed under reduced pressure, then the residue was diluted with ethyl acetate, and filtered. The filtrate was concentrated in vacuo. The residue was chromatographed on silica gel using hexane/ethyl acetate as an eluent to give the title compound (4.74 g).

¹H-NMR (DMSO-d₆) δ: 1.84 (3H, s), 2.18 (3H, s), 2.81 (2H, t, J=7.0 Hz), 4.14 (2H, t, J=7.0 Hz), 6.13 (1H, s), 7.33 (2H, dd, J=8.9, 4.5 Hz), 7.52 (2H, dd, J=8.9, 5.5 Hz).

### Preparation 10

### 4-Bromo-5-(4-fluorophenyl)-3-methyl-1-[2-(methylsulfanyl)ethyl]-1H-pyrazole

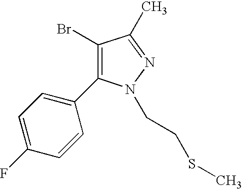

The title compound was obtained from 5-(4-fluorophenyl)-3-methyl-1-[2-(methylsulfanyl)ethyl]-1H-pyrazole (4.74 g) according to the similar procedure described for Preparation 3 (quant.).

¹H-NMR (DMSO-d₆) δ: 1.80 (3H, s), 2.20 (3H, s), 2.78 (2H, t, J=6.9 Hz), 4.12 (2H, t, J=6.9 Hz), 7.40 (2H, t, J=8.9 Hz), 7.48-7.59 (2H, m).

### Preparation 11

### 4-Bromo-5-(4-fluorophenyl)-3-methyl-1-[2-(methylsulfinyl)ethyl]-1H-pyrazole

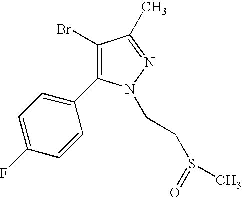

A mixture of 4-bromo-5-(4-fluorophenyl)-3-methyl-1-[2-(methylsulfanyl)ethyl]-1H-pyrazole (2 g) and 3-chlorobenzenecarboperoxoic acid (1.15 g) in dichloromethane (5 ml) was stirred at room temperature for 15 h. The reaction mixture was diluted with ethyl acetate, and washed with aqueous sodium bicarbonate, and saturated brine. The organic layer was separated, dried over anhydrous magnesium sulfate and concentrated. The residue was chromatographed on silica gel using hexane/ethyl acetate as an eluent to give the title compound (1.97 g).

¹H-NMR (DMSO-d₆) δ: 2.20 (3H, s), 2.51 (3H, s), 2.95-3.10 (1H, m), 3.21-3.31 (1H, m), 4.25-4.38 (2H, m), 7.34-7.46 (2H, m), 7.49-7.60 (2H, m).

### Preparation 12

### 4-Bromo-5-(4-fluorophenyl)-3-methyl-1-[2-(methylsulfonyl)ethyl]-1H-pyrazole

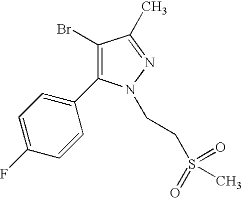

The title compound was obtained from 4-bromo-5-(4-fluorophenyl)-3-methyl-1-[2-(methylsulfinyl)ethyl]-1H-pyrazole (400 mg) according to the similar procedure described for Preparation 11 (228 mg).

¹H-NMR (DMSO-d₆) δ: 2.20 (3H, s), 2.91 (3H, s), 3.65 (2H, t, J=7.1 Hz), 4.33 (2H, t, J=7.1 Hz), 7.41 (2H, t, J=8.9 Hz), 7.50-7.61 (2H, m).

### Preparation 13

### 3-Hydrazinylpropan-1-ol

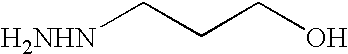

A mixture of hydrazine hydrate (25 g) and solid sodium hydroxide was heated to 95° C. The heat source was removed, and to the mixture was added 3-chloropropan-1-ol (9.63 g). The mixture was stirred for 3 h, and concentrated. The residue was suspended in ethanol, and filtered. The filtrate was concentrated to give the title compound (7.42 g) as crude. The crude compound was used to the next reaction without further purification.

### Preparation 14

### 3-[5-(4-Fluorophenyl)-3-methyl-1H-pyrazol-1-yl]propan-1-ol

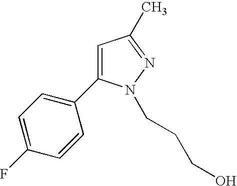

The title compound was obtained from 3-hydrazinylpropan-1-ol (7.42 g) according to the similar procedure described for Preparation 2 (4.56 g).

¹H-NMR (DMSO-d₆) δ: 1.85 (2H, quin, J=6.7 Hz), 3.25-3.39 (2H, m), 4.03 (2H, t, J=7.2 Hz), 4.50 (1H, m), 6.11 (1H, s), 7.31 (2H, t, J=8.7 Hz), 7.49 (2H, t, J=2.8 Hz).

### Preparation 15

### 3-[4-Bromo-5-(4-fluorophenyl)-3-methyl-1H-pyrazol-1-yl]propan-1-ol

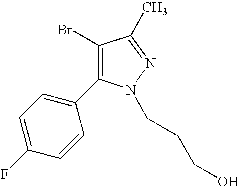

The title compound was obtained from 3-[5-(4-fluorophenyl)-3-methyl-1H-pyrazol-1-yl]propan-1-ol (3 g) according to the similar procedure described for Preparation 3 (3.80 g).

¹H-NMR (DMSO-d₆) δ: 1.80 (2H, quin, J=6.5 Hz), 2.18 (3H, s), 3.22-3.34 (2H, m), 3.99 (2H, t, J=7.3 Hz), 4.48 (1H, m), 7.34-7.44 (2H, m), 7.50 (2H, dd, J=8.7, 5.5 Hz).

### Preparation 16

### 2-[4-Bromo-5-(4-fluorophenyl)-3-methyl-1H-pyrazol-1-yl]ethyl-4-methylbenzenesulfonate

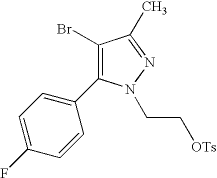

The title compound was obtained from 2-[5-(4-fluorophenyl)-3-methyl-1H-pyrazol-1-yl]ethyl-4-methylbenzenesulfonate (2 g) according to the similar procedure described for Preparation 3 (quant.).

¹H-NMR (DMSO-d₆) δ: 2.09 (3H, s), 2.43 (3H, s), 4.15 (2H, t, J=4.8 Hz), 4.28 (2H, t, J=4.8 Hz), 7.34-7.39 (4H, m), 7.41 (2H, d, J=8.1 Hz), 7.55 (2H, d, J=8.3 Hz).

### Preparation 17

### 4-Bromo-5-(4-fluorophenyl)-3-methyl-1-[2-(1H-pyrazol-1-yl)ethyl]-1H-pyrazole

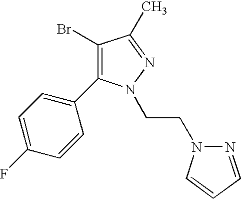

A mixture of 2-[4-bromo-5-(4-fluorophenyl)-3-methyl-1H-pyrazol-1-yl]ethyl-4-methylbenzenesulfonate (500 mg), 1H-pyrazole (150 mg) and potassium carbonate (305 mg) in dimethyl sulfoxide was stirred at 75° C. for 8 h. The reaction mixture was diluted with ethyl acetate, and washed with water, and saturated brine. The organic layer was separated, dried over anhydrous magnesium sulfate and concentrated. The residue was chromatographed on basic silica gel using hexane/ethyl acetate as an eluent to give the title compound (103 mg).

¹H-NMR (DMSO-d₆) δ: 2.22 (3H, s), 4.27 (2H, t, J=5.7 Hz), 4.48 (2H, t, J=5.5 Hz), 6.08-6.23 (1H, m), 7.02-7.15 (2H, m), 7.22-7.50 (4H, m).

### Preparation 18

### 4-Bromo-5-(4-fluorophenyl)-3-methyl-1-(3,3,3-trifluoropropyl)-1H-pyrazole

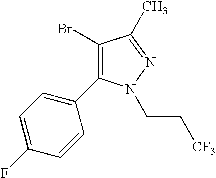

To a mixture of 4-bromo-5-(4-fluorophenyl)-3-methyl-1H-pyrazole (319 mg), 3,3,3-trifluoropropan-1-ol (200 mg), triphenylphosphine (460 mg) in tetrahydrofuran was added diethyl (E)-diazene-1,2-dicarboxylate (40% in toluene, 800 μl), and stirred at room temperature for 4 h. The solvent was removed under reduced pressure, then the residue was chromatographed on basic silica gel using hexane/ethyl acetate as an eluent to give the title compound (118 mg).

¹H-NMR (DMSO-d₆) δ: 2.20 (3H, s), 2.78 (2H, qt, J=11.2, 6.8 Hz), 4.17 (2H, t, J=7.0 Hz), 7.37-7.46 (2H, m), 7.47-7.55 (2H, m).

### Preparation 19

### 3-[4-Bromo-5-(4-fluorophenyl)-3-methyl-1H-pyrazol-1-yl]propanal

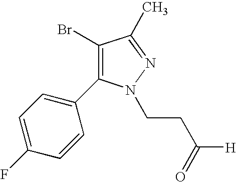

To the solution of 3-[4-bromo-5-(4-fluorophenyl)-3-methyl-1H-pyrazol-1-yl]propan-1-ol (400 mg) in dichloromethane (4 ml) was added Dess-Martin periodinane (464 mg), and stirred at room temperature for 4 h. The reaction mixture was diluted with ethyl acetate, and washed with aqueous sodium thiosulfate/sodium bicarbonate, and saturated brine. The organic layer was separated, dried over anhydrous magnesium sulfate and concentrated. The residue was chromatographed on silica gel using hexane/ethyl acetate as an eluent to give the title compound (453 mg).

¹H-NMR (300 MHz, DMSO-d₆) δ: 2.16 (3H, s), 2.92 (2H, t, J=6.6 Hz), 4.21 (2H, t, J=6.4 Hz), 7.40 (2H, t, J=8.9 Hz), 7.47-7.58 (2H, m), 9.61 (1H, s).

### Preparation 20

### 4-Bromo-1-(3,3-difluoropropyl)-5-(4-fluorophenyl)-3-methyl-1H-pyrazole

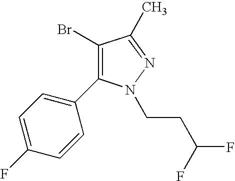

To a cold solution (−78° C.) of 3-[4-bromo-5-(4-fluorophenyl)-3-methyl-1H-pyrazol-1-yl]propanal (450 mg) in dichloromethane (5 ml) was added (diethylamino)sulfur trifluoride (932 mg), and stirred at room temperature for 5 h. The reaction mixture was diluted with ethyl acetate, and washed with water, and saturated brine. The organic layer was separated, dried over anhydrous magnesium sulfate and concentrated. The residue was chromatographed on silica gel using hexane/ethyl acetate as an eluent to give the title compound (35 mg).

¹H-NMR (300 MHz, DMSO-d₆) δ: 2.20 (3H, s), 2.21-2.40 (2H, m), 4.08 (2H, t, J=7.0 Hz), 6.07 (1H, tt, J=56.0, 4.2 Hz), 7.35-7.45 (2H, m), 7.45-7.57 (2H, m).

### Preparation 21

### 4-Bromo-5-(4-fluorophenyl)-1-(3-fluoropropyl)-3-methyl-1H-pyrazole

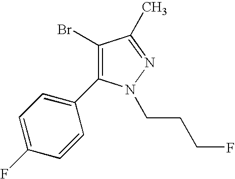

To a hot mixture (40° C.) of 4-bromo-5-(4-fluorophenyl)-3-methyl-1H-pyrazole (500 mg), 3-fluoropropan-1-ol (306 mg), triphenylphosphine (1.03 g) in tetrahydrofuran was added diisopropyl azodicarboxylate (40% in toluene, 2.06 ml), and stirred at 60° C. for 5 h. The reaction mixture was diluted with ethyl acetate, and washed with aqueous sodium bicarbonate, and saturated brine. The organic layer was separated, dried over anhydrous magnesium sulfate and concentrated. The residue was chromatographed on basic silica gel using hexane/ethyl acetate as an eluent to give the title compound (603 mg).

¹H-NMR (300 MHz, DMSO-d₆) δ: 1.93-2.04 (1H, m), 2.04-2.12 (1H, m), 2.19 (3H, s), 4.04 (2H, t, J=7.1 Hz), 4.41 (2H, t, J=5.8 Hz), 7.35-7.45 (2H, m), 7.45-7.55 (2H, m).

### Preparation 22

### 4-Bromo-5-(4-fluorophenyl)-3-methyl-1-(thiophen-2-ylmethyl)-1H-pyrazole

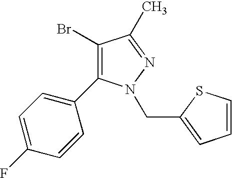

The title compound was obtained from thiophen-2-ylmethanol (448 mg) according to the similar procedure described for Preparation 21 (295 mg).

¹H-NMR (300 MHz, DMSO-d₆) δ: 2.19 (3H, s), 5.37 (2H, s), 6.76 (1H, d, J=3.4 Hz), 6.89 (1H, dd, J=5.3, 3.4 Hz), 7.33-7.56 (5H, m).

### Preparation 23

### 5-{[4-Bromo-5-(4-fluorophenyl)-3-methyl-1H-pyrazol-1-yl]methyl}-1,3-thiazole

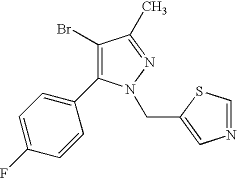

The title compound was obtained from 1,3-thiazol-5-ylmethanol (451 mg) according to the similar procedure described for Preparation 21 (237 mg).

¹H-NMR (300 MHz, DMSO-d₆) δ: 2.19 (3H, s), 5.46 (2H, s), 7.28-7.53 (4H, m), 7.58 (1H, s), 8.97 (1H, s).

### Preparation 24

### 4-Bromo-5-(4-fluorophenyl)-3-methyl-1-(4,4,4-trifluorobutyl)-1H-pyrazole

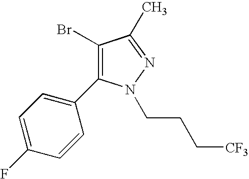

The title compound was obtained from 4,4,4-trifluorobutan-1-ol (703 mg) according to the similar procedure described for Preparation 21 (305 mg).

¹H-NMR (300 MHz, DMSO-d₆) δ: 1.86 (2H, quin, J=7.5 Hz), 2.07-2.18 (2H, m), 2.19 (3H, s), 4.02 (2H, t, J=7.1 Hz), 7.28-7.45 (2H, m), 7.46-7.56 (2H, m).

### Preparation 25

### 1-(4-Fluorobutyl)-5-(4-fluorophenyl)-3-methyl-1H-pyrazole

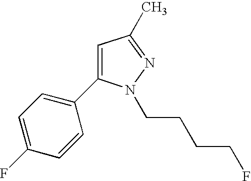

A mixture of 1-bromo-4-fluorobutane (1 g), tert-butyl hydrazinecarboxylate (1.02 g), sodium bicarbonate (650 mg) and sodium iodide (4.8 mg) in acetonitrile (20 ml) was refluxed for 24 h. The reaction mixture was concentrated, and the residue was diluted with ethyl acetate, and filtered. The filtrate was concentrated, and the residue was dissolved with toluene. To the solution was added trifluoroacetic acid (5 ml), and stirred at room temperature for 30 min. The reaction mixture was concentrated, and the residue and 1-(4-fluorophenyl)butane-1,3-dione (1.16 g) was dissolved with methanol (15 ml). To the mixture was added concentrated hydrochloric acid (1.08 ml), and stirred at 40° C. for 18 h. The reaction mixture was diluted with ethyl acetate, and washed with aqueous sodium bicarbonate, and saturated brine. The organic layer was separated, dried over anhydrous magnesium sulfate and concentrated. The residue was chromatographed on basic silica gel using hexane/ethyl acetate as an eluent to give the title compound (550 mg).

¹H-NMR (300 MHz, DMSO-d₆) δ: 1.38-1.62 (2H, m), 1.74 (2H, quin, J=7.4 Hz), 2.18 (3H, s), 4.01 (2H, t, J=7.0 Hz), 4.25 (2H, dt, J=47.7, 5.7 Hz), 6.12 (1H, s), 7.26-7.39 (2H, m), 7.47 (2H, dd, J=8.9, 5.5 Hz).

### Preparation 26

### 4-Bromo-1-(4-fluorobutyl)-5-(4-fluorophenyl)-3-methyl-1H-pyrazole

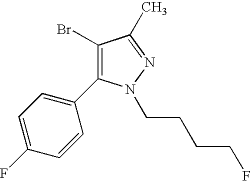

The title compound was obtained from 1-(4-fluorobutyl)-5-(4-fluorophenyl)-3-methyl-1H-pyrazole (550 mg) according to the similar procedure described for Preparation 3 (645 mg).

¹H-NMR (300 MHz, DMSO-d₆) δ: 1.34-1.60 (2H, m), 1.70 (2H, quin, J=7.3 Hz), 2.19 (3H, s), 3.98 (2H, t, J=7.1 Hz), 4.24 (2H, dt, J=47.3, 6.0 Hz), 7.35-7.44 (2H, m), 7.44-7.53 (2H, m).

### Preparation 27

### 4-Bromo-1-(3,3-difluorobutyl)-5-(4-fluorophenyl)-3-methyl-1H-pyrazole

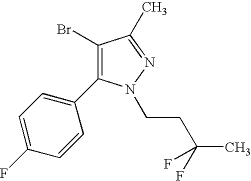

The title compound was obtained from 4-[4-bromo-5-(4-fluorophenyl)-3-methyl-1H-pyrazol-1-yl]butan-2-one (240 mg) according to the similar procedure described for Preparation 20 (73 mg).

¹H-NMR (300 MHz, DMSO-d₆) δ: 1.51 (3H, t, J=19.2 Hz), 2.19 (3H, s), 2.26-2.46 (2H, m), 4.10 (2H, dd, J=8.4, 6.7 Hz), 7.40 (2H, t, J=8.9 Hz), 7.46-7.56 (2H, m).

### Preparation 28

### 1-Hydrazinylbutan-2-ol

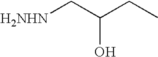

To heated (70° C.) hydrazine hydrate (44.5 g) was added 2-ethyloxirane (8.9 g), and stirred at 45° C. for 18 h. The reaction mixture was concentrated to give the title compound (12 g) as crude. The crude compound was used to next reaction without further purification.

### Preparation 29

### 1-[4-Bromo-5-(4-fluorophenyl)-3-methyl-1H-pyrazol-1-yl]butan-2-ol

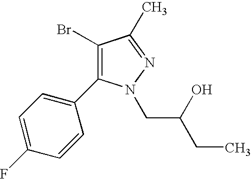

To a mixture of 1-hydrazinylbutan-2-ol (12 g) and 1-(4-fluorophenyl)butane-1,3-dione (18.87 g) was added concentrated hydrochloric acid (17 ml), and stirred at room temperature for 18 h. The reaction mixture was diluted with ethyl acetate, and washed with aqueous sodium bicarbonate, and saturated brine. The organic layer was separated, dried over anhydrous magnesium sulfate and concentrated. The residue and N-bromosuccinimide (4.37 g) in acetonitrile (50 ml) was stirred at room temperature for 1 h. The mixture was diluted with ethyl acetate, and washed with aqueous sodium bicarbonate, and saturated brine. The organic layer was separated, dried over anhydrous magnesium sulfate and concentrated. The residue was chromatographed on basic silica gel using hexane/ethyl acetate as an eluent to give the title compound (1.19 g).

¹H-NMR (300 MHz, DMSO-d₆) δ: 0.79 (3H, t, J=7.3 Hz), 1.11-1.41 (2H, m), 2.19 (3H, s), 3.69-3.88 (3H, m), 4.87 (1H, d, J=5.1 Hz), 7.37 (2H, t, J=8.9 Hz), 7.56 (2H, dd, J=8.9, 5.6 Hz).

### Preparation 30

### 1-[4-Bromo-5-(4-fluorophenyl)-3-methyl-1H-pyrazol-1-yl]butan-2-one

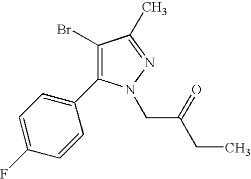

The title compound was obtained from 1-[4-bromo-5-(4-phenyl)-3-methyl-1H-pyrazol-1-yl]butan-2-ol (1.28 g) according to the similar procedure described for Preparation 19 (1.19 g).

¹H-NMR (300 MHz, DMSO-d₆) δ: 0.85 (3H, t, J=7.2 Hz), 2.18 (3H, s), 2.37 (2H, q, J=7.2 Hz), 4.99 (2H, s), 7.31-7.47 (4H, m).

### Preparation 31

### 4-Bromo-1-(2,2-difluorobutyl)-5-(4-fluorophenyl)-3-methyl-1H-pyrazole

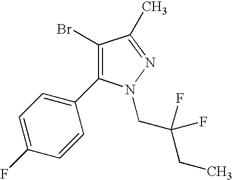

The title compound was obtained from 1-[4-bromo-5-(4-fluorophenyl)-3-methyl-1H-pyrazol-1-yl]butan-2-one (1.19 g) according to the similar procedure described for Preparation 20 (903 mg).

¹H-NMR (300 MHz, DMSO-d₆) δ: 0.84 (3H, t, J=7.4 Hz), 1.79 (2H, tt, J=17.7, 7.5 Hz), 2.21 (3H, s), 4.45 (2H, t, J=13.4 Hz), 7.34-7.43 (2H, m), 7.44-7.51 (2H, m).

### Preparation 32

### 4-Bromo-1-(cyclopropylmethyl)-5-(4-fluorophenyl)-3-methyl-1H-pyrazole

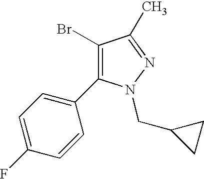

The title compound was obtained from cyclopropylmethanol (452 mg) according to the similar procedure described for Preparation 21 (205 mg).

¹H-NMR (300 MHz, DMSO-d₆) δ: 0.03-0.44 (4H, m), 0.89-1.11 (1H, m), 2.19 (3H, s), 3.83 (2H, d, J=6.8 Hz), 7.34-7.43 (2H, m), 7.44-7.55 (2H, m).

### Preparation 33

### 4-Bromo-1-(2-cyclopropylethyl)-5-(4-fluorophenyl)-3-methyl-1H-pyrazole

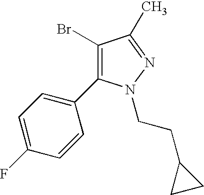

The title compound was obtained from 2-cyclopropylethanol (540 mg) according to the similar procedure described for Preparation 21 (196 mg).

¹H-NMR (300 MHz, DMSO-d₆) δ: −0.23-−0.12 (2H, m), 0.19-0.31 (2H, m), 0.33-0.50 (1H, m), 1.52 (2H, q, J=6.8 Hz), 2.18 (3H, s), 3.99 (2H, t, J=7.0 Hz), 7.39 (2H, t, J=8.9 Hz), 7.44-7.53 (2H, m).

### Preparation 34

### 4-[4-Bromo-5-(4-fluorophenyl)-3-methyl-1H-pyrazol-1-yl]-2,2-difluorobutyl acetate

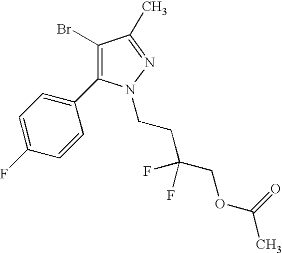

To a mixture of 4-[4-bromo-5-(4-fluorophenyl)-3-methyl-1H-pyrazol-1-yl]butan-2-one (470 mg) and triethylamine (4 ml) in toluene (4 ml) was added tert-butyl(dimethyl)silyl methanesulfonate (535 mg), and stirred at 0° C. for 30 min. The reaction mixture was diluted with ethyl acetate, and washed with aqueous sodium bicarbonate, and saturated brine. The organic layer was separated, dried over anhydrous magnesium sulfate and concentrated. To a solution of the residue in toluene (4 ml) was added lead(IV) acetate (962 mg) and potassium bicarbonate (579 mg), and stirred at room temperature for 3 h. The reaction mixture was diluted with ethyl acetate, and washed with aqueous sodium bicarbonate, and saturated brine. The organic layer was separated, dried over anhydrous magnesium sulfate and concentrated. To a solution of the residue in tetrahydrofuran (4 ml) was added 1M N,N,N-tributylbutan-1-aminium fluoride in tetrahydrofuran (2.9 ml), and stirred at room temperature for 1 h. The reaction mixture was diluted with ethyl acetate, and washed with aqueous sodium bicarbonate, and saturated brine. The organic layer was separated, dried over anhydrous magnesium sulfate and concentrated. A mixture of the residue and (diethylamino)sulfur trifluoride (436 mg) in toluene (3 ml) was stirred at 40° C. for 24 h. The reaction mixture was poured into ice-water, and diluted with ethyl acetate. The organic layer was separated, washed with aqueous sodium bicarbonate, and saturated brine, dried over anhydrous magnesium sulfate and concentrated. The residue was chromatographed on silica gel using hexane/ethyl acetate as an eluent to give the title compound (60 mg).

¹H-NMR (300 MHz, DMSO-d₆) δ: 2.05 (3H, s), 2.19 (3H, s), 2.33-2.49 (2H, m), 4.13 (2H, t, J=7.3 Hz), 4.23 (2H, t, J=13.8 Hz), 7.35-7.46 (2H, m), 7.47-7.56 (2H, m).

### Preparation 35

### 4,4,4-Trifluoro-1-[5-(4-fluorophenyl)-3-methyl-1H-pyrazol-1-yl]butan-2-ol

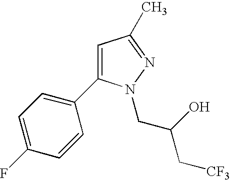

The title compound was obtained from 2-(2,2,2-trifluoroethyl)oxirane (4.73 g) according to the similar procedure described for Preparation 28 and 2 (7.45 g).

¹H-NMR (300 MHz, DMSO-d₆) δ: 2.19 (3H, s), 2.22-2.47 (2H, m), 3.87-4.11 (2H, m), 4.17-4.33 (1H, m), 5.51 (1H, d, J=6.1 Hz), 6.14 (1H, s), 7.32 (2H, t, J=8.9 Hz), 7.58 (2H, dd, J=8.7, 5.3 Hz).

### Preparation 36

### 1-[4-Bromo-5-(4-fluorophenyl)-3-methyl-1H-pyrazol-1-yl]-4,4,4-trifluorobutan-2-ol

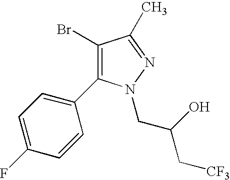

The title compound was obtained from 4,4,4-trifluoro-1-[5-(4-fluorophenyl)-3-methyl-1H-pyrazol-1-yl]butan-2-ol (7.45 g) according to the similar procedure described for Preparation 3 (4.04 g).

¹H-NMR (300 MHz, DMSO-d₆) δ: 2.20 (3H, s), 2.22-2.49 (2H, m), 3.80-4.01 (2H, m), 4.19 (1H, br. s), 5.46 (1H, d, J=6.1 Hz), 7.38 (2H, t, J=8.9 Hz), 7.49-7.65 (2H, m).

### Preparation 37

### 2-{[4-Bromo-5-(4-fluorophenyl)-3-methyl-1H-pyrazol-1-yl]methyl}-1-methylcyclopropanol

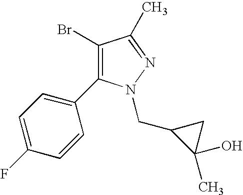

To a cold (0° C.) solution of 1-[4-bromo-5-(4-fluorophenyl)-3-methyl-1H-pyrazol-1-yl]propan-2-one (800 mg) in toluene/triethylamine (8 ml/8 ml) was added tert-butyl(dimethyl)silyl methanesulfonate (952 mg), and stirred at 0° C. to room temperature for 15 h. The reaction mixture was diluted with ethyl acetate, and washed with saturated brine. The organic layer was separated, dried over anhydrous magnesium sulfate and concentrated to give the silylether. To a cold (0° C.) mixture of diethylzinc (1M in hexane, 12.86 ml) and toluene (8 ml) was added chloro(iodo)methane (2.27 g), and stirred at the temperature for 10 min. To the mixture was added a solution of the silylether in toluene (8 ml), and stirred at 0° C. to room temperature for 18 h. The reaction mixture was diluted with ethyl acetate, and washed with saturated brine. The organic layer was separated, dried over anhydrous magnesium sulfate and concentrated. The residue was dissolved in tetrahydrofuran (3 ml), and N,N,N-tributylbutan-1-aminium fluoride (1M in tetrahydrofuran, 330 μl) was added to the mixture at 0° C., and stirred for 1 h at the temperature. The mixture was diluted with ethyl acetate, and washed with saturated brine. The organic layer was separated, dried over anhydrous magnesium sulfate and concentrated. The residue was chromatographed on silica gel using hexane/ethyl acetate as an eluent to give the title compound (49 mg).

¹H-NMR (300 MHz, DMSO-d₆) δ: 0.21 (1H, t, J=5.7 Hz), 0.44 (1H, dd, J=4.9, 9.1 Hz), 0.86-1.05 (1H, m), 1.23 (3H, s), 2.19 (3H, s), 3.88 (1H, dd, J=8.3, 14.4 Hz), 4.15 (1H, dd, J=6.4, 14.4 Hz), 5.19 (1H, s), 7.33-7.44 (2H, m), 7.45-7.53 (2H, m).

### Preparation 38

### 2-[4-Bromo-5-(4-fluorophenyl)-3-methyl-1H-pyrazol-1-yl]ethanesulfonic acid

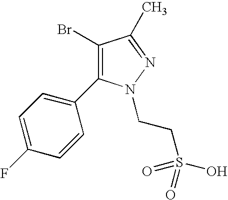

To a solution of 2-[4-bromo-5-(4-fluorophenyl)-3-methyl-1H-pyrazol-1-yl]ethyl4-methylbenzenesulfonate (4 g) in ethanol (40 ml) was added a solution of sodium sulfite (2.11 g) in water (40 ml), and refluxed for 24 h. The organic layer was concentrated, and the resulting precipitate was corrected by filtration, and washed with cold (0° C.) acetone to give the title compound (1.89 g).

¹H-NMR (300 MHz, DMSO-d₆) δ: 2.18 (3H, s), 2.75-2.84 (2H, m), 4.10-4.19 (2H, m), 7.32-7.44 (2H, m), 7.54 (2H, dd, J=8.9, 5.6 Hz), 1H unconfirmed.

### Preparation 39

### 2-[4-Bromo-5-(4-fluorophenyl)-3-methyl-1H-pyrazol-1-yl]-N,N-dimethylethanesulfonamide

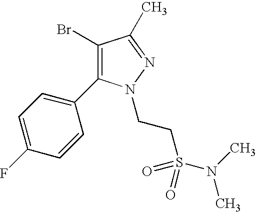

A solution of 2-[4-bromo-5-(4-fluorophenyl)-3-methyl-1H-pyrazol-1-yl]ethanesulfonic acid (100 mg) in thionyl chloride (3 ml) was refluxed for 12 h, and concentrated to give the sulfuryl chloride. To a solution of dimethylamine (2M in tetrahydrofuran, 1.38 ml) was added the sulfuryl chloride solution in tetrahydrofuran (3 ml), and stirred at room temperature for 4 h. The mixture was diluted with ethyl acetate, and washed with saturated brine. The organic layer was separated, dried over anhydrous magnesium sulfate and concentrated. The residue was chromatographed on silica gel using hexane/ethyl acetate as an eluent to give the title compound (44 mg).

¹H-NMR (300 MHz, DMSO-d₆) δ: 2.21 (3H, s), 2.66 (6H, s), 3.53 (2H, t, J=7.2 Hz), 4.27 (2H, t), 7.36-7.48 (2H, m), 7.50-7.62 (2H, m).

### Preparation 40

### 2-[4-Bromo-5-(4-fluorophenyl)-3-methyl-1H-pyrazol-1-yl]-N-methylethanesulfonamide

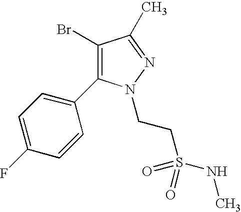

The title compound was obtained from methylamine (2M in tetrahydrofuran, 2.75 ml) according to the similar procedure described for Preparation 39 (44 mg).

¹H-NMR (300 MHz, DMSO-d₆) δ: 2.20 (3H, s), 2.51 (3H, s), 3.49 (2H, dd, J=9.3, 5.6 Hz), 4.23 (2H, dd, J=9.2, 5.7 Hz), 7.04 (1H, s), 7.36-7.47 (2H, m), 7.51-7.62 (2H, m).

### Preparation 41

### (2E)-3-(Dimethylamino)-1-(4-fluorophenyl)prop-2-en-1-one

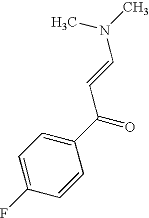

A mixture of 1-(4-fluorophenyl)ethanone (2 g) and 1,1-dimethoxy-N,N-dimethylmethanamine (4 ml) was refluxed for 2 h. The solvent was removed. To the residue was added hexane to give the title compound as crystals (889 mg).

¹H-NMR (300 MHz, DMSO-d₆) δ: 2.92 (3H, s), 3.14 (3H, s), 5.82 (1H, d, J=12.2 Hz), 7.24 (2H, m), 7.71 (1H, d, J=12.2 Hz), 7.96 (2H, dd, J=8.9, 5.7 Hz).

### Preparation 42

### 5-(4-Fluorophenyl)-1-methyl-1H-pyrazole

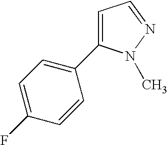

A mixture of (2E)-3-(dimethylamino)-1-(4-fluorophenyl)prop-2-en-1-one (800 mg) and methylhydrazine (257 μl) in ethanol was refluxed for 4 h. The solvent was removed, and the residue was chromatographed on basic silica gel using hexane/ethyl acetate as an eluent to give the title compound (592 mg).

¹H-NMR (300 MHz, DMSO-d₆) δ: 3.83 (3H, s), 6.39 (1H, d, J=1.9 Hz), 7.34 (2H, dd), 7.46 (1H, d, J=1.5 Hz), 7.58 (2H, dd, J=8.9, 5.5 Hz).

### Preparation 43

### 4-Bromo-5-(4-fluorophenyl)-1-methyl-1H-pyrazole

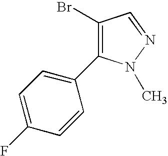

The title compound was obtained from 5-(4-fluorophenyl)-1-methyl-1H-pyrazole (2.77 g) according to the similar procedure described for Preparation 3(1.57 g).

¹H-NMR (300 MHz, DMSO-d₆) δ: 3.76 (3H, s), 7.40 (2H, t, J=8.9 Hz), 7.56 (2H, dd, J=8.9, 5.5 Hz), 7.66 (1H, s).

### Preparation 44

### 3-Fluoro-2-nitrophenol

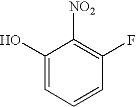

To a mixture of potassium tert-butoxide (7.76 g) in dimethyl sulfoxide (150 ml) was added 1,3-difluoro-2-nitrobenzene (10 g), and stirred at room temperature for 18 h. The reaction mixture was diluted with 1N hydrochloric acid, and extracted with 1,1′-oxydiethane. The combined organic layer was washed with water, dried over anhydrous magnesium sulfate and concentrated. The residue was dissolved in trifluoroacetic acid (100 ml), and stirred at room temperature for 1 h. The mixture was concentrated, and the residue was diluted with 1N sodium hydroxide, washed with 1,1′-oxydiethane. The aqueous layer was acidified with 1N hydrochloric acid, and extracted with 1,1′-oxydiethane. The combined organic layer was washed with water, dried over anhydrous magnesium sulfate and concentrated to give the title compound (7.59 g) as crude. The crude compound was used to next reaction without further purification.

### Preparation 45

### Methyl(3-fluoro-2-nitrophenoxy)acetate

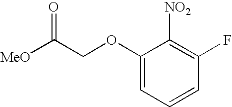

A mixture of 3-fluoro-2-nitrophenol (7.59 g), methylbromoacetate (29.56 g) and potassium carbonate (26.71 g) in dimethyl sulfoxide (30 ml) was stirred at 50° C. for 18 h. The mixture was diluted with ethyl acetate, and washed with saturated brine. The organic layer was separated, dried over anhydrous magnesium sulfate and concentrated. The residue was chromatographed on silica gel using hexane/ethyl acetate as an eluent to give the title compound (4.09 g).

¹H-NMR (300 MHz, DMSO-d₆) δ: 3.65 (3H, s), 5.07 (2H, s), 7.17 (2H, d, J=8.7 Hz), 7.61 (1H, td, J=8.7, 6.8 Hz).

### Preparation 46

### 5-Fluoro-2H-1,4-benzoxazin-3(4H) -one

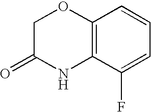

To a solution of methyl(3-fluoro-2-nitrophenoxy)acetate (4.09 g) in acetic acid/tetrahydrofuran (57 ml/115 ml) was added zinc powder (16.4 g) at 45° C., refluxed for 3 h, and filtered. The filtrate was concentrated, and the residue was diluted with ethyl acetate, washed with aqueous sodium bicarbonate, and saturated brine. The organic layer was separated, dried over anhydrous magnesium sulfate and concentrated. The residue was washed with hexane, and water to give the title compound (1.51 g).

¹H-NMR (300 MHz, DMSO-d₆) δ: 4.61 (2H, s), 6.82 (1H, d, J=6.8 Hz), 6.85-7.02 (2H, m), 10.88 (1H, br. s).

### Preparation 47

### 5-Fluoro-6-(4,4,5,5-tetramethyl-1,3,2-dioxaborolan-2-yl)-2H-1,4-benzoxazin-3(4H)-one

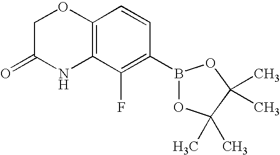

The title compound was obtained from 5-fluoro-2H-1,4-benzoxazin-3(4H)-one (1.61 g) according to the similar procedure described for Preparation 5 and 6 (747 mg).

¹H-NMR (300 MHz, DMSO-d₆) δ: 1.28 (12H, s), 4.65 (2H, s), 6.80 (1H, dd, J=8.3, 0.8 Hz), 7.17 (1H, dd, J=8.2, 6.3 Hz), 10.87 (1H, s).

### Preparation 48

### 3-Chloro-2-nitrophenol

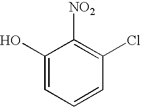

A mixture of 1,3-dichloro-2-nitrobenzene (10 g) and sodium methoxide (28% in methanol, 12.06 g) was refluxed for 18 h. The mixture was diluted with ethyl acetate, and washed with 1N hydrochloric acid, and saturated brine. The organic layer was separated, dried over anhydrous magnesium sulfate and concentrated. To a solution of the residue in dichloromethane (50 ml) was added tribromoborane (1M in dichloromethane, 104.2 ml) at −20° C. Then, the reaction mixture was warmed to room temperature, and stirred for 1 h at the temperature. The mixture was poured into ice-water, and extracted with ethyl acetate. The organic layer was dried over anhydrous magnesium sulfate and concentrated to give the title compound (9.56 g).

¹H-NMR (300 MHz, DMSO-d₆) δ: 7.06-7.14 (2H, m), 7.41 (1H, t, J=8.3 Hz), 11.53 (1H, s).

### Preparation 49

### 2-[4-Bromo-5-(4-fluorophenyl)-3-methyl-1H-pyrazol-1-yl]-N,N-dimethylethanamine

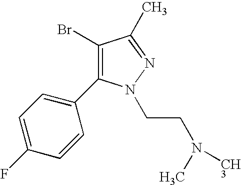

A mixture of 2-[4-bromo-5-(4-fluorophenyl)-3-methyl-1H-pyrazol-1-yl]ethyl4-methylbenzenesulfonate (300 mg) and dimethylamine (2M in tetrahydrofuran, 3.3 ml) in dimethyl sulfoxide was stirred at room temperature for 18 h. The mixture was diluted with ethyl acetate, and washed with saturated brine. The organic layer was separated, dried over anhydrous magnesium sulfate and concentrated. The residue was chromatographed on basic silica gel using hexane/ethyl acetate as an eluent to give the title compound (145 mg).

¹H-NMR (300 MHz, DMSO-d₆) δ: 1.97 (6H, s), 2.18 (3H, s), 2.45-2.50 (2H, m), 4.00 (2H, t, J=6.5 Hz), 7.34-7.46 (2H, m), 7.47-7.56 (2H, m).

### Preparation 50

### 4-{2-[4-Bromo-5-(4-fluorophenyl)-3-methyl-1H-pyrazol-1-yl]ethyl}morpholine

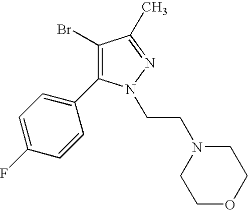

A mixture of 2-[4-bromo-5-(4-fluorophenyl)-3-methyl-1H-pyrazol-1-yl]ethyl4-methylbenzenesulfonate (300 mg), morpholine (115 mg) and potassium carbonate (183 mg) in N,N-dimethylformamide (5 ml) was stirred at 75° C. for 4 h. The mixture was diluted with ethyl acetate, and washed with saturated brine. The organic layer was separated, dried over anhydrous magnesium sulfate and concentrated. The residue was chromatographed on basic silica gel using hexane/ethyl acetate as an eluent to give the title compound (53 mg).

¹H-NMR (300 MHz, DMSO-d₆) δ: 2.10-2.22 (7H, m), 2.57 (2H, t, J=6.2 Hz), 3.42 (4H, t, J=4.5 Hz), 4.03 (2H, t, J=6.2 Hz), 7.39 (2H, t, J=8.9 Hz), 7.47-7.58 (2H, m).

### Preparation 51

### 3-[4-Bromo-5-(4-fluorophenyl)-3-methyl-1H-pyrazol-1-yl]propyl4-methylbenzenesulfonate

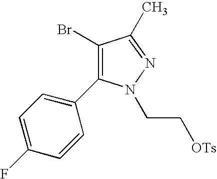

The title compound was obtained from 3-[4-bromo-5-(4-fluorophenyl)-3-methyl-1H-pyrazol-1-yl]propan-1-ol (2 g) according to the similar procedure described for Preparation 8 (1.7 g) as crude. The crude compound was used to next reaction without further purification.

### Preparation 52

### 4-Bromo-5-(4-fluorophenyl)-3-methyl-1-[3-(methylsulfanyl)propyl]-1H-pyrazole

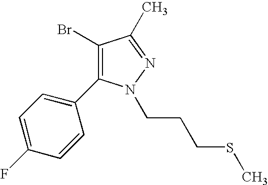

The title compound was obtained from 3-[4-bromo-5-(4-fluorophenyl)-3-methyl-1H-pyrazol-1-yl]propyl4-methylbenzenesulfonate (1 g) according to the similar procedure described for Preparation 9 (258 mg).

¹H-NMR (300 MHz, DMSO-d₆) δ: 1.81-1.91 (2H, m), 1.92 (3H, s), 2.19 (3H, s), 2.31 (2H, t, J=7.0 Hz), 4.03 (2H, t, J=7.0 Hz), 7.33-7.44 (2H, m), 7.45-7.55 (2H, m).

### Preparation 53

### 5-Chloro-2H-1,4-benzoxazin-3(4H)-one

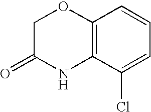

The title compound was obtained from 3-chloro-2-nitrophenol (9.56 g) according to the similar procedure described for Preparation 45 and 46 (6.53 g).

¹H-NMR (300 MHz, DMSO-d₆) δ: 4.61 (2H, s), 6.91-7.03 (2H, m), 7.10 (1H, dd, J=6.0, 3.2 Hz), 10.50 (1H, br. s).

### Preparation 54

### 6-Bromo-5-chloro-2H-1,4-benzoxazin-3(4H)-one

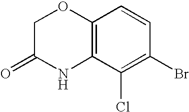

The title compound was obtained from 5-chloro-2H-1,4-benzoxazin-3(4H)-one (2 g) according to the similar procedure described for Preparation 5 (2.06 g).

¹H-NMR (300 MHz, DMSO-d₆) δ: 4.62 (2H, s), 6.96 (1H, d, J=8.7 Hz), 7.33 (1H, d, J=8.9 Hz), 10.65 (1H, s).

### Preparation 55

### 5-Chloro-6-(4,4,5,5-tetramethyl-1,3,2-dioxaborolan-2-yl)-2H-1,4-benzoxazin-3(4H)-one

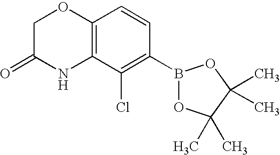

The title compound was obtained from 6-bromo-5-chloro-2H-1,4-benzoxazin-3(4H)-one (2 g) according to the similar procedure described for Preparation 6 (662 mg).

¹H-NMR (300 MHz, DMSO-d₆) δ: 1.29 (12H, s), 4.63 (2H, s), 6.96 (1H, d, J=8.1 Hz), 7.24 (1H, d, J=8.1 Hz), 10.36 (1H, br. s).

### Preparation 56

### 4,4,4-Trifluoro-1-(4-fluorophenyl)butane-1,3-dione

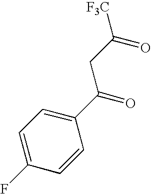

The title compound was obtained from ethyl trifluoroacetate (6.91 ml) according to the similar procedure described for Preparation 1 (quant.) as crude. The crude compound was used to next reaction without further purification.

### Preparation 57

### 5-(4-Fluorophenyl)-1-methyl-3-(trifluoromethyl)-1H-pyrazole

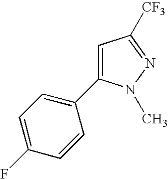

The title compound was obtained from 4,4,4-trifluoro-1-(4-fluorophenyl)butane-1,3-dione (2 g) according to the similar procedure described for Preparation 2 (710 mg).

¹H-NMR (300 MHz, DMSO-d₆) δ: 3.91 (3H, s), 6.91 (1H, s), 7.38 (2H, dd), 7.67 (2H, dd, J=8.9, 5.5 Hz).

### Preparation 58

### 4-Bromo-5-(4-fluorophenyl)-1-methyl-3-(trifluoromethyl)-1H-pyrazole

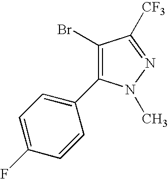

The title compound was obtained from 5-(4-fluorophenyl)-1-methyl-3-(trifluoromethyl)-1H-pyrazole (710 mg) according to the similar procedure described for Preparation 3 (797 mg).

¹H-NMR (300 MHz, DMSO-d₆) δ: 3.83 (3H, s), 7.44 (2H, m), 7.63 (2H, dd, J=8.9, 5.5 Hz).

### Preparation 59

### 5-(2,4-Difluorophenyl)-1,3-dimethyl-1H-pyrazole

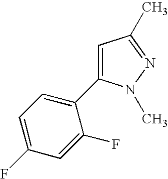

A solution of 1-(2,4-difluorophenyl)ethanone (2 g) in tetrahydrofuran (100 ml) was added lithium bis(trimethylsilyl)amide (1.1 M in tetrahydrofuran, 12.8 ml) at −25° C. dropwise, and stirred for 1 h at the temperature. After cooling to −78° C., to the mixture was added acetyl chloride (1.19 ml), and stirred for 3 h. The reaction mixture was acidified by addition of 1N hydrochloric acid, and added to ethyl acetate. The organic layer was separated, washed with saturated brine, dried over anhydrous magnesium sulfate and concentrated. A mixture of the residue, methyl hydrazine (1.35 ml), trifluoroacetic acid (1.99 ml), and triethylamine (3.61 ml) in isopropanol (100 ml) was heated at 80° C. for 30 min. The solvent was removed under reduced pressure. Then, the residue was diluted with ethyl acetate, and washed with aqueous sodium bicarbonate, and saturated brine. The organic layer was separated, washed with water, dried over anhydrous magnesium sulfate and concentrated. The residue was chromatographed on silica gel using hexane/ethyl acetate as an eluent to give the title compound (625 mg).

¹H-NMR (300 MHz, DMSO-d₆) δ: 2.18 (3H, s), 3.62 (3H, s), 6.17 (1H, s), 7.23 (1H, td, J=8.4, 2.5 Hz), 7.44 (1H, m), 7.53 (1H, m).

### Preparation 60

### 4-Bromo-5-(2,4-difluorophenyl)-1,3-dimethyl-1H-pyrazole

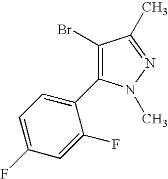

The title compound was obtained from 5-(2,4-difluorophenyl)-1,3-dimethyl-1H-pyrazole (625 mg) according to the similar procedure described for Preparation 3 (704 mg).

¹H-NMR (300 MHz, DMSO-d₆) δ: 2.18 (3H, s), 3.63 (3H, s), 7.30 (1H, td, J=8.5, 1.9 Hz), 7.43-7.62 (2H, m).

### Preparation 61

### 4-Bromo-5-(2-fluorophenyl)-1,3-dimethyl-1H-pyrazole

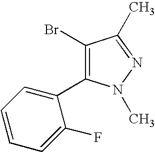

The title compound was obtained from 1-(2-fluorophenyl)ethanone (5 g) according to the similar procedure described for Preparation 1, 2 and 3 (3.07 g).

¹H-NMR (300 MHz, DMSO-d₆) δ: 2.18 (3H, s), 3.64 (3H, s), 7.17-7.54 (3H, m), 7.53-7.73 (1H, m).

### Preparation 62

### 4-Bromo-5-(3-fluorophenyl)-1,3-dimethyl-1H-pyrazole

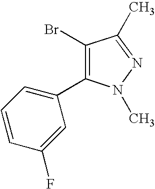

The title compound was obtained from 1-(3-fluorophenyl)ethanone (5 g) according to the similar procedure described for Preparation 1, 2 and 3 (7.85 g).

¹H-NMR (300 MHz, DMSO-d₆) δ: 2.18 (3H, s), 3.72 (3H, s), 7.27-7.44 (3H, m), 7.44-7.76 (1H, m).

### Preparation 63

### 5-(3,4-Difluorophenyl)-1,3-dimethyl-1H-pyrazole

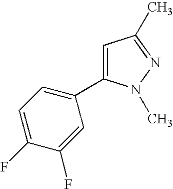

The title compound was obtained from 1-(3,4-difluorophenyl)ethanone (10 g) according to the similar procedure described for Preparation 59 (2.42 g).

¹H-NMR (300 MHz, DMSO-d₆) δ: 2.16 (3H, s), 3.76 (3H, s), 6.22 (1H, s), 7.20-7.45 (1H, m), 7.46-7.73 (2H, m).

### Preparation 64

### 5-(3,5-Difluorophenyl)-1,3-dimethyl-1H-pyrazole

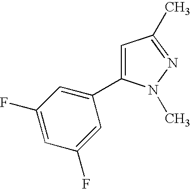

The title compound was obtained from 1-(3,5-difluorophenyl)ethanone (10 g) according to the similar procedure described for Preparation 59 (3.55 g).

¹H-NMR (300 MHz, DMSO-d₆) δ: 2.16 (3H, s), 3.80 (3H, s), 6.30 (1H, s), 7.07-7.46 (3H, m).

### Preparation 65

### 5-(2,3-Difluorophenyl)-1,3-dimethyl-1H-pyrazole

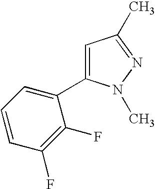

The title compound was obtained from 1-(2,3-difluorophenyl)ethanone (5 g) according to the similar procedure described for Preparation 59 (1.53 g).

¹H-NMR (300 MHz, DMSO-d₆) δ: 2.19 (3H, s), 3.67 (3H, s), 6.23 (1H, s), 7.25-7.42 (2H, m), 7.46-7.62 (1H, m).

### Preparation 66

### 5-(2,5-Difluorophenyl)-1,3-dimethyl-1H-pyrazole

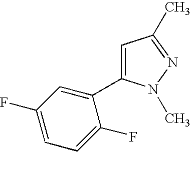

The title compound was obtained from 1-(2,5-difluorophenyl)ethanone (10 g) according to the similar procedure described for Preparation 59 (2.70 g).

¹H-NMR (300 MHz, DMSO-d₆) δ: 2.18 (3H, s), 3.67 (3H, s), 6.22 (1H, s), 7.27-7.53 (3H, m).

### Preparation 67

### 4-Bromo-5-(3,4-difluorophenyl)-1,3-dimethyl-1H-pyrazole

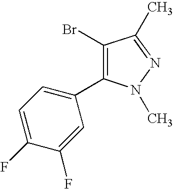

The title compound was obtained from 5-(3,4-difluorophenyl)-1,3-dimethyl-1H-pyrazole (2.42 g) according to the similar procedure described for Preparation 3 (2.98 g).

¹H-NMR (300 MHz, DMSO-d₆) δ: 2.17 (3H, s), 3.71 (3H, s), 7.28-7.46 (1H, m), 7.51-7.78 (2H, m).

### Preparation 68

### 4-Bromo-5-(3,5-difluorophenyl)-1,3-dimethyl-1H-pyrazole

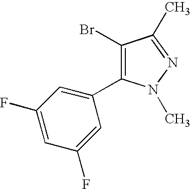

The title compound was obtained from 5-(3,5-difluorophenyl)-1,3-dimethyl-1H-pyrazole (3.55 g) according to the similar procedure described for Preparation 3 (3.12 g).

¹H-NMR (300 MHz, DMSO-d₆) δ: 2.18 (3H, s), 3.74 (3H, s), 7.22-7.36 (2H, m), 7.44 (1H, tt, J=9.5, 2.3 Hz).

### Preparation 69

### 5-(2,3-Difluorophenyl)-4-iodo-1,3-dimethyl-1H-pyrazole

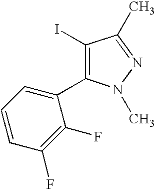

The title compound was obtained from 5-(2,3-difluorophenyl)-1,3-dimethyl-1H-pyrazole (700 mg) and N-iodosuccinimide (908 mg) according to the similar procedure described for Preparation 3 (quant.).

¹H-NMR (300 MHz, DMSO-d₆) δ: 2.19 (3H, s), 3.68 (3H, s), 7.18-7.33 (1H, m), 7.33-7.50 (1H, m), 7.53-7.77 (1H, m).

### Preparation 70

### 5-(2,5-Difluorophenyl)-4-iodo-1,3-dimethyl-1H-pyrazole

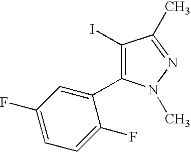

The title compound was obtained from 5-(2,5-difluorophenyl)-1,3-dimethyl-1H-pyrazole (700 mg) and N-iodosuccinimide (908 mg) according to the similar procedure described for Preparation 3 (830 mg).

¹H-NMR (300 MHz, DMSO-d₆) δ: 2.18 (3H, s), 3.67 (3H, s), 7.39 (1H, ddd, J=11.3, 2.8, 1.9 Hz), 7.48 (2H, dt, J=7.2, 4.5 Hz).

### Preparation 71

### Methyl(4-bromo-3,6-difluoro-2-nitrophenoxy)acetate

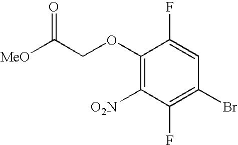

To a solution of 4-bromo-2,5-difluorophenol (6.5 g) in acetic acid (13 ml) was added concentrated nitric acid (1.68 ml) at −10° C., and stirred at room temperature for 18 h. The reaction mixture was diluted with ethyl acetate, and washed with water, and saturated brine. The organic layer was separated, dried over anhydrous magnesium sulfate and concentrated. To the solution of the residue in dimethyl sulfoxide (60 ml) was added potassium carbonate (12.9 g) and methyl bromoacetate (9.52 g), and stirred at 40° C. for 15 h. The reaction mixture was diluted with ethyl acetate, and washed with aqueous sodium bicarbonate, and saturated brine. The organic layer was separated, dried over anhydrous magnesium sulfate and concentrated. The residue was chromatographed on silica gel using ethyl acetate/hexane as an eluent to give the title compound (1.78 g).

¹H-NMR (300 MHz, DMSO-d₆) δ: 3.68 (3H, s), 5.05 (2H, d, J=1.9 Hz), 8.26 (1H, dd, J=11.7, 6.4 Hz).

### Preparation 72

### 6-Bromo-5,8-difluoro-2H-1,4-benzoxazin-3(4H)-one

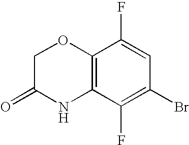

The title compound was obtained from methyl(4-bromo-3,6-difluoro-2-nitrophenoxy)acetate (1.78 g) according to the similar procedure described for Preparation 46 (1.09 g).

¹H-NMR (300 MHz, DMSO-d₆) δ: 4.72 (2H, s), 7.40 (1H, dd, J=9.8, 6.1 Hz), 11.28 (1H, br. s).

### Preparation 73

### 5,8-Difluoro-6-(4,4,5,5-tetramethyl-1,3,2-dioxaborolan-2-yl)-2H-1,4-benzoxazin-3(4H)-one

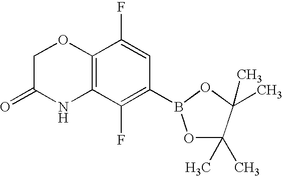

The title compound was obtained from 6-bromo-5,8-difluoro-2H-1,4-benzoxazin-3(4H)-one (1 g) according to the similar procedure described for Preparation 6 (1.03 g).

¹H-NMR (300 MHz, DMSO-d₆) δ: 1.28 (12H, s), 4.75 (2H, s), 6.97 (1H, dd, J=10.6, 4.5 Hz), 11.08 (1H, s).

### Preparation 74

### 3-(Difluoromethyl)-5-(4-fluorophenyl)-1-methyl-1H-pyrazole

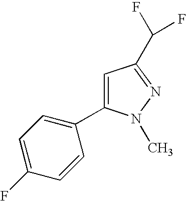

The title compound was obtained from ethyl difluoroacetate (10 g) according to the similar procedure described for Preparation 1 and 2 (2.14 g).

¹H-NMR (300 MHz, DMSO-d₆) δ: 3.87 (3H, s), 6.68 (1H, s), 6.99 (1H, t, J=54.5 Hz), 7.37 (2H, t, J=8.9 Hz), 7.64 (2H, dd, J=9.1, 5.3 Hz).

### Preparation 75

### 4-Bromo-3-(difluoromethyl)-5-(4-fluorophenyl)-1-methyl-1H-pyrazole

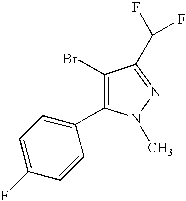

The title compound was obtained from 3-(difluoromethyl)-5-(4-fluorophenyl)-1-methyl-1H-pyrazole (2.14 g) according to the similar procedure described for Preparation 3 (2.85 g).

¹H-NMR (300 MHz, DMSO-d₆) δ: 3.80 (3H, s), 7.05 (1H, t, J=53.0 Hz), 7.43 (2H, t, J=8.9 Hz), 7.61 (2H, dd, J=8.7, 5.3 Hz).

### Preparation 76

### 4-Bromo-5-(4-fluorophenyl)-3-methyl-1-[2-(propan-2-ylsulfanyl)ethyl]-1H-pyrazole

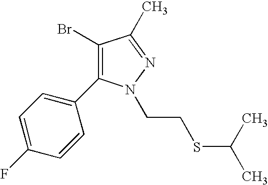

A mixture of 2-[4-bromo-5-(4-fluorophenyl)-3-methyl-1H-pyrazol-1-yl]ethyl4-methylbenzenesulfonate (300 mg), propane-2-thiol (60 mg) and potassium carbonate (110 mg) in N,N-dimethylformamide (10 ml) was stirred at 60° C. for 4 h. The reaction mixture was diluted with ethyl acetate, and washed with water, and saturated brine. The organic layer was separated, dried over anhydrous magnesium sulfate and concentrated. The residue was chromatographed on basic silica gel using ethyl acetate/hexane as an eluent to give the title compound (127 mg).

¹H-NMR (300 MHz, DMSO-d₆) δ: 1.04 (6H, d, J=6.6 Hz), 2.19 (3H, s), 2.61 (1H, dt, J=13.4, 6.7 Hz), 2.80 (2H, t, J=7.0 Hz), 4.09 (2H, t, J=7.0 Hz), 7.40 (2H, t, J=8.9 Hz), 7.47-7.64 (2H, m).

### Preparation 77

### 4-Bromo-5-(4-fluorophenyl)-3-methyl-1-(thiophen-3-ylmethyl)-1H-pyrazole

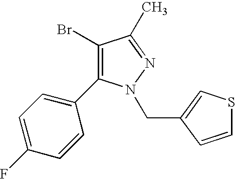

The title compound was obtained from thiophen-3-ylmethanol (448 mg) according to the similar procedure described for Preparation 21 (314 mg).

¹H-NMR (300 MHz, DMSO-d₆) δ: 2.20 (3H, s), 5.18 (2H, s), 6.77 (1H, d, J=4.9 Hz), 7.09 (1H, d, J=3.0 Hz), 7.29-7.52 (5H, m)

### Preparation 78

### 4-{[4-Bromo-5-(4-fluorophenyl)-3-methyl-1H-pyrazol-1-yl]methyl}-1,3-thiazole

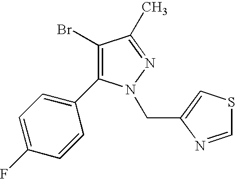

The title compound was obtained from 1,3-thiazol-4-ylmethanol (451 mg) according to the similar procedure described for Preparation 21 (131 mg).

¹H-NMR (300 MHz, DMSO-d₆) δ: 2.17 (3H, s), 5.28 (2H, s), 7.37 (2H, t, J=8.9 Hz), 7.45 (1H, d, J=1.9 Hz), 7.61 (2H, dd, J=8.9, 5.5 Hz), 9.03 (1H, d, J=2.3 Hz).

### Preparation 79

### 5-{[4-Bromo-5-(4-fluorophenyl)-3-methyl-1H-pyrazol-1-yl]methyl}-2,4-dimethyl-1,3-thiazole

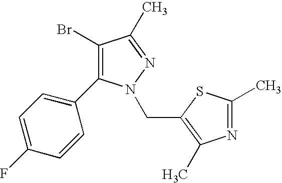

The title compound was obtained from (2,4-dimethyl-1,3-thiazol-5-yl)methanol (786 mg) according to the similar procedure described for Preparation 21 (515 mg).

¹H-NMR (300 MHz, DMSO-d₆) δ: 1.94 (3H, s), 2.18 (3H, s), 2.48 (3H, s), 5.29 (2H, s), 7.35-7.60 (4H, m).

### Preparation 80

### 5-{[4-Bromo-5-(4-fluorophenyl)-3-methyl-1H-pyrazol-1-yl]methyl}-2-methyl-1,3-thiazole

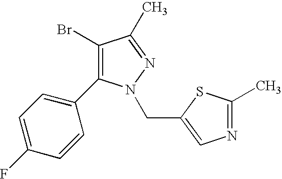

The title compound was obtained from (2-methyl-1,3-thiazol-5-yl)methanol (500 mg) according to the similar procedure described for Preparation 21 (185 mg).

¹H-NMR (300 MHz, DMSO-d₆) δ: 2.19 (3H, s), 2.55 (3H, s), 5.36 (2H, s), 7.27 (1H, s), 7.35-7.52 (4H, m).

### Preparation 81

### 5,5,5-Trifluoropentan-1-ol

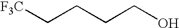

To a cold (−78° C.) solution of 5,5,5-trifluoropentanoic acid (2 g) in diethyl ether (15 ml) was added lithium aluminum hydride (729 mg), and stirred at −78° C. to room temperature for 18 h. The reaction mixture was quenched at 0° C. with solid sodium sulfate decahydrate until white. The precipitate was filtered, and washed with diethyl ether. The ether layer was concentrated to give the title compound (1.6 g)

¹H-NMR (300 MHz, DMSO-d₆) δ: 1.40-1.60 (4H, m), 2.10-2.35 (2H, m), 3.41 (2H, q, J=5.4 Hz), 4.45 (1H, t, J=5.1 Hz).

### Preparation 82

### 4-Bromo-5-(4-fluorophenyl)-3-methyl-1-(5,5,5-trifluoropentyl)-1H-pyrazole

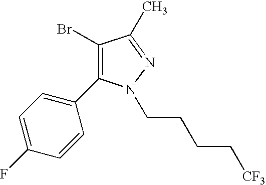

The title compound was obtained from 5,5,5-trifluoropentan-1-ol (557 mg) according to the similar procedure described for Preparation 21 (306 mg).

¹H-NMR (300 MHz, DMSO-d₆) δ: 1.23-1.40 (2H, m), 1.69 (2H, qd, J=7.4, 7.2 Hz), 2.05-2.17 (2H, m), 2.18 (3H, s), 3.98 (2H, t, J=7.1 Hz), 7.41 (2H, d, J=8.9 Hz), 7.44-7.53 (2H, m).

### Preparation 83

### (2E)-1-(2,4-Difluorophenyl)-3-(dimethylamino)prop-2-en-1-one

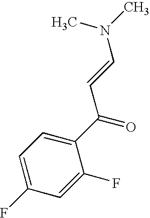

A mixture of 1-(2,4-difluorophenyl)ethanone (2 g) and 1,1-dimethoxy-N,N-dimethylmethanamine (4 ml) was refluxed for 1 h. The solvent was removed. The residue was chromatographed on silica gel using ethyl acetate/hexane as an eluent to give the title compound (1.06 g).

¹H-NMR (300 MHz, DMSO-d₆) δ: 2.85 (3H, s), 3.13 (3H, s), 5.44 (1H, d, J=12.5 Hz), 7.12 (1H, td, J=8.6, 2.1 Hz), 7.27 (1H, td, J=11.4, 1.9 Hz), 7.48-7.80 (2H, m).

### Preparation 84

### 5-(2,4-Difluorophenyl)-1-methyl-1H-pyrazole

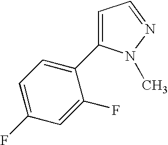

A mixture of (2E)-1-(2,4-difluorophenyl)-3-(dimethylamino)prop-2-en-1-one (1.05 g), methyl hydrazine (515 μl) and triethylamine (1.38 ml) in isopropanol (25 ml) was heated at 80° C. for 4 h. The solvent was removed under reduced pressure. The residue was chromatographed on basic silica gel using hexane/ethyl acetate as an eluent to give the title compound (761 mg).

¹H-NMR (300 MHz, DMSO-d₆) δ: 3.72 (3H, s), 6.40 (1H, d, J=1.1 Hz), 7.20-7.29 (1H, m), 7.47 (1H, ddd, J=10.5, 9.3, 2.4 Hz), 7.52 (1H, d, J=1.9 Hz), 7.57 (1H, td, J=8.7, 6.6 Hz).

### Preparation 85

### 4-Bromo-5-(2,4-difluorophenyl)-1-methyl-1H-pyrazole

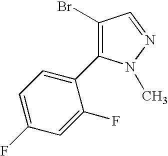

The title compound was obtained from 5-(2,4-difluorophenyl)-1-methyl-1H-pyrazole (761 mg) according to the similar procedure described for Preparation 3 (981 mg).

¹H-NMR (300 MHz, DMSO-d₆) δ: 3.71 (3H, s), 7.31 (1H, ddd, J=8.6, 7.3, 1.3 Hz), 7.44-7.65 (2H, m), 7.71 (1H, s).

### Preparation 86

### 2-[5-(4-Fluorophenyl)-1H-pyrazol-1-yl]ethanol

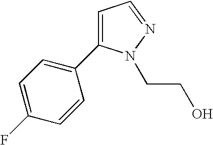

The title compound was obtained from (2E)-3-(dimethylamino)-1-(4-fluorophenyl)prop-2-en-1-one (2.63 g) and 2-hydrazinylethanol (1.55 g) according to the similar procedure described for Preparation 2 (2.52 g).

¹H-NMR (300 MHz, DMSO-d₆) δ: 3.77 (2H, q, J=5.8 Hz), 4.08 (2H, m), 4.93 (1H, t, J=5.4 Hz), 6.34 (1H, d, J=1.9 Hz), 7.33 (2H, t, J=8.9 Hz), 7.52 (1H, d, J=1.9 Hz), 7.62 (2H, m).

### Preparation 87

### 2-[4-Bromo-5-(4-fluorophenyl)-1H-pyrazol-1-yl]ethanol

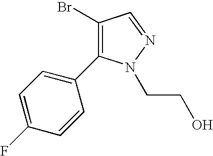

The title compound was obtained from 2-[5-(4-Fluorophenyl)-1H-pyrazol-1-yl]ethanol (2.52 g) according to the similar procedure described for Preparation 3 (3.39 g).

¹H-NMR (300 MHz, DMSO-d₆) δ: 3.70 (2H, q, J=5.6 Hz), 4.02 (2H, m), 4.89 (1H, t, J=5.6 Hz), 7.39 (2H, t, J=8.9 Hz), 7.57 (2H, dd, J=8.9, 5.5 Hz), 7.70 (1H, s).

### Preparation 88

### 4-Bromo-5-(4-fluorophenyl)-1-[2-(methylsulfanyl)ethyl]-1H-pyrazole

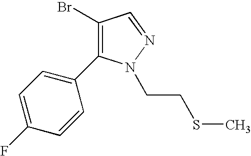

The title compound was obtained from 2-[4-Bromo-5-(4-fluorophenyl)-1H-pyrazol-1-yl]ethanol (2 g) according to the similar procedure described for Preparation 8, 9 and 3 (1.63 g).

¹H-NMR (300 MHz, DMSO-d₆) δ: 1.79 (3H, s), 2.79 (2H, t, J=6.8 Hz), 4.19 (2H, t, J=6.8 Hz), 7.35-7.47 (2H, m), 7.50-7.58 (2H, m), 7.72 (1H, s).

### Preparation 89

### 1-[5-(4-Fluorophenyl)-1H-pyrazol-1-yl]propan-2-ol

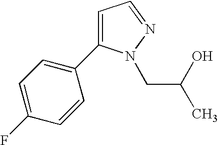

The title compound was obtained from (2E)-3-(dimethylamino)-1-(4-fluorophenyl)prop-2-en-1-one (3.31 g) and 1-hydrazinylpropan-2-ol (1.7 g) according to the similar procedure described for Preparation 2 (3.49 g).

¹H-NMR (300 MHz, DMSO-d₆) δ: 0.96 (3H, d, J=6.0 Hz), 3.83-3.95 (1H, m), 3.96-4.14 (2H, m), 4.93 (1H, d, J=4.7 Hz), 6.34 (1H, d, J=1.9 Hz), 7.32 (2H, dd, J=8.9, 4.6 Hz), 7.51 (1H, d, J=1.9 Hz), 7.61 (2H, dd, J=8.9, 5.5 Hz).

### Preparation 90

### 1-[4-Bromo-5-(4-fluorophenyl)-1H-pyrazol-1-yl]propan-2-ol

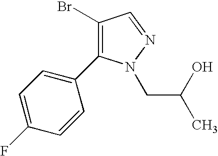

The title compound was obtained from 1-[5-(4-fluorophenyl)-1H-pyrazol-1-yl]propan-2-ol (3.49 g) according to the similar procedure described for Preparation 3 (4.13 g).

¹H-NMR (300 MHz, DMSO-d₆) δ: 0.95 (3H, d, J=6.0 Hz), 3.84-4.02 (3H, m), 4.91 (1H, d, J=4.9 Hz), 7.39 (2H, dd, J=8.9, 4.6 Hz), 7.56 (2H, dd, J=8.9, 5.5 Hz), 7.69 (1H, s).

### Preparation 91

### 1-[4-Bromo-5-(4-fluorophenyl)-1H-pyrazol-1-yl]propan-2-one

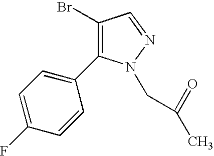

The title compound was obtained from 1-[4-bromo-5-(4-fluorophenyl)-1H-pyrazol-1-yl]propan-2-ol (4.13 g) according to the similar procedure described for Preparation 19 (3.32 g).

¹H-NMR (300 MHz, DMSO-d₆) δ: 2.04 (3H, s), 5.09 (2H, s), 7.29-7.46 (4H, m), 7.73 (1H, s).

### Preparation 92

### 4-Bromo-1-(2,2-difluoropropyl)-5-(4-fluorophenyl)-1H-pyrazole

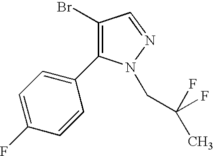

The title compound was obtained from 1-[4-Bromo-5-(4-fluorophenyl)-1H-pyrazol-1-yl]propan-2-one (3.32 g) according to the similar procedure described for Preparation 20 (2.98 g).

¹H-NMR (300 MHz, DMSO-d₆) δ: 1.53 (3H, t, J=19.3 Hz), 4.54 (2H, t, J=13.1 Hz), 7.33-7.45 (2H, m), 7.45-7.55 (2H, m), 7.81 (1H, s).

### Preparation 93

### 4-Bromo-5-(4-fluorophenyl)-1-(2-iodoethyl)-3-methyl-1H-pyrazole

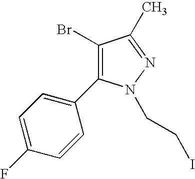

To a mixture of 2-[4-bromo-5-(4-fluorophenyl)-3-methyl-1H-pyrazol-1-yl]ethanol (5 g) and triethyl amine (3.37 ml) in ethyl acetate (50 ml) was added methanesulfonyl chloride (1.62 ml), and stirred at room temperature for 6 h. The reaction mixture was diluted with ethyl acetate, and washed with water, and saturated brine. The organic layer was separated, dried over anhydrous magnesium sulfate and concentrated under reduced pressure. To the solution of the residue in acetone (50 ml) was added sodium iodide (3.76 g), and refluxed for 18 h. The reaction mixture was diluted with ethyl acetate, and washed with water, and saturated brine. The organic layer was separated, dried over anhydrous magnesium sulfate and concentrated. The residue was chromatographed on silica gel using hexane/ethyl acetate as an eluent to give the title compound (5 g).

¹H-NMR (300 MHz, DMSO-d₆) δ: 2.20 (3H, s), 3.47 (2H, t, J=6.6 Hz), 4.26 (2H, t, J=6.6 Hz), 7.35-7.47 (2H, m), 7.47-7.59 (2H, m).

### Preparation 94

### [4-(Benzyloxy)butyl]hydrazine hydrochloride

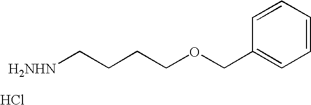

To a mixture of hydrazine hydrate (11.6 g) and sodium hydroxide (2.01 g) was added [(4-chlorobutoxy)methyl]benzene (10 g) at 95° C., and the mixture was stirred for 2 h at the temperature. The solvent was removed to give oil. To the oil was added concentrated hydrochloric acid (5 ml) and water (120 ml). The mixture was extracted with diethyl ether, and the ether solution was concentrated to give the title compound (9.6 g).

¹H-NMR (300 MHz, DMSO-d₆) δ: 1.50-1.77 (4H, m), 2.86-3.05 (2H, m), 3.43 (2H, t, J=5.4 Hz), 4.45 (2H, s), 6.60 (3H, br. s), 7.23-7.37 (5H, m).

### Preparation 95

### 1-[4-(Benzyloxy)butyl]-5-(4-fluorophenyl)-3-methyl-1H-pyrazole

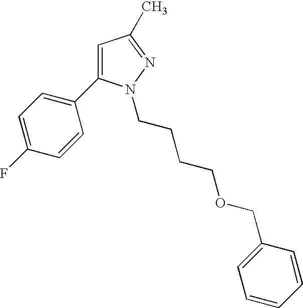

A mixture of 1-(4-fluorophenyl)butane-1,3-dione (0.5 g), [4-(benzyloxy)butyl]hydrazine hydrochloride (0.77 g) and concentrated hydrochloric acid (0.56 ml) in ethanol (20 ml) was heated at 40° C. for 14 h. The solvent was removed under reduced pressure, and the residue was dissolved with ethyl acetate. The solution was washed with saturated aqueous sodium bicarbonate, water, and saturated brine successively, dried over anhydrous magnesium sulfate, and concentrated under reduced pressure. The residue was chromatographed on silica gel using hexane/ethyl acetate as an eluent to give the title compound (0.46 g).

¹H-NMR (300 MHz, CDCl₃) δ: 1.40-1.65 (2H, m), 1.81-1.93 (2H, m), 2.29 (3H, s), 3.37 (2H, t, J=6.3 Hz), 4.02 (2H, t, J=7.2 Hz), 4.41 (2H, s), 6.00 (1H, s), 7.03-7.12 (2H, m), 7.23-7.35 (7H, m).

### Preparation 96

### 1-[4-(Benzyloxy)butyl]-4-bromo-5-(4-fluorophenyl)-3-methyl-1H-pyrazole

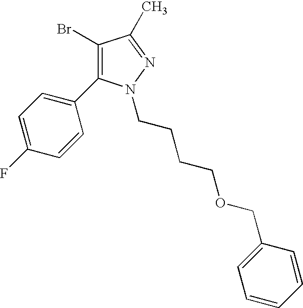

The title compound was obtained from 1-[4-(benzyloxy)butyl]-5-(4-fluorophenyl)-3-methyl-1H-pyrazole (3.74 g) according to the similar procedure described for Preparation 3 (4.0 g).

¹H-NMR (300 MHz, CDCl₃) δ: 1.42-1.55 (2H, m), 1.75-1.87 (2H, m), 2.28 (3H, s), 3.36 (2H, t, J=6.3 Hz), 3.90 (2H, t, J=7.2 Hz), 4.41 (2H, s), 7.09-7.18 (2H, m), 7.23-7.36 (7H, m).

### Example 1

### 6-[5-(4-Fluorophenyl)-1,3-dimethyl-1H-pyrazol-4-yl]-2H-1,4-benzoxazin-3(4H)-one

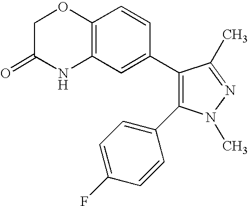

A mixture of 6-(4,4,5,5-tetramethyl-1,3,2-dioxaborolan-2-yl)-2H-1,4-benzoxazin-3(4H)-one (245 mg), 4-bromo-5-(4-fluorophenyl)-1,3-dimethyl-1H-pyrazole (200 mg), Cesium carbonate (726 mg), and [1,1′-bis(diphenylphosphino)ferrocene]dichloropalladium(II) dichloromethane adduct (61 mg) in tetrahydrofuran/H₂O (5/1) (4 ml) was exposed to microwave irradiation at 150 to 160° C. for 15 min. The reaction mixture was diluted with ethyl acetate, and washed with saturated brine. The organic layer was separated, dried over anhydrous magnesium sulfate and concentrated. The residue was chromatographed on silica gel using ethyl acetate/hexane as an eluent and followed by recrystallization from ethyl acetate/hexane to give the title compound (55 mg).

¹H-NMR (DMSO-d₆) δ: 2.17 (3H, s), 3.64 (3H, s), 4.53 (2H, s), 6.57 (1H, dd, J=8.1, 2.1 Hz), 6.65 (1H, d, J=1.9 Hz), 6.83 (1H, d, J=8.3 Hz), 7.18-7.42 (4H, m), 10.58 (1H, s).

LCMS (ESI⁺) M+H⁺: 338.03.

### Example 2

### 8-Fluoro-6-[5-(4-fluorophenyl)-1,3-dimethyl-1H-pyrazol-4-yl]-2H-1,4-benzoxazin-3(4H)-one

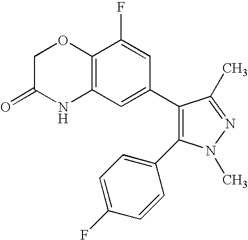

The title compound was obtained from 8-fluoro-6-(4,4,5,5-tetramethyl-1,3,2-dioxaborolan-2-yl)-2H-1,4-benzoxazin-3(4H)-one (500 mg) according to the similar procedure described for Example 1 (185 mg).

¹H-NMR (DMSO-d₆) δ: 2.18 (3H, s), 3.64 (3H, s), 4.63 (2H, s), 6.43-6.48 (1H, m), 6.53 (1H, dd, J=11.7, 1.9 Hz), 7.21-7.41 (4H, m), 10.79 (1H, br. s).

LCMS (ESI⁺) M+H⁺: 356.06.

### Example 3

### 6-[5-(4-Fluorophenyl)-1,3-dimethyl-1H-pyrazol-4-yl]-5-methyl-2H-1,4-benzoxazin-3(4H)-one

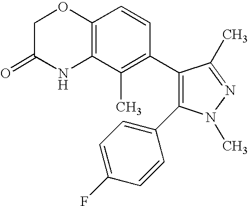

A mixture of 4-bromo-5-(4-fluorophenyl)-1,3-dimethyl-1H-pyrazole (1.43 g), 5-methyl-6-(4,4,5,5-tetramethyl-1,3,2-dioxaborolan-2-yl)-2H-1,4-benzoxazin-3(4H)-one (2 g), phenylallylchloro-[1,3-bis(diisopropylphenyl)-2-imidazolidinylidene]palladium(II) (346 mg) and potassium tert-butoxide (1.19 g) in 1,2-dimethoxyethane (55 ml) was well evacuated, and stirred at 100° C. for 15 h. The reaction mixture was diluted with ethyl acetate, and washed with saturated brine. The organic layer was separated, dried over anhydrous magnesium sulfate and concentrated. The residue was chromatographed on silica gel, and basic silica gel using hexane/ethyl acetate as an eluent, and crystallized from hexane/ethyl acetate to give the title compound (222 mg).

¹H-NMR (DMSO-d₆) δ: 1.82 (3H, s), 1.96 (3H, s), 3.70 (3H, s), 4.50 (2H, s), 6.67 (1H, d, J=8.3 Hz), 6.77 (1H, d, J=8.3 Hz), 7.13-7.29 (4H, m), 10.09 (1H, s).

LCMS (ESI⁺) M+H⁺: 352.0.

### Example 4

### 6-{5-(4-Fluorophenyl)-3-methyl-1-[2-(methylsulfanyl)ethyl]-1H-pyrazol-4-yl}-2H-1,4-benzoxazin-3(4H)-one

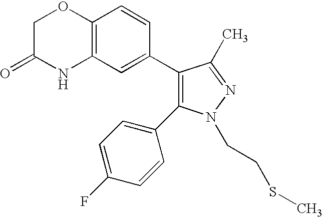

The title compound was obtained from 4-bromo-5-(4-fluorophenyl)-3-methyl-1-[2-(methylsulfanyl)ethyl]-1H-pyrazole (300 mg) according to the similar procedure described for Example 1 (190 mg).

¹H-NMR (DMSO-d₆) δ: 1.83 (3H, s), 2.19 (3H, s), 2.80 (2H, t, J=7.0 Hz), 4.06 (2H, t, J=7.0 Hz), 4.53 (2H, s), 6.57 (1H, dd, J=8.3, 2.3 Hz), 6.66 (1H, d, J=1.9 Hz), 6.82 (1H, d, J=8.3 Hz), 7.20-7.42 (4H, m), 10.61 (1H, s).

LCMS (ESI⁺) M+H⁺: 397.84.

### Example 5

### 6-{5-(4-Fluorophenyl)-3-methyl-1-[2-(methylsulfinyl)ethyl]-1H-pyrazol-4-yl}-2H-1,4-benzoxazin-3(4H)-one

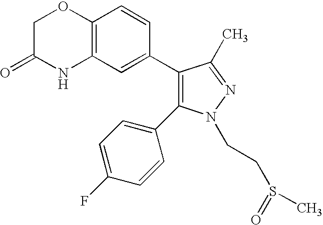

The title compound was obtained from 4-bromo-5-(4-fluorophenyl)-3-methyl-1-[2-(methylsulfinyl)ethyl]-1H-pyrazole (300 mg) according to the similar procedure described for Example 1 (203 mg).

¹H-NMR (DMSO-d₆) δ: 2.20 (3H, s), 2.52 (3H, br. s), 2.98-3.12 (1H, m), 3.25-3.31 (1H, m), 4.21-4.32 (2H, m), 4.54 (2H, s), 6.58 (1H, dd, J=8.3, 1.9 Hz), 6.65 (1H, d, J=1.9 Hz), 6.83 (1H, d, J=8.3 Hz), 7.22-7.41 (4H, m), 10.61 (1H, s).

LCMS (ESI⁺) M+H⁺: 413.75.

### Example 6

### 6-{5-(4-Fluorophenyl)-3-methyl-1-[2-(methylsulfonyl)ethyl]-1H-pyrazol-4-yl}-2H-1,4-benzoxazin-3(4H)-one

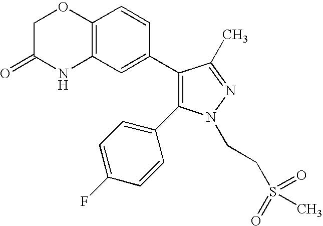

The title compound was obtained from 4-bromo-5-(4-fluorophenyl)-3-methyl-1-[2-(methylsulfonyl)ethyl]-1H-pyrazole (125 mg) according to the similar procedure described for Example 1 (75 mg).

¹H-NMR (DMSO-d₆) δ: 2.20 (3H, s), 2.92 (3H, s), 3.66 (2H, t, J=7.2 Hz), 4.29 (2H, t, J=7.2 Hz), 4.54 (2H, s), 6.58 (1H, dd, J=8.3, 1.9 Hz), 6.65 (1H, d, J=2.3 Hz), 6.83 (1H, d, J=8.3 Hz), 7.23-7.43 (4H, m), 10.61 (1H, s).

LCMS (ESI⁺) M+H⁺: 429.78.

### Example 7

### 6-[5-(4-Fluorophenyl)-1-(3-hydroxypropyl)-3-methyl-1H-pyrazol-4-yl]-2H-1,4-benzoxazin-3(4H)-one

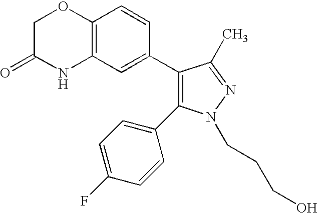

The title compound was obtained from 3-[4-bromo-5-(4-fluorophenyl)-3-methyl-1H-pyrazol-1-yl]propan-1-ol (100 mg) according to the similar procedure described for Example 1 (21 mg).

¹H-NMR (DMSO-d₆) δ: 1.73-1.90 (2H, m), 2.18 (3H, s), 3.27-3.40 (2H, m), 3.94 (2H, t, J=7.2 Hz), 4.47 (1H, t, J=4.9 Hz), 4.53 (2H, s), 6.56 (1H, dd, J=8.1, 2.1 Hz), 6.64 (1H, d, J=1.9 Hz), 6.81 (1H, d, J=8.0 Hz), 7.17-7.41 (4H, m), 10.60 (1H, s).

LCMS (ESI⁺) M+H⁺: 381.96.

### Example 8

### 6-{5-(4-Fluorophenyl)-3-methyl-1-[2-(1H-pyrazol-1-yl)ethyl]-1H-pyrazol-4-yl}-2H-1,4-benzoxazin-3(4H)-one

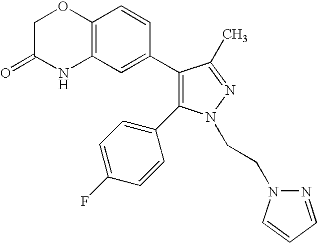

The title compound was obtained from 4-bromo-5-(4-fluorophenyl)-3-methyl-1-[2-(1H-pyrazol-1-yl)ethyl]-1H-pyrazole (103 mg) according to the similar procedure described for Example 1 (50 mg).

¹H-NMR (DMSO-d₆) δ: 2.22 (3H, s), 4.20 (2H, t, J=5.9 Hz), 4.41-4.60 (4H, m), 6.12-6.23 (1H, m), 6.52 (1H, dd, J=8.3, 1.9 Hz), 6.60 (1H, s), 6.80 (1H, d, J=8.0 Hz), 6.93 (2H, dd, J=8.3, 5.7 Hz), 7.18 (2H, t, J=8.7 Hz), 7.31-7.44 (2H, m), 10.57 (1H, s).

LCMS (ESI⁺) M+H⁺: 417.88.

### Example 9

### 6-[5-(4-Fluorophenyl)-3-methyl-1-(3,3,3-trifluoropropyl)-1H-pyrazol-4-yl]-2H-1,4-benzoxazin-3(4H)-one

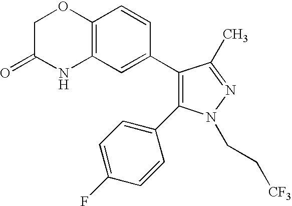

The title compound was obtained from 4-bromo-5-(4-fluorophenyl)-3-methyl-1-(3,3,3-trifluoropropyl)-1H-pyrazole (116 mg) according to the similar procedure described for Example 1 (46 mg).

¹H-NMR (DMSO-d₆) δ: 2.20 (3H, s), 2.81 (2H, m), 4.12 (2H, t, J=7.0 Hz), 4.54 (2H, s), 6.57 (1H, dd, J=8.1, 2.1 Hz), 6.65 (1H, d, J=1.9 Hz), 6.83 (1H, d, J=8.3 Hz), 7.20-7.39 (4H, m), 10.61 (1H, s).

LCMS (ESI⁺) M+H⁺: 419.75.

### Example 10

### 6-[1-(3,3-Difluoropropyl)-5-(4-fluorophenyl)-3-methyl-1H-pyrazol-4-yl]-2H-1,4-benzoxazin-3(4H)-one

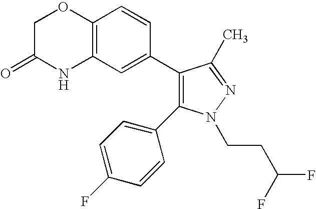

The title compound was obtained from 4-bromo-1-(3,3-difluoropropyl)-5-(4-fluorophenyl)-3-methyl-1H-pyrazole (34 mg) according to the similar procedure described for Example 1 (7 mg).

¹H-NMR (CDCl₃) δ: 2.30 (3H, s), 2.31-2.47 (1H, m), 4.13 (2H, t, J=7.0 Hz), 4.59 (2H, m), 5.87 (1H, tt, J=56.0, 4.2 Hz), 6.44 (1H, d, J=1.9 Hz), 6.68 (1H, dd, J=8.3, 1.9 Hz), 6.86 (1H, d, J=8.3 Hz), 7.05-7.14 (2H, m), 7.14-7.22 (2H, m), 7.67 (1H, br. s).

LCMS (ESI⁺) M+H⁺: 402.1.

### Example 11

### 6-[5-(4-Fluorophenyl)-1-(3-fluoropropyl)-3-methyl-1H-pyrazol-4-yl]-2H-1,4-benzoxazin-3(4H)-one

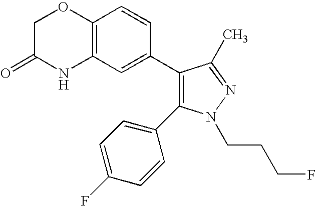

The title compound was obtained from 4-bromo-5-(4-fluorophenyl)-1-(3-fluoropropyl)-3-methyl-1H-pyrazole (600 mg) according to the similar procedure described for Example 1 (77 mg).

¹H-NMR (300 MHz, DMSO-d₆) δ: 1.94-2.16 (2H, m), 2.19 (3H, s), 3.99 (2H, t, J=7.0 Hz), 4.40 (2H, dt, J=47.0, 5.8 Hz), 4.53 (2H, s), 6.57 (1H, dd, J=8.3, 1.9 Hz), 6.65 (1H, d, J=1.9 Hz), 6.82 (1H, d, J=8.3 Hz), 7.19-7.39 (4H, m), 10.59 (1H, s).

LCMS (ESI⁺) M+H⁺: 384.0.

### Example 12

### 6-[5-(4-Fluorophenyl)-3-methyl-1-(thiophen-2-ylmethyl)-1H-pyrazol-4-yl]-2H-1,4-benzoxazin-3(4H)-one

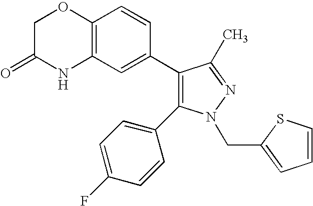

The title compound was obtained from 4-bromo-5-(4-fluorophenyl)-3-methyl-1-(thiophen-2-ylmethyl)-1H-pyrazole (295 mg) according to the similar procedure described for Example 1 (113 mg).

¹H-NMR (300 MHz, DMSO-d₆) δ: 2.20 (3H, s), 4.53 (2H, s), 5.31 (2H, s), 6.58 (1H, dd, J=8.3, 1.9 Hz), 6.66 (1H, d, J=1.9 Hz), 6.77 (1H, d, J=2.7 Hz), 6.82 (1H, d, J=8.3 Hz), 6.87-6.96 (1H, m), 7.21-7.34 (4H, m), 7.39 (1H, d, J=4.2 Hz), 10.59 (1H, s).

LCMS (ESI⁺) M+H⁺: 419.74.

### Example 13

### 6-[5-(4-Fluorophenyl)-3-methyl-1-(1,3-thiazol-5-ylmethyl)-1H-pyrazol-4-yl]-2H-1,4-benzoxazin-3(4H)-one

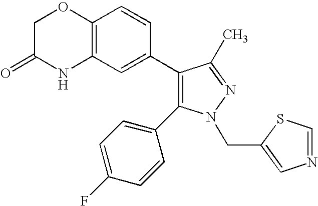

The title compound was obtained from 5-{[4-bromo-5-(4-fluorophenyl)-3-methyl-1H-pyrazol-1-yl]methyl}-1,3-thiazole (235 mg) according to the similar procedure described for Example 1 (174 mg).

¹H-NMR (300 MHz, DMSO-d₆) δ: 2.20 (3H, s), 4.53 (2H, s), 5.40 (2H, s), 6.58 (1H, dd, J=8.3, 2.1 Hz), 6.65 (1H, d, J=2.1 Hz), 6.82 (1H, d, J=8.3 Hz), 7.27 (2H, s), 7.30 (2H, d, J=1.1 Hz), 7.57 (1H, s), 8.97 (1H, d, J=0.8 Hz), 10.59 (1H, s).

LCMS (ESI⁺) M+H⁺: 420.80.

### Example 14

### 6-[5-(4-Fluorophenyl)-3-methyl-1-(4,4,4-trifluorobutyl)-1H-pyrazol-4-yl]-2H-1,4-benzoxazin-3(4H)-one

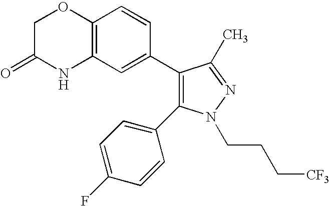

The title compound was obtained from 4-bromo-5-(4-fluorophenyl)-3-methyl-1-(4,4,4-trifluorobutyl)-1H-pyrazole (303 mg) according to the similar procedure described for Example 1 (18 mg).

¹H-NMR (300 MHz, DMSO-d₆) δ: 1.89 (2H, m), 2.10-2.33 (5H, m), 3.96 (2H, t, J=7.0 Hz), 4.53 (2H, s), 6.58 (1H, dd, J=8.3, 1.9 Hz), 6.66 (1H, d, J=1.9 Hz), 6.82 (1H, d, J=8.3 Hz), 7.22-7.40 (4H, m), 10.60 (1H, s).

LCMS (ESI⁺) M+H⁺: 433.75.

### Example 15

### 6-[1-(4-Fluorobutyl)-5-(4-fluorophenyl)-3-methyl-1H-pyrazol-4-yl]-2H-1,4-benzoxazin-3(4H)-one

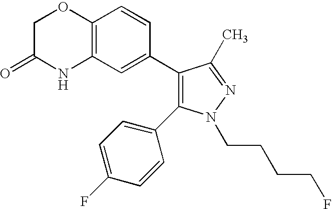

A mixture of 4-bromo-1-(4-fluorobutyl)-5-(4-fluorophenyl)-3-methyl-1H-pyrazole (640 mg), 6-(4,4,5,5-tetramethyl-1,3,2-dioxaborolan-2-yl)-2H-1,4-benzoxazin-3(4H)-one (802 mg), 2M aqueous cesium carbonate (1.94 ml), and [1,1′-bis(diphenylphosphino)ferrocene]dichloropalladium(II) dichloromethane adduct (318 mg) in 1,2-dimethoxyethane (6.5 ml) was well evacuated, and refluxed for 8 h under argon atmosphere. The reaction mixture was diluted with ethyl acetate, and washed with water, and saturated brine. The organic layer was separated, dried over anhydrous magnesium sulfate and concentrated. The residue was chromatographed on basic silica gel using hexane/ethyl acetate as an eluent, and crystallized from hexane/ethyl acetate to give the title compound (247 mg).

¹H-NMR (300 MHz, DMSO-d₆) δ: 1.40-1.63 (2H, m), 1.73 (2H, qd, J=7.3, 7.0 Hz), 2.19 (3H, s), 3.92 (2H, t, J=7.0 Hz), 4.33 (2H, dt, J=47.3, 5.9 Hz), 4.53 (2H, s), 6.57 (1H, d, J=8.7 Hz), 6.65 (1H, s), 6.81 (1H, d, J=8.3 Hz), 7.21-7.36 (4H, m), 10.59 (1H, s).

LCMS (ESI⁺) M+H⁺: 397.82.

### Example 16

### 6-[1-(3,3-Difluorobutyl)-5-(4-fluorophenyl)-3-methyl-1H-pyrazol-4-yl]-2H-1,4-benzoxazin-3(4H)-one

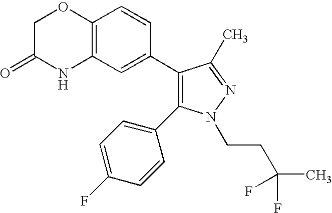

The title compound was obtained from 4-bromo-1-(3,3-difluorobutyl)-5-(4-fluorophenyl)-3-methyl-1H-pyrazole (70 mg) according to the similar procedure described for Example 15 (25 mg).

¹H-NMR (300 MHz, DMSO-d₆) δ: 1.53 (3H, t, J=19.2 Hz), 2.19 (3H, s), 2.38 (2H, m), 4.00-4.11 (2H, m), 4.53 (2H, s), 6.57 (1H, dd, J=8.3, 2.1 Hz), 6.65 (1H, d, J=2.1 Hz), 6.82 (1H, d, J=8.3 Hz), 7.24-7.39 (4H, m), 10.59 (1H, s).

### Example 17

### 4-[5-(4-Fluorophenyl)-3-methyl-4-(3-oxo-3,4-dihydro-2H-1,4-benzoxazin-6-yl)-1H-pyrazol-1-yl]butanal

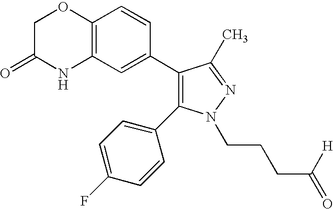

To a mixture of 6-[5-(4-fluorophenyl)-1-(4-hydroxybutyl)-3-methyl-1H-pyrazol-4-yl]-2H-1,4-benzoxazin-3(4H)-one (1 g) and triethylamine (5 ml) in dimethyl sulfoxide (7 ml) was added pyridine sulfur trioxide (1.61 g), and stirred at room temperature for 3 h. The mixture was diluted with ethyl acetate, and washed with water, and saturated brine. The organic layer was separated, dried over anhydrous magnesium sulfate and concentrated. The residue was chromatographed on silica gel using hexane/ethyl acetate as an eluent to give the title compound (496 mg).

¹H-NMR (300 MHz, DMSO-d₆) δ: 1.89 (2H, quin, J=7.0 Hz), 2.19 (3H, s), 2.38 (2H, t, J=7.1 Hz), 3.89 (2H, t, J=7.0 Hz), 4.53 (2H, s), 6.56 (1H, dd, J=8.2, 2.0 Hz), 6.65 (1H, d, J=2.1 Hz), 6.81 (1H, d, J=8.3 Hz), 7.22-7.36 (4H, m), 9.52 (1H, s), 10.59 (1H, s).

LCMS (ESI⁺) M+H⁺: 393.85.

### Example 18

### 6-[5-(4-Fluorophenyl)-3-methyl-1-(5,5,5-trifluoro-4-hydroxypentyl)-1H-pyrazol-4-yl]-2H-1,4-benzoxazin-3(4H)-one

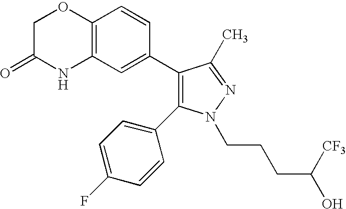

To a cold (0° C.) mixture of 4-[5-(4-fluorophenyl)-3-methyl-4-(3-oxo-3,4-dihydro-2H-1,4-benzoxazin-6-yl)-1H-pyrazol-1-yl]butanal (80 mg) and trimethyl(trifluoromethyl)silane (289 mg) in tetrahydrofuran (2 ml) was added a N,N,N-tributylbutan-1-aminium fluoride solution in tetrahydrofuran (1 ml), and stirred at room temperature for 1 h. The mixture was diluted with ethyl acetate, and washed with water, and saturated brine. The organic layer was separated, dried over anhydrous magnesium sulfate and concentrated. The residue was chromatographed on silica gel using hexane/ethyl acetate as an eluent to give the title compound (43 mg).

¹H-NMR (300 MHz, DMSO-d₆) δ: 1.26-1.51 (2H, m), 1.64-1.94 (2H, m), 2.19 (3H, s), 3.81 (1H, br. s), 3.93 (2H, t, J=7.1 Hz), 4.53 (2H, s), 6.06 (1H, d, J=4.7 Hz), 6.57 (1H, dd, J=8.2, 2.0 Hz), 6.65 (1H, d, (7=1.9 Hz), 6.81 (1H, d, J=8.1 Hz), 7.23-7.33 (4H, m), 10.59 (1H, s).

LCMS (ESI⁺) M+H⁺: 463.81.

### Example 19

### 6-[5-(4-Fluorophenyl)-1-(4-hydroxypentyl)-3-methyl-1H-pyrazol-4-yl]-2H-1,4-benzoxazin-3(4H)-one

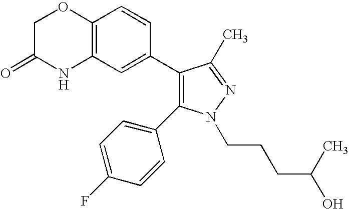

To a cold (−78° C.) solution of 6-[5-(4-fluorophenyl)-1-(4-hydroxybutyl)-3-methyl-1H-pyrazol-4-yl]-2H-1,4-benzoxazin-3(4H)-one (387 mg) in tetrahydrofuran (5 ml) was added 1M bromo(methyl)magnesium in tetrahydrofuran (2.35 ml) dropwise, and stirred at −78° C. to room temperature for 18 h. The reaction mixture was poured into ice-water, and extracted with ethyl acetate. The organic layer was washed with saturated brine, dried over anhydrous magnesium sulfate and concentrated. The residue was chromatographed on silica gel using hexane/ethyl acetate as an eluent, and crystallized from hexane/ethyl acetate to give the title compound (35 mg).

¹H-NMR (300 MHz, DMSO-d₆) δ: 0.95 (3H, d, J=6.2 Hz), 1.10-1.24 (2H, m), 1.54-1.81 (2H, m), 2.19 (3H, s), 3.39-3.52 (1H, m), 3.87 (2H, t, J=7.3 Hz), 4.32 (1H, d, J=4.7 Hz), 4.53 (2H, s), 6.56 (1H, dd, J=8.3, 2.1 Hz), 6.65 (1H, d, J=1.9 Hz), 6.81 (1H, d, J=8.3 Hz), 7.24-7.32 (4H, m), 10.59 (1H, s).

LCMS (ESI⁺) M+H⁺: 409.80.

### Example 20

### 6-[5-(4-Fluorophenyl)-1-(2-hydroxybutyl)-3-methyl-1H-pyrazol-4-yl]-2H-1,4-benzoxazin-3(4H)-one

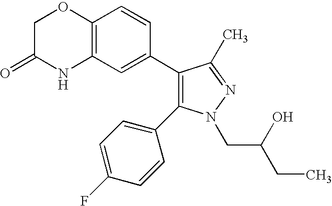

The title compound was obtained from 1-[4-bromo-5-(4-fluorophenyl)-3-methyl-1H-pyrazol-1-yl]butan-2-ol (1.19 g) according to the similar procedure described for Example 15 (726 mg).

¹H-NMR (300 MHz, DMSO-d₆) δ: 0.80 (3H, t, J=7.3 Hz), 1.08-1.43 (2H, m), 2.19 (3H, s), 3.71-3.86 (3H, m), 4.53 (2H, s), 4.87 (1H, d, J=5.1 Hz), 6.55 (1H, dd, J=8.3, 2.1 Hz), 6.64 (1H, d, J=2.1 Hz), 6.81 (1H, d, J=8.3 Hz), 7.25 (2H, t, J=8.9 Hz), 7.30-7.45 (2H, m), 10.58 (1H, s).

LCMS (ESI⁺) M+H⁺: 395.88.

### Example 21

### 6-[5-(4-Fluorophenyl)-3-methyl-1-(2-oxobutyl)-1H-pyrazol-4-yl]-2H-1,4-benzoxazin-3(4H)-one

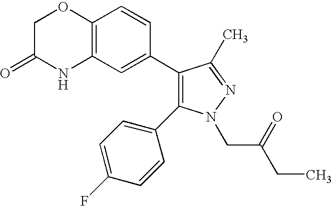

To a solution of 6-[5-(4-fluorophenyl)-1-(2-hydroxybutyl)-3-methyl-1H-pyrazol-4-yl]-2H-1,4-benzoxazin-3(4H)-one (680 mg) in DMSO/triethylamine (5 ml/3 ml) was added pyridine sulfur trioxide (1.09 g), and stirred at room temperature for 8 h. The mixture was diluted with ethyl acetate, and washed with water, and saturated brine. The organic layer was separated, dried over anhydrous magnesium sulfate and concentrated. The residue was purified by HPLC, and crystallized from hexane/ethyl acetate to give the title compound (332 mg).

¹H-NMR (300 MHz, DMSO-d₆) δ: 0.86 (3H, t, J=7.2 Hz), 2.19 (3H, s), 2.36 (2H, q, J=7.2 Hz), 4.54 (2H, s), 4.90 (2H, s), 6.58 (1H, dd, J=8.3, 1.9 Hz), 6.67 (1H, d, J=1.9 Hz), 6.83 (1H, d, J=8.3 Hz), 7.10-7.33 (4H, m), 10.60 (1H, s).

LCMS (ESI⁺) M+H⁺: 393.87.

### Example 22

### 6-[1-(2,2-Difluorobutyl)-5-(4-fluorophenyl)-3-methyl-1H-pyrazol-4-yl]-2H-1,4-benzoxazin-3(4H)-one

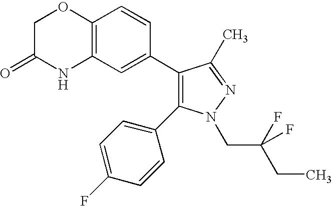

The title compound was obtained from 4-bromo-1-(2,2-difluorobutyl)-5-(4-fluorophenyl)-3-methyl-1H-pyrazole (903 mg) according to the similar procedure described for Example (427 mg).

¹H-NMR (300 MHz, DMSO-d₆) δ: 0.86 (3H, t, J=7.4 Hz), 1.84 (2H, m), 2.20 (3H, s), 4.38 (2H, t, J=13.3 Hz), 4.53 (2H, s), 6.58 (1H, dd, J=8.1, 2.1 Hz), 6.65 (1H, d, J=1.9 Hz), 6.82 (1H, d, J=8.3 Hz), 7.21-7.35 (4H, m), 10.61 (1H, s).

LCMS (ESI⁺) M+H⁺: 416.1.

### Example 23

### 6-[1-(Cyclopropylmethyl)-5-(4-fluorophenyl)-3-methyl-1H-pyrazol-4-yl]-2H-1,4-benzoxazin-3(4H)-one

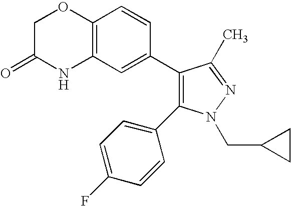

The title compound was obtained from 4-bromo-1-(cyclopropylmethyl)-5-(4-fluorophenyl)-3-methyl-1H-pyrazole (205 mg) according to the similar procedure described for Example 15 (90 mg).

¹H-NMR (300 MHz, DMSO-d₆) δ: 0.03-0.45 (4H, m), 0.92-1.13 (1H, m), 2.19 (3H, s), 3.77 (2H, d, J=6.8 Hz), 4.53 (2H, s), 6.58 (1H, dd, J=8.1, 2.1 Hz), 6.65 (1H, d, J=1.9 Hz), 6.81 (1H, d, J=8.3 Hz), 7.22-7.37 (4H, m), 10.61 (1H, s). LCMS (ESI⁺) M+H⁺: 377.91.

### Example 24

### 6-[1-(2-Cyclopropylethyl)-5-(4-fluorophenyl)-3-methyl-1H-pyrazol-4-yl]-2H-1,4-benzoxazin-3(4H)-one

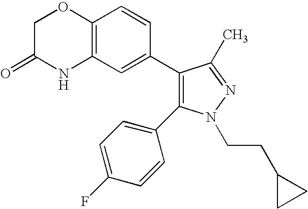

The title compound was obtained from 4-bromo-1-(2-cyclopropylethyl)-5-(4-fluorophenyl)-3-methyl-1H-pyrazole (196 mg) according to the similar procedure described for Example 15 (96 mg).

¹H-NMR (300 MHz, DMSO-d₆) δ: −0.18-−0.08 (2H, m), 0.23-0.36 (2H, m), 0.40-0.55 (1H, m), 1.55 (2H, q, J=6.9 Hz), 2.18 (3H, s), 3.94 (2H, t, J=7.0 Hz), 4.53 (2H, s), 6.56 (1H, dd, J=8.3, 1.9 Hz), 6.65 (1H, d, J=1.9 Hz), 6.81 (1H, d, J=8.0 Hz), 7.22-7.36 (4H, m), 10.60 (1H, s).

LCMS (ESI⁺) M+H⁺: 391.93.

### Example 25

### 6-[1-(3,3-Difluoro-4-hydroxybutyl)-5-(4-fluorophenyl)-3-methyl-1H-pyrazol-4-yl]-2H-1,4-benzoxazin-3(4H)-one

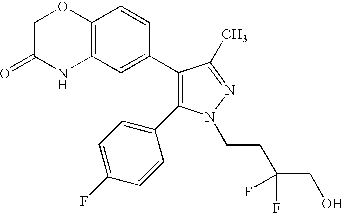

The title compound was obtained from 4-[4-bromo-5-(4-fluorophenyl)-3-methyl-1H-pyrazol-1-yl]-2,2-difluorobutyl acetate (60 mg) according to the similar procedure described for Example 15 (22 mg).

¹H-NMR (300 MHz, DMSO-d₆) δ: 2.19 (3H, s), 2.25-2.48 (2H, m), 3.51 (2H, td, J=13.7, 6.3 Hz), 4.00-4.14 (2H, m), 4.53 (2H, s), 5.51 (1H, t, J=6.2 Hz), 6.57 (1H, dd, J=8.3, 2.1 Hz), 6.65 (1H, d, J=2.1 Hz), 6.82 (1H, d, J=8.3 Hz), 7.22-7.42 (4H, m), 10.60 (1H, s).

LCMS (ESI⁺) M+H⁺: 431.75.

### Example 26

### 2,2-Difluoro-3-[5-(4-fluorophenyl)-3-methyl-4-(3-oxo-3,4-dihydro-2H-1,4-benzoxazin-6-yl)-1H-pyrazol-1-yl]propyl acetate

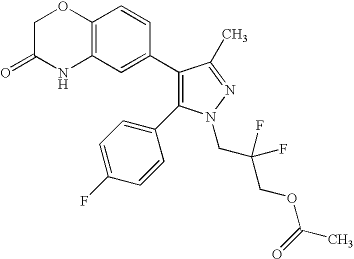

A mixture of 6-[1-(2,2-difluoro-3-hydroxypropyl)-5-(4-fluorophenyl)-3-methyl-1H-pyrazol-4-yl]-2H-1,4-benzoxazin-3(4H)-one (466 mg) and acetic anhydride (16 ml) was stirred at 60° C. for 6 h. The mixture was diluted with ethyl acetate, and washed with aqueous sodium bicarbonate and saturated brine. The organic layer was separated, dried over anhydrous magnesium sulfate and concentrated. The residue was purified by HPLC, and crystallized from hexane/ethyl acetate to give the title compound (373 mg).

¹H-NMR (300 MHz, DMSO-d₆) δ: 2.01 (3H, s), 2.20 (3H, s), 4.35 (2H, t, J=13.9 Hz), 4.45-4.57 (4H, m), 6.59 (1H, dd, J=8.1, 2.1 Hz), 6.66 (1H, d, J=2.1 Hz), 6.83 (1H, d, J=8.3 Hz), 7.17-7.39 (4H, m), 10.61 (1H, s).

LCMS (ESI⁺) M+H⁺: 459.82.

### Example 27

### 6-[5-(4-Fluorophenyl)-3-methyl-1-(4,4,4-trifluoro-2-hydroxybutyl)-1H-pyrazol-4-yl]-2H-1,4-benzoxazin-3(4H)-one

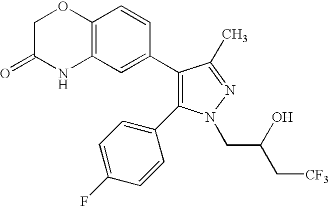

The title compound was obtained from 1-[4-bromo-5-(4-fluorophenyl)-3-methyl-1H-pyrazol-1-yl]-4,4,4-trifluorobutan-2-ol (4.04 g) according to the similar procedure described for Example 15 (1.76 g).

¹H-NMR (300 MHz, DMSO-d₆) δ: 2.20 (3H, s), 2.22-2.47 (2H, m), 3.77-3.97 (2H, m), 4.23 (1H, br. s), 4.53 (2H, s), 5.47 (1H, d, J=6.1 Hz), 6.56 (1H, dd, J=8.3, 1.9 Hz), 6.65 (1H, d), 6.82 (1H, d, J=8.3 Hz), 7.26 (2H, t, J=8.7 Hz), 7.32-7.41 (2H, m), 10.60 (1H, s).

LCMS (ESI⁺) M+H⁺: 449.90.

### Example 28

### 6-[5-(4-Fluorophenyl)-3-methyl-1-(4,4,4-trifluoro-2-oxobutyl)-1H-pyrazol-4-yl]-2H-1,4-benzoxazin-3(4H)-one

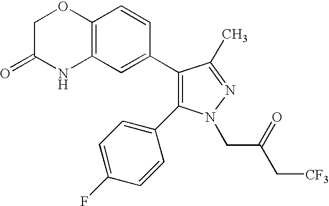

To a solution of 6-[5-(4-fluorophenyl)-3-methyl-1-(4,4,4-trifluoro-2-hydroxybutyl)-1H-pyrazol-4-yl]-2H-1,4-benzoxazin-3(4H)-one (219 mg) in acetonitrile (20 ml) was added 1,1,1-tris(acetyloxy)-1lambda˜5˜,2-benziodoxol-3(1H)-one (310 mg), and stirred at room temperature for 3 h. The reaction mixture was poured into ice-water, and extracted with ethyl acetate. The organic layer was washed with saturated brine, dried over anhydrous magnesium sulfate and concentrated. The residue was chromatographed on silica gel using hexane/ethyl acetate as an eluent, and crystallized from hexane/ethyl acetate to give the title compound (15 mg).

¹H-NMR (300 MHz, DMSO-d₆) δ: 2.19 (3H, s), 3.82 (2H, q, J=11.0 Hz), 4.54 (2H, s), 5.03 (2H, s), 6.59 (1H, dd, J=8.3, 1.9 Hz), 6.68 (1H, d, J=2.3 Hz), 6.83 (1H, d, J=8.3 Hz), 7.12-7.33 (4H, m), 10.62 (1H, s).

LCMS (ESI⁺) M+H⁺: 447.85.

### Example 29

### 6-[5-(4-Fluorophenyl)-1-{[(1SR,2SR)-2-hydroxy-2-methylcyclopropyl]methyl}-3-methyl-1H-pyrazol-4-yl]-2H-1,4-benzoxazin-3(4H)-one

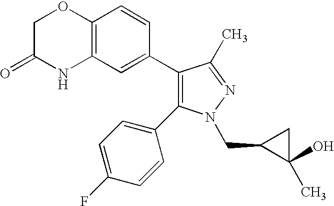

The title compound was obtained from 2-{[4-bromo-5-(4-fluorophenyl)-3-methyl-1H-pyrazol-1-yl]methyl}-1-methylcyclopropanol (90 mg) according to the similar procedure described for Example 15 (18 mg).

¹H-NMR (300 MHz, DMSO-d₆) δ: 0.20-0.56 (2H, m), 0.93-1.09 (1H, m), 1.25 (3H, s), 2.20 (3H, br. s), 3.75-4.17 (2H, m), 4.53 (2H, s), 5.27 (1H, s), 6.58 (1H, d, J=8.3 Hz), 6.65 (1H, br. s), 6.81 (1H, d, J=8.0 Hz), 7.16-7.40 (4H, m), 10.61 (1H, br. s).

LCMS (ESI⁺) M+H⁺: 407.91.

### Example 30

### 2-[5-(4-Fluorophenyl)-3-methyl-4-(3-oxo-3,4-dihydro-2H-1,4-benzoxazin-6-yl)-1H-pyrazol-1-yl]-N,N-dimethylethanesulfonamide

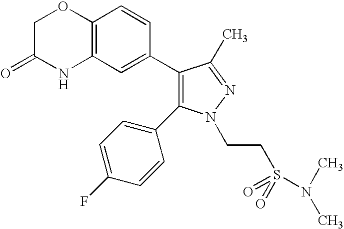

The title compound was obtained from 2-[4-bromo-5-(4-fluorophenyl)-3-methyl-1H-pyrazol-1-yl]-N,N-dimethylethanesulfonamide (128 mg) according to the similar procedure described for Example 15 (24 mg).

¹H-NMR (300 MHz, DMSO-d₆) δ: 2.21 (3H, s), 2.68 (6H, s), 3.55 (2H, dd, J=8.4, 6.3 Hz), 4.23 (2H, dd, J=8.2, 6.5 Hz), 4.54 (2H, s), 6.57 (1H, dd, J=8.3, 2.1 Hz), 6.65 (1H, d, J=2.1 Hz), 6.83 (1H, d, J=8.3 Hz), 7.19-7.48 (4H, m), 10.60 (1H, s).

LCMS (ESI⁺) M+H⁺: 458.89.

### Example 31

### 2-[5-(4-Fluorophenyl)-3-methyl-4-(3-oxo-3,4-dihydro-2H-1,4-benzoxazin-6-yl)-1H-pyrazol-1-yl]-N-methylethanesulfonamide

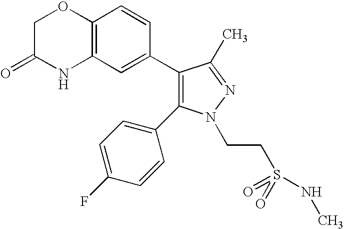

The title compound was obtained from 2-[4-bromo-5-(4-fluorophenyl)-3-methyl-1H-pyrazol-1-yl]-N-methylethanesulfonamide (282 mg) according to the similar procedure described for Example 1 (28 mg).

¹H-NMR (300 MHz, DMSO-d₆) δ: 2.20 (3H, s), 3.41-3.55 (2H, m), 4.11-4.24 (2H, m), 4.53 (2H, s), 6.57 (1H, d, J=8.3 Hz), 6.65 (1H, m), 6.83 (1H, d, J=8.0 Hz), 7.04 (1H, m), 7.22-7.49 (4H, m), 10.61 (1H, s), 1H unconfirmed.

LCMS (ESI⁺) M+H⁺: 444.86.

### Example 32

### 8-Chloro-6-[5-(4-fluorophenyl)-1,3-dimethyl-1H-pyrazol-4-yl]-2H-1,4-benzoxazin-3(4H)-one

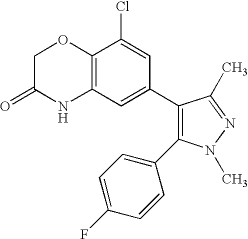

The title compound was obtained from 8-chloro-6-(4,4,5,5-tetramethyl-1,3,2-dioxaborolan-2-yl)-2H-1,4-benzoxazin-3(4H)-one (2.07 g) according to the similar procedure described for Example 1 (105 mg).

Mp 260.2-260.5° C.

¹H-NMR (300 MHz, DMSO-d₆) δ: 2.18 (3H, s), 3.64 (3H, s), 4.67 (2H, s), 6.58 (1H, d, J=1.9 Hz), 6.68 (1H, d, J=1.9 Hz), 7.18-7.47 (4H, m), 10.78 (1H, br. s).

LCMS (ESI⁺) M+H⁺: 372.08.

### Example 33

### 6-[5-(4-Fluorophenyl)-1-methyl-1H-pyrazol-4-yl]-2H-1,4-benzoxazin-3(4H)-one

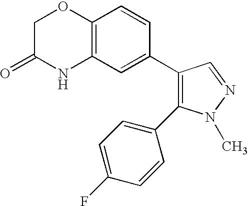

A mixture of 6-(4,4,5,5-tetramethyl-1,3,2-dioxaborolan-2-yl)-2H-1,4-benzoxazin-3(4H)-one (647 mg), 4-bromo-5-(4-fluorophenyl)-1-methyl-1H-pyrazole (500 mg), Cesium carbonate (1.92 g), and [1,1′-bis(diphenylphosphino)ferrocene]dichloropalladium(II) dichloromethane adduct (160 mg) in tetrahydrofuran/H₂O (5/1) (10 ml) was well evacuated, and refluxed for 18 h. The reaction mixture was diluted with ethyl acetate, and washed with saturated brine. The organic layer was separated, dried over anhydrous magnesium sulfate and concentrated. The residue was chromatographed on basic silica gel using ethyl acetate/hexane as an eluent and followed by recrystallization from ethyl acetate/hexane to give the title compound (196 mg).

¹H-NMR (300 MHz, DMSO-d₆) δ: 3.68 (3H, s), 4.51 (2H, s), 6.59-6.73 (2H, m), 6.82 (1H, d, J=8.9 Hz), 7.24-7.49 (4H, m), 7.66 (1H, s), 10.62 (1H, s).

LCMS (ESI⁺) M+H⁺: 324.08.

### Example 34

### 6-[5-(4-Fluorophenyl)-1,3-dimethyl-1H-pyrazol-4-yl]-8-methyl-2H-1,4-benzoxazin-3(4H)-one

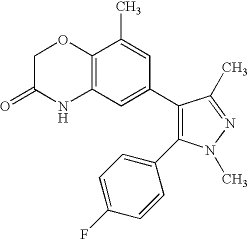

The title compound was obtained from 8-methyl-6-(4,4,5,5-tetramethyl-1,3,2-dioxaborolan-2-yl)-2H-1,4-benzoxazin-3(4H)-one (1.12 g) according to the similar procedure described for Example 33 (135 mg).

Mp 272.1-272.5° C.

¹H-NMR (300 MHz, DMSO-d₆) δ: 2.06 (3H, s), 2.16 (3H, s), 3.64 (3H, s), 4.54 (2H, s), 6.45 (1H, d, J=1.9 Hz), 6.51 (1H, d), 7.18-7.41 (4H, m), 10.51 (1H, s).

LCMS (ESI⁺) M+H⁺: 352.12.

### Example 35

### 5-Fluoro-6-[5-(4-fluorophenyl)-1,3-dimethyl-1H-pyrazol-4-yl]-2H-1,4-benzoxazin-3(4H)-one

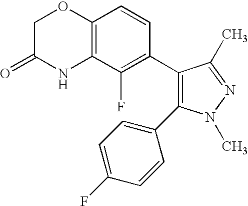

The title compound was obtained from 5-fluoro-6-(4,4,5,5-tetramethyl-1,3,2-dioxaborolan-2-yl)-2H-1,4-benzoxazin-3(4H)-one (400 mg) according to the similar procedure described for Example 1 (88 mg).

¹H-NMR (300 MHz, DMSO-d₆) δ: 2.07 (3H, s), 3.69 (3H, s), 4.59 (2H, s), 6.66 (1H, dd, J=8.5, 7.7 Hz), 6.74 (1H, dd, J=8.5, 1.1 Hz), 7.15-7.39 (4H, m), 10.79 (1H, s).

LCMS (ESI⁺) M+H⁺: 356.03.

### Example 36

### 5-Chloro-6-[5-(4-fluorophenyl)-1,3-dimethyl-1H-pyrazol-4-yl]-2H-1,4-benzoxazin-3(4H)-one

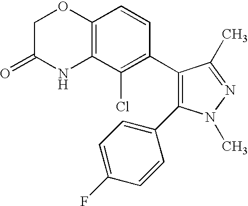

A mixture of 5-chloro-6-(4,4,5,5-tetramethyl-1,3,2-dioxaborolan-2-yl)-2H-1,4-benzoxazin-3(4H)-one (191 mg), 4-bromo-5-(4-fluorophenyl)-1,3-dimethyl-1H-pyrazole (249 mg), 2-Dicyclohexylphosphino-2′,4′,6′-triisopropylbiphenyl (235 mg) and K₃PO₄ (393 mg) in 1,2-dimethoxyethane (6 ml) was well evacuated, and refluxed for 15 h. The reaction mixture was diluted with ethyl acetate, and washed with saturated brine. The organic layer was separated, dried over anhydrous magnesium sulfate and concentrated. The residue was chromatographed on and basic silica gel using hexane/ethyl acetate as an eluent, and crystallized from hexane/ethyl acetate to give the title compound (9 mg).

¹H-NMR (300 MHz, DMSO-d₆) δ: 2.00 (3H, s), 3.70 (3H, s), 4.59 (2H, s), 6.77 (1H, d, J=8.3 Hz), 6.91 (1H, d, J=8.3 Hz), 7.12-7.36 (4H, m), 10.36 (1H, br. s).

LCMS (ESI⁺) M+H⁺: 371.83.

### Example 37

### N-{2-[5-(4-Fluorophenyl)-3-methyl-4-(5-methyl-3-oxo-3,4-dihydro-2H-1,4-benzoxazin-6-yl)-1H-pyrazol-1-yl]ethyl}methanesulfonamide

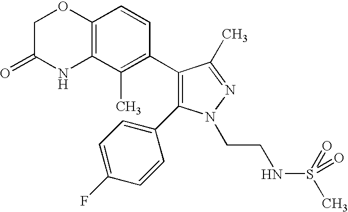

The title compound was obtained from 5-methyl-6-(4,4,5,5-tetramethyl-1,3,2-dioxaborolan-2-yl)-2H-1,4-benzoxazin-3(4H)-one (768 mg) and N-{2-[4-bromo-5-(4-fluorophenyl)-3-methyl-1H-pyrazol-1-yl]ethyl}methanesulfonamide (1.5 g) according to the similar procedure described for Example 36 (142 mg).

¹H-NMR (300 MHz, DMSO-d₆) δ: 1.84 (3H, s), 1.99 (3H, s), 2.81 (3H, s), 3.37 (2H, m), 4.02 (2H, t, J=6.7 Hz), 4.50 (2H, s), 6.66 (1H, d, J=8.3 Hz), 6.76 (1H, d, J=8.3 Hz), 7.11-7.32 (4H, m), 10.09 (1H, br. s).

LCMS (ESI⁺) M+H⁺: 458.82.

### Example 38

### 6-{1-[2-(Dimethylamino)ethyl]-5-(4-fluorophenyl)-3-methyl-1H-pyrazol-4-yl}-2H-1,4-benzoxazin-3(4H)-one

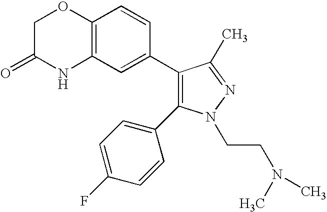

The title compound was obtained from 2-[4-bromo-5-(4-fluorophenyl)-3-methyl-1H-pyrazol-1-yl]-N,N-dimethylethanamine (145 mg) according to the similar procedure described for Example 1 (82 mg).

¹H-NMR (300 MHz, DMSO-d₆) δ: 2.00 (6H, s), 2.18 (3H, s), 2.50-2.57 (2H, m), 3.95 (2H, t, J=6.6 Hz), 4.53 (2H, s), 6.56 (1H, dd, J=8.3, 1.9 Hz), 6.65 (1H, d, J=1.9 Hz), 6.81 (1H, d, J=8.0 Hz), 7.19-7.39 (4H, m), 10.60 (1H, s).

LCMS (ESI⁺) M+H⁺: 394.92.

### Example 39

### 6-[5-(4-Fluorophenyl)-3-methyl-1-(2-morpholin-4-ylethyl)-1H-pyrazol-4-yl]-2H-1,4-benzoxazin-3(4H)-one

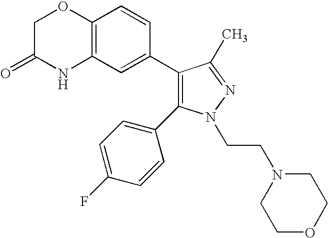

The title compound was obtained from 4-{2-[4-bromo-5-(4-fluorophenyl)-3-methyl-1H-pyrazol-1-yl]ethyl}morpholine (53 mg) according to the similar procedure described for Example 1 (24 mg).

¹H-NMR (300 MHz, DMSO-d₆) δ: 2.14-2.26 (7H, m), 2.61 (2H, t, J=6.6 Hz), 3.47 (4H, t, J=4.2 Hz), 3.98 (2H, t, J=6.6 Hz), 4.53 (2H, s), 6.56 (1H, dd, J=8.3, 1.9 Hz), 6.65 (1H, d, J=1.9 Hz), 6.81 (1H, d, J=8.0 Hz), 7.20-7.42 (4H, m), 10.60 (1H, s).

LCMS (ESI⁺) M+H⁺: 436.82.

### Example 40

### 6-{5-(4-Fluorophenyl)-3-methyl-1-[3-(methylsulfanyl)propyl]-1H-pyrazol-4-yl}-2H-1,4-benzoxazin-3(4H)-one

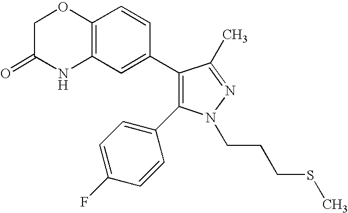

The title compound was obtained from 4-bromo-5-(4-fluorophenyl)-3-methyl-1-[3-(methylsulfanyl)propyl]-1H-pyrazole (258 mg) according to the similar procedure described for Example 1 (181 mg).

¹H-NMR (300 MHz, DMSO-d₆) δ: 1.84-1.97 (5H, m), 2.19 (3H, s), 2.36 (2H, t, J=7.0 Hz), 3.98 (2H, t, J=7.0 Hz), 4.53 (2H, s), 6.57 (1H, dd, J=8.3, 1.9 Hz), 6.65 (1H, d, J=1.9 Hz), 6.81 (1H, d, J=8.3 Hz), 7.22-7.37 (4H, m), 10.61 (1H, s).

LCMS (ESI⁺) M+H⁺: 411.85.

### Example 41

### 6-{5-(4-Fluorophenyl)-3-methyl-1-[3-(methylsulfinyl)propyl]-1H-pyrazol-4-yl}-2H-1,4-benzoxazin-3(4H)-one

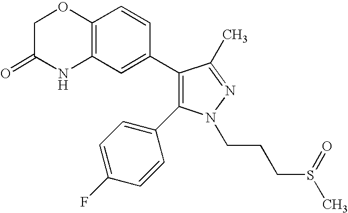

A mixture of 6-{5-(4-Fluorophenyl)-3-methyl-1-[3-(methylsulfanyl)propyl]-1H-pyrazol-4-yl}-2H-1,4-benzoxazin-3(4H)-one (85 mg) and 3-chlorobenzenecarboperoxoic acid (39 mg) in dichloromethane (1 ml) was stirred at room temperature for 3 h. The reaction mixture was diluted with ethyl acetate, and washed with aqueous sodium bicarbonate, and saturated brine. The organic layer was separated, dried over anhydrous magnesium sulfate and concentrated. The residue was chromatographed on silica gel using hexane/ethyl acetate as an eluent, and crystallized from hexane/ethyl acetate to give the title compound (53 mg).

¹H-NMR (300 MHz, DMSO-d₆) δ: 2.03 (2H, q), 2.20 (3H, s), 2.47 (3H, s), 2.52-2.76 (2H, m), 4.01 (2H, t, J=6.8 Hz), 4.53 (2H, s), 6.57 (1H, d, J=8.3 Hz), 6.66 (1H, s), 6.82 (1H, d, J=8.3 Hz), 7.18-7.42 (4H, m), 10.62 (1H, s).

LCMS (ESI⁺) M+H⁺: 427.78.

### Example 42

### 6-{5-(4-Fluorophenyl)-3-methyl-1-[3-(methylsulfonyl)propyl]-1H-pyrazol-4-yl}-2H-1,4-benzoxazin-3(4H)-one

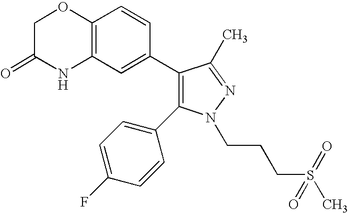

The title compound was obtained using 2 eq. amount of 3-chlorobenzenecarboperoxoic acid according to the similar procedure described for Example 41 (70 mg).

¹H-NMR (300 MHz, DMSO-d₆) δ: 2.03-2.16 (2H, m), 2.20 (3H, s), 2.93 (3H, s), 3.07 (1H, t, J=7.6 Hz), 4.01 (2H, t, J=6.8 Hz), 4.53 (2H, s), 6.58 (1H, m), 6.66 (1H, m), 6.82 (1H, d, J=8.3 Hz), 7.21-7.39 (4H, m), 10.62 (1H, s).

LCMS (ESI⁺) M+H⁺: 443.78.

### Example 43

### 6-[5-(4-Fluorophenyl)-1-methyl-3-(trifluoromethyl)-1H-pyrazol-4-yl]-2H-1,4-benzoxazin-3(4H)-one

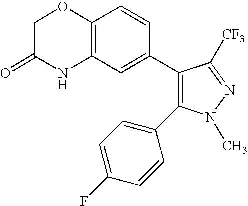

The title compound was obtained from 4-Bromo-5-(4-fluorophenyl)-1-methyl-3-(trifluoromethyl)-1H-pyrazole (797 mg) according to the similar procedure described for Example 1 (555 mg).

¹H-NMR (300 MHz, DMSO-d₆) δ: 3.80 (3H, s), 4.56 (2H, s), 6.60-6.75 (2H, m), 6.86 (1H, d, J=8.3 Hz), 7.29 (2H, m), 7.42 (2H, m), 10.68 (1H, s).

LCMS (ESI⁺) M+H⁺: 392.03.

### Example 44

### 6-[5-(2,4-Difluorophenyl)-1,3-dimethyl-1H-pyrazol-4-yl]-2H-1,4-benzoxazin-3(4H)-one

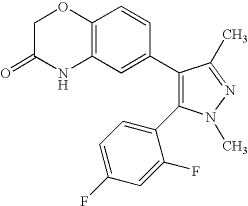

The title compound was obtained from 4-bromo-5-(2,4-difluorophenyl)-1,3-dimethyl-1H-pyrazole (500 mg) according to the similar procedure described for Example 1 (300 mg).

¹H-NMR (300 MHz, DMSO-d₆) δ: 2.20 (3H, s), 3.59 (3H, s), 4.53 (2H, s), 6.58 (1H, dd, J=8.1, 2.1 Hz), 6.64 (1H, d, J=2.3 Hz), 6.83 (1H, d, J=8.3 Hz), 7.18 (1H, td, J=8.6, 2.1 Hz), 7.31-7.47 (2H, m), 10.59 (1H, s).

LCMS (ESI⁺) M+H⁺: 356.11.

### Example 45

### 6-[5-(2-Fluorophenyl)-1,3-dimethyl-1H-pyrazol-4-yl]-2H-1,4-benzoxazin-3(4H)-one

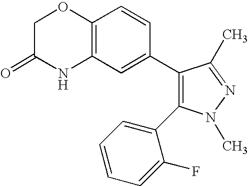

The title compound was obtained from 4-bromo-5-(2-fluorophenyl)-1,3-dimethyl-1H-pyrazole (800 mg) according to the similar procedure described for Example 1 (318 mg).

¹H-NMR (300 MHz, DMSO-d₆) δ: 2.19 (3H, s), 3.59 (3H, s), 4.53 (2H, s), 6.56 (1H, dd, J=8.3, 2.1 Hz), 6.68 (1H, d, J=2.1 Hz), 6.81 (1H, d, J=8.1 Hz), 7.17-7.40 (3H, m), 7.41-7.62 (1H, m), 10.59 (1H, s).

LCMS (ESI⁺) M+H⁺: 338.03.

### Example 46

### 6-[5-(3-Fluorophenyl)-1,3-dimethyl-1H-pyrazol-4-yl]-2H-1,4-benzoxazin-3(4H)-one

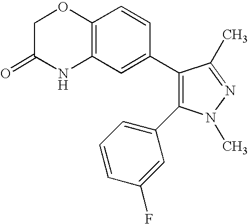

The title compound was obtained from 4-bromo-5-(3-fluorophenyl)-1,3-dimethyl-1H-pyrazole (800 mg) according to the similar procedure described for Example 1 (91 mg).

¹H-NMR (300 MHz, DMSO-d₆) δ: 2.17 (3H, s) 3.67 (3H, s) 4.54 (2H, s) 6.59 (1H, dd, J=8.3, 2.0 Hz) 6.66 (1H, d, J=2.0 Hz) 6.84 (1H, d, J=8.3 Hz) 7.08 (1H, d, J=7.6 Hz) 7.14-7.31 (2H, m) 7.40-7.51 (1H, m) 10.59 (1H, s).

LCMS (ESI⁺) M+H⁺: 338.03.

### Example 47

### 6-[5-(3,4-Difluorophenyl)-1,3-dimethyl-1H-pyrazol-4-yl]-2H-1,4-benzoxazin-3(4H)-one

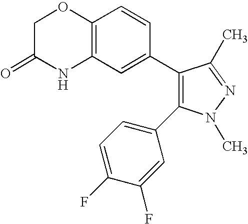

The title compound was obtained from 4-bromo-5-(3,4-difluorophenyl)-1,3-dimethyl-1H-pyrazole (400 mg) according to the similar procedure described for Example 1 (259 mg).

¹H-NMR (300 MHz, DMSO-d₆) δ: 2.17 (3H, s), 3.66 (3H, s), 4.54 (2H, s), 6.53-6.73 (2H, m), 6.86 (1H, d, J=8.9 Hz), 7.08 (1H, ddd, J=8.3, 4.2, 2.0 Hz), 7.36-7.59 (2H, m), 10.59 (1H, s).

LCMS (ESI⁺) M+H⁺: 356.08.

### Example 48

### 6-[5-(3,5-Difluorophenyl)-1,3-dimethyl-1H-pyrazol-4-yl]-2H-1,4-benzoxazin-3(4H)-one

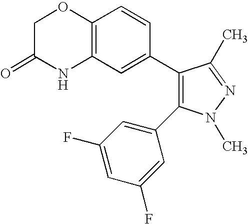

The title compound was obtained from 4-bromo-5-(3,5-difluorophenyl)-1,3-dimethyl-1H-pyrazole (400 mg) according to the similar procedure described for Example 1 (260 mg).

¹H-NMR (300 MHz, DMSO-d₆) δ: 2.17 (3H, s), 3.69 (3H, s), 4.55 (2H, s), 6.54-6.73 (2H, m), 6.87 (1H, d, J=8.1 Hz), 7.05 (2H, dd, J=8.3, 2.3 Hz), 7.32 (1H, tt, J=9.5, 2.3 Hz), 10.61 (1H, s).

LCMS (ESI⁺) M+H⁺: 356.01.

### Example 49

### 6-[5-(2,5-Difluorophenyl)-1,3-dimethyl-1H-pyrazol-4-yl]-2H-1,4-benzoxazin-3(4H)-one

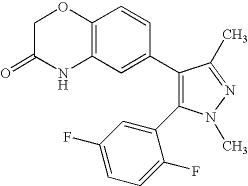

The title compound was obtained from 5-(2,5-difluorophenyl)-4-iodo-1,3-dimethyl-1H-pyrazole (500 mg) according to the similar procedure described for Example 1 (363 mg).

¹H-NMR (300 MHz, DMSO-d₆) δ: 2.19 (3H, s), 3.62 (3H, s), 4.54 (2H, s), 6.60 (1H, dd, J=8.3, 1.9 Hz), 6.66 (1H, d, J=2.3 Hz), 6.84 (1H, d, J=8.3 Hz), 7.21-7.32 (1H, m), 7.38 (2H, t, J=6.4 Hz), 10.60 (1H, s).

LCMS (ESI⁺) M+H⁺: 356.03.

### Example 50

### 6-[5-(2,3-Difluorophenyl)-1,3-dimethyl-1H-pyrazol-4-yl]-2H-1,4-benzoxazin-3(4H)-one

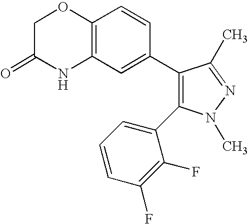

The title compound was obtained from 5-(2,3-difluorophenyl)-4-iodo-1,3-dimethyl-1H-pyrazole (300 mg) according to the similar procedure described for Example 1 (156 mg).

¹H-NMR (300 MHz, DMSO-d₆) δ: 2.20 (3H, s), 3.62 (3H, s), 4.53 (2H, s), 6.60 (1H, dd, J=8.1, 2.1 Hz), 6.64 (1H, d, J=1.9 Hz), 6.84 (1H, d, J=8.3 Hz), 7.12 (1H, t, J=7.0 Hz), 7.21-7.36 (1H, m), 7.55 (1H, q, J=8.3 Hz), 10.59 (1H, s).

LCMS (ESI⁺) M+H⁺: 356.03.

### Example 51

### 5,8-Difluoro-6-[5-(4-fluorophenyl)-1,3-dimethyl-1H-pyrazol-4-yl]-2H-1,4-benzoxazin-3(4H)-one

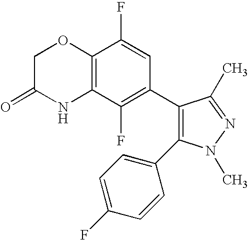

The title compound was obtained from 5,8-difluoro-6-(4,4,5,5-tetramethyl-1,3,2-dioxaborolan-2-yl)-2H-1,4-benzoxazin-3(4H)-one (200 mg) according to the similar procedure described for Example 1 (26 mg).

¹H-NMR (300 MHz, DMSO-d₆) δ: 2.09 (3H, s), 3.69 (3H, s), 4.67 (2H, s), 6.70 (1H, dd, J=11.2, 6.2 Hz), 7.20-7.41 (4H, m), 10.99 (1H, br. s).

LCMS (ESI⁺) M+H⁺: 373.90.

### Example 52

### 6-[3-(Difluoromethyl)-5-(4-fluorophenyl)-1-methyl-1H-pyrazol-4-yl]-2H-1,4-benzoxazin-3(4H)-one

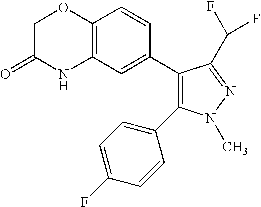

The title compound was obtained from 4-bromo-3-(difluoromethyl)-5-(4-fluorophenyl)-1-methyl-1H-pyrazole (500 mg) according to the similar procedure described for Example 1 (411 mg).

¹H-NMR (300 MHz, DMSO-d₆) δ: 3.76 (3H, s), 4.55 (2H, s), 6.66 (1H, dd, J=8.1, 1.9 Hz), 6.72 (1H, d, J=1.9 Hz), 6.85 (1H, d, J=8.1 Hz), 6.90 (1H, t, J=54.0 Hz), 7.29 (2H, t, J=8.9 Hz), 7.35-7.45 (2H, m), 10.66 (1H, s).

LCMS (ESI⁺) M+H⁺: 373.89.

### Example 53

### 6-[5-(4-Fluorophenyl)-3-methyl-1-{2-[(1-methylethyl)sulfanyl]ethyl}-1H-pyrazol-4-yl]-2H-1,4-benzoxazin-3(4H)-one

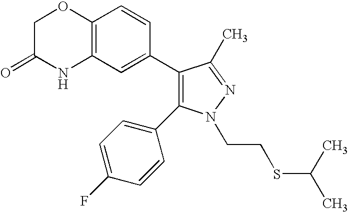

The title compound was obtained from 4-bromo-5-(4-fluorophenyl)-3-methyl-1-[2-(propan-2-ylsulfanyl)ethyl]-1H-pyrazole (125 mg) according to the similar procedure described for Example 1 (65 mg).

¹H-NMR (300 MHz, DMSO-d₆) δ: 1.06 (6H, d, J=6.4 Hz), 2.19 (3H, s), 2.64 (1H, m), 2.81 (2H, t, J=7.0 Hz), 4.03 (2H, t, J=6.8 Hz), 4.53 (2H, s), 6.57 (1H, d, J=8.3 Hz), 6.65 (1H, s), 6.82 (1H, d, J=8.3 Hz), 7.19-7.44 (4H, m), 10.59 (1H, s).

LCMS (ESI⁺) M+H⁺: 425.79.

### Example 54

### 6-[5-(4-Fluorophenyl)-3-methyl-1-{2-[(1-methylethyl)sulfinyl]ethyl}-1H-pyrazol-4-yl]-2H-1,4-benzoxazin-3(4H)-one

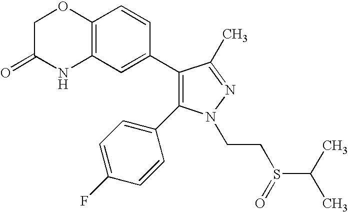

The title compound was obtained from 6-[5-(4-fluorophenyl)-3-methyl-1-(2-[(1-methylethyl)sulfanyl]ethyl-1H-pyrazol-4-yl]-2H-1,4-benzoxazin-3(4H)-one (25 mg) according to the similar procedure described for Example 41 (10 mg).

¹H-NMR (300 MHz, DMSO-d₆) δ: 1.12 (6H, m), 2.20 (3H, s), 2.80 (1H, m), 2.88-3.01 (1H, m), 3.22 (1H, dt, J=13.3, 7.7 Hz), 4.20-4.33 (2H, m), 4.53 (2H, s), 6.58 (1H, dd, J=8.3, 2.1 Hz), 6.65 (1H, d, J=1.9 Hz), 6.83 (1H, d, J=8.1 Hz), 7.23-7.32 (2H, m), 7.33-7.41 (2H, m), 10.60 (1H, s).

LCMS (ESI⁺) M+H⁺: 441.75.

### Example 55

### 6-[5-(4-Fluorophenyl)-3-methyl-1-{2-[(1-methylethyl)sulfonyl]ethyl}-1H-pyrazol-4-yl]-2H-1,4-benzoxazin-3(4H)-one

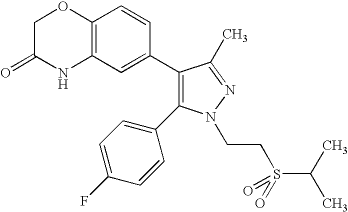

The title compound was obtained using 2 eq. amount of 3-chlorobenzenecarboperoxoic acid from 6-[5-(4-fluorophenyl)-3-methyl-1-{2-[(1-methylethyl)sulfanyl]ethyl}-1H-pyrazol-4-yl]-2H-1,4-benzoxazin-3(4H)-one (25 mg) according to the similar procedure described for Example 41 (9 mg).

¹H-NMR (300 MHz, DMSO-d₆) δ: 1.18 (6H, d, J=6.8 Hz), 2.20 (3H, s), 3.05-3.22 (1H, m), 3.60 (2H, t, J=7.3 Hz), 4.28 (2H, t, J=7.1 Hz), 4.53 (2H, s), 6.58 (1H, d, J=8.1 Hz), 6.65 (1H, s), 6.83 (1H, d, J=8.3 Hz), 7.21-7.46 (4H, m), 10.60 (1H, br. s).

LCMS (ESI⁺) M+H⁺: 457.76.

### Example 56

### 6-[5-(4-Fluorophenyl)-3-methyl-1-(thiophen-3-ylmethyl)-1H-pyrazol-4-yl]-2H-1,4-benzoxazin-3(4H)-one

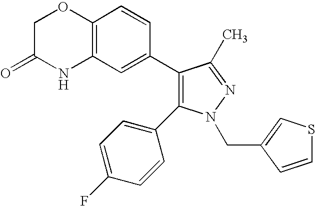

The title compound was obtained from 4-bromo-5-(4-fluorophenyl)-3-methyl-1-(thiophen-3-ylmethyl)-1H-pyrazole (312 mg) according to the similar procedure described for Example 1 (141 mg).

¹H-NMR (300 MHz, DMSO-d₆) δ: 2.20 (3H, s), 4.53 (2H, s), 5.12 (2H, s), 6.58 (1H, dd, J=8.1, 2.1 Hz), 6.66 (1H, d, J=1.9 Hz), 6.70-6.87 (2H, m), 7.09 (1H, dd, J=2.8, 0.9 Hz), 7.18-7.30 (4H, m), 7.45 (1H, dd, J=5.1, 2.8 Hz), 10.60 (1H, s).

LCMS (ESI⁺) M+H⁺: 419.81.

### Example 57

### 6-[5-(4-Fluorophenyl)-3-methyl-1-(1,3-thiazol-4-ylmethyl)-1H-pyrazol-4-yl]-2H-1,4-benzoxazin-3(4H)-one

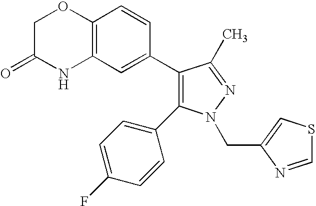

The title compound was obtained from 4-{[4-bromo-5-(4-fluorophenyl)-3-methyl-1H-pyrazol-1-yl]methyl}-1,3-thiazole (130 mg) according to the similar procedure described for Example 1 (12 mg).

¹H-NMR (300 MHz, CDCl₃) δ: 2.32 (3H, s), 4.59 (2H, s), 5.34 (2H, s), 6.46 (1H, d, J=1.9 Hz), 6.71 (1H, dd, J=8.3, 1.9 Hz), 6.87 (1H, d, J=8.3 Hz), 7.04 (2H, t, J=8.8 Hz), 7.10 (1H, d, J=2.1 Hz), 7.19-7.31 (2H, m), 7.72 (1H, s), 8.77 (1H, d, J=1.9 Hz).

LCMS (ESI⁺) M+H⁺: 420.79.

### Example 58

### 6-{1-[(2,4-Dimethyl-1,3-thiazol-5-yl)methyl]-5-(4-fluorophenyl)-3-methyl-1H-pyrazol-4-yl}-2H-1,4-benzoxazin-3(4H)-one

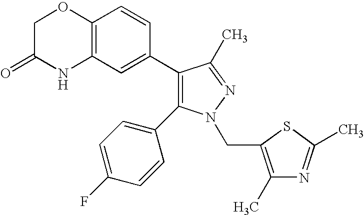

The title compound was obtained from 5-{[4-bromo-5-(4-fluorophenyl)-3-methyl-1H-pyrazol-1-yl]methyl}-2,4-dimethyl-1,3-thiazole (513 mg) according to the similar procedure described for Example 1 (71 mg).

¹H-NMR (300 MHz, DMSO-d₆) δ: 1.94 (3H, s), 2.19 (3H, s), 2.49 (3H, br. s), 4.53 (2H, s), 5.23 (2H, s), 6.56 (1H, dd, J=8.3, 2.1 Hz), 6.64 (1H, d, J=2.1 Hz), 6.81 (1H, d, J=8.3 Hz), 7.21-7.35 (4H, m), 10.59 (1H, s).

LCMS (ESI⁺) M+H⁺: 448.75.

### Example 59

### 6-{5-(4-Fluorophenyl)-3-methyl-1-[(2-methyl-1,3-thiazol-5-yl)methyl]-1H-pyrazol-4-yl}-2H-1,4-benzoxazin-3(4H)-one

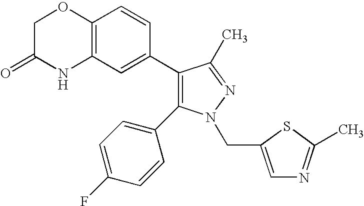

The title compound was obtained from 5-{[4-bromo-5-(4-fluorophenyl)-3-methyl-1H-pyrazol-1-yl]methyl}-2-methyl-1,3-thiazole (185 mg) according to the similar procedure described for Example 1 (111 mg).

¹H-NMR (300 MHz, DMSO-d₆) δ: 2.19 (3H, s), 2.56 (3H, s), 4.53 (2H, s), 5.30 (2H, s), 6.57 (1H, dd, J=8.2, 2.0 Hz), 6.65 (1H, d, J=2.1 Hz), 6.82 (1H, d, J=8.1 Hz), 7.21-7.36 (5H, m), 10.59 (1H, s).

LCMS (ESI⁺) M+H⁺: 434.76.

### Example 60

### 6-[5-(4-Fluorophenyl)-3-methyl-1-(5,5,5-trifluoropentyl)-1H-pyrazol-4-yl]-2H-1,4-benzoxazin-3(4H)-one

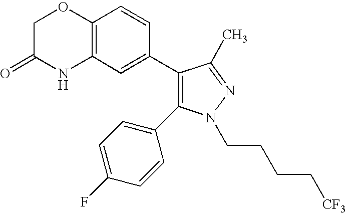

The title compound was obtained from 4-bromo-5-(4-fluorophenyl)-3-methyl-1-(5,5,5-trifluoropentyl)-1H-pyrazole (304 mg) according to the similar procedure described for Example 15 (156 mg).

¹H-NMR (300 MHz, DMSO-d₆) δ: 1.36 (2H, quin, J=7.7 Hz), 1.72 (2H, quin, J=7.4 Hz), 2.03-2.24 (5H, m), 3.92 (2H, t, J=7.1 Hz), 4.53 (2H, s), 6.57 (1H, dd, J=8.3, 2.1 Hz), 6.65 (1H, d, J=1.9 Hz), 6.81 (1H, d, J=8.3 Hz), 7.23-7.34 (4H, m), 10.59 (1H, s).

LCMS (ESI⁺) M+H⁺: 447.82.

### Example 61

### 6-[5-(2,4-Difluorophenyl)-1-methyl-1H-pyrazol-4-yl]-2H-1,4-benzoxazin-3(4H)-one

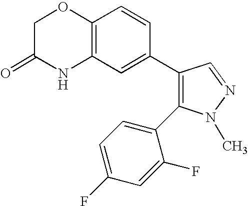

The title compound was obtained from 4-bromo-5-(2,4-difluorophenyl)-1-methyl-1H-pyrazole (900 mg) according to the similar procedure described for Example 1 (158 mg).

¹H-NMR (300 MHz, DMSO-d₆) δ: 3.64 (3H, s), 4.52 (2H, s), 6.65 (1H, d, J=2.1 Hz), 6.72 (1H, dd, J=8.3, 2.1 Hz), 6.84 (1H, d, J=8.3 Hz), 7.25 (1H, td, J=8.6, 1.7 Hz), 7.41-7.56 (2H, m), 7.74 (1H, s), 10.64 (1H, s).

LCMS (ESI⁺) M+H⁺: 342.10.

### Example 62

### 8-Chloro-6-[5-(4-fluorophenyl)-1-methyl-1H-pyrazol-4-yl]-2H-1,4-benzoxazin-3(4H)-one

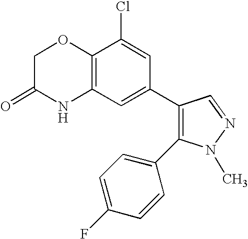

The title compound was obtained from 8-chloro-6-(4,4,5,5-tetramethyl-1,3,2-dioxaborolan-2-yl)-2H-1,4-benzoxazin-3(4H)-one (728 mg) according to the similar procedure described for Example 33 (154 mg).

¹H-NMR (300 MHz, DMSO-d₆) δ: 3.67 (3H, s), 4.64 (2H, s), 6.57 (1H, d, J=2.1 Hz), 6.84 (1H, d, J=2.1 Hz), 7.25-7.51 (4H, m), 7.74 (1H, s), 10.81 (1H, s).

LCMS (ESI⁺) M+H⁺: 358.05.

### Example 63

### 8-Fluoro-6-[5-(4-fluorophenyl)-1-methyl-1H-pyrazol-4-yl]-2H-1,4-benzoxazin-3(4H)-one

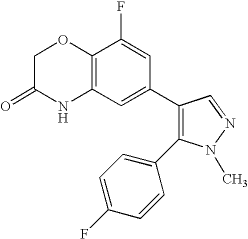

The title compound was obtained from 8-fluoro-6-(4,4,5,5-tetramethyl-1,3,2-dioxaborolan-2-yl)-2H-1,4-benzoxazin-3(4H)-one (689 mg) according to the similar procedure described for Example 33 (118 mg).

¹H-NMR (300 MHz, DMSO-d₆) δ: 3.66 (3H, s), 4.60 (2H, s), 6.45 (1H, m), 6.66 (1H, m), 7.28-7.49 (4H, m), 7.73 (1H, s), 10.81 (1H, br. s).

LCMS (ESI⁺) M+H⁺: 342.10.

### Example 64

### 6-[5-(4-Fluorophenyl)-1-methyl-1H-pyrazol-4-yl]-8-methyl-2H-1,4-benzoxazin-3(4H)-one

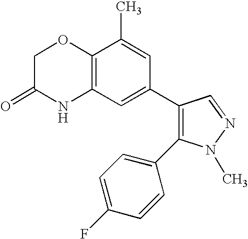

The title compound was obtained from 8-methyl-6-(4,4,5,5-tetramethyl-1,3,2-dioxaborolan-2-yl)-2H-1,4-benzoxazin-3(4H)-one (1.18 g) according to the similar procedure described for Example 33 (325 mg).

¹H-NMR (300 MHz, DMSO-d₆) δ: 2.06 (3H, s), 3.67 (3H, s), 4.52 (2H, s), 6.45 (1H, d, J=1.9 Hz), 6.65 (1H, d, J=1.5 Hz), 7.24-7.51 (4H, m), 7.65 (1H, s), 10.53 (1H, s).

LCMS (ESI⁺) M+H⁺: 338.11.

### Example 65

### 5-Fluoro-6-[5-(4-fluorophenyl)-1-methyl-1H-pyrazol-4-yl]-2H-1,4-benzoxazin-3(4H)-one

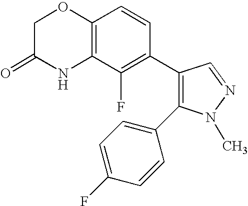

The title compound was obtained from 5-fluoro-6-(4,4,5,5-tetramethyl-1,3,2-dioxaborolan-2-yl)-2H-1,4-benzoxazin-3(4H)-one (340 mg) and 4-bromo-5-(4-fluorophenyl)-1-methyl-1H-pyrazole (444 mg) according to the similar procedure described for Example 1 (91 mg).

¹H-NMR (300 MHz, DMSO-d₆) δ: 3.74 (3H, s), 4.58 (2H, s), 6.56 (1H, t, J=8.2 Hz), 6.70 (1H, dd, J=8.5, 0.9 Hz), 7.21-7.49 (4H, m), 7.62 (1H, d, J=1.9 Hz), 10.82 (1H, s).

LCMS (ESI⁺) M+H⁺: 342.01.

### Example 66

### 6-[5-(4-Fluorophenyl)-1-methyl-1H-pyrazol-4-yl]-5-methyl-2H-1,4-benzoxazin-3(4H)-one

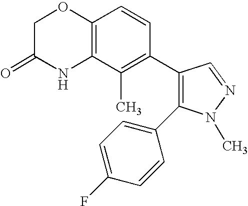

The title compound was obtained from 5-methyl-6-(4,4,5,5-tetramethyl-1,3,2-dioxaborolan-2-yl)-2H-1,4-benzoxazin-3(4H)-one (200 mg) and 4-bromo-5-(4-fluorophenyl)-1-methyl-1H-pyrazole (265 mg) according to the similar procedure described for Example 36 (38 mg).

¹H-NMR (300 MHz, DMSO-d₆) δ: 1.88 (3H, s), 3.78 (3H, s), 4.48 (2H, s), 6.64 (1H, d, J=8.3 Hz), 6.75 (1H, d, J=8.3 Hz), 7.14-7.39 (4H, m), 7.46 (1H, s), 10.10 (1H, br. s).

LCMS (ESI⁺) M+H⁺: 338.01.

### Example 67

### 5,8-Difluoro-6-[5-(4-fluorophenyl)-1-methyl-1H-pyrazol-4-yl]-2H-1,4-benzoxazin-3(4H)-one

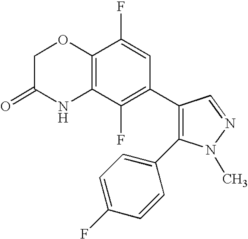

The title compound was obtained from 5,8-difluoro-6-(4,4,5,5-tetramethyl-1,3,2-dioxaborolan-2-yl)-2H-1,4-benzoxazin-3(4H)-one (200 mg) and 4-bromo-5-(4-fluorophenyl)-1-methyl-1H-pyrazole (260 mg) according to the similar procedure described for Example 1 (8 mg).

¹H-NMR (300 MHz, DMSO-d₆) δ: 3.74 (3H, s), 4.67 (2H, s), 6.55 (1H, dd, J=11.2, 6.2 Hz), 7.28-7.37 (2H, m), 7.37-7.47 (2H, m), 7.66 (1H, s), 11.02 (1H, br. s).

LCMS (ESI⁺) M+H⁺: 359.88.

### Example 68

### 5-Chloro-6-[5-(4-fluorophenyl)-1-methyl-1H-pyrazol-4-yl]-2H-1,4-benzoxazin-3(4H)-one

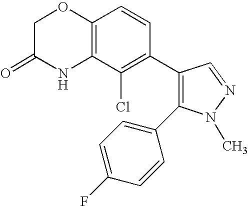

The title compound was obtained from 4-bromo-5-(4-fluorophenyl)-1-methyl-1H-pyrazole (494 mg) according to the similar procedure described for Example 36 (195 mg).

¹H-NMR (300 MHz, DMSO-d₆) δ: 3.77 (3H, s), 4.58 (2H, s), 6.72 (1H, d, J=8.3 Hz), 6.88 (1H, d, J=8.5 Hz), 7.19-7.39 (4H, m), 7.56 (1H, s), 10.36 (1H, br. s).

LCMS (ESI⁺) M+H⁺: 357.82.

### Example 69

### 6-{5-(4-Fluorophenyl)-1-[2-(methylsulfanyl)ethyl]-1H-pyrazol-4-yl}-2H-1,4-benzoxazin-3(4H)-one

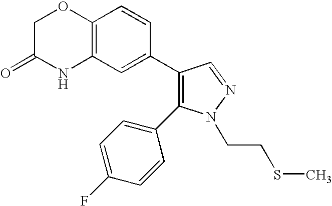

The title compound was obtained from 4-bromo-5-(4-fluorophenyl)-1-[2-(methylsulfanyl)ethyl]-1H-pyrazole (1.63 g) according to the similar procedure described for Example 15 (1.14 g).

¹H-NMR (300 MHz, DMSO-d₆) δ: 1.81 (3H, s), 2.80 (2H, t, J=6.9 Hz), 4.10 (2H, t, J=6.9 Hz), 4.51 (2H, s), 6.65-6.72 (2H, m), 6.82 (1H, d, J=8.7 Hz), 7.30-7.47 (4H, m), 7.74 (1H, s), 10.63 (1H, s).

LCMS (ESI⁺) M+H⁺: 383.80.

### Example 70

### 6-{5-(4-Fluorophenyl)-1-[2-(methylsulfinyl)ethyl]-1H-pyrazol-4-yl}-2H-1,4-benzoxazin-3(4H)-one

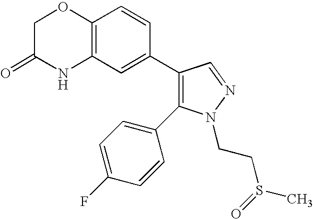

The title compound was obtained from 6-{5-(4-fluorophenyl)-1-[2-(methylsulfanyl)ethyl]-1H-pyrazol-4-yl}-2H-1,4-benzoxazin-3(4H)-one (300 mg) according to the similar procedure described for Example 41 (247 mg).

¹H-NMR (300 MHz, DMSO-d₆) δ: 2.51 (3H, br. s), 2.98-3.35 (2H, m), 4.29 (2H, t, J=6.1 Hz), 4.51 (2H, s), 6.63-6.72 (2H, m), 6.82 (1H, d, J=8.1 Hz), 7.29-7.39 (2H, m), 7.39-7.49 (2H, m), 7.77 (1H, s), 10.63 (1H, s).

LCMS (ESI⁺) M+H⁺: 499.78.

### Example 71

### 6-{5-(4-Fluorophenyl)-1-[2-(methylsulfonyl)ethyl]-1H-pyrazol-4-yl}-2H-1,4-benzoxazin-3(4H)-one

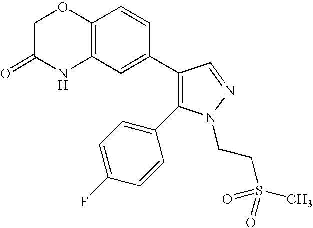

The title compound was obtained using 2 eq. amount of 3-chlorobenzenecarboperoxoic acid from 6-{5-(4-fluorophenyl)-1-[2-(methylsulfanyl)ethyl]-1H-pyrazol-4-yl}-2H-1,4-benzoxazin-3(4H)-one (800 mg) according to the similar procedure described for Example 41 (316 mg).

¹H-NMR (300 MHz, DMSO-d₆) δ: 2.90 (3H, s), 3.66 (2H, t, J=7.2 Hz), 4.32 (2H, t, J=7.1 Hz), 4.51 (2H, s), 6.64-6.74 (2H, m), 6.83 (1H, d, J=8.3 Hz), 7.32-7.41 (2H, m), 7.41-7.50 (2H, m), 7.79 (1H, s), 10.63 (1H, s).

LCMS (ESI⁺) M+H⁺: 415.75.

### Example 72

### 6-[1-(2,2-Difluoropropyl)-5-(4-fluorophenyl)-1H-pyrazol-4-yl]-2H-1,4-benzoxazin-3(4H)-one

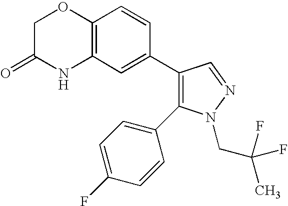

The title compound was obtained from 4-bromo-1-(2,2-difluoropropyl)-5-(4-fluorophenyl)-1H-pyrazole (2.98 g) according to the similar procedure described for Example 15 (1.49 g).

¹H-NMR (300 MHz, DMSO-d₆) δ: 1.57 (3H, t, J=19.1 Hz), 4.43 (2H, t, J=12.9 Hz), 4.52 (2H, s), 6.63-6.73 (2H, m), 6.82 (1H, d, J=8.0 Hz), 7.31-7.42 (4H, m), 7.82 (1H, s), 10.65 (1H, s).

LCMS (ESI⁺) M+H³⁰ : 388.0.

### Preparation 97

### 6-{1-[4-(Benzyloxy)butyl]-5-(4-fluorophenyl)-3-methyl-1H-pyrazol-4-yl}-2H-1,4-benzoxazin-3(4H)-one

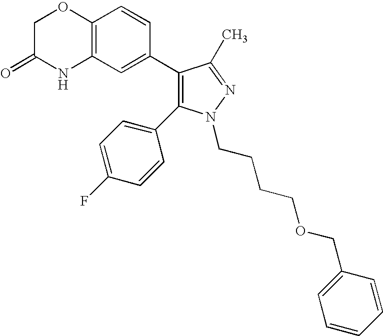

The title compound was obtained from 1-[4-(benzyloxy)butyl]-4-bromo-5-(4-fluorophenyl)-3-methyl-1H-pyrazole (1 g) according to the similar procedure described for Example 33 (510 mg).

¹H-NMR (300 MHz, CDCl₃) δ: 1.47-1.59 (2H, m), 1.81-1.92 (2H, m), 2.30 (3H, s), 3.38 (2H, t, J=6.3 Hz), 3.99 (2H, t, J=7.5 Hz), 4.22 (2H, s), 4.57 (2H, s), 6.43 (1H, d, J=2.1 Hz), 6.65 (1H, dd, J=8.4, 2.1 Hz), 6.84 (1H, d, J=8.4 Hz), 6.98-7.08 (2H, m), 7.11-7.18 (2H, m), 7.22-7.36 (5H, m), 7.97 (1H, s).

### Example 73

### 6-[5-(4-Fluorophenyl)-1-(4-hydroxybutyl)-3-methyl-1H-pyrazol-4-yl]-2H-1,4-benzoxazin-3(4H)-one

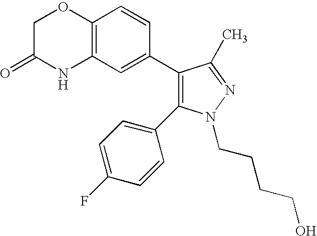

A mixture of 6-{1-[4-(benzyloxy)butyl]-5-(4-fluorophenyl)-3-methyl-1H-pyrazol-4-yl}-2H-1,4-benzoxazin-3(4H)-one (0.49 g) and 10% palladium-carbon (0.1 g) in ethanol (30 ml) was stirred at room temperature for 5 h, and at 50° C. for 18 h. The unsoluble material was filtered off, and washed with ethyl acetate. The ethyl acetate solution was concentrated, and crystallized from ethyl acetate/hexane to give the title compound (0.35 g).

Mp 194.5-196.0° C.

¹H-NMR (300 MHz, DMSO-d₆) δ: 1.10-1.37 (2H, m), 1.60-1.73 (2H, m), 2.18 (3H, s), 3.23-3.34 (2H, m), 3.88 (2H, t, J=7.2 Hz), 4.34 (1H, t, J=5.1 Hz), 4.52 (2H, s), 6.55 (1H, dd, J=8.4, 1.8 Hz), 6.64 (1H, d, J=1.8 Hz), 6.80 (1H, d, J=8.4 Hz), 7.21-7.32 (4H, m), 10.58 (1H, s).

### Preparation 98

### 1-Ethyl-5-(4-fluorophenyl)-1H-pyrazole

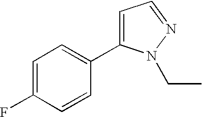

A solution of 3-(dimethylamino)-1-(4-fluorophenyl)prop-2-en-1-one (8.00 g), ethylhydrazine (3.73 g), trifluoroacetic acid (4.6 ml) and Et₃N (8.7 ml) in 2-propanol (70 ml) was stirred for 7 h at 80° C. The reaction mixture was treated with ethyl acetate and H₂O. The organic layer was separated, washed with brine, dried over Na₂SO₄ and concentrated in vacuo. The residue was purified by silica gel column chromatography using n-hexane/ethyl acetate as an eluent to give the title compound (6.62 g, 90%).

¹H-NMR (300 MHz, DMSO-d₆) δ: 1.29 (3H, t, J=7.0 Hz), 4.10 (2H, q, J=7.0 Hz), 6.34 (1H, d, J=2.0 Hz), 7.27-7.41 (2H, m), 7.45-7.57 (3H, m).

### Preparation 99

### 4-Bromo-1-ethyl-5-(4-fluorophenyl)-1H-pyrazole

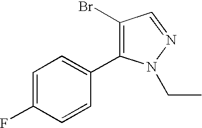

To a solution of 1-ethyl-5-(4-fluorophenyl)-1H-pyrazole (6.62 g) in N,N-dimethylformamide (70 ml) was added N-bromosuccinimide (7.43 g) at 0° C. After stirring for 15 min. at 0° C., the reaction was quenched with Na₂S₂O₃ aqueous solution. The solution was treated with ethyl acetate and H₂O. The organic layer was separated, washed with brine, dried over Na₂SO₄ and concentrated in vacuo. The residue was purified by silica gel column chromatography using n-hexane/ethyl acetate as an eluent to give the title compound (9.13 g, 98%).

¹H-NMR (300 MHz, DMSO-d₆) δ: 1.23 (3H, t, J=7.0 Hz), 4.03 (2H, q, J=7.0 Hz), 7.35-7.45 (2H, m), 7.47-7.56 (2H, m), 7.68 (1H, s).

### Example 74

### 6-[1-Ethyl-5-(4-fluorophenyl)-1H-pyrazol-4-yl]-2H-1,4-benzoxazin-3(4H)-one

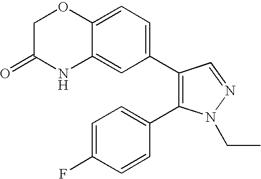

A mixture of 4-bromo-1-ethyl-5-(4-fluorophenyl)-1H-pyrazole (588 mg), 6-(4,4,5,5-tetramethyl-1,3,2-dioxaborolan-2-yl)-2H-1,4-benzoxazin-3(4H)-one (500 mg), [1,1′-bis(diphenylphosphino)ferrocene]dichloropalladium(II) dichloromethane adduct (297 mg) and Cs₂CO₃ (1.80 g) in tetrahydrofuran (15 ml) and H₂O (3 ml) was stirred for 15 min. at 150° C. under microwave irradiation. After cooling, the reaction mixture was treated with ethyl acetate and H₂O. The organic layer was separated, washed with brine, dried over Na₂SO₄ and concentrated in vacuo. The residue was purified by silica gel column chromatography using n-hexane/ethyl acetate as an eluent to give the title compound (39 mg, 5%).

¹H-NMR (300 MHz, DMSO-d₆) δ: 1.24 (3 H, t, J=7.0 Hz), 3.94 (2H, q, J=7.0 Hz), 4.51 (2H, s), 6.61-6.72 (2H, m), 6.74-6.87 (2H, m), 7.24-7.46 (4H, m), 7.69 (1H, s), 10.63 (1H, br. s).

### Preparation 100

### 5-(4-Fluorophenyl)-1-(2,2,2-trifluoroethyl)-1H-pyrazole

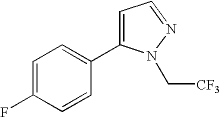

The title compound (9.50 g) was synthesized from (2,2,2-trifluoroethyl)hydrazine (9.50 g) according to the similar procedure described for preparation 98.

¹H-NMR (300 MHz, DMSO-d₆) δ: 5.02 (2H, q, J=9.0 Hz), 6.49 (1H, d, J=2.0 Hz), 7.30-7.41 (2H, m), 7.47-7.59 (2H, m), 7.66 (1H, d, J=2.0 Hz).

### Preparation 101

### 4-Bromo-5-(4-fluorophenyl)-1-(2,2,2-trifluoroethyl)-1H-pyrazole

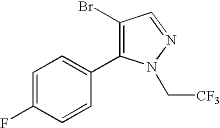

The title compound (10.87 g) was synthesized from 5-(4-fluorophenyl)-1-(2,2,2-trifluoroethyl)-1H-pyrazole (9.48 g) according to the similar procedure described for preparation 99.

¹H-NMR (300 MHz, DMSO-d₆) δ: 4.99 (2H, q, J=8.5 Hz), 7.42 (2H, t, J=9.0 Hz), 7.51 (2H, dd, J=9.0, 5.5 Hz), 7.88 (1H, s).

### Example 75

### 6-[5-(4-Fluorophenyl)-1-(2,2,2-trifluoroethyl)-1H-pyrazol-4-yl]-2H-1,4-benzoxazin-3(4H)-one

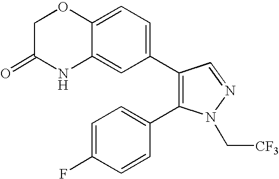

The title compound (420 mg) was synthesized from 4-bromo-5-(4-fluorophenyl)-1-(2,2,2-trifluoroethyl)-1H-pyrazole (500 mg) according to the similar procedure described for Example 74. Obtained compound was recrystallized from ethyl acetate and hexane.

Mp 227.0-227.4° C.

¹H-NMR (300 MHz, DMSO-d₆) δ: 4.52 (2H, s), 4.87 (2H, q, J=9.0 Hz), 6.66 (1H, d, J=2.0 Hz), 6.72 (1H, dd, J=8.5, 2.0 Hz), 6.84 (1H, d, J=8.5 Hz), 7.31-7.43 (4H, m), 7.88 (1H, s), 10.66 (1H, br. s).

### Example 76

### 6-[5-(4-Fluorophenyl)-3-methyl-1H-pyrazol-4-yl]-2H-1,4-benzoxazin-3(4H)-one

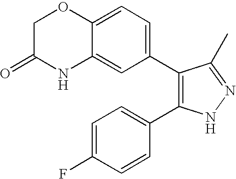

The title compound (146 mg) was synthesized from 4-bromo-5-(4-fluorophenyl)-3-methyl-1H-pyrazole (557 mg) according to the similar procedure described for Example 74. Obtained compound was recrystallized from ethyl acetate and hexane.

Mp 294.6-297.7° C.

¹H-NMR (300 MHz, DMSO-d₆) δ: 2.17 (3H, br. s), 4.59 (2H, s), 6.63-6.76 (2H, m), 6.87-7.45 (5H, m), 10.62 (1H, s), 12.67-12.96 (1H, m).

### Preparation 102

### 1-Ethyl-5-(4-fluorophenyl)-3-methyl-1H-pyrazole

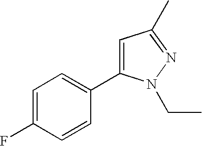

The title compound (7.16 g) was synthesized from ethylhydrazine (3.50 g) according to the similar procedure described for preparation 98.

¹H-NMR (300 MHz, DMSO-d₆) δ: 1.26 (3H, t, J=7.0 Hz), 2.17 (3H, s), 3.99 (2H, q, J=7.0 Hz), 6.11 (1H, s), 7.27-7.37 (2H, m), 7.43-7.54 (2H, m).

### Preparation 103

### 4-Bromo-1-ethyl-5-(4-fluorophenyl)-3-methyl-1H-pyrazole

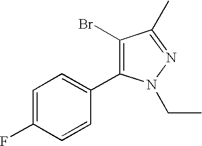

The title compound (9.89 g) was synthesized from 1-ethyl-5-(4-fluorophenyl)-3-methyl-1H-pyrazole (7.16 g) according to the similar procedure described for preparation 99.

¹H-NMR (300 MHz, DMSO-d₆) δ: 1.21 (3H, t, J=7.0 Hz), 2.18 (3H, s), 3.96 (2H, q, J=7.0 Hz), 7.34-7.45 (2H, m), 7.45-7.54 (2H, m).

### Example 77

### 6-[1-Ethyl-5-(4-fluorophenyl)-3-methyl-1H-pyrazol-4-yl]-2H-1,4-benzoxazin-3(4H)-one

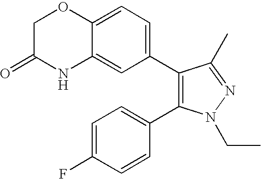

The title compound (213 mg) was synthesized from 4-bromo-1-ethyl-5-(4-fluorophenyl)-3-methyl-1H-pyrazole (618 mg) according to the similar procedure described for Example 74. Obtained compound was recrystallized from ethyl acetate and hexane.

Mp 160.7-160.9° C.

¹H-NMR (300 MHz, DMSO-d₆) δ: 1.24 (3H, t, J=7.0 Hz), 2.19 (3H, s), 3.91 (2H, q, J=7.0 Hz), 4.53 (2H, s), 6.57 (1H, dd, J=8.0, 2.0 Hz), 6.65 (1H, d, J=2.0 Hz), 6.81 (1H, d, J=8.0 Hz), 7.19-7.36 (4H, m), 10.60 (1H, s).

### Preparation 104

### 5-(4-Fluorophenyl)-3-methyl-1-prop-2-en-1-yl-1H-pyrazole

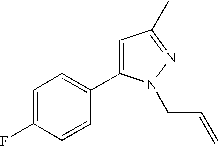

The title compound (19.48 g) was synthesized from 70% aqueous allylhydrazine (14.6 g) according to the similar procedure described for preparation 98.

¹H-NMR (300 MHz, DMSO-d₆) δ: 2.18 (3H, s), 4.57-4.69 (2H, m), 4.86 (1H, dd, J=17.0, 1.5 Hz), 5.11 (1H, dd, J=10.5, 1.5 Hz), 5.85-6.01 (1H, m), 6.19 (1H, s), 7.31 (2H, t, J=9.0 Hz), 7.43-7.53 (2H, m).

### Preparation 105

### 4-Bromo-5-(4-fluorophenyl)-3-methyl-1-propyl-1H-pyrazole

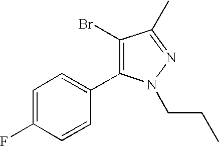

A suspension of 5-(4-fluorophenyl)-3-methyl-1-prop-2-en-1-yl-1H-pyrazole (2.0 g) and 10% Pd on carbon (300 mg) in MeOH (50 ml) was stirred for 12 h under H₂ atmosphere at rt. The reaction mixture was filtered through filter paper. The filtrate was concentrated in vacuo. The residue was dissolved in N,N-dimethylformamide (30 ml). To this solution was added N-bromosuccinimide (1.98 g) at rt. After stirring for 30 min, the reaction mixture was quenched with Na₂S₂O₃ aqueous solution. The solution was treated with ethyl acetate and H₂O. The organic layer was separated, washed with brine, dried over Na₂SO₄ and concentrated in vacuo. The residue was purified by silica gel column chromatography using n-hexane/ethyl acetate as an eluent to give the title compound (2.81 g, quant.).

¹H-NMR (300 MHz, CDCl₃) δ: 0.69 (3H, t, J=7.5 Hz), 1.54-1.70 (2H, m), 2.18 (3 H, s), 3.90 (2H, t, J=7.0 Hz), 7.39 (2H, t, J=9.0 Hz), 7.48 (2H, dd, J=9.0, 5.5 Hz).

### Example 78

### 6-[5-(4-Fluorophenyl)-3-methyl-1-propyl-1H-pyrazol-4-yl]-2H-1,4-benzoxazin-3(4H)-one

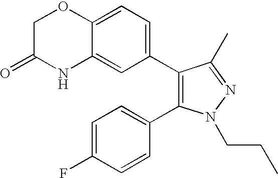

The title compound (264 mg) was synthesized from 4-bromo-5-(4-fluorophenyl)-3-methyl-1-propyl-1H-pyrazole (594 mg) according to the similar procedure described for Example 74. Obtained compound was recrystallized from ethyl acetate and hexane.

Mp 190.8-190.9° C.

¹H-NMR (300 MHz, DMSO-d₆) δ: 0.73 (3H, t, J=7.5 Hz), 1.57-1.73 (2H, m), 2.19 (3H, s), 3.84 (2H, t, J=7.5 Hz), 4.53 (2H, s), 6.56 (1H, dd, J=8.5, 2.0 Hz), 6.65 (1H, d, J=2.0 Hz), 6.81 (1H, d, J=8.5 Hz), 7.22-7.35 (4H, m), 10.60 (1H, br. s).

### Preparation 106

### 5-(4-Fluorophenyl)-3-methyl-1-(2,2,2-trifluoroethyl)-1H-pyrazole

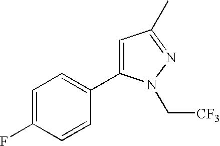

The title compound (8.94 g) was synthesized from 70% aqueous (2,2,2-trifluoroethyl)hydrazine (9.50 g) according to the similar procedure described for preparation 98.

¹H-NMR (300 MHz, DMSO-d₆) δ: 2.21 (3H, s), 4.90 (2H, q, J=9.0 Hz), 6.28 (1H, s), 7.28-7.40 (2H, m), 7.43-7.55 (2H, m).

### Preparation 107

### 4-Bromo-5-(4-fluorophenyl)-3-methyl-1-(2,2,2-trifluoroethyl)-1H-pyrazole

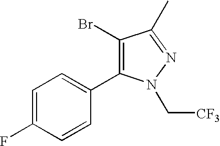

The title compound (11.78 g) was synthesized from 5-(4-fluorophenyl)-3-methyl-1-(2,2,2-trifluoroethyl)-1H-pyrazole (8.94 g) according to the similar procedure described for preparation 99.

¹H-NMR (300 MHz, DMSO-d₆) δ: 2.22 (3H, s), 4.90 (2H, q, J=8.5 Hz), 7.35-7.55 (4H, m).

### Example 79

### 6-[5-(4-Fluorophenyl)-3-methyl-1-(2,2,2-trifluoroethyl)-1H-pyrazole-4-yl]-2H-1,4-benzoxazin-3(4H)-one

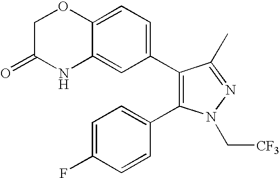

The title compound (610 mg) was synthesized from 4-bromo-5-(4-fluorophenyl)73-methyl-1-(2,2,2-trifluoroethyl)-1H-pyrazole (736 mg) according to the similar procedure described for Example 74. Obtained compound was recrystallized from ethyl acetate and hexane.

Mp 173.5-173.9° C.

¹H-NMR (300 MHz, DMSO-d₆) δ: 2.21 (3H, s), 4.54 (2H, s), 4.82 (2H, q, J=9.0 Hz), 6.61 (1H, dd, J=8.5, 2.0 Hz), 6.66 (1H, d, J=2.0 Hz), 6.83 (1H, d, J=8.5 Hz), 7.22-7.37 (4H, m), 10.61 (1H, br. s).

### Preparation 108

### 2-[5-(4-Fluorophenyl)-3-methyl-1H-pyrazol-1-yl]ethane-1,1-diol

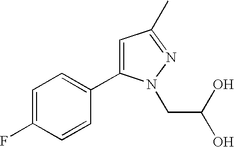

To a solution of 5-(4-fluorophenyl)-3-methyl-1-prop-2-en-1-yl-1H-pyrazole (4.69 g) in acetone (60 ml) and H₂O (15 ml) were added potassium osmate (VI) dehydrate (1.00 g) and N-methylmorpholine-N-oxide (3.80 g) at rt. After stirring for 12 h at rt, the reaction was quenched with Na₂S₂O₃ aqueous solution. The mixture was extracted with ethyl acetate. The organic layer was washed with brine, dried over Na₂SO₄ and concentrated in vacuo. The one-eighth of resulting residue was dissolved with a mixture of tetrahydrofuran (72 ml), MeOH (24 ml) and H₂O (36 ml). To this solution was added NaIO₄ (2.61 g) at rt. After stirring for 3 days at rt, the mixture was treated with ethyl acetate and H₂O. The organic layer was separated, washed with brine, dried over Na₂SO₄ and concentrated in vacuo. The residue was purified by silica gel column chromatography using n-hexane/ethyl acetate as an eluent to give the title compound (660 mg, quant.).

¹H-NMR (300 MHz, DMSO-d₆) δ: 2.17 (3H, s), 3.87 (2H, d, J=5.5 Hz), 5.22 (1H, t, J=5.5 Hz), 6.10 (1H, s), 6.13 (1H, br. s.), 6.15 (1H, br. s.), 7.31 (2H, t, J=9.0 Hz), 7.64 (2H, dd, J=9.0, 5.5 Hz).

### Preparation 109

### 1-(2,2-Difluoroethyl)-5-(4-fluorophenyl)-3-methyl-1H-pyrazole

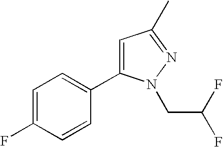

To a solution of 2-[5-(4-fluorophenyl)-3-methyl-1H-pyrazol-1-yl]ethane-1,1-diol (660 mg) in CH₂Cl₂ (20 ml) was added diethylaminosulfur trifluoride (1.80 g) in CH₂Cl₂ (4 ml) at −78° C. After stirring for 12 h at rt, the mixture was treated with ethyl acetate and H₂O. The organic layer was separated, washed with brine, dried over Na₂SO₄ and concentrated in vacuo. The residue was purified by silica gel column chromatography using n-hexane/ethyl acetate as an eluent to give the title compound (532 mg, 79%).

¹H-NMR (300 MHz, DMSO-d₆) δ: 2.20 (3H, s), 4.41 (2H, td, J=14.5, 4.0 Hz), 6.33 (1H, tt, J=55.0, 4.0 Hz), 6.22 (1H, s), 7.34 (2H, t, J=9.0 Hz), 7.49 (2H, dd, J=9.0, 5.5 Hz).

### Preparation 110

### 4-Bromo-1-(2,2-difluoroethyl)-5-(4-fluorophenyl)-3-methyl-1H-pyrazole

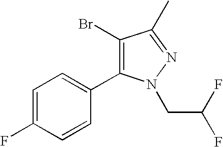

The title compound (977 mg) was synthesized from 1-(2,2-difluoroethyl)-5-(4-fluorophenyl)-3-methyl-1H-pyrazole (760 mg) according to the similar procedure described for preparation 99.

¹H-NMR (300 MHz, DMSO-d₆) δ: 2.21 (3H, s), 4.40 (2H, td, J=14.5, 3.5 Hz), 6.28 (1H, tt, J=54.5, 3.5 Hz), 7.41 (2H, t, J=9.0 Hz), 7.48 (2H, dd, J=9.0, 5.5 Hz).

### Example 80

### 6-[1-(2,2-Difluoroethyl)-5-(4-fluorophenyl)-3-methyl-1H-pyrazol-4-yl]-2H-1,4-benzoxazin-3(4H)-one

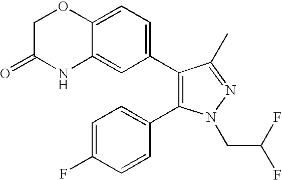

The title compound (102 mg) was synthesized from 4-bromo-1-(2,2-difluoroethyl)-5-(4-fluorophenyl)-3-methyl-1H-pyrazole (638 mg) according to the similar procedure described for Example 74. Obtained compound was recrystallized from ethyl acetate and hexane.

Mp 167.9-168.2° C.

¹H-NMR (300 MHz, DMSO-d₆) δ: 2.21 (3H, s), 4.33 (2H, td, J=14.5, 4.0 Hz), 4.54 (2H, s), 6.32 (1H, tt, J=55.0, 4.0 Hz), 6.59 (1H, dd, J=8.5, 2.0 Hz), 6.66 (1H, d, J=2.0 Hz), 6.83 (1H, d, J=8.5 Hz), 7.26-7.34 (4H, m), 10.61 (1H, br. s).

### Preparation 111

### 2-[5-(4-Fluorophenyl)-3-methyl-1H-pyrazol-1-yl]ethanol

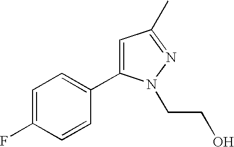

The title compound (8.28 g) was synthesized from 2-hydrazinoethanol (4.40 g) according to the similar procedure described for preparation 98.

¹H-NMR (300 MHz, DMSO-d₆) δ: 2.18 (3H, s), 3.75 (2H, q, J=5.5 Hz), 3.98 (2H, t, J=5.5 Hz), 4.92 (1H, t, J=5.0 Hz), 6.12 (1H, s), 7.25-7.40 (2H, m), 7.51-7.67 (2H, m).

### Preparation 112

### 2-[4-Bromo-5-(4-fluorophenyl)-3-methyl-1H-pyrazol-1-yl]ethanol

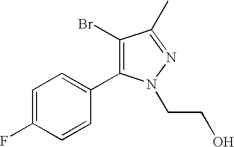

The title compound (9.43 g) was synthesized from 2-[5-(4-fluorophenyl)-3-methyl-1H-pyrazol-1-yl]ethanol (8.28 g) according to the similar procedure described for preparation 99.

¹H-NMR (300 MHz, DMSO-d₆) δ: 2.19 (3H, s), 3.68 (2H, q, J=5.5 Hz), 3.95 (2H, t, J=5.5 Hz), 4.89 (1H, t, J=5.5 Hz), 7.32-7.43 (2H, m), 7.50-7.61 (2H, m).

### Preparation 113

### 4-Bromo-1-(2-fluoroethyl)-5-(4-fluorophenyl)-3-methyl-1H-pyrazole

### 4-Bromo-1-(2-chloroethyl)-5-(4-fluorophenyl)-3-methyl-1H-pyrazole

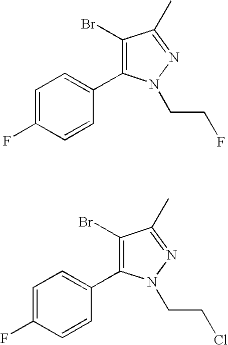

To a solution of 2-[4-bromo-5-(4-fluorophenyl)-3-methyl-1H-pyrazol-1-yl]ethanol (1.40 g) in CH₂Cl₂ (15 ml) was added bis(2-methoxyethyl)aminosulfur trifluoride (1.24 g) in CH₂Cl₂ (5 ml) at −78° C. After stirring for 1 h at rt, the mixture was treated with ethyl acetate and H₂O. The organic layer was separated, washed with brine, dried over Na₂SO₄ and concentrated in vacuo. The residue was purified by silica gel column chromatography using n-hexane/ethyl acetate as an eluent to give 4-bromo-1-(2-fluoroethyl)-5-(4-fluorophenyl)-3-methyl-1H-pyrazole (330 mg, 23%) and 4-bromo-1-(2-chloroethyl)-5-(4-fluorophenyl)-3-methyl-1H-pyrazole (580 mg, 39%).

### 4-bromo-1-(2-fluoroethyl)-5-(4-fluorophenyl)-3-methyl-1H-pyrazole

¹H-NMR (300 MHz, DMSO-d₆) δ: 2.21 (3H, s), 4.23 (2H, dt, J=27.0, 4.5 Hz), 4.70 (2H, dt, J=47.0, 4.5 Hz), 7.33-7.53 (4H, m).

### 4-bromo-1-(2-chloroethyl)-5-(4-fluorophenyl)-3-methyl-1H-pyrazole

¹H-NMR (300 MHz, DMSO-d_{6 . . .} δ: 2.21 (3H, s), 3.92 (2H, t, J=5.5 Hz), 4.25 (2H, t, J=5.5 Hz), 7.40 (2H, t, J=9.0 Hz), 7.50 (2H, dd, J=9.0, 5.5 Hz).

### Example 81

### 6-[1-(2-Fluoroethyl)-5-(4-fluorophenyl)-3-methyl-1H-pyrazol-4-yl]-2H-1,4-benzoxazin-3(4H)-one

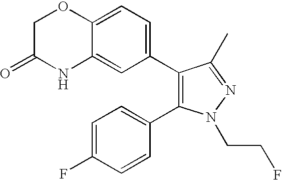

The title compound (273 mg) was synthesized from 4-bromo-1-(2-fluoroethyl)-5-(4-fluorophenyl)-3-methyl-1H-pyrazole (330 mg) according to the similar procedure described for Example 74. Obtained compound was recrystallized from ethyl acetate and hexane.

Mp 186.1-187.1° C.

¹H-NMR (300 MHz, DMSO-d₆) δ: 2.21 (3H, s), 4.18 (2H, dt, J=26.5, 4.5 Hz), 4.53 (2H, s), 4.73 (2H, dt, J=47.0, 4.5 Hz), 6.58 (1H, dd, J=8.5, 2.0 Hz), 6.66 (1H, d, J=2.0 Hz), 6.82 (1H, d, J=8.5 Hz), 7.23-7.33 (4H, m), 10.61 (1 H, br. s).

### Example 82

### 6-[1-(2-Chloroethyl)-5-(4-fluorophenyl)-3-methyl-1H-pyrazole-4-yl]-2H-1,4-benzoxazin-3(4H)-one

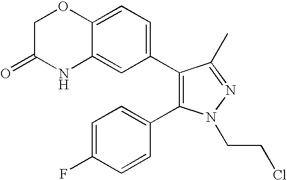

The title compound (363 mg) was synthesized from 4-bromo-1-(2-chloroethyl)-5-(4-fluorophenyl)-3-methyl-1H-pyrazole (580 mg) according to the similar procedure described for Example 74. Obtained compound was recrystallized from ethyl acetate and hexane.

Mp 216.0-216.5° C.

¹H-NMR (300 MHz, DMSO-d₆) δ: 2.21 (3H, s), 3.95 (2H, t, J=5.5 Hz), 4.19 (2H, t, J=5.5 Hz), 4.53 (2H, s), 6.58 (1H, dd, J=8.5, 2.0 Hz), 6.66 (1H, d, J=2.0 Hz), 6.82 (1H, d, J=8.5 Hz), 7.20-7.39 (4H, m), 10.60 (1H, br. s).

### Preparation 114

### Ethyl[5-(4-fluorophenyl)-3-methyl-1H-pyrazol-1-yl]acetate

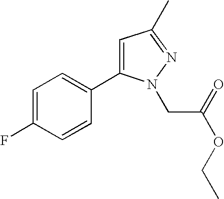

A solution of 1-(4-Fluorophenyl)butane-1,3-dione (7.00 g), ethyl hydrazinoacetate (9.0 g), trifluoroacetic acid (4.3 ml) and Et₃N (8.2 ml) in 2-propanol (70 ml) was stirred for 7 h at 80° C. The reaction mixture was treated with ethyl acetate and H₂O. The organic layer was separated, washed with brine, dried over Na₂SO₄ and concentrated in vacuo. The residue was purified by silica gel column chromatography using n-hexane/ethyl acetate as an eluent to give the title compound (6.42 g, 63%).

¹H-NMR (300 MHz, DMSO-d₆) δ: 1.12 (3H, t, J=7.0 Hz),2.18 (3H, s), 4.08 (2H, q, J=7.0 Hz), 4.89 (2H, s), 6.21 (1H, s), 7.26-7.36 (2H, m), 7.40-7.52 (2H, m).

### Preparation 115

### Ethyl[4-bromo-5-(4-fluorophenyl)-3-methyl-1H-pyrazol-1-yl]acetate

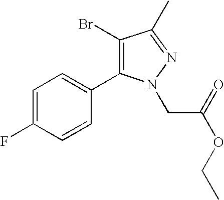

To a solution of ethyl [5-(4-fluorophenyl)-3-methyl-1H-pyrazol-1-yl]acetate (6.42 g) in N,N-dimethylformamide (50 ml) was added N-bromosuccinimide (5.23 g) at 0° C. After stirring for 15 min. at 0° C., the reaction was quenched with Na₂S₂O₃ aqueous solution. The solution was treated with ethyl acetate and H₂O. The organic layer was separated, washed with brine, dried over Na₂SO₄ and concentrated in vacuo. The residue was purified by silica gel column chromatography using n-hexane/ethyl acetate as an eluent to give the title compound (7.85 g, 94%).

¹H-NMR (300 MHz, DMSO-d₆) δ: 1.10 (3H, t, J=7.5 Hz),2.19 (3H, s), 4.06 (2H, q, J=7.5 Hz), 4.89 (2H, s), 7.30-7.55 (4H, m).

### Preparation 116

### 2-[4-Bromo-5-(4-fluorophenyl)-3-methyl-1H-pyrazol-1-yl]-N-methoxy-N-methylacetamide

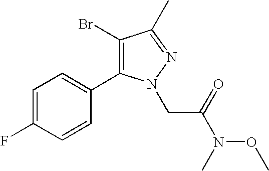

A solution of ethyl [4-bromo-5-(4-fluorophenyl)-3-methyl-1H-pyrazol-1-yl]acetate (2.40 g) in 1N NaOH aqueous solution (25 ml) and Me0H (25 ml) was stirred for 3 h at rt. The reaction mixture was acidified with 1N HCl aqueous solution and then extracted with ethyl acetate. The organic layer was washed with brine, dried over Na₂SO₄ and concentrated in vacuo. The residue was dissolved with N,N-dimethylformamide (25 ml). To this solution were added N,O-dimethylhydroxylamine hydrochloride (1.07 g), WSC (2.11 g) and HOBt (1.49 g) at rt. After stirring for 12 h at rt, the mixture was treated with ethyl acetate and 1N HCl aqueous solution. The organic layer was separated, washed with brine, dried over Na₂SO₄ and concentrated in vacuo. The residue was purified by silica gel column chromatography using n-hexane/ethyl acetate as an eluent to give the title compound (2.07 g, 79%).

¹H-NMR (300 MHz, DMSO-d₆) δ: 2.18 (3H, s), 3.07 (3H, s), 3.61 (3H, s), 4.97 (2H, s), 7.37 (2H, t, J=9.0 Hz), 7.46 (2H, dd, J=9.0, 5.5 Hz).

### Preparation 117

### 1-[4-Bromo-5-(4-fluorophenyl)-3-methyl-1H-pyrazol-1-yl]propan-2-one

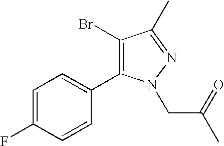

To a solution of 2-[4-bromo-5-(4-fluorophenyl)-3-methyl-1H-pyrazol-1-yl]-N-methoxy-N-methylacetamide (2.07 g) in dry tetrahydrofuran (50 ml) was added 3M MeMgBr (2.32 ml, Et₂O solution) at 0° C. After stirring for 3 h at rt, the mixture was treated with ethyl acetate and 1N HCl aqueous solution. The organic layer was separated, washed with brine, dried over Na₂SO₄ and concentrated in vacuo. The residue was purified by silica gel column chromatography using n-hexane/ethyl acetate as an eluent to give the title compound (995 mg, 55%).

¹H-NMR (300 MHz, DMSO-d₆) δ: 2.03 (3H, s), 3.32 (3H, s), 5.00 (2H, s), 7.35 (2H, d, J=2.5 Hz), 7.38 (2H, s).

### Preparation 118

### 4-Bromo-1-(2,2-difluoropropyl)-5-(4-fluorophenyl)-3-methyl-1H-pyrazole

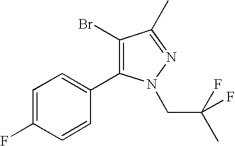

To a solution of 1-[4-bromo-5-(4-fluorophenyl)-3-methyl-1H-pyrazol-1-yl]propan-2-one (500 mg) in CH₂Cl₂ (10 ml) was added diethylaminosulfur trifluoride (1.03 g) in CH₂Cl₂ (2 ml) at −78° C. After stirring for 12 h at rt, the mixture was treated with ethyl acetate and H₂O. The organic layer was separated, washed with brine, dried over Na₂SO₄ and concentrated in vacuo. The residue was purified by silica gel column chromatography using n-hexane/ethyl acetate as an eluent to give the title compound (544 mg, quant.).

¹H-NMR (300 MHz, DMSO-d₆) δ: 1.53 (3H, t, J=19.0 Hz), 2.21 (3H, s), 4.45 (2H, t, J=13.0 Hz), 7.39 (2H, t, J=9.0 Hz), 7.47 (2H, dd, J=9.0, 5.5 Hz).

### Example 83

### 6-[1-(2,2-Difluoropropyl)-5-(4-fluorophenyl)-3-methyl-1H-pyrazol-4-yl]-2H-1,4-benzoxazin-3(4H)-one

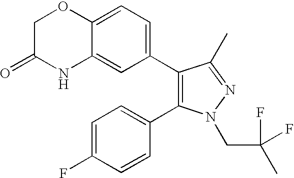

A mixture of 4-bromo-1-(2,2-difluoropropyl)-5-(4-fluorophenyl)-3-methyl-1H-pyrazole (535 mg), 6-(4,4,5,5-tetramethyl-1,3,2-dioxaborolan-2-yl)-2H-1,4-benzoxazin-3(4H)-one (528 mg), [1,1′-bis(diphenylphosphino)ferrocene]dichloropalladium(II) dichloromethane adduct (261 mg) and Cs₂CO₃ (1.56 g) in tetrahydrofuran (15 ml) and H₂O (3 ml) was stirred for 15 min. at 130° C. under microwave irradiation. After cooling, the reaction mixture was treated with ethyl acetate and H₂O. The organic layer was separated, washed with brine, dried over Na₂SO₄ and concentrated in vacuo. The residue was purified by silica gel column chromatography using n-hexane/ethyl acetate as an eluent to give the title compound (400 mg, 62%). The obtained compound was recrystallized from ethyl acetate and hexane.

¹H-NMR (300 MHz, DMSO-d₆) δ: 1.57 (3H, t, J=19.0 Hz), 2.21 (3H, s), 4.38 (2H, t, J=13.0 Hz), 4.54 (2H, s), 6.58 (1H, dd, J=8.5, 2.0 Hz), 6.65 (1H, d, J=2.0 Hz), 6.82 (1H, d, J=8.5 Hz), 7.22-7.35 (4H, m), 10.61 (1H, br. s).

### Example 84

### 6-[5-(4-Fluorophenyl)-1-(2-hydroxyethyl)-3-methyl-1H-pyrazol-4-yl]-2H-1,4-benzoxazin-3(4H)-one

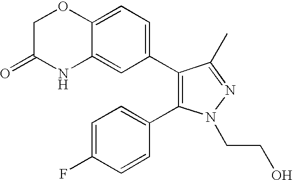

The title compound (223 mg) was synthesized from 2-[4-bromo-5-(4-fluorophenyl)-3-methyl-1H-pyrazol-1-yl]ethanol (653 mg) according to the similar procedure described for Example 74. Obtained compound was recrystallized from ethyl acetate and hexane.

Mp 177.6-178.7° C.

¹H-NMR (300 MHz, DMSO-d₆) δ: 2.19 (3H, s), 3.71 (2H, q, J=5.5 Hz), 3.85-3.95 (2H, m), 4.88 (1H, t, J=5.5 Hz), 6.56 (1H, dd, J=8.5, 2.0 Hz), 6.64 (1H, d, J=2.0 Hz), 6.82 (1H, d, J=8.5 Hz), 7.20-7.31 (2H, m), 7.31-7.41 (2H, m), 10.59 (1H, br. s).

### Example 85

### 2-[5-(4-Fluorophenyl)-3-methyl-4-(3-oxo-3,4-dihydro-2H-1,4-benzoxazin-6-yl)-1H-pyrazol-1-yl]ethyl acetate

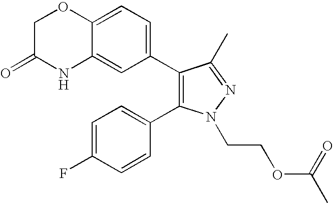

A solution of 6-[5-(4-fluorophenyl)-1-(2-hydroxyethyl)-3-methyl-1H-pyrazol-4-yl]-2H-1,4-benzoxazin-3(4H)-one (72 mg) in Pyridine (4 ml) and Ac₂O (2 ml) was stirred for 2 days. The reaction solvent was removed in vacuo. The residue was purified by silica gel column chromatography using n-hexane/ethyl acetate as an eluent to give the title compound (50 mg, 63%). Obtained compound was recrystallized from ethyl acetate and hexane.

Mp 159.8-160.0° C.

¹H-NMR (300 MHz, DMSO-d₆) δ: 1.89 (3H, s), 2.20 (3H, s), 4.12 (2H, t, J=5.0 Hz), 4.26 (2H, t, J=5.0 Hz), 4.53 (2H, s), 6.57 (1H, dd, J=8.5, 2.0 Hz), 6.66 (1H, d, J=2.0 Hz), 6.82 (1H, d, J=8.5 Hz), 7.22-7.38 (4H, m), 10.61 (1H, br. s).

### Preparation 119

### 1-[4-Bromo-5-(4-fluorophenyl)-3-methyl-1H-pyrazol-1-yl]-2-methylpropan-2-ol

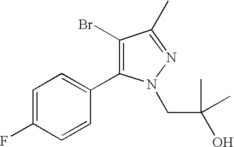

To a solution of ethyl [4-bromo-5-(4-fluorophenyl)-3-methyl-1H-pyrazol-1-yl]acetate (1.85 g) in dry tetrahydrofuran (60 ml) was added 3M MeMgBr (7.2 ml, tetrahydrofuran solution) at −78° C. After stirring for 3 h at rt, the mixture was treated with ethyl acetate and 1N HCl aqueous solution. The organic layer was separated, washed with brine, dried over Na₂SO₄ and concentrated in vacuo. The residue was purified by silica gel column chromatography using n-hexane/ethyl acetate as an eluent to give the title compound (928 mg, 52%).

¹H-NMR (300 MHz, DMSO-d₆) δ: 0.96 (6H, s), 2.19 (3H, s), 3.88 (2H, s), 4.62 (1H, s), 7.36 (2H, t, J=8.5 Hz), 7.52 (2H, dd, J=8.5, 5.5 Hz).

### Example 86

### 6-[5-(4-Fluorophenyl)-1-(2-hydroxy-2-methylpropyl)-3-methyl-1H-pyrazol-4-yl]-2H-1,4-benzoxazin-3(4H)-one

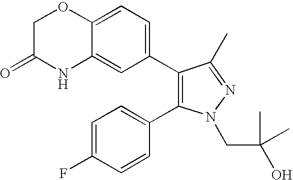

The title compound (415 mg) was synthesized from 1-[4-bromo-5-(4-fluorophenyl)-3-methyl-1H-pyrazol-1-yl]-2-methylpropan-2-ol (655 mg) according to the similar procedure described for Example 74. Obtained compound was recrystallized from ethyl acetate and hexane.

Mp 166.0-166.7° C.

¹H-NMR (300 MHz, DMSO-d₆) δ: 0.99 (6H, s), 2.20 (3H, s), 3.84 (2H, s), 4.53 (2H, s), 4.72 (1H, s), 6.55 (1H, dd, J=8.5, 2.0 Hz), 6.64 (1H, d, J=2.0 Hz), 6.81 (1H, d, J=8.5 Hz), 7.17-7.38 (4H, m), 10.60 (1H, br. s).

### Example 87

### 6-[5-(4-Fuorophenyl)-3-methyl-1-(2-oxopropyl)-1H-pyrazol-4-yl]-2H-1,4-benzoxazin-3(4H)-one

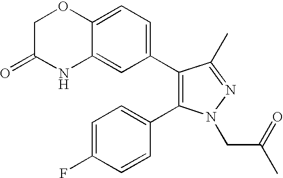

The title compound (254 mg) was synthesized from 1-[4-bromo-5-(4-fluorophenyl)-3-methyl-1H-pyrazol-1-yl]propan-2-one (490 mg) according to the similar procedure described for Example 74. The obtained compound was recrystallized from ethyl acetate and hexane.

Mp 216.5-217.5° C.

¹H-NMR (300 MHz, DMSO-d₆) δ: 2.03 (3H, s), 2.19 (3H, s), 4.54 (2H, s), 4.91 (2H, s), 6.58 (1H, dd, J=8.5, 2.0 Hz), 6.67 (1H, d, J=2.0 Hz), 6.83 (1H, d, J=8.5 Hz), 7.15-7.30 (4H, m), 10.61 (1H, br. s).

### Preparation 120

### 4-Bromo-5-(4-fluorophenyl)-1-(2-methoxyethyl)-3-methyl-1H-pyrazole

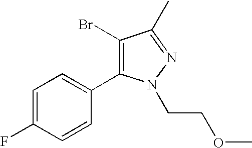

To a solution of 2-[4-bromo-5-(4-fluorophenyl)-3-methyl-1H-pyrazol-1-yl]ethanol (1.16 g) in N,N-dimethylformamide (20 ml) was added NaH (310 mg, 60% oil suspension) at 0° C. The mixture was stirred for 10 min. at the same temperature, and MeI (361 μM) was added. The reaction mixture was then allowed to warm up to rt and stirred for 2 h. The reaction mixture was treated with ethyl acetate and H₂O. The organic layer was separated, washed with brine, dried over Na₂SO₄ and concentrated in vacuo. The residue was purified by silica gel column chromatography using n-hexane/ethyl acetate as an eluent to give the title compound (1.18 g, 98%).

¹H-NMR (300 MHz, DMSO-d₆) δ: 2.19 (3H, s), 3.11 (3H, s), 3.61 (2H, t, J=5.5 Hz), 4.06 (2H, t, J=5.5 Hz), 7.34-7.43 (2H, m), 7.46-7.56 (2H, m).

### Example 88

### 6-[5-(4-Fluorophenyl)-1-(2-methoxyethyl)-3-methyl-1H-pyrazol-4-yl]-2H-1,4-benzoxazin-3(4H)-one

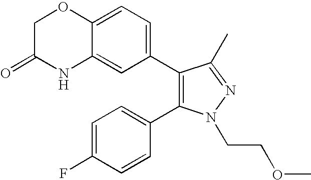

The title compound (322 mg) was synthesized from 4-bromo-5-(4-fluorophenyl)-1-(2-methoxyethyl)-3-methyl-1H-pyrazole (685 mg) according to the similar procedure described for Example 74. Obtained compound was recrystallized from ethyl acetate and hexane.

Mp 174.0-174.4° C.

¹H-NMR (300 MHz, DMSO-d₆) δ: 2.19 (3H, s), 3.13 (3H, s), 3.65 (2H, t, J=5.5 Hz), 4.01 (2H, t, J=5.5 Hz), 4.53 (2H, s), 6.56 (1H, dd, J=8.0, 2.0 Hz), 6.64 (1H, d, J=2.0 Hz), 6.82 (1H, d, J=8.0 Hz), 7.20-7.37 (4H, m), 10.60 (1H, br. s).

### Example 89

### Ethyl[5-(4-fluorophenyl)-3-methyl-4-(3-oxo-3,4-dihydro-2H-1,4-benzoxazin-6-yl)-1H-pyrazol-1-yl]acetate

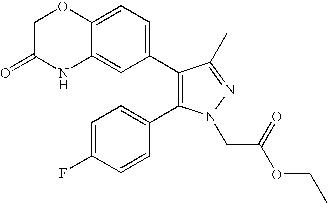

The title compound (400 mg) was synthesized from ethyl [4-bromo-5-(4-fluorophenyl)-3-methyl-1H-pyrazol-1-yl]acetate (745 mg) according to the similar procedure described for Example 74.

¹H-NMR (300 MHz, DMSO-d₆) δ: 1.12 (3H, t, J=7.0 Hz), 2.19 (3H, s), 4.06 (2H, q, J=7.0 Hz), 4.54 (2H, s), 4.81 (2H, s), 6.59 (1H, dd, J=8.0, 2.0 Hz), 6.67 (1H, d, J=2.0 Hz), 6.84 (1H, d, J=8.5 Hz), 7.26 (4H, d, J=7.0 Hz), 10.61 (1H, br. s).

### Preparation 121

### 1-(2-Azidoethyl)-5-(4-fluorophenyl)-3-methyl-1H-pyrazole

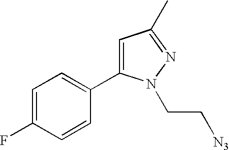

A solution of 2-[5-(4-fluorophenyl)-3-methyl-1H-pyrazol-1-yl]ethanol (5.35 g) and TsCl (6.0 g) in pyridine (30 ml) was stirred for 12 h at rt. The mixture was treated with ethyl acetate and 1N HCl aqueous solution. The organic layer was separated, washed with 1N HCl aqueous solution and brine, dried over Na₂SO₄ and concentrated in vacuo. The residue was dissolved in N,N-dimethylformamide (30 ml). To this solution was added NaN₃ (2.05 g) at rt. The mixture was stirred for 6 h at 50° C. and then allowed to cool down to rt. After stirring for 2 days at rt, the reaction mixture was treated with ethyl acetate and saturated NaHCO₃ aqueous solution. The organic layer was separated, washed with brine, dried over Na₂SO₄ and concentrated in vacuo. The residue was purified by silica gel column chromatography using n-hexane/ethyl acetate as an eluent to give the title compound (4.94g, 83%).

¹H-NMR (300 MHz, DMSO-d₆) δ: 2.20 (3H, s), 3.66 (2H, t, J=5.5 Hz), 4.14 (2H, t, J=5.5 Hz), 6.18 (1H, s), 7.34 (2H, t, J=9.0 Hz), 7.51 (2H, dd, J=9.0, 5.5 Hz).

### Preparation 122

### N-{2-[4-Bromo-5-(4-fluorophenyl)-3-methyl-1H-pyrazol-1-yl]ethyl}acetamide

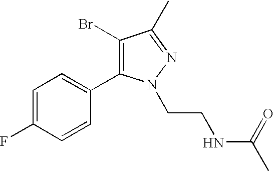

To a solution of 1-(2-azidoethyl)-5-(4-fluorophenyl)-3-methyl-1H-pyrazole (1.00 g) in tetrahydrofuran (25 ml) was added PPh₃ (1.28 g) at rt. After stirring for 3 h at rt, H₂O (360 μl) was added to the reaction mixture. The resulting mixture was stirred for additional 12 h at 50° C. The mixture was treated with ethyl acetate and 1N HCl aqueous solution. The aqueous layer was separated and then basified with 8N NaOH aqueous solution. The solution was extracted with ethyl acetate. The organic layer was washed with brine, dried over Na₂SO₄ and concentrated in vacuo. The residue was dissolved in pyridine (15 ml) and Ac₂O (10 ml) and the mixture was stirred for 3 h at rt. The reaction solvent was removed in vacuo. The residue was dissolved in N,N-dimethylformamide (30 ml). To the resulting solution was added N-bromosuccinimide (871 mg) at 0° C. After stirring for 30 min. at rt, the reaction was quenched with aqueous Na₂S₂O₃ solution. The solution was treated with ethyl acetate and H₂O. The organic layer was separated, washed with brine, dried over Na₂SO₄ and concentrated in vacuo. The residue was purified by silica gel column chromatography using n-hexane/ethyl acetate as an eluent to give the title compound (1.30 g, 94%).

¹H-NMR (300 MHz, DMSO-d₆) δ: 1.65 (3H, s), 2.20 (3H, s), 3.27 (2H, q, J=6.0 Hz), 3.97 (2H, t, J=6.0 Hz), 7.37 (2H, t, J=9.0 Hz), 7.45 (2H, dd, J=9.0, 5.5 Hz), 7.87 (1H, t, J=6.0 Hz).

### Example 90

### N-{2-[5-(4-Fluorophenyl)-3-methyl-4-(3-oxo-3,4-dihydro-2H-1,4-benzoxazin-6-yl)-1H-pyrazol-1-yl]ethyl}acetamide

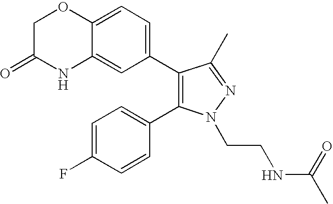

The title compound (359 mg) was synthesized from N-{2-[4-bromo-5-(4-fluorophenyl)-3-methyl-1H-pyrazol-1-yl]ethyl}acetamide (680 mg) according to the similar procedure described for Example 74.

¹H-NMR (300 MHz, DMSO-d₆) δ: 1.69 (3H, s), 2.20 (3H, s), 3.32 (2H, q, J=6.0 Hz), 3.90 (2H, t, J=6.0 Hz), 4.53 (2H, s), 6.56 (1H, dd, J=8.5, 2.0 Hz), 6.66 (1H, d, J=2.0 Hz), 6.81 (1H, d, J=8.5 Hz), 7.20-7.34 (4H, m), 7.94 (1H, t, J=6.0 Hz), 10.61 (1H, br. s).

### Preparation 123

### 1-(2-Azidoethyl)-4-bromo-5-(4-fluorophenyl)-3-methyl-1H-pyrazole

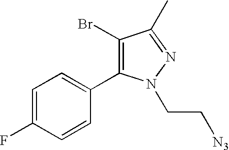

The title compound (4.94 g) was synthesized from 1-(2-azidoethyl)-5-(4-fluorophenyl)-3-methyl-1H-pyrazole (3.85 g) according to the similar procedure described for preparation 99.

¹H-NMR (300 MHz, DMSO-d₆) δ: 2.21 (3H, s), 3.63 (2H, t, J=5.5 Hz), 4.11 (2H, t, J=5.5 Hz), 7.41 (2H, t, J=8.5 Hz), 7.51 (2H, dd, J=8.5, 6.0 Hz).

### Preparation 124

### N-{2-[4-Bromo-5-(4-fluorophenyl)-3-methyl-1H-pyrazol-1-yl]ethyl}methanesulfonamide

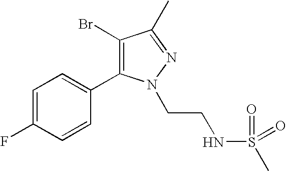

To a solution of 1-(2-azidoethyl)-4-bromo-5-(4-fluorophenyl)-3-methyl-1H-pyrazole (800 mg) in tetrahydrofuran (20 ml) was added PPh₃ (774 mg) at rt. After stirring for 3 h at rt, H₂O (220 μl ) was added to the reaction mixture. The resulting mixture was stirred for additional 12 h at 50° C. The mixture was treated with ethyl acetate and 1N HCl aqueous solution. The aqueous layer was separated and then basified with 8N NaOH aqueous solution. The solution was extracted with ethyl acetate. The organic layer was washed with brine, dried over Na₂SO₄ and concentrated in vacuo. The residue was dissolved in pyridine (5 ml) and MsCl (285 μl) and the mixture was stirred for 12 h at rt. The reaction mixture was treated with ethyl acetate and 1N HCl aqueous solution. The organic layer was separated, washed with brine, dried over Na₂SO₄ and concentrated in vacuo. The residue was purified by silica gel column chromatography using n-hexane/ethyl acetate as an eluent to give the title compound (760 mg, 82%)

¹H-NMR (300 MHz, DMSO-d₆) δ: 2.20 (3H, s), 2.79 (3H, s), 3.27 (2H, t, J=7.0 Hz), 4.02 (2H, t, J=7.0 Hz), 7.14 (1H, br. s.), 7.38 (2H, t, J=9.0 Hz), 7.51 (2H, dd, J=9.0, 5.5 Hz).

### Example 91

### N-{2-[5-(4-Fluorophenyl)-3-methyl-4-(3-oxo-3,4-dihydro-2H-1,4-benzoxazin-6-yl)-1H-pyrazol-1-yl]ethyl}methanesulfonamide

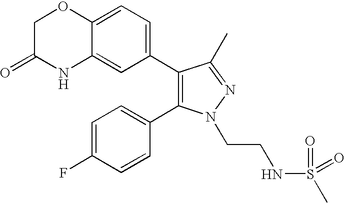

The title compound (300 mg) was synthesized from N-{2-[4-bromo-5-(4-fluorophenyl)-3-methyl-1H-pyrazol-1-yl]ethyl}methanesulfonamide (760 mg) according to the similar procedure described for Example 74. Obtained compound was recrystallized from ethyl acetate and hexane.

Mp 220.5-221.0° C.

¹H-NMR (300 MHz, DMSO-d₆) δ: 2.20 (3H, s), 2.81 (3H, s), 3.25-3.33 (2H, m), 3.97 (2H, t, J=6.5 Hz), 4.53 (2H, s), 6.57 (1H, dd, J=8.5, 2.0 Hz), 6.61-6.68 (1H, m), 6.82 (1H, d, J=8.5 Hz), 7.18 (1H, t, J=6.0 Hz), 7.22-7.41 (4H, m), 10.60 (1H, br. s).

### Example 92

### 3-[5-(4-Fluorophenyl)-3-methyl-4-(3-oxo-3,4-dihydro-2H-1,4-benzoxazin-6-yl)-1H-pyrazol-1-yl]propanenitrile

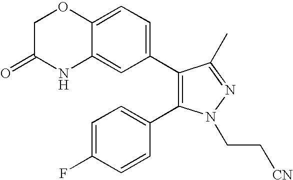

### Example 93

### Ethyl{2-[5-(4-fluorophenyl)-3-methyl-4-(3-oxo-3,4-dihydro-2H-1,4-benzoxazin-6-yl)-1H-pyrazol-1-yl]ethoxy}acetate

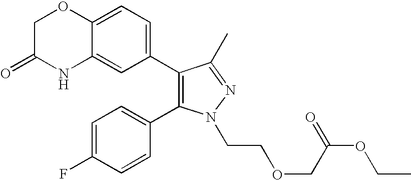

### Example 94

### {2-[5-(4-Fluorophenyl)-3-methyl-4-(3-oxo-3,4-dihydro-2H-1,4-benzoxazin-6-yl) -1H-pyrazol-1-yl]ethoxy}acetic acid

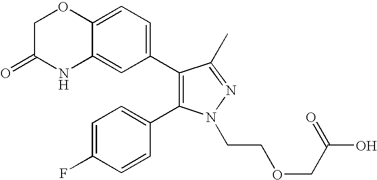

### Example 95

### 2-{2-[5-(4-Fluorophenyl)-3-methyl-4-(3-oxo-3,4-dihydro-2H-1,4-benzoxazin-6-yl)-1H-pyrazol-1-yl]ethoxy}acetamide

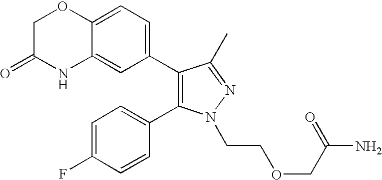

### Example 96

### 2-[5-(4-Fluorophenyl)-3-methyl-4-(3-oxo-3,4-dihydro-2H-1,4-benzoxazin-6-yl)-1H-pyrazol-1-yl]acetamide

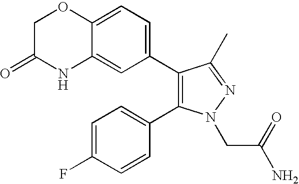

### Example 97

### 2-[5-(4-Fluorophenyl)-3-methyl-4-(3-oxo-3,4-dihydro-2H-1,4-benzoxazin-6-yl)-1H-pyrazol-1-yl]-N,N-dimethylacetamide

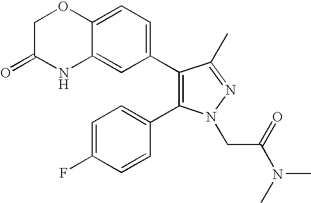

### Example 98

### 2-[5-(4-Fluorophenyl)-3-methyl-4-(3-oxo-3,4-dihydro-2H-1,4-benzoxazin-6-yl)-1H-pyrazol-1-yl]-N-(2,2,2-trifluoroethyl)acetamide

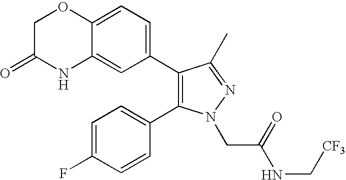

### Example 99

### [5-(4-Fluorophenyl)-3-methyl-4-(3-oxo-3,4-dihydro-2H-1,4-benzoxazin-6-yl)-1H-pyrazol-1-yl]acetonitrile

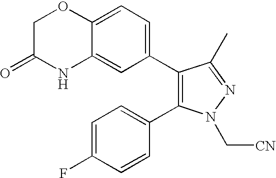

### Example 100

### 2,2,2-Trifluoro-N-{2-[5-(4-fluorophenyl)-3-methyl-4-(3-oxo-3,4-dihydro-2H-1,4-benzoxazin-6-yl)-1H-pyrazol-1-yl]ethyl}acetamide

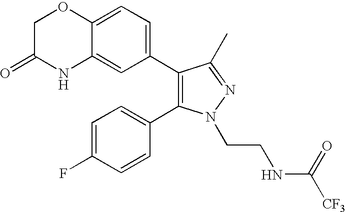

### Example 101

### N-{2-[5-(4-Fluorophenyl)-3-methyl-4-(3-oxo-3,4-dihydro-2H-1,4-benzoxazin-6-yl)-1H-pyrazol-1-yl]ethyl}-N-methylacetamide

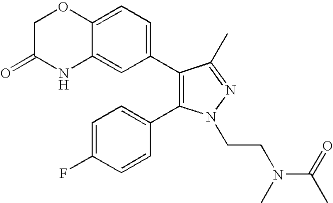

### Example 102

### N-{2-[5-(4-Fluorophenyl)-3-methyl-4-(3-oxo-3,4-dihydro-2H-1,4-benzoxazin-6-yl)-1H-pyrazol-1-yl]ethyl}ethanesulfonamide

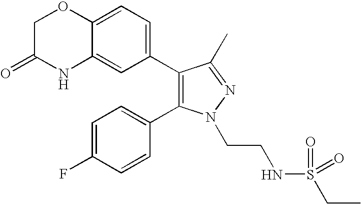

### Preparation 125

### 1-(Benzyloxy)-3-hydrazinopropan-2-ol

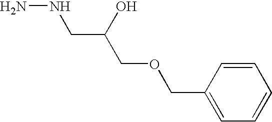

Benzyl glycidyl ether (25.0 g) was added to hydrazine monohydrate (100 g) dropwise at 60-65° C. and the mixture was stirred at 60-65° C. for 3 h. The mixture was concentrated in vacuo to give the title compound as an oil (29.6 g).

¹H-NMR (300 MHz, CDCl₃) δ: 2.75-2.95 (2H, m), 3.17-3.60 (6H, m), 3.95-4.11 (1H, m), 4.56 (2H, s), 7.23-7.41 (5H, m).

### Preparation 126

### 1-(Benzyloxy)-3-[5-(4-fluorophenyl)-3-methyl-1H-pyrazol-1-yl]propan-2-ol

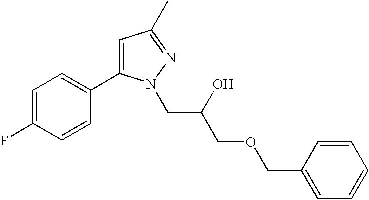

To a solution of 1-(4-fluorophenyl)butane-1,3-dione (5.00 g) in methanol (50 ml) was added conc. hydrochloric acid (2.80 ml) under ice-cooling and then 1-(benzyloxy)-3-hydrazinopropan-2-ol (6.60 g) was added under ice-cooling. The mixture was allowed to warm to room temperature and stirred for 12 h at room temperature. The mixture was concentrated in vacuo and then water and ethyl acetate was added to the residue. Potassium carbonate was added to the mixture to basify the aqueous layer and then organic layer was separated. The organic layer was washed with brine, dried over Na₂SO₄, and concentrated in vacuo. The residue was purified by chromatography on silica gel (hexane:ethyl acetate=10:1 to 1:1) to give the title compound as an oil (6.21 g).

¹H-NMR (300 MHz, CDCl₃) δ: 2.29 (3H, s), 3.29-3.38 (1H, m, J=9.5, 6.7 Hz), 3.48-3.56 (1H, m), 4.06-4.25 (3H, m), 4.45 (2H, s), 4.53 (1H, s), 6.07 (1H, s), 7.00-7.10 (2H, m), 7.15-7.22 (2H, m), 7.25-7.41 (5H, m).

### Preparation 127

### 1-(Benzyloxy)-3-[4-bromo-5-(4-fluorophenyl)-3-methyl-1H-pyrazol-1-yl]propan-2-ol

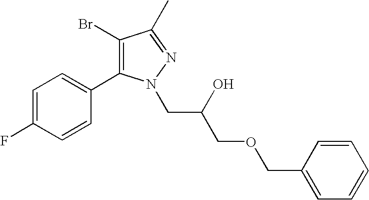

To a solution of 1-(benzyloxy)-3-[5-(4-fluorophenyl)-3-methyl-1H-pyrazol-1-yl]propan-2-ol (6.21 g) in acetonitrile (62 ml) was added N-bromosuccinimide (3.40 g) under ice-cooling and then the mixture was allowed to warm to room temperature. The mixture was concentrated in vacuo, and then toluene was added to the residue. Resulting crystals were filtered off and filtrate was concentrated in vacuo. The residue was purified by chromatography on silica gel (hexane:ethyl acetate=10:1 to 3:2) to give the title compound as an oil (7.47 g).

¹H-NMR (300 MHz, CDCl₃) δ: 2.28 (3H, s), 3.33-3.42 (1H, m), 3.45-3.53 (1H, m), 3.90-4.22 (4H, m), 4.46 (2H, s), 7.04-7.44 (9H, m).

### Preparation 128

### 1-[3-(Benzyloxy)-2-fluoropropyl]-4-bromo-5-(4-fluorophenyl)-3-methyl-1H-pyrazole

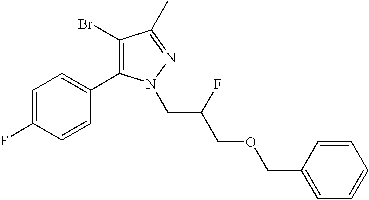

To a solution of 1-(benzyloxy)-3-[4-bromo-5-(4-fluorophenyl)-3-methyl-1H-pyrazol-1-yl]propan-2-ol (500 mg) in toluene (4 ml) was added a solution of diethylaminosulfur trifluoride (220 mg) in toluene (1 ml) dropwise under ice-cooling and the mixture was allowed to warm to 50° C. After stirring for 6 h, diethylaminosulfur trifluoride (220 mg) was added to the mixture and the mixture was stirred for 12 h at 50° C. Saturated aqueous sodium bicarbonate solution was added to the mixture under ice-cooling and the mixture was allowed to warm to room temperature. After stirring for 0.5 h, water was added to the mixture and the mixture was extracted with ethyl acetate. The organic layer was washed with water, brine, dried over Na₂SO₄, and concentrated in vacuo. The residue was purified by chromatography on silica gel (hexane:ethyl acetate=10:1 to 2:1) to give the title compound as an oil (250 mg).

¹H-NMR (300 MHz, CDCl₃) δ: 2.29 (3H, s), 3.51-3.78 (2H, m), 4.06-4.40 (2H, m), 4.48 (1H, d, J=11.7 Hz), 4.54 (1H, d, J=11.7 Hz), 4.96-5.22 (1H, m), 7.09-7.44 (9H, m).

### Preparation 129

### 1-(Benzyloxy)-3-[4-bromo-5-(4-fluorophenyl)-3-methyl-1H-pyrazol-1-yl]propan-2-one

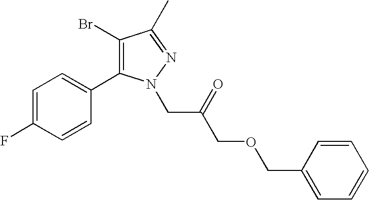

To a solution of 1-(Benzyloxy)-3-[4-bromo-5-(4-fluorophenyl)-3-methyl-1H-pyrazol-1-yl]propan-2-ol (1.50 g) in toluene (30 ml) was added Dess-Martin periodinane (2.00 g) under ice-cooling and the mixture was allowed to warm to room temperature. After stirring for 12 h, ethyl acetate (60 ml) and then a solution of sodium thiosulfate pentahydrate (6.90 g) in saturated aqueous sodium bicarbonate solution (55 ml) were added to the mixture at room temperature and the mixture was stirred for 0.5 h. The organic layer was separated, washed with water, brine, dried over Na₂SO₄ and concentrated in vacuo. The residue was purified by chromatography on silica gel (hexane:ethyl acetate=10:1 to 3:2) to give the title compound as an oil (1.39 g).

¹H-NMR (300 MHz, CDCl₃) δ: 2.29 (3H, s), 4.05 (2H, s), 4.51 (2H, s), 4.98 (2H, s), 7.08-7.17 (2H, m), 7.22-7.43 (7H, m).

### Preparation 130

### 1-[3-(Benzyloxy)-2,2-difluoropropyl]-4-bromo-5-(4-fluorophenyl)-3-methyl-1H-pyrazole

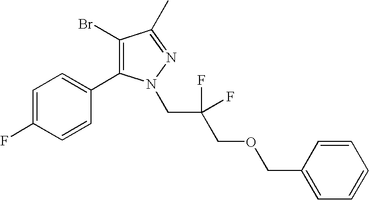

To a solution of 1-(benzyloxy)-3-[4-bromo-5-(4-fluorophenyl)-3-methyl-1H-pyrazol-1-yl]propan-2-one (890 mg) in toluene (20 ml) was added diethylaminosulfur trifluoride (700 mg) dropwise under ice-cooling and the mixture was allowed to warm to 40° C. After stirring for 6 h, diethylaminosulfur trifluoride (350 mg) was added to the mixture and the mixture was stirred for 24 h at 40° C. Saturated aqueous sodium bicarbonate solution was added to the mixture under ice-cooling and the mixture was allowed to warm to room temperature. After stirring for 0.5 h, water was added to the mixture and the mixture was extracted with ethyl acetate. The organic layer was washed with water, brine, dried over Na₂SO₄, and concentrated in vacuo. The residue was purified by chromatography on silica gel (hexane to hexane:ethyl acetate=3:1) to give the title compound as crystals (622 mg).

¹H-NMR (300 MHz, CDCl₃) δ: 2.30 (3H, s), 3.68 (2H, t, J=12.5 Hz), 4.44 (2H, t, J=12.7 Hz), 4.58 (2H, s), 7.09-7.19 (2H, m), 7.23-7.40 (7H, m).

### Preparation 131

### 3-[4-Bromo-5-(4-fluorophenyl)-3-methyl-1H-pyrazol-1-yl]propanal

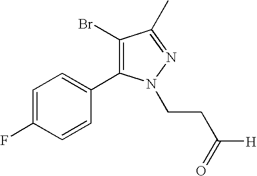

To a solution of 3-[4-bromo-5-(4-fluorophenyl)-3-methyl-1H-pyrazol-1-yl]propan-1-ol (4.20 g) in DMSO (42 ml) was added triethylamine (30 ml) at room temperature and then pyridine sulfur trioxide complex (17.1 g) was added to the mixture at room temperature. The mixture was stirred at room temperature for 1 and then poured into the ice cooled water. Potassium carbonate was added to the mixture to basify the aqueous layer and then the mixture was extracted with ethyl acetate. The organic layer was washed with water, brine, dried over Na₂SO₄, and concentrated in vacuo. The residue was purified by chromatography on silica gel (hexane:ethyl acetate=10:1 to 1:1) to give the title compound as an oil (2.95 g).

¹H-NMR (300 MHz, CDCl₃) δ: 2.26 (3H, s), 2.99 (2H, td, J=6.7, 0.9 Hz), 4.27 (2H, t, J=6.7 Hz), 7.15-7.25 (2H, m), 7.35-7.44 (2H, m), 9.74 (1H, t, J=0.9 Hz).

### Preparation 132

### 4-[4-Bromo-5-(4-fluorophenyl)-3-methyl-1H-pyrazol-1-yl]butan-2-one

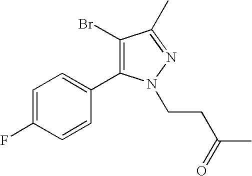

To a solution of 3-[4-bromo-5-(4-fluorophenyl)-3-methyl-1H-pyrazol-1-yl]propanal (1.00 g) in tetrahydrofuran (10 ml) was added methylmagnesium bromide in diethyl ether (3M, 1.60 ml) at room temperature and the mixture was stirred at room temperature for 0.5 h. The mixture was added to the ice cooled water. The aqueous layer was acidified with 10% HCl and then basified with 28% ammonia solution. The mixture was extracted with ethyl acetate. The organic layer was washed with water, brine, dried over Na₂SO₄, and concentrated in vacuo to give crude 4-[4-bromo-5-(4-fluorophenyl)-3-methyl-1H-pyrazol-1-yl]butan-2-ol. The crude 4-[4-bromo-5-(4-fluorophenyl)-3-methyl-1H-pyrazol-1-yl]butan-2-ol was dissolved in toluene (20 ml) and then Dess-Martin periodinane (1.80 g) was added to the solution under ice-cooling. The mixture was allowed to warm to room temperature and stirred for 12 h. Ethyl acetate and then a solution of sodium thiosulfate pentahydrate (5.80 g) in saturated aqueous sodium bicarbonate solution (46 ml) were added to the mixture at room temperature and the mixture was stirred for 0.5 h. The organic layer was separated, washed with water, brine, dried over Na₂SO₄ and concentrated in vacuo. The residue was purified by chromatography on silica gel (hexane:ethyl acetate=10:1 to 1:1) to give the title compound as an oil (800 mg).

¹H-NMR (300 MHz, CDCl₃) δ: 2.13 (3H, s), 2.27 (3H, s), 3.00 (2H, t, J=6.9 Hz), 4.19 (2H, t, J=6.9 Hz), 7.14-7.24 (2H, m), 7.36-7.44 (2H, m).

### Preparation 133

### 4-[4-Bromo-5-(4-fluorophenyl)-3-methyl-1H-pyrazol-1-yl]-1,1,1-trifluorobutan-2-ol

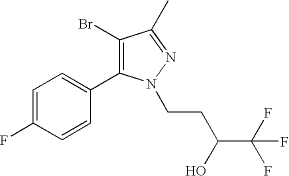

To a solution of 3-[4-bromo-5-(4-fluorophenyl)-3-methyl-1H-pyrazol-1-yl]propanal (1.17 g) and trifluoromethyltrimethylsilane (660 mg) in tetrahydrofuran (12 ml) was added tetra-n-butylammonium fluoride in tetrahydrofuran (1M, 0.70 ml) dropwise at room temperature. The mixture was stirred at room temperature for 0.5 h and then concentrated in vacuo. The residue was purified by chromatography on silica gel (hexane:ethyl acetate=10:1 to 3:1) to give the title compound as crystals (1.04 g).

¹H-NMR (300 MHz, CDCl₃) δ: 1.91-2.20 (2H, m), 2.28 (3H, s), 3.94-4.19 (2H, m), 4.21-4.35 (1H, m), 5.07 (1H, d, J=4.9 Hz), 7.15-7.27 (2H, m), 7.30-7.41 (2H, m).

### Preparation 134

### [(But-3-en-1-yloxy)methyl]benzene

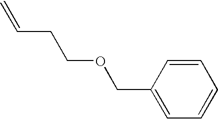

A mixture of 3-buten-1-ol (5.00 g), triethylamine (0.46 g), sodium hydroxide (4.10 g) and hexane (50 ml) was stirred at 50° C. for 0.5 h and then benzyl bromide (12.9 g) was added to the mixture dropwise below 60° C. The mixture was allowed to warm to reflux and refluxed for 3 h. The mixture was poured into ice cooled water and the mixture was extracted with hexane. The organic layer was washed with water, brine, dried over Na₂SO₄ and concentrated in vacuo. The residue was purified by chromatography on silica gel (hexane to hexane:ethyl acetate=10:1) to give the title compound as an oil (10.7 g).

¹H-NMR (300 MHz, CDCl₃) δ: 2.38 (2H, qt, J=6.8, 1.2 Hz), 3.53 (2H, t, J=6.8 Hz), 4.52 (2H, s), 5.00-5.16 (2H, m), 5.76-5.92 (1H, m), 7.26-7.42 (5H, m).

### Preparation 135

### 2-[2-(Benzyloxy)ethyl]oxirane

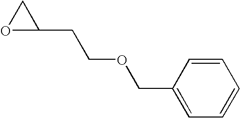

To a solution of [(but-3-en-1-yloxy)methyl]benzene (10.6 g) in toluene (200 ml) was added m-chloroperbenzoic acid with water (69-75%, 20.0 g) under ice-cooling and then the mixture was allowed to warm to room temperature. After stirring for 18 h at room temperature, the mixture was filtered. The filtrate was diluted with hexane, washed with a solution of sodium thiosulfate pentahydrate (8.1 g) in 5% aqueous sodium bicarbonate solution (200 ml), 3% aqueous sodium bicarbonate solution (200 ml), water (200 ml), brine, dried over Na₂SO₄ and concentrated in vacuo. The residue was purified by chromatography on silica gel (hexane:ethyl acetate=20:1 to 4:1) to give the title compound as an oil (10.2 g).

¹H-NMR (300 MHz, CDCl₃) δ: 1.71-1.85 (1H, m), 1.86-1.99 (1H, m), 2.53 (1H, dd, J=4.9, 2.7 Hz), 2.79 (1H, dd, J=4.9, 4.2 Hz), 3.03-3.12 (1H, m), 3.56-3.70 (2H, m), 4.53 (2H, s), 7.24-7.40 (5H, m).

### Preparation 136

### 4-(Benzyloxy)-1-hydrazinobutan-2-ol

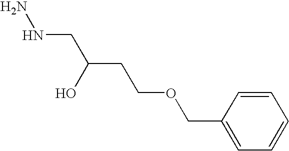

2-[2-(Benzyloxy)ethyl]oxirane (10.1 g) was added to hydrazine monohydrate (40.0 g) dropwise at 60-65° C. and the mixture was stirred for 1 h at 60-65° C. The mixture was concentrated in vacuo to give the title compound as an oil (11.7 g).

¹H-NMR (300 MHz, CDCl₃) δ: 1.67-1.87 (2H, m), 2.67-2.78 (1H, m), 2.83 (1H, dd, J=12.3, 3.2 Hz), 3.31 (4H, s.), 3.58-3.77 (2H, m), 3.96-4.07 (1H, m), 4.52 (2H, s), 7.22-7.40 (5H, m).

### Preparation 137

### 4-(Benzyloxy)-1-[5-(4-fluorophenyl)-3-methyl-1H-pyrazol-1-yl]butan-2-ol

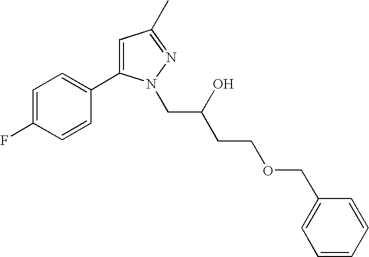

To a solution of 1-(4-fluorophenyl)butane-1,3-dione (5.00 g) in methanol (50 ml) was added conc. hydrochloric acid (4.20 ml) under ice-cooling and then 4-(benzyloxy)-1-hydrazinobutan-2-ol (7.10 g) was added under ice-cooling. After stirring for 3 h under ice-cooling, the mixture was allowed to warm to room temperature and stirred for 6 h at room temperature. The mixture was concentrated in vacuo and then ethyl acetate and saturated aqueous sodium bicarbonate solution were added to the residue. The organic layer was separated, washed with water, brine, dried over Na₂SO₄, and concentrated in vacuo. The residue was purified by chromatography on silica gel (hexane:ethyl acetate=10:1 to 3:2) and basic silica gel (hexane to hexane:ethyl acetate=2:1) to give the title compound as an oil (7.02 g)

¹H-NMR (300 MHz, CDCl₃) δ: 1.62-1.73 (2H, m), 2.29 (3H, s), 3.53-3.69 (2H, m), 3.91-4.01 (1H, m), 4.10 (1H, dd, J=14.0, 3.0 Hz), 4.15-4.26 (1H, m), 4.45 (2H, s), 4.48 (1H, d, J=3.4 Hz), 6.06 (1H, s), 7.03-7.14 (2H, m), 7.22-7.41 (7H, m).

### Preparation 138

### 4-(Benzyloxy)-1-[4-bromo-5-(4-fluorophenyl)-3-methyl-1H-pyrazol-1-yl]butan-2-ol

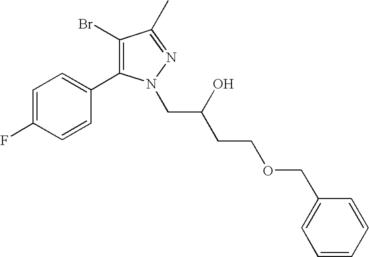

To a solution of 1-(benzyloxy)-3-[5-(4-fluorophenyl)-3-methyl-1H-pyrazol-1-yl]propan-2-ol (2.27 g) in acetonitrile (23 ml) was added N-bromosuccinimide (1.20 g) under ice-cooling and then the mixture was allowed to warm to room temperature. After stirring for 0.5 h, the mixture was concentrated in vacuo. The residue was diluted with toluene and the resulting crystals were filtered off. The filtrate was concentrated in vacuo and then the residue was purified by chromatography on silica gel (hexane:ethyl acetate=10:1 to 3:2) to give the title compound as an oil (2.68 g).

¹H-NMR (300 MHz, CDCl₃) δ: 1.62-1.72 (2H, m), 2.28 (3H, s), 3.53-3.68 (2H, m), 3.88-3.98 (1H, m), 4.03 (1H, dd, J=13.8, 3.2 Hz), 4.14-4.26 (1H, m), 4.44 (2H, s), 7.09-7.19 (2H, m), 7.21-7.44 (7H, m), 1H unconfirmed.

### Preparation 139

### 4-(Benzyloxy)-1-[4-bromo-5-(4-fluorophenyl)-3-methyl-1H-pyrazol-1-yl]butan-2-one

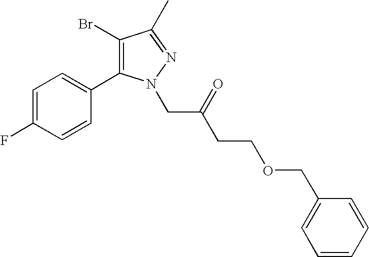

To a solution of 4-(benzyloxy)-1-[4-bromo-5-(4-fluorophenyl)-3-methyl-1H-pyrazol-1-yl]butan-2-ol (2.63 g) in toluene (50 ml) was added Dess-Martin periodinane (3.35 g) under ice-cooling and the mixture was allowed to warm to room temperature. After stirring for 12 h, ethyl acetate (100 ml) and then a solution of sodium thiosulfate pentahydrate (11.0 g) in saturated aqueous sodium bicarbonate solution (88 ml) were added to the mixture at room temperature and the mixture was stirred for 0.5 h. The organic layer was separated, washed with water, brine, dried over Na₂SO₄ and concentrated in vacuo. The residue was purified by chromatography on silica gel (hexane:ethyl acetate=10:1 to 3:2) to give the title compound as an oil (2.58 g).

¹H-NMR (300 MHz, CDCl₃) δ: 2.30 (3H, s), 2.61 (2H, t, J=6.0 Hz), 3.69 (2H, t, J=6.0 Hz), 4.43 (2H, s), 4.82 (2H, s), 6.99-7.10 (2H, m), 7.19-7.38 (7H, m).

### Preparation 140

### 1-[4-(Benzyloxy)-2,2-difluorobutyl]-4-bromo-5-(4-fluorophenyl)-3-methyl-1H-pyrazole

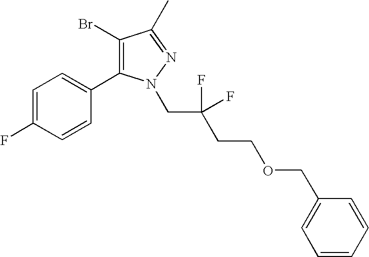

To a solution of 4-(benzyloxy)-1-[4-bromo-5-(4-fluorophenyl)-3-methyl-1H-pyrazol-1-yl]butan-2-one (2.53 g) in toluene (50 ml) was added diethylaminosulfur trifluoride (2.40 g) dropwise under ice-cooling and the mixture was allowed to warm to 40° C. After stirring for 24 h, diethylaminosulfur trifluoride (1.20 g) was added to the mixture at 40° C. and the mixture was allowed to warm to 50° C. The mixture was stirred at 50° C. for 60 h. The mixture was added to the saturated aqueous sodium bicarbonate solution under ice-cooling and then the mixture was extracted with ethyl acetate. The organic layer was washed with water, brine, dried over Na₂SO₄, and concentrated in vacuo. The residue was purified by chromatography on silica gel (hexane to hexane:ethyl acetate=3:1) to give the title compound as an oil (1.05 g).

¹H-NMR (300 MHz, CDCl₃) δ: 2.20 (2H, tt, J=16.3, 6.3 Hz), 2.30 (3H, s), 3.60 (2H, t, J=6.3 Hz), 4.31-4.47 (4H, m), 7.09-7.20 (2H, m), 7.22-7.39 (7H, m).

### Preparation 141

### 2-({2-[4-Bromo-5-(4-fluorophenyl)-3-methyl-1H-pyrazol-1-yl]ethyl}sulfanyl)ethanol

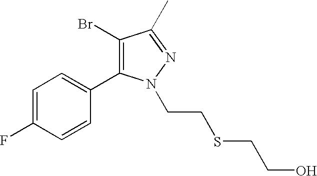

To a mixture of 4-bromo-5-(4-fluorophenyl)-1-(2-iodoethyl)-3-methyl-1H-pyrazole (2.50 g), potassium carbonate (1.30 g) and N,N-dimethylformamide (30 ml) was added 2-mercaptoethanol (600 mg) at room temperature and the mixture was stirred at room temperature for 4 h under argon atmosphere. The mixture was diluted with ethyl acetate and the mixture was filtered. The filtrate was concentrated in vacuo. The residue was diluted with toluene and the mixture was filtered. The filtrate was concentrated in vacuo. The residue was purified by chromatography on silica gel (hexane:ethyl acetate=10:1 to 1:2) to give the title compound as an oil (1.94 g).

¹H-NMR (300 MHz, CDCl₃) δ: 2.29 (3H, s), 2.49-2.57 (3H, m), 2.88 (2H, t, J=7.0 Hz), 3.61-3.70 (2H, m), 4.17 (2H, t, J=7.0 Hz), 7.20 (2H, t, J=8.7 Hz), 7.39 (2H, dd, J=8.7, 5.3 Hz).

### Preparation 142

### 2-({2-[4-Bromo-5-(4-fluorophenyl)-3-methyl-1H-pyrazol-1-yl]ethyl}sulfonyl)ethanol

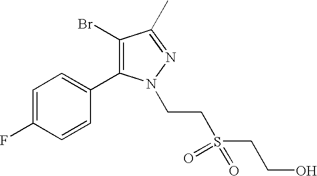

To a solution of 2-({2-[4-bromo-5-(4-fluorophenyl)-3-methyl-1H-pyrazol-1-yl]ethyl}sulfanyl)ethanol (500 mg) in toluene (10 ml) was added m-chloroperbenzoic acid with water (69-75%, 860 mg) under ice-cooling and then the mixture was allowed to warm to room temperature. Resulting crystals were filtered off and the filtrate was concentrated in vacuo. The residue was purified by chromatography on basic silica gel (hexane to ethyl acetate) to give the title compound as crystals (470 mg).

¹H-NMR (300 MHz, CDCl₃) δ: 2.29 (3H, s), 2.94 (1H, t, J=5.7 Hz), 2.99-3.06 (2H, m), 3.71 (2H, t, J=6.6 Hz), 4.01-4.10 (2H, m), 4.44 (2H, t, J=6.6 Hz), 7.17-7.27 (2H, m), 7.36-7.45 (2H, m).

### Preparation 143

### 6-{1-[3-(Benzyloxy)-2-fluoropropyl]-5-(4-fluorophenyl)-3-methyl-1H-pyrazol-4-yl}-2H-1,4-benzoxazin-3(4H)-one

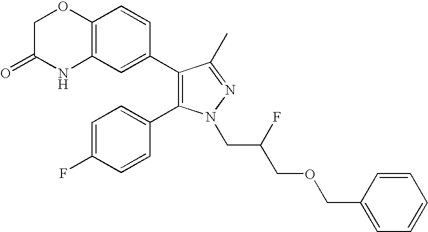

A mixture of 1-[3-(benzyloxy)-2-fluoropropyl]-4-bromo-5-(4-fluorophenyl)-3-methyl-1H-pyrazole (730 mg), 6-(4,4,5,5-tetramethyl-1,3,2-dioxaborolan-2-yl)-2H-1,4-benzoxazin-3(4H)-one (500 mg), [1,1′-bis(diphenylphosphino)ferrocene]dichloropalladium(II) dichloromethane adduct (150 mg), cesium carbonate (1.20 g), water (4 ml) and tetrahydrofuran (20 ml) was refluxed for 12 h under argon atmosphere. Water and ethyl acetate was added to the mixture and the mixture was filtered. The organic layer was separated, washed with water, brine, dried over Na₂SO₄ and concentrated in vacuo. The residue was purified by chromatography on silica gel (hexane:ethyl acetate=4:1 to 1:2) followed by crystallization from ethyl acetate/hexane to give the title compound as crystals (375 mg).

Mp 138-140° C.

¹H-NMR (300 MHz, DMSO-d₆) δ: 2.20 (3H, s), 3.48-3.82 (2H, m), 4.09-4.32 (2H, m), 4.47 (2H, s), 4.53 (2H, s), 4.91-5.23 (1H, m), 6.58 (1H, dd, J=8.3, 2.1 Hz), 6.65 (1H, d, J=2.1 Hz), 6.82 (1H, d, J=8.3 Hz), 7.16-7.42 (9H, m), 10.60 (1H, s).

### Preparation 144

### 6-{1-[3-(Benzyloxy)-2-oxopropyl]-5-(4-fluorophenyl)-3-methyl-1H-pyrazol-4-yl}-2H-1,4-benzoxazin-3(4H)-one

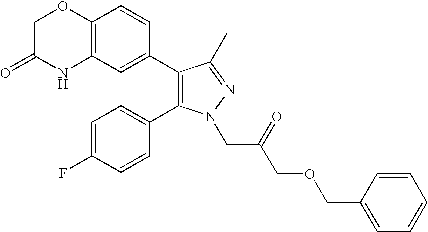

A mixture of 1-(benzyloxy)-3-[4-bromo-5-(4-fluorophenyl)-3-methyl-1H-pyrazol-1-yl]propan-2-one (500 mg), 6-(4,4,5,5-tetramethyl-1,3,2-dioxaborolan-2-yl)-2H-1,4-benzoxazin-3(4H)-one (350 mg), [1,1′-bis(diphenylphosphino)ferrocene]dichloropalladium(II) dichloromethane adduct (110 mg), cesium carbonate (850 mg), water (3 ml) and tetrahydrofuran (15 ml) was refluxed for 12 h under argon atmosphere. Water and ethyl acetate was added to the mixture and the mixture was filtered. The organic layer was separated, washed with water, brine, dried over Na₂SO₄ and concentrated in vacuo. The residue was purified by chromatography on silica gel (hexane:ethyl acetate=4:1 to 1:3) followed by crystallization from ethyl acetate/hexane to give the title compound as crystals (240 mg).

Mp 154-155° C.

¹H-NMR (300 MHz, DMSO-d₆) δ: 2.19 (3H, s), 4.19 (2H, s), 4.43 (2H, s), 4.54 (2H, s), 4.99 (2H, s), 6.59 (1H, dd, J=8.1, 1.9 Hz), 6.68 (1H, d, J=1.9 Hz), 6.83 (1H, d, J=8.1 Hz), 7.16-7.42 (9H, m), 10.61 (1H s).

### Preparation 145

### 6-{1-[3-(Benzyloxy)-2,2-difluoropropyl]-5-(4-fluorophenyl)-3-methyl-1H-pyrazol-4-yl}-2H-1,4-benzoxazin-3(4H)-one

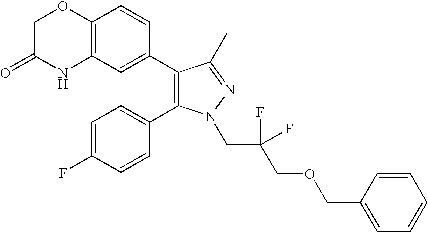

A mixture of 1-[3-(benzyloxy)-2,2-difluoropropyl]-4-bromo-5-(4-Fluorophenyl)-3-methyl-1H-pyrazole (600 mg), 6-(4,4,5,5-tetramethyl-1,3,2-dioxaborolan-2-yl)-2H-1,4-benzoxazin-3(4H)-one (490 mg), [1,1′-bis(diphenylphosphino)ferrocene]dichloropalladium(II) dichloromethane adduct (220 mg), cesium carbonate (1.20 g), water (5 ml) and tetrahydrofuran (25 ml) was refluxed for 24 h under argon atmosphere. Water and ethyl acetate was to added to the mixture and the mixture was filtered. The organic layer was separated, washed with brine, dried over Na₂SO₄ and concentrated in vacuo. The residue was purified by chromatography on silica gel (hexane:ethyl acetate=10:1 to 2:3) followed by crystallization from diisopropyl ether/hexane to give the title compound as crystals (420 mg).

Mp 154-155° C.

¹H-NMR (300 MHz, DMSO-d₆) δ: 2.20 (3H, s), 3.74 (2H, t, J=13.3 Hz), 4.41-4.57 (6H, m), 6.58 (1H, dd, J=8.2, 2.0 Hz), 6.66 (1H, d, J=2.0 Hz), 6.82 (1H, d, J=8.2 Hz), 7.18-7.40 (9H, m), 10.61 (1H, s).

### Example 103

### 6-[5-(4-Fluorophenyl)-3-methyl-1-(3-oxobutyl)-1H-pyrazol-4-yl]-2H-1,4-benzoxazin-3(4H)-one

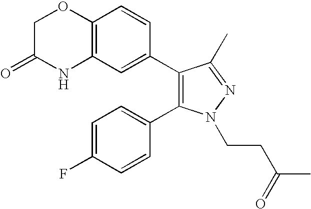

A mixture of 4-[4-bromo-5-(4-fluorophenyl)-3-methyl-1H-pyrazol-1-yl]butan-2-one (780 mg), 6-(4,4,5,5-tetramethyl-1,3,2-dioxaborolan-2-yl)-2H-1,4-benzoxazin-3(4H)-one (700 mg), [1,1′-bis(diphenylphosphino)ferrocene]dichloropalladium(II) dichloromethane adduct (210 mg), cesium carbonate (1.70 g), water (6 ml) and tetrahydrofuran (30 ml) was refluxed for 12 h under argon atmosphere. Water and ethyl acetate was added to the mixture and the mixture was filtered. The organic layer was separated, washed with water, brine, dried over Na₂SO₄ and concentrated in vacuo. The residue was purified by chromatography on silica gel (hexane:ethyl acetate=4:1 to ethyl acetate). Resulting crystals were washed with diethyl ether/diisopropyl ether to give the title compound as crystals (260 mg).

Mp 148-151° C.

¹H-NMR (300 MHz, DMSO-d₆) δ: 2.08 (3H, s), 2.17 (3H, s), 2.99 (2H, t, J=6.9 Hz), 4.04 (2H, t, J=6.9 Hz), 4.53 (2H, s), 6.56 (1H, dd, J=8.3, 2.1 Hz), 6.63 (1H, d, J=2.1 Hz), 6.81 (1H, d, J=8.3 Hz), 7.22-7.38 (4H, m), 10.59 (1H, s).

### Example 104

### 6-[5-(4-Fluorophenyl)-3-methyl-1-(4,4,4-trifluoro-3-hydroxybutyl)-1H-pyrazol-4-yl]-2H-1,4-benzoxazin-3(4H)-one

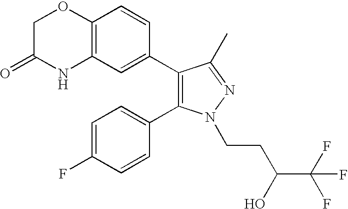

The title compound was obtained from 4-[4-bromo-5-(4-fluorophenyl)-3-methyl-1H-pyrazol-1-yl]-1,1,1-trifluorobutan-2-ol (500 mg) and 6-(4,4,5,5-tetramethyl-1,3,2-dioxaborolan-2-yl)-2H-1,4-benzoxazin-3(4H)-one (470 mg) according to the similar procedure described for 6-[5-(4-fluorophenyl)-3-methyl-1-(3-oxobutyl)-1H-pyrazol-4-yl]-2H-1,4-benzoxazin-3(4H)-one as crystals (328 mg).

Mp 200-202° C. (diisopropyl ether).

¹H-NMR (300 MHz, DMSO-d₆) δ: 1.75-1.92 (1H, m), 2.01-2.17 (1H, m), 2.20 (3H, s), 3.88-4.10 (3H, m), 4.53 (2H, s), 6.26 (1H, d, J=6.4 Hz), 6.53-6.62 (1H, m), 6.65 (1H, d, J=1.9 Hz), 6.82 (1H, d, J=8.3 Hz), 7.21-7.39 (4H, m), 10.60 (1H, s).

### Preparation 146

### 6-{1-[4-(Benzyloxy)-2,2-difluorobutyl]-5-(4-fluorophenyl)-3-methyl-1H-pyrazol-4-yl}-2H-1,4-benzoxazin-3(4H)-one

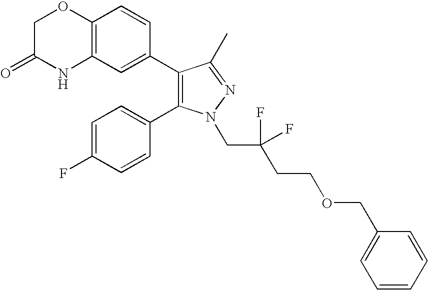

The title compound was obtained from 1-[4-(benzyloxy)-2,2-difluorobutyl]-4-bromo-5-(4-fluorophenyl)-3-methyl-1H-pyrazole (1.00 g) and 6-(4,4,5,5-tetramethyl-1,3,2-dioxaborolan-2-yl)-2H-1,4-benzoxazin-3(4H)-one (800 mg) according to the similar procedure described for 6-[5-(4-fluorophenyl)-3-methyl-1-(3-oxobutyl)-1H-pyrazol-4-yl]-2H-1,4-benzoxazin-3(4H)-one as crystals (630 mg).

Mp 108-109° C. (diisopropyl ether).

¹H-NMR (300 MHz, DMSO-d₆) δ: 2.11-2.31 (5H, m), 3.51 (2H, t, J=6.4 Hz), 4.36-4.56 (6H, m), 6.58 (1H, dd, J=8.3, 1.9 Hz), 6.65 (1H, d, J=1.9 Hz), 6.82 (1H, d, J=8.3 Hz), 7.18-7.39 (9H, m), 10.61 (1H, s).

### Example 105

### 6-[5-(4-Fluorophenyl)-1-{2-[(2-hydroxyethyl)sulfanyl]ethyl}-3-methyl-1H-pyrazol-4-yl]-2H-1,4-benzoxazin-3(4H)-one

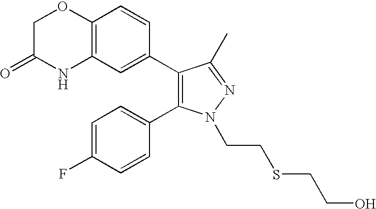

The title compound was obtained from 2-({2-[4-bromo-5-(4-fluorophenyl)-3-methyl-1H-pyrazol-1-yl]ethyl}sulfanyl)ethanol (480 mg) and 6-(4,4,5,5-tetramethyl-1,3,2-dioxaborolan-2-yl)-2H-1,4-benzoxazin-3(4H)-one (480 mg) according to the similar procedure described for 6-[5-(4-fluorophenyl)-3-methyl-1-(3-oxobutyl)-1H-pyrazol-4-yl]-2H-1,4-benzoxazin-3(4H)-one as crystals (217 mg).

Mp 158-160° C. (ethyl acetate).

¹H-NMR (300 MHz, DMSO-d₆) δ: 2.19 (3H, s), 2.37 (2H, t, J=6.8 Hz), 2.80-2.90 (2H, m), 3.36-3.47 (2H, m), 3.99-4.09 (2H, m), 4.53 (2H, s), 4.74 (1H, t, J=5.3 Hz), 6.57 (1H, dd, J=8.3, 1.9 Hz), 6.66 (1H, d, J=1.9 Hz), 6.82 (1H, d, J=8.3 Hz), 7.22-7.40 (4H, m), 10.60 (1H, s).

### Example 106

### 6-[5-(4-Fluorophenyl)-1-{2-[(2-hydroxyethyl)sulfonyl]ethyl}-3-methyl-1H-pyrazol-4-yl]-2H-1,4-benzoxazin-3(4H)-one

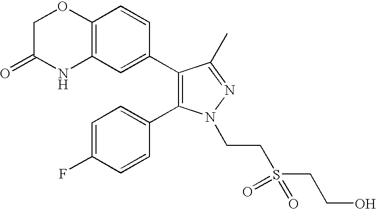

The title compound was obtained from 2-({2-[4-bromo-5-(4-fluorophenyl)-3-methyl-1H-pyrazol-1-yl]ethyl}sulfonyl)ethanol (450 mg) and 6-(4,4,5,5-tetramethyl-1,3,2-dioxaborolan-2-yl)-2H-1,4-benzoxazin-3(4H)-one (420 mg) according to the similar procedure described for 6-[5-(4-fluorophenyl)-3-methyl-1-(3-oxobutyl)-1H-pyrazol-4-yl]-2H-1,4-benzoxazin-3(4H)-one as crystals (210 mg).

Mp 229-231° C. (ethyl acetate).

¹H-NMR (300 MHz, DMSO-d₆) δ: 2.20 (3H, s), 3.19 (2H, t, J=5.7 Hz), 3.62-3.70 (2H, m), 3.71-3.79 (2H, m), 4.25-4.34 (2H, m), 4.53 (2H, s), 5.13 (1H, t, J=4.9 Hz), 6.57 (1H, dd, J=8.3, 2.1 Hz), 6.65 (1H, d, J=2.1 Hz), 6.83 (1H, d, J=8.3 Hz), 7.24-7.40 (4H, m), 10.60 (1H, s).

### Example 107

### 6-[1-(2-Fluoro-3-hydroxypropyl)-5-(4-fluorophenyl)-3-methyl-1H-pyrazol-4-yl]-2H-1,4-benzoxazin-3(4H)-one

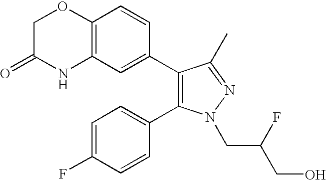

A mixture of 6-{1-[3-(benzyloxy)-2-fluoropropyl]-5-(4-fluorophenyl)-3-methyl-1H-pyrazol-4-yl}-2H-1,4-benzoxazin-3(4H)-one (300 mg), 10% palladium-carbon (300 mg), methanol (3 ml) and tetrahydrofuran (3 ml) was stirred under hydrogen atmosphere (1 atm) at 40° C. for 12 h. The catalyst was filtered off and the filtrate was concentrated in vacuo. The residue was crystallized from diethyl ether to give the title compound as crystals (123 mg).

Mp 193-196° C.

¹H-NMR (300 MHz, DMSO-d₆) δ: 2.20 (3H, s), 3.41-3.72 (2H, m), 4.03-4.27 (2H, m), 4.53 (2H, s), 4.74-5.20 (2H, m), 6.57 (1H, dd, J=8.3, 1.9 Hz), 6.65 (1H, d, J=1.9 Hz), 6.82 (1H, d, J=8.3 Hz), 7.19-7.37 (4H, m), 10.61 (1H, s).

### Example 108

### 6-[1-(2,3-Dihydroxypropyl)-5-(4-fluorophenyl)-3-methyl-1H-pyrazol-4-yl]-2H-1,4-benzoxazin-3(4H)-one

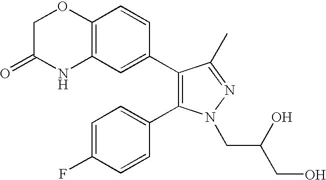

A mixture of 6-{1-[3-(benzyloxy)-2-oxopropyl]-5-(4-fluorophenyl)-3-methyl-1H-pyrazol-4-yl}-2H-1,4-benzoxazin-3(4H)-one (200 mg), 10% palladium-carbon (300 mg), methanol (4 ml) and tetrahydrofuran (4 ml) was stirred under hydrogen atmosphere (1 atm) at room temperature for 24 h and then at 40° C. for12 h. The catalyst was filtered off and the filtrate was concentrated in vacuo. The residue was crystallized from ethyl acetate to give the title compound as crystals (50 mg).

Mp 193-194° C.

¹H-NMR (300 MHz, DMSO-d₆) δ: 2.19 (3H, s), 3.21-3.38 (2H, m), 3.69-3.83 (1H, m), 3.87-4.02 (2H, m), 4.53 (2H, s), 4.63 (1H, t, J=5.7 Hz), 4.96 (1H, d, J=4.9 Hz), 6.55 (1H, dd, J=8.3, 2.1 Hz), 6.64 (1H, d, J=2.1 Hz), 6.82 (1H, d, J=8.3 Hz), 7.17-7.31 (2H, m), 7.32-7.42 (2H, m), 10.59 (1H, s).

### Example 109

### 6-[1-(2,2-Difluoro-3-hydroxypropyl)-5-(4-fluorophenyl)-3-methyl-1H-pyrazol-4-yl]-2H-1,4-benzoxazin-3(4H)-one

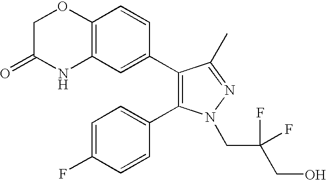

A mixture of 6-{1-[3-(benzyloxy)-2,2-difluoropropyl]-5-(4-fluorophenyl)-3-methyl-1H-pyrazol-4-yl}-2H-1,4-benzoxazin-3(4H)-one (380 mg), 10% palladium-carbon (400 mg), methanol (8 ml) and tetrahydrofuran (8 ml) was stirred under hydrogen atmosphere (1 atm) at 50° C. for 12 h. The catalyst was filtered off and the filtrate was concentrated in vacuo. The resulting crystals were washed with diisopropyl ether to give the title compound as crystals (275 mg).

¹H-NMR (300 MHz, DMSO-d₆) δ: 2.20 (3H, s), 3.60 (2H, td, J=13.9, 6.3 Hz), 4.42 (2H, t, J=14.0 Hz), 4.53 (2H, s), 5.53 (1H, t, J=6.3 Hz), 6.58 (1H, dd, J=8.3, 2.1 Hz), 6.65 (1H, d, J=2.1 Hz), 6.82 (1H, d, J=8.3 Hz), 7.21-7.35 (4H, m), 10.60 (1H, s).

### Example 110

### 6-[5-(4-Fluorophenyl)-1-(3-hydroxybutyl)-3-methyl-1H-pyrazol-4-yl]-2H-1,4-benzoxazin-3(4H)-one

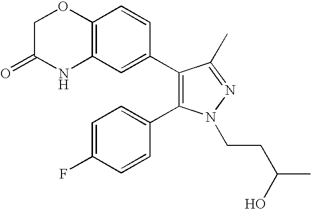

To a solution of 6-[5-(4-fluorophenyl)-3-methyl-1-(3-oxobutyl)-1H-pyrazol-4-yl]-2H-1,4-benzoxazin-3(4H)-one (120 mg) in tetrahydrofuran (4 ml) was added methanol (4 ml) at room temperature and then sodium tetrahydroborate (40 mg) was added to the mixture under ice-cooling. The mixture was allowed to warm to room temperature and stirred for 0.5 h at room temperature. Water was added to the mixture under ice-cooling and the mixture was extracted with ethyl acetate. The organic layer washed with water, brine, dried over Na₂SO₄ and concentrated in vacuo. The residue was diluted with a solution of ethyl acetate and methanol and the mixture was filtered through charcoal. The filtrate was concentrated in vacuo. The residue was crystallized from diisopropyl ether to give the title compound as crystals (53.0 mg).

Mp 158-159° C.

¹H-NMR (300 MHz, DMSO-d₆) δ: 0.98 (3H, d, J=6.4 Hz), 1.56-1.90 (2H, m), 2.18 (3H, s), 3.45-3.64 (1H, m), 3.82-4.06 (2H, m), 4.47 (1H, d, J=4.5 Hz), 4.53 (2H, s), 6.49-6.70 (2H, m), 6.81 (1H, d, J=8.3 Hz), 7.18-7.39 (4H, m), 10.59 (1H, s).

### Example 111

### 6-[5-(4-Fluorophenyl)-1-(3-hydroxy-3-methylbutyl)-3-methyl-1H-pyrazol-4-yl]-2H-1,4-benzoxazin-3(4H)-one

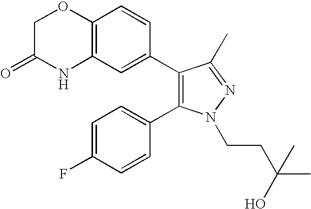

To a solution of 6-[5-(4-fluorophenyl)-3-methyl-1-(3-oxobutyl)-1H-pyrazol-4-yl]-2H-1,4-benzoxazin-3(4H)-one (120 mg) in tetrahydrofuran (6 ml) was added methylmagnesium bromide in diethyl ether (3M, 0.3 ml) at room temperature and the mixture was stirred at room temperature for 0.5 h. Methylmagnesium bromide in diethyl ether (3M, 0.3 ml) was added to the mixture at room temperature and the mixture was stirred at room temperature for 0.5 h. The mixture was added to the ice cooled water. The aqueous layer was acidified with 10% HCl and then basified with 28% ammonia solution. The mixture was extracted with ethyl acetate. The organic layer was washed with water, brine, dried over Na₂SO₄ and concentrated in vacuo. The residue was purified by chromatography on silica gel (hexane:ethyl acetate=4:1 to ethyl acetate) followed by crystallization from diisopropyl ether to give the title compound as crystals (62 mg).

Mp 213-215° C.

¹H-NMR (300 MHz, DMSO-d₆) δ: 1.01 (6H, s), 1.70-1.84 (2H, m), 2.18 (3H, s), 3.89-4.03 (2H, m), 4.31 (1H, s), 4.53 (2H, s), 6.51-6.69 (2H, m), 6.81 (1H, d, J=8.3 Hz), 7.21-7.37 (4H, m), 10.59 (1H, s).

### Example 112

### 6-[1-(2,2-Difluoro-4-hydroxybutyl)-5-(4-fluorophenyl)-3-methyl-1H-pyrazol-4-yl]-2H-1,4-benzoxazin-3(4H)-one

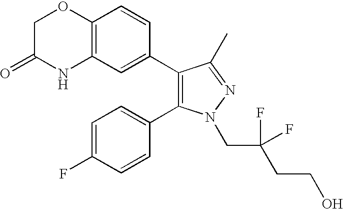

A mixture of 6-{1-[4-(benzyloxy)-2,2-difluorobutyl]-5-(4-fluorophenyl)-3-methyl-1H-pyrazol-4-yl}-2H-1,4-benzoxazin-3(4H)-one (600 mg), 10% palladium-carbon (600 mg) and methanol (24 ml) was stirred under hydrogen atmosphere (1 atm) at 50° C. for 6 h. The catalyst was filtered off and the filtrate was passed through charcoal filter. The filtrate was concentrated in vacuo. The residue was crystallized from ethyl acetate to give the title compound as crystals (348 mg).

Mp 176-177° C.

¹H-NMR (300 MHz, DMSO-d₆) δ: 1.93-2.14 (2H, m), 2.21 (3H, s), 3.45-3.55 (2H, m), 4.43 (2H, t, J=14.2 Hz), 4.53 (2H, s), 4.69 (1H, t, J=5.1 Hz), 6.58 (1H, dd, J=8.2, 2.2 Hz), 6.66 (1H, d, J=2.2 Hz), 6.82 (1H, d, J=8.2 Hz), 7.21-7.33 (4H, m), 10.61 (1H, s).

### Preparation 147

### 1-[4-Bromo-5-(4-fluorophenyl)-3-methyl-1H-pyrazol-1-yl]pentan-3-ol

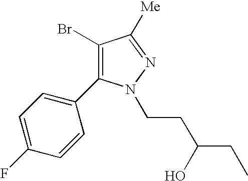

To a stirred solution of 3-[4-bromo-5-(4-fluorophenyl)-3-methyl-1H-pyrazol-1-yl] propanal (200 mg) in tetrahydrofuran (2 mL) was added dropwise ethylmagnesium bromide in tetrahydrofuran (3M, 214 mL) at −78° C. The mixture was stirred for 1 h, treated with aqueous NH₄Cl solution, and extracted with ethyl acetate. The organic layer was dried over MgSO₄ and concentrated in vacuo. The residue was purified by preparative HPLC to give the title compound as an oil (88 mg).

¹H-NMR (300 MHz, DMSO-d₆) δ: 0.76 (3H, t, J=7.4 Hz), 1.13-1.35 (2H, m), 1.50-1.68 (1H, m), 1.72-1.87 (1H, m),2.18 (3H, s), 3.13-3.30 (1H, m), 3.88-4.13 (2H, m), 4.42 (1H, d, J=5.3 Hz), 7.30-7.44 (2H, m), 7.44-7.65 (2H, m).

LCMS (ESI⁺) M+H⁺: 341, 343.

### Preparation 148

### 1-[5-(Benzyloxy)pentyl]-4-bromo-5-(4-fluorophenyl)-3-methyl-1H-pyrazole

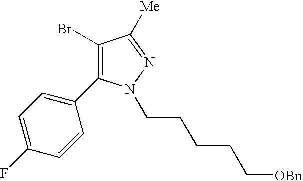

To a stirred mixture of 4-bromo-5-(4-fluorophenyl)-3-methyl-1H-pyrazole (678 mg), 5-(benzyloxy)pentan-1-ol (1.55 g) and triphenylphosphine (2.1 g) in tetrahydrofuran (7 mL) was added dropwise diisopropyl azodicarboxylate in toluene (1.9M, 4.2 mL) at 60° C. The mixture was stirred for 30 min, treated with water and extracted with ethyl acetate. The organic layer was dried over MgSO₄ and concentrated in vacuo. The residue was dissolved in diisopropyl ether and sonicated. The resulting precipitates were filtered off and the filtrate was evaporated. The residue was chromatographed on silica gel eluting with hexane/ethyl acetate to give the title compound as an oil (410 mg).

¹H-NMR (300 MHz, DMSO-d₆) δ: 1.11-1.25 (2H, m), 1.31-1.46 (2H, m), 1.53-1.68 (2H, s), 2.18 (3H, s), 3.27-3.32 (2H, m), 3.94 (2H, t, J=7.1 Hz), 4.38 (2H, s), 7.22-7.42 (7H, m), 7.43-7.51 (2H, m).

LCMS (ESI⁺) M+H⁺: 431, 433.

### Preparation 149

### 4-Bromo-5-(4-fluorophenyl)-3-methyl-1-(2-methylpropyl)-1H-pyrazole

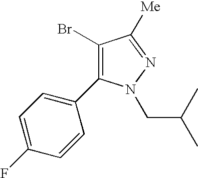

To a stirred mixture of 4-bromo-5-(4-fluorophenyl)-3-methyl-1H-pyrazole (500 mg), 2-methylpropan-1-ol (436 mg) and triphenylphosphine (1.5 g) in tetrahydrofuran (5 mL) was added dropwise diisopropyl azodicarboxylate in toluene (1.9M, 3.1 mL) at 60° C. The mixture was stirred for 30 min, treated with water and extracted with ethyl acetate. The organic layer was dried over MgSO₄ and concentrated in vacuo. The residue was dissolved in diisopropyl ether and sonicated. The precipitate was filtered off and the filtrate was evaporated. The residue was chromatographed on silica gel eluting with hexane/ethyl acetate to give the title compound as an oil (100 mg).

¹H-NMR (300 MHz, DMSO-d₆) δ: 0.67 (6H, d, J=6.8 Hz), 1.87-2.02 (1H, m), 2.19 (3H, s), 3.77 (2H, d, J=7.3 Hz), 7.31-7.43 (2H, m), 7.43-7.53 (2H, m).

LCMS (ESI⁺) M+H⁺: 311, 313.

### Preparation 150

### 4-Bromo-5-(4-fluorophenyl)-3-methyl-1-(2-methylbutyl)-1H-pyrazole

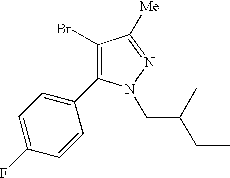

To a stirred mixture of 4-bromo-5-(4-fluorophenyl)-3-methyl-1H-pyrazole (600 mg), 2-methylbutan-1-ol (621 mg) and triphenylphosphine (1.8 g) in tetrahydrofuran (6 mL) was added dropwise diisopropyl azodicarboxylate in toluene (1.9M, 3.7 mL) at 60° C. The mixture was stirred for 30 min, treated with water and extracted with ethyl acetate. The organic layer was dried over MgSO₄ and concentrated in vacuo. The residue was dissolved in diisopropyl ether and sonicated. The precipitate was filtered off and the filtrate was evaporated. The residue was chromatographed on silica gel eluting with hexane/ethyl acetate to give the title compound as an oil (206 mg).

¹H-NMR (300 MHz, DMSO-d₆) δ: 0.58-0.76 (6H, m), 0.81-1.00 (1H, m), 1.05-1.18 (1H, m), 1.65-1.81 (1H, m), 2.18 (3H, s), 3.66-3.78 (1H, m), 3.83-3.95 (1H, m), 7.28-7.43 (2H, m), 7.43-7.52 (2H, m).

LCMS (ESI⁺) M+H⁺: 325, 327.

### Preparation 151

### 4-Bromo-1-(2-ethylbutyl)-5-(4-fluorophenyl)-3-methyl-1H-pyrazole

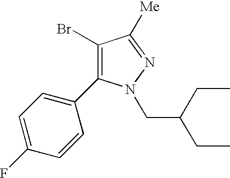

To a stirred mixture of 4-bromo-5-(4-fluorophenyl)-3-methyl-1H-pyrazole (600 mg), 2-ethylbutan-1-ol (721 mg) and triphenylphosphine (1.8 g) in tetrahydrofuran (6 mL) was added dropwise diisopropyl azodicarboxylate in toluene (1.9M, 3.7 mL) at 60° C. The mixture was stirred for 30 min, treated with water and extracted with ethyl acetate. The organic layer was dried over MgSO₄ and concentrated in vacuo. The residue was dissolved in diisopropyl ether and sonicated. The precipitate was filtered off and the filtrate was evaporated. The residue was chromatographed on silica gel eluting with hexane/ethyl acetate to give the title compound as an oil (180 mg).

¹H-NMR (300 MHz, DMSO-d₆) δ: 0.62 (6H, t, J=7.5 Hz), 0.97-1.13 (4H, m), 1.47-1.63 (1H, m), 2.18 (3H, s), 3.87 (2H, d, J=7.2 Hz), 7.32-7.42 (2H, m), 7.43-7.55 (2H, m).

LCMS (ESI⁺) M+H⁺: 339, 341.

### Preparation 152

### 4-Bromo-5-(4-fluorophenyl)-3-methyl-1-(3-methylbutyl)-1H-pyrazole

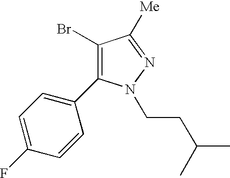

To a stirred mixture of 4-bromo-5-(4-fluorophenyl)-3-methyl-1H-pyrazole (600 mg), 3-methylbutan-1-ol (622 mg) and triphenylphosphine (1.8 g) in tetrahydrofuran (6 mL) was added dropwise diisopropyl azodicarboxylate in toluene (1.9M, 3.7 mL) at 60° C. The mixture was stirred for 30 min, treated with water and extracted with ethyl acetate. The organic layer was dried over MgSO₄ and concentrated in vacuo. The residue was dissolved in diisopropyl ether and sonicated. The precipitate was filtered off and the filtrate was evaporated. The residue was chromatographed on silica gel eluting with hexane/ethyl acetate to give the title compound as an oil (45 mg).

¹H-NMR (300 MHz, DMSO-d₆) δ: 0.71 (6H, d, J=6.4 Hz), 1.31-1.43 (1H, m), 1.43-1.54 (2H, m), 2.18 (3H, s), 3.95 (2H, t, J=7.5 Hz), 7.36-7.43 (2H, m), 7.44-7.55 (2H, m).

LCMS (ESI⁺) M+H⁺: 325, 327.

### Preparation 153

### 4-Bromo-1-butyl-5-(4-fluorophenyl)-3-methyl-1H-pyrazole

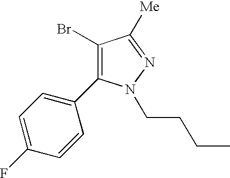

To a stirred mixture of 4-bromo-5-(4-fluorophenyl)-3-methyl-1H-pyrazole (600 mg), butan-1-ol (523 mg) and triphenylphosphine (1.8 g) in tetrahydrofuran (6 mL) was added dropwise diisopropyl azodicarboxylate in toluene (1.9M, 3.7 mL) at 60° C. The mixture was stirred for 30 min, treated with water and extracted with ethyl acetate. The organic layer was dried over MgSO₄ and concentrated in vacuo. The residue was dissolved in diisopropyl ether and sonicated. The precipitate was filtered off and the filtrate was evaporated. The residue was chromatographed on silica gel eluting with hexane/ethyl acetate to give the title compound as an oil (211 mg).

¹H-NMR (300 MHz, DMSO-d₆) δ: 0.73 (3H, t, J=7.2 Hz), 1.03-1.17 (2H, m), 1.52-1.63 (2H, m), 2.18 (3H, s), 3.94 (2H, t, J=7.0 Hz), 7.33-7.43 (2H, m), 7.45-7.54 (2H, m).

LCMS (ESI⁺) M+H⁺: 311, 313.

### Preparation 154

### 4-Bromo-5-(4-fluorophenyl)-3-methyl-1-pentyl-1H-pyrazole

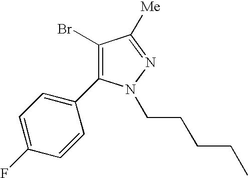

To a stirred mixture of 4-bromo-5-(4-fluorophenyl)-3-methyl-1H-pyrazole (600 mg), pentan-1-ol (622 mg) and triphenylphosphine (1.8 g) in tetrahydrofuran (6 mL) was added dropwise diisopropyl azodicarboxylate in toluene (1.9M, 3.7 mL) at 60° C. The mixture was stirred for 30 min, treated with water and extracted with ethyl acetate. The organic layer was dried over MgSO₄ and concentrated in vacuo. The residue was dissolved in diisopropyl ether and sonicated. The precipitate was filtered off and the filtrate was evaporated. The residue was chromatographed on silica gel eluting with hexane/ethyl acetate to give the title compound as an oil (161 mg).

¹H-NMR (300 MHz, DMSO-d₆) δ: 0.75 (3H, t, J=7.1 Hz), 0.97-1.19 (4H, m), 1.52-1.67 (2H, m), 2.18 (3H, s), 3.93 (2H, t, J=7.3 Hz), 7.33-7.44 (2H, m), 7.44-7.54 (2H, m).

LCMS (ESI⁺) M+H⁺: 325, 327.

### Preparation 155

### 4-Bromo-5-(4-fluorophenyl)-3-methyl-1-[(1-methylcyclopropyl)methyl]-1H-pyrazole

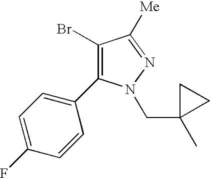

To a stirred mixture of 4-bromo-5-(4-fluorophenyl)-3-methyl-1H-pyrazole (500 mg), (1-methylcyclopropyl)methanol (518 mg) and triphenylphosphine (1.5 g) in tetrahydrofuran (5 mL) was added dropwise diisopropyl azodicarboxylate in toluene (1.9M, 3.1 mL) at 60° C. The mixture was stirred for 30 min, treated with water and extracted with ethyl acetate. The organic layer was dried over MgSO₄ and concentrated in vacuo. The residue was dissolved in diisopropyl ether and sonicated. The precipitate was filtered off and the filtrate was evaporated. The residue was chromatographed on silica gel eluting with hexane/ethyl acetate to give the title compound as an oil (119 mg).

¹H-NMR (300 MHz, DMSO-d₆) δ: 0.09-0.28 (4H, m), 0.74 (3H, s), 2.19 (3H, s), 3.88 (2H, s), 7.32-7.43 (2H, m), 7.42-7.51 (2H, m).

LCMS (ESI⁺) M+H⁺: 323, 325.

### Preparation 156

### 4-Bromo-5-(4-fluorophenyl)-3-methyl-1-[(2-methylcyclopropyl)methyl]-1H-pyrazole

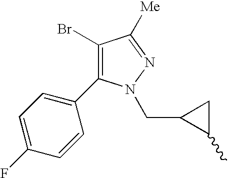

(cis/trans=¼)

To a stirred mixture of 4-bromo-5-(4-fluorophenyl)-3-methyl-1H-pyrazole (600 mg), (2-methylcyclopropyl)methanol (608 mg) and triphenylphosphine (1.8 g) in tetrahydrofuran (6 mL) was added dropwise diisopropyl azodicarboxylate in toluene (1.9M, 3.7 mL) at 60° C. The mixture was stirred for 30 min, treated with water and extracted with ethyl acetate. The organic layer was dried over MgSO₄ and concentrated in vacuo. The residue was dissolved in diisopropyl ether and sonicated. The precipitate was filtered off and the filtrate was evaporated. The residue was chromatographed on silica gel eluting with hexane/ethyl acetate to give the title compound as an oil (304 mg, cis/trans=¼ mixture).

¹H-NMR (300 MHz, DMSO-d₆) δ: 0.07-0.27 (2H, m) 0.34-0.61 (1H, m) 0.62-0.77 (1H, m) 0.81-0.90 (3H, m) 2.19 (3H, s) 3.77-4.06 (2H, m) 7.29-7.56 (4H, m).

LCMS (ESI⁺) M+H⁺: 323, 325.

### Preparation 157

### 4-Bromo-5-(4-fluorophenyl)-3-methyl-1-(2-methylprop-2-en-1-yl)-1H-pyrazole

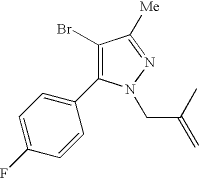

To a stirred mixture of 4-bromo-5-(4-fluorophenyl)-3-methyl-1H-pyrazole (500 mg), 2-methylprop-2-en-1-ol (424 mg) and triphenylphosphine (1.5 g) in tetrahydrofuran (5 mL) was added dropwise diisopropyl azodicarboxylate in toluene (1.9M, 3.1 mL) at 60° C. The mixture was stirred for 30 min, treated with water and extracted with ethyl acetate. The organic layer was dried over MgSO₄ and concentrated in vacuo. The residue was dissolved in diisopropyl ether and sonicated. The precipitate was filtered off and the filtrate was evaporated. The residue was chromatographed on silica gel eluting with hexane/ethyl acetate to give the title compound as an oil (134 mg).

¹H-NMR (300 MHz, DMSO-d₆) δ: 1.50 (3H, s), 2.19 (3H, s), 4.35 (1H, s), 4.54 (2H, s), 4.77 (1H, s), 7.28-7.42 (2H, m), 7.43-7.53 (2H, m).

LCMS (ESI⁺) M+H⁺: 309, 311.

### Preparation 158

### 3-[4-Bromo-5-(4-fluorophenyl)-3-methyl-1H-pyrazol-1-yl]-2-methylpropan-1-ol

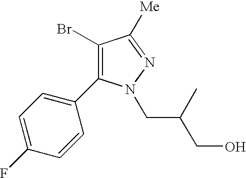

Under nitrogen atmosphere, to a stirred solution of 4-bromo-5-(4-fluorophenyl)-3-methyl-1-(2-methylprop-2-en-1-yl)-1H-pyrazole (134 mg) in tetrahydrofuran (1.5 mL) was added 0.5 M 9-Borabicyclo[3.3.1]nonane tetrahydrofuran—solution (3.46 mL) at 0° C. The mixture was stirred at room temperature for 12 h. To the mixture were added water (0.5 mL), 8N NaOH (0.3 mL) and 30% hydrogen peroxide (1 mL). The mixture was stirred at room temperature for 3 h, treated with water and extracted with ethyl acetate. The organic layer was dried over MgSO₄ and concentrated in vacuo. The residue was chromatographed on silica gel eluting with hexane/ethyl acetate to give the title compound as an oil (106 mg).

¹H-NMR (300 MHz, DMSO-d₆) δ: 0.62 (3H, d, J=6.8 Hz), 1.90-2.03 (1H, m), 2.18 (3H, s), 3.15 (2H, t, J=5.5 Hz), 3.64-3.75 (1H, m), 3.99-4.10 (1H, m), 4.49 (1H, t, J=5.2 Hz), 7.32-7.41 (2H, m), 7.43-7.56 (2H, m).

LCMS (ESI⁺) M+H⁺: 327, 329.

### Preparation 159

### Methyl 3-[4-Bromo-5-(4-fluorophenyl)-3-methyl-1H-pyrazol-1-yl]-2,2-dimethylpropanoate

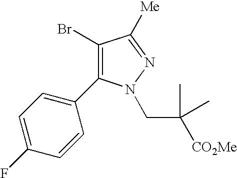

To a stirred mixture of 4-bromo-5-(4-fluorophenyl)-3-methyl-1H-pyrazole (1.0 g), methyl 3-hydroxy-2,2-dimethylpropanoate (1.7 g) and triphenylphosphine (3.1 g) in tetrahydrofuran (10 mL) was added dropwise diisopropyl azodicarboxylate in toluene (1.9M, 6.2 mL) at 60° C. The mixture was stirred for 30 min, treated with water and extracted with ethyl acetate. The organic layer was dried over MgSO₄ and concentrated in vacuo. The residue was dissolved in diisopropyl ether and sonicated. The precipitate was filtered off and the filtrate was evaporated. The residue was chromatographed on silica gel eluting with hexane/ethyl acetate to give the title compound as an oil (199 mg).

¹H-NMR (300 MHz, DMSO-d₆) δ: 0.92 (6H, s), 2.15 (3H, s), 3.53 (3H, s), 4.12 (2H, s), 7.29-7.42 (2H, m), 7.44-7.53 (2H, m).

LCMS (ESI⁺) M+H⁺: 369, 371.

### Preparation 160

### 1-({1-[(Benzyloxy)methyl]cyclopropyl}methyl)-4-bromo-5-(4-fluorophenyl)-3-methyl-1H-pyrazole

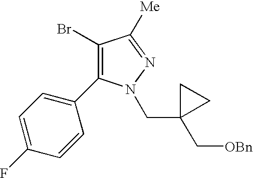

To a stirred mixture of 4-bromo-5-(4-fluorophenyl)-3-methyl-1H-pyrazole (1.66 g), {1-[(benzyloxy)methyl]cyclopropyl}methanol (2.5 g. Org. lett. 2000, 2, 2323-2326) and triphenylphosphine (4.3 g) in tetrahydrofuran (16 mL) was added dropwise diisopropyl azodicarboxylate in toluene (1.9M, 8.6 mL) at 60° C. The mixture was stirred for 30 min, treated with water and extracted with ethyl acetate. The organic layer was dried over MgSO₄ and concentrated in vacuo. The residue was dissolved in diisopropyl ether and sonicated. The precipitate was filtered off and the filtrate was evaporated. The residue was chromatographed on silica gel eluting with hexane/ethyl acetate to give the title compound as an oil (846 mg).

¹H-NMR (300 MHz, DMSO-d₆) δ: 0.32-0.47 (4H, m), 2.18 (3H, s), 3.04 (2H, s), 4.04 (2H, s), 4.21 (2H, s), 7.14 (2H, d, J=1.9 Hz), 7.22-7.38 (5H, m), 7.42-7.51 (2H, m).

LCMS (ESI⁺) M+H⁺: 429, 431.

### Preparation 161

### 1-({1-[(Benzyloxy)methyl]cyclobutyl}methyl)-4-bromo-5-(4-fluorophenyl)-3-methyl-1H-pyrazole

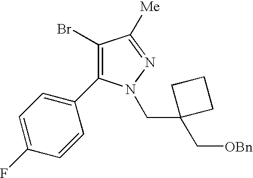

To a stirred mixture of 4-bromo-5-(4-fluorophenyl)-3-methyl-1H-pyrazole (2.0 g), {1-[(benzyloxy)methyl]cyclobutyl}methanol (3.2 g. J. Med. Chem. 2004, 47, 5057-5068) and triphenylphosphine (5.1 g) in tetrahydrofuran (20 mL) was added dropwise diisopropyl azodicarboxylate in toluene (1.9M, 10.3 mL) at 60° C. The mixture was stirred for 30 min, treated with water and extracted with ethyl acetate. The organic layer was dried over MgSO₄ and concentrated in vacuo. The residue was dissolved in diisopropyl ether and sonicated. The precipitate was filtered off and the filtrate was evaporated. The residue was chromatographed on silica gel eluting with hexane/ethyl acetate to give the title compound as an oil (341 mg).

¹H-NMR (300 MHz, DMSO-d₆) δ: 1.52-1.88 (6H, m), 2.18 (3H, s), 3.32 (2H, s), 4.10 (2H, s), 4.30 (2H, s), 7.14-7.17 (2H, m), 7.19-7.37 (5H, m), 7.41-7.52 (2H, m).

LCMS (ESI⁺) M+H⁺: 443, 445.

### Example 113

### 6-[5-(4-Fluorophenyl)-1-(3-hydroxypentyl)-3-methyl-1H-pyrazol-4-yl]-2H-1,4-benzoxazin-3(4H)-one

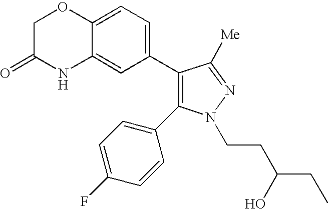

A mixture of 6-(4,4,5,5-tetramethyl-1,3,2-dioxaborolan-2-yl)-2H-1,4-benzoxazin-3(4H)-one (92 mg), 1-[4-bromo-5-(4-fluorophenyl)-3-methyl-1H-pyrazol-1-yl]pentan-3-ol (88 mg), [1,1′-bis(diphenylphosphino)ferrocene]dichloropalladium(II) dichloromethane adduct (42 mg), cesium carbonate (252 mg) in tetrahydrofuran/water (2/1 mL) was exposed to microwave irradiation at 150° C. for 1 h, treated with water and extracted with ethyl acetate. The organic layer was dried over MgSO₄, and concentrated in vacuo. The residue was chromatographed on silica gel eluting with ethyl acetate/hexane. Crystallization from ethyl acetate/hexane gave the title compound (51 mg).

¹H-NMR (300 MHz, DMSO-d₆) δ: 0.77 (3H, t, J=7.4 Hz), 1.17-1.37 (2H, m), 1.51-1.74 (1H, m), 1.72-1.94 (1H, m), 2.18 (3H, s), 3.16-3.31 (1H, m), 3.77-4.11 (2H, m), 4.43 (1H, d, J=5.3 Hz), 4.53 (2H, s), 6.56 (1H, dd, J=8.3, 1.9 Hz), 6.64 (1H, d, J=1.9 Hz), 6.81 (1H, d, J=8.3 Hz), 7.14-7.39 (4H, m), 10.59 (1H, s).

LCMS (ESI⁺) M+H⁺: 410.

### Example 114

### 6-[5-(4-Fluorophenyl)-3-methyl-1-(3-oxopentyl)-1H-pyrazol-4-yl]-2H-1,4-benzoxazin-3(4H)-one

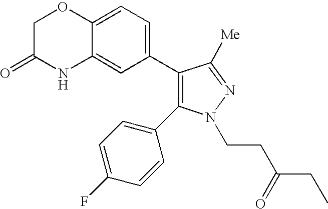

To a stirred solution of 6-[5-(4-fluorophenyl)-1-(3-hydroxypentyl)-3-methyl-1H-pyrazol-4-yl]-2H-1,4-benzoxazin-3(4H)-one (29 mg) in CH3CN (1.5 mL) was added 1,1,1-tris(acetyloxy)-1,1-dihydro-1,2-benziodoxol-3-(1H)-one (60 mg) at room temperature. The mixture was stirred for 2 h, treated with water and extracted with ethyl acetate. The organic layer was dried over MgSO₄ and concentrated in vacuo. The residue was chromatographed on silica gel eluting with hexane/ethyl acetate. Crystallization from ethyl acetate/hexane gave the title compound (18 mg).

¹H-NMR (300 MHz, DMSO-d₆) δ: 0.89 (3H, t, J=7.3 Hz), 2.17 (3H, s), 2.42 (2H, q, J=7.3 Hz), 2.97 (2H, t, J=7.0 Hz), 4.05 (2H, t, J=7.0 Hz), 4.53 (2H, s), 6.56 (1H, dd, J=8.3, 2.1 Hz), 6.63 (1H, d, J=2.1 Hz), 6.81 (1H, d, J=8.3 Hz), 7.22-7.38 (4H, m), 10.59 (1H, s).

LCMS (ESI⁺) M+H⁺: 408.

### Preparation 162

### 6-{1-[5-(Benzyloxy)pentyl]-5-(4-fluorophenyl)-3-methyl-1H-pyrazol-4-yl}-2H-1,4-benzoxazin-3(4H)-one

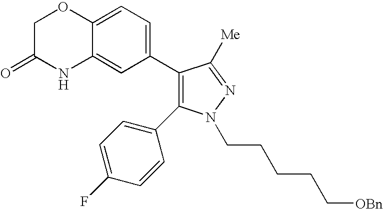

A mixture of 6-(4,4,5,5-tetramethyl-1,3,2-dioxaborolan-2-yl)-2H-1,4-benzoxazin-3(4H)-one (314 mg), 1-[5-(benzyloxy)pentyl]-4-bromo-5-(4-fluorophenyl)-3-methyl-1H-pyrazole (410 mg), [1,1′-bis(diphenylphosphino)ferrocene]dichloropalladium(II) dichloromethane adduct (155 ml), cesium carbonate (929 mg) in tetrahydrofuran/water (12/5 mL) was exposed to microwave irradiation at 150° C. for 1 h, treated with water and extracted with ethyl acetate. The organic layer was dried over MgSO₄, and concentrated in vacuo. The residue was chromatographed on silica gel eluting with ethyl acetate/hexane to give the title compound as an oil (210 mg).

¹H-NMR (300 MHz, DMSO-d₆) δ: 1.11-1.29 (2H, m), 1.32-1.47 (2H, m), 1.55-1.72 (2H, m), 2.18 (3H, s), 3.27-3.36 (2H, m), 3.88 (2H, t, J=7.2 Hz), 4.39 (2H, s), 4.53 (2H, s), 6.56 (1H, dd, J=8.1, 2.1 Hz), 6.65 (1H, d, J=2.1 Hz), 6.81 (1H, d, J=8.1 Hz), 7.20-7.37 (9H, m), 10.60 (1H, s).

LCMS (ESI⁺) M+H⁺: 500.

### Example 115

### 6-[5-(4-Fluorophenyl)-1-(5-hydroxypentyl)-3-methyl-1H-pyrazol-4-yl]-2H-1,4-benzoxazin-3(4H)-one

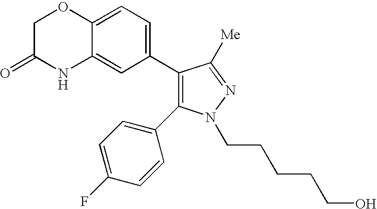

Under hydrogen atmosphere a mixture of 6-{1-[5-(benzyloxy)pentyl]-5-(4-fluorophenyl)-3-methyl-1H-pyrazol-4-yl}-2H-1,4-benzoxazin-3(4H)-one (210 mg) and 10 wt % palladium carbon (400 mg) in ethanol (20 mL) was stirred at 60° C. for 12 h, passed through celite pad and concentrated in vacuo. The residue was chromatographed on silica gel eluting with hexane/ethyl acetate. Crystallization from ethyl acetate/hexane gave the title compound (120 mg).

¹H-NMR (300 MHz, DMSO-d₆) δ: 1.08-1.21 (2H, m), 1.27 (2H, s), 1.57-1.72 (2H, m), 2.19 (3H, s), 3.25-3.32 (2H, m), 3.86 (2H, t, J=7.3 Hz), 4.31 (1H, t, J=5.2 Hz), 4.53 (2H, s), 6.56 (1H, dd, J=8.3, 2.1 Hz), 6.65 (1H, d, J=2.1 Hz), 6.81 (1H, d, J=8.3 Hz), 7.24-7.32 (4H, m), 10.60 (1H, s).

LCMS (ESI⁺) M+H⁺: 410.

Anal. Calcd for C23H24N3O3F (0.1 ethyl acetate): C, 67.20; H, 5.98; N, 10.05. Found: C, 66.98; H, 5.88; N, 10.05

### Example 116

### 6-[5-(4-Fluorophenyl)-3-methyl-1-(2-methylpropyl)-1H-pyrazol-4-yl]-2H-1,4-benzoxazin-3(4H)-one

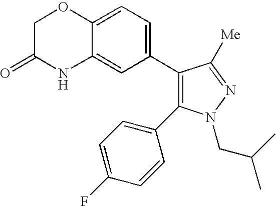

A mixture of 6-(4,4,5,5-tetramethyl-1,3,2-dioxaborolan-2-yl)-2H-1,4-benzoxazin-3(4H)-one (136 mg), 4-bromo-5-(4-fluorophenyl)-3-methyl-1-(2-methylpropyl)-1H-pyrazole (100 mg), [1,1′-bis(diphenylphosphino)ferrocene]dichloropalladium(II) dichloromethane adduct (52.5 mg), cesium carbonate (314 mg) in tetrahydrofuran/water (3/1 mL) was exposed to microwave irradiation at 150° C. for 1 h, treated with water and extracted with ethyl acetate. The organic layer was dried over MgSO₄, and concentrated in vacuo. The residue was chromatographed on silica gel eluting with ethyl acetate/hexane. Crystallization from ethyl acetate/hexane gave the title compound (52 mg).

¹H-NMR (300 MHz, DMSO-d₆) δ: 0.70 (6H, d, J=6.8 Hz), 1.88-2.16 (1H, m), 2.19 (3H, s), 3.71 (2H, d, J=7.2 Hz), 4.53 (2H, s), 6.56 (1H, dd, J=8.3, 1.9 Hz), 6.65 (1H, d, J=1.9 Hz), 6.81 (1H, d, J=8.3 Hz), 7.24-7.32 (4H, m), 10.60 (1H, s).

LCMS (ESI⁺) M+H⁺: 380.

Anal. Calcd for C22H22N3O2F: C, 69.64; H, 5.84; N, 11.07. Found: C, 69.45; H, 5.84; N, 11.05

### Example 117

### 6-[5-(4-Fluorophenyl)-3-methyl-1-(2-methylbutyl)-1H-pyrazol-4-yl]-2H-1,4-benzoxazin-3(4H)-one

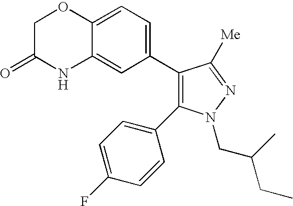

A mixture of 6-(4,4,5,5-tetramethyl-1,3,2-dioxaborolan-2-yl)-2H-1,4-benzoxazin-3(4H)-one (209 mg), 4-bromo-5-(4-fluorophenyl)-3-methyl-1-(2-methylbutyl)-1H-pyrazole (206 mg), [1,1′-bis(diphenylphosphino)ferrocene]dichloropalladium(II) dichloromethane adduct (103 mg), cesium carbonate (619 mg) in tetrahydrofuran/water (3/1 mL) was exposed to microwave irradiation at 150° C. for 1 h, and then passed through a celite pad, treated with water, and extracted with ethyl acetate. The organic layer was washed with brine, dried over MgSO₄, and concentrated in vacuo. The residue was chromatographed on silica gel eluting with hexane/ethyl acetate. Crystallization from ethyl acetate/hexane gave the title compound (113 mg).

¹H-NMR (300 MHz, DMSO-d₆) δ: 0.60-0.77 (6H, m), 0.84-1.04 (1H, m), 1.07-1.28 (1H, m), 1.65-1.88 (1H, m), 2.19 (3H, s), 3.58-3.74 (1H, m), 3.75-3.91 (1H, m), 4.53 (2H, s), 6.56 (1H, dd, J=8.3, 1.9 Hz), 6.65 (1H, d, J=1.9 Hz), 6.81 (1H, d, J=8.3 Hz), 7.22-7.40 (4H, m), 10.59 (1H, s).

LCMS (ESI⁺) M+H⁺: 394.

Anal. Calcd for C23H24N3O2F: C, 70.21; H, 6.15; N, 10.68. Found: C, 70.11; H, 6.13; N, 10.4.

### Example 118

### 6-[1-(2-Ethylbutyl)-5-(4-fluorophenyl)-3-methyl-1H-pyrazol-4-yl]-2H-1,4-benzoxazin-3(4H)-one

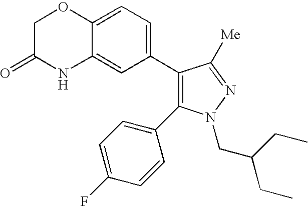

A mixture of 6-(4,4,5,5-tetramethyl-1,3,2-dioxaborolan-2-yl)-2H-1,4-benzoxazin-3(4H)-one (314 mg), 4-bromo-1-(2-ethylbutyl)-5-(4-fluorophenyl)-3-methyl-1H-pyrazole (323 mg), [1,1′-bis(diphenylphosphino)ferrocene]dichloropalladium(II) dichloromethane adduct (156 mg), cesium carbonate (931 mg) in tetrahydrofuran/water (3/1 mL) was exposed to microwave irradiation at 150° C. for 1 h, and then passed through a celite pad, treated with water, and extracted with ethyl acetate. The organic layer was washed with brine, dried over MgSO₄, and concentrated in vacuo. The residue was chromatographed on silica gel eluting with hexane/ethyl acetate. Crystallization from ethyl acetate/hexane gave the title compound (97 mg).

¹H-NMR (300 MHz, DMSO-d₆) δ: 0.63 (6H, t, J=7.4 Hz), 0.97-1.22 (4H, m), 1.51-1.68 (1H, m), 2.19 (3H, s), 3.82 (2H, d, J=7.2 Hz), 4.53 (2H, s), 6.56 (1H, dd, J=8.1, 1.9 Hz), 6.65 (1H, d, J=1.9 Hz), 6.81 (1H, d, J=8.1 Hz), 7.16-7.39 (4H, m), 10.59 (1H, s).

LCMS (ESI⁺) M+H⁺: 408.

Anal. Calcd for C24H26N3O2F: C, 70.74; H, 6.43; N, 10.31. Found: C, 70.67; H, 6.3; N, 10.26.

### Example 119

### 6-[5-(4-Fluorophenyl)-3-methyl-1-(3-methylbutyl)-1H-pyrazol-4-yl]-2H-1,4-benzoxazin-3(4H)-one

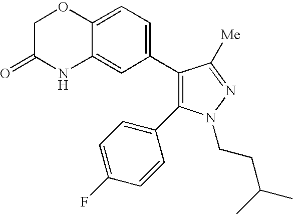

A mixture of 6-(4,4,5,5-tetramethyl-1,3,2-dioxaborolan-2-yl)-2H-1,4-benzoxazin-3(4H)-one (45.7 mg), 4-bromo-5-(4-fluorophenyl)-3-methyl-1-(3-methylbutyl)-1H-pyrazole (45.0 mg), [1,1′-bis(diphenylphosphino)ferrocene]dichloropalladium(II) dichloromethane adduct (23 mg), cesium carbonate (135 mg) in tetrahydrofuran/water (1.2/0.5 mL) was exposed to microwave irradiation at 150° C. for 1 h, and then passed through a celite pad, treated with water, and extracted with ethyl acetate. The organic layer was washed with brine, dried over MgSO₄, and concentrated in vacuo. The residue was chromatographed on silica gel eluting with hexane/ethyl acetate. Crystallization from ethyl acetate/hexane gave the title compound (17 mg).

¹H-NMR (300 MHz, DMSO-d₆) δ: 0.73 (6H, d, J=6.4 Hz), 1.29-1.47 (1H, m), 1.47-1.57 (2H, m), 2.18 (3H, s), 3.80-3.99 (2H, m) 4.53 (2H, s), 6.56 (1H, dd, J=8.1, 1.9 Hz), 6.65 (1H, d, J=1.9 Hz), 6.81 (1H, d, J=8.1 Hz), 7.20-7.40 (4H, m), 10.59 (1H, s).

LCMS (ESI⁺) M+H⁺: 394.

### Example 120

### 6-[1-Butyl-5-(4-fluorophenyl)-3-methyl-1H-pyrazol-4-yl]-2H-1,4-benzoxazin-3(4H)-one

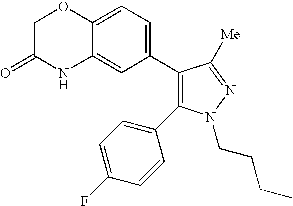

A mixture of 6-(4,4,5,5-tetramethyl-1,3,2-dioxaborolan-2-yl)-2H-1,4-benzoxazin-3(4H)-one (224 mg), 4-bromo-1-butyl-5-(4-fluorophenyl)-3-methyl-1H-pyrazole (211 mg), [1,1′-bis(diphenylphosphino)ferrocene]dichloropalladium(II) dichloromethane adduct (110 mg), cesium carbonate (663 mg) in tetrahydrofuran/water (3/1 mL) was exposed to microwave irradiation at 150° C. for 30 min, and then passed through a celite pad, treated with water, and extracted with ethyl acetate. The organic layer was washed with brine, dried over MgSO₄, and concentrated in vacuo. The residue was chromatographed on silica gel eluting with hexane/ethyl acetate. Crystallization from ethyl acetate/hexane gave the title compound (110 mg).

¹H-NMR (300 MHz, DMSO-d₆) 0.75 (3H, t, J=7.3 Hz), 1.04-1.25 (2H, m), 1.52-1.69 (2H, m), 2.19 (3H, s), 3.88 (2H, t, J=7.2 Hz), 4.53 (2H, s), 6.56 (1H, dd, J=8.3, 2.1 Hz), 6.65 (1H, d, J=2.1 Hz), 6.81 (1H, d, J=8.3 Hz), 7.21-7.34 (4H, m), 10.60 (1H, s).

LCMS (ESI⁺) M+H⁺: 380.

Anal. Calcd for C22H22N3O2F: C, 69.64; H, 5.84; N, 11.07. Found: C, 69.63; H, 5.72; N, 11.1.

### Example 121

### 6-[5-(4-Fluorophenyl)-3-methyl-1-pentyl-1H-pyrazol-4-yl]-2H-1,4-benzoxazin-3(4H)-one

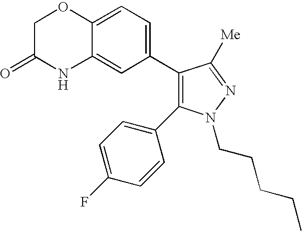

A mixture of 6-(4,4,5,5-tetramethyl-1,3,2-dioxaborolan-2-yl)-2H-1,4-benzoxazin-3(4H)-one (163 mg), 4-bromo-5-(4-fluorophenyl)-3-methyl-1-pentyl-1H-pyrazole (161 mg), [1,1′-bis(diphenylphosphino)ferrocene]dichloropalladium(II) dichloromethane adduct (81 mg), cesium carbonate (483 mg) in tetrahydrofuran/water (3/1 mL) was exposed to microwave irradiation at 150° C. for 30 min, and then passed through a celite pad, treated with water, and extracted with ethyl acetate. The organic layer was washed with brine, dried over MgSO₄, and concentrated in vacuo. The residue was chromatographed on silica gel eluting with hexane/ethyl acetate. Crystallization from ethyl acetate/hexane gave the title compound (68 mg).

¹H-NMR (300 MHz, DMSO-d₆) δ: 0.77 (3H, t, J=7.2 Hz), 1.01-1.21 (4H, m), 1.56-1.70 (2H, m), 2.19 (3H, s), 3.87 (2H, t, J=7.4 Hz), 4.53 (2H, s), 6.56 (1H, dd, J=8.3, 1.9 Hz), 6.65 (1H, d, J=1.9 Hz), 6.81 (1H, d, J=8.3 Hz), 7.24-7.32 (4H, m), 10.59 (1H, s).

LCMS (ESI⁺) M+H⁺: 394.

Anal. Calcd for C23H24N3O2F: C, 70.21; H, 6.15; N, 10.68. Found: C, 70.08; H, 6.07; N, 10.59.

### Example 122

### 6-{5-(4-Fluorophenyl)-3-methyl-1-[(1-methylcyclopropyl)methyl]-1H-pyrazol-4-yl}-2H-1,4-benzoxazin-3(4H)-one

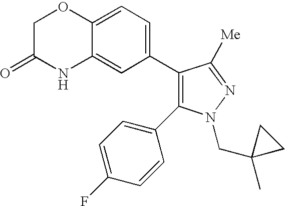

A mixture of 6-(4,4,5,5-tetramethyl-1,3,2-dioxaborolan-2-yl)-2H-1,4-benzoxazin-3(4H)-one (122 mg), 4-bromo-5-(4-fluorophenyl)-3-methyl-1-[(1-methylcyclopropyl)methyl]-1H-pyrazole (119 mg), [1,1′-bis(diphenylphosphino)ferrocene]dichloropalladium(II) dichloromethane adduct (60 mg), cesium carbonate (360 mg) in tetrahydrofuran/water (3/1 mL) was exposed to microwave irradiation at 150° C. for 1 h, and then passed through a celite pad, treated with water, and extracted with ethyl acetate. The organic layer was washed with brine, dried over MgSO₄, and concentrated in vacuo. The residue was chromatographed on silica gel eluting with hexane/ethyl acetate. Crystallization from ethyl acetate/hexane gave the title compound (67.5 mg).

¹H-NMR (300 MHz, DMSO-d₆) δ: 0.10-0.29 (4H, m), 0.78 (3H, s), 2.20 (3H, s), 3.84 (2H, s), 4.53 (2H, s), 6.56 (1H, dd, J=8.3, 1.9 Hz), 6.65 (1H, d, J=1.9 Hz), 6.81 (1H, d, J=8.3 Hz), 7.21-7.36 (4H, m), 10.60 (1 H, s).

LCMS (ESI⁺) M+H⁺: 392.

Anal. Calcd for C23H22N3O2F (0.1 ethyl acetate): C, 70.22; H, 5.74; N, 10.50. Found: C, 70.2; H, 5.55; N, 10.7.

### Example 123

### 6-{5-(4-Fluorophenyl)-3-methyl-1-[(2-methylcyclopropyl)methyl]-1H-pyrazol-4-yl}-2H-1,4-benzoxazin-3(4H)-one

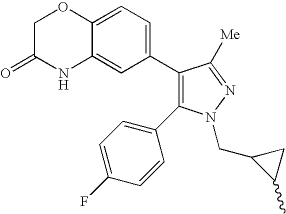

(cis/trans=¼)

A mixture of 6-(4,4,5,5-tetramethyl-1,3,2-dioxaborolan-2-yl)-2H-1,4-benzoxazin-3(4H)-one (311 mg), 4-bromo-5-(4-fluorophenyl)-3-methyl-1-[(2-methylcyclopropyl)methyl]-1H-pyrazole (304 mg), [1,1′-bis(diphenylphosphino)ferrocene]dichloropalladium(II) dichloromethane adduct (154 mg), cesium carbonate (919 mg) in tetrahydrofuran/water (3/1 mL) was exposed to microwave irradiation at 150° C. for 30 min, and then filtered, treated with water, and extracted with ethyl acetate. The organic layer was washed with brine, dried over MgSO₄, and concentrated in vacuo. The residue was chromatographed on silica gel eluting with hexane/ethyl acetate. Crystallization from ethyl acetate/hexane gave the title compound (155 mg).

¹H-NMR (300 MHz, DMSO-d₆) δ: 0.07-0.28 (2H, m) 0.35-0.64 (1H, m) 0.68-0.82 (1H, m) 0.84-0.95 (3H, m) 2.19 (3H, s) 3.63-4.01 (2H, m) 4.53 (2H, s) 6.49-6.62 (1H, m) 6.61-6.70 (1H, m) 6.76-6.86 (1H, m) 7.19-7.43 (4H, m) 10.60 (1H, s).

LCMS (ESI⁺) M+H⁺: 392.

Anal. Calcd for C23H22N3O2F: C, 70.57; H, 5.66; N, 10.73. Found: C, 70.64; H, 5.65; N, 10.75.

### Example 124

### 6-[5-(4-Fluorophenyl)-1-(3-hydroxy-2-methylpropyl)-3-methyl-1H-pyrazol-4-yl]-2H-1,4-benzoxazin-3(4H)-one

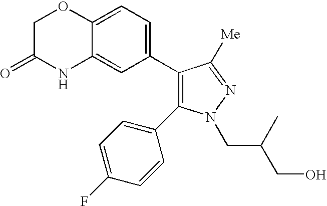

A mixture of 6-(4,4,5,5-tetramethyl-1,3,2-dioxaborolan-2-yl)-2H-1,4-benzoxazin-3(4H)-one (107 mg), 3-[4-bromo-5-(4-fluorophenyl)-3-methyl-1H-pyrazol-1-yl]-2-methylpropan-1-ol (106 mg), [1,1′-bis(diphenylphosphino)ferrocene]dichloropalladium(II) dichloromethane adduct (53 mg), cesium carbonate (317 mg) in tetrahydrofuran/water (3/1 mL) was exposed to microwave irradiation at 150° C. for 30 min, and then filtered, treated with water, and extracted with ethyl acetate. The organic layer was washed with brine, dried over MgSO₄, and concentrated in vacuo. The residue was chromatographed on silica gel eluting with hexane/ethyl acetate. Crystallization from ethyl acetate/hexane gave the title compound (52 mg).

¹H-NMR (300 MHz, DMSO-d₆) δ: 0.65 (3H, d, J=6.4 Hz), 1.94-2.11 (1H, m), 2.19 (3H, s), 3.07-3.24 (2H, m), 3.56-3.74 (1H, m), 3.90-4.06 (1H, m), 4.49 (1H, t, J=5.1 Hz), 4.53 (2H, s), 6.56 (1H, dd, J=8.1, 1.9 Hz), 6.64 (1H, d, J=1.9 Hz), 6.81 (1H, d, J=8.1 Hz), 7.22-7.32 (4H, m), 10.60 (1H, s).

LCMS (ESI⁺) M+H⁺: 396.

Anal. Calcd for C22H22N3O3F: C, 66.82; H, 5.61; N, 10.63. Found: C, 66.57; H, 5.53; N, 10.42.

### Example 125

### 6-[5-(4-Fluorophenyl)-1-(3-hydroxy-2,2-dimethylpropyl)-3-methyl-1H-pyrazol-4-yl]-2H-1,4-benzoxazin-3(4H)-one

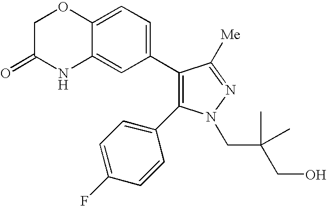

To a suspension of lithiumalminum hydride (20 mg) in tetrahydrofuran (1 mL) was added dropwise a solution of methyl 3-[4-bromo-5-(4-fluorophenyl)-3-methyl-1H-pyrazol-1-yl]-2,2-dimethylpropanoate (199 mg) in tetrahydrofuran (1 mL) at room temperature. The mixture was stirred for 1 h, treated with aqueous Rochelle salt solution and extracted with ethyl acetate. The organic layer was dried over MgSO₄ and concentrated in vacuo to give crude 3-[4-bromo-5-(4-fluorophenyl)-3-methyl-1H-pyrazol-1-yl]-2,2-dimethylpropan-1-ol as oil (161 mg). A mixture of 6-(4,4,5,5-tetramethyl-1,3,2-dioxaborolan-2-yl)-2H-1,4-benzoxazin-3(4H)-one (156 mg), crude material of 3-[4-Bromo-5-(4-fluorophenyl)-3-methyl-1H-pyrazol-1-yl]-2,2-dimethylpropan-1-ol (161 mg), [1,1′-bis(diphenylphosphino)ferrocene]dichloropalladium(II) dichloromethane adduct (77 mg), cesium carbonate (461 mg) in tetrahydrofuran/water (3 mL/1 mL) was exposed to microwave irradiation at 150° C. for 30 min, and then filtered. The filtrate was treated with water, and extracted with ethyl acetate. The organic layer was washed with brine, dried over MgSO₄, and concentrated in vacuo. The residue was chromatographed on silica gel eluting with hexane/ethyl acetate. Crystallization from ethyl acetate/hexane gave the title compound (69 mg).

¹H-NMR (300 MHz, DMSO-d₆) δ: 0.63 (6H, s), 2.19 (3H, s), 3.07 (2H, d, J=5.5 Hz), 3.86 (2H, s), 4.49-4.57 (3H, m), 6.54 (1H, dd, J=8.3, 1.9 Hz), 6.63 (1H, d, J=1.9 Hz), 6.80 (1H, d, J=8.3 Hz), 7.23-7.31 (4H, m), 10.59 (1H, s).

LCMS (ESI⁺) M+H⁺: 410.

Anal. Calcd for C23H24N3O3F 0.3 ethyl acetate): C, 66.7; H, 6.10; N, 9.64. Found: C, 66.47; H, 5.98; N, 9.75.

### Preparation 163

### 6-[1-({1-[(Benzyloxy)methyl]cyclopropyl}methyl)-5-(4-fluorophenyl)-3-methyl-1H-pyrazol-4-yl]-2H-1,4-benzoxazin-3(4H)-one

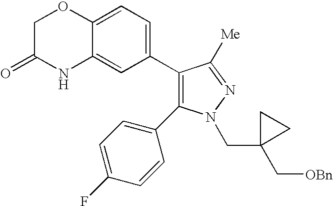

A mixture of 6-(4,4,5,5-tetramethyl-1,3,2-dioxaborolan-2-yl)-2H-1,4-benzoxazin-3(4H)-one (651 mg), 1-({1-[(benzyloxy)methyl]cyclopropyl}methyl)-4-bromo-5-(4-fluorophenyl)-3-methyl-1H-pyrazole (846 mg), [1,1′-bis(diphenylphosphino)ferrocene]dichloropalladium(II) dichloromethane adduct (322 mg), cesium carbonate (1.90 g) in tetrahydrofuran/water (12/5 mL) was exposed to microwave irradiation at 150° C. for 30 min, and then filtered, treated with water, and extracted with ethyl acetate. The organic layer was washed with brine, dried over MgSO₄, and concentrated in vacuo. The residue was chromatographed on silica gel eluting with hexane/ethyl acetate. Crystallization from ethyl acetate/hexane gave the title compound (370 mg).

¹H-NMR (300 MHz, DMSO-d₆) δ: 0.33-0.51 (4H, m), 2.19 (3H, s), 3.10 (2H, s), 4.01 (2H, s), 4.24 (2H, s), 4.53 (2H, s), 6.54 (1H, dd, J=8.3, 2.1 Hz), 6.64 (1H, d, J=2.1 Hz), 6.81 (1H, d, J=8.3 Hz), 7.10-7.22 (4H, m), 7.24-7.36 (5H, m), 10.59 (1H, s).

LCMS (ESI⁺) M+H⁺: 498.

Anal. Calcd for C30H28N3O3F: C, 72.42; H, 5.67; N, 8.45. Found: C, 72.63; H, 5.71; N, 8.2.

### Example 126

### 6-[5-(4-Fluorophenyl)-1-{[1-(hydroxymethyl)cyclopropyl]methyl}-3-methyl-1H-pyrazol-4-yl]-2H-1,4-benzoxazin-3(4H)-one

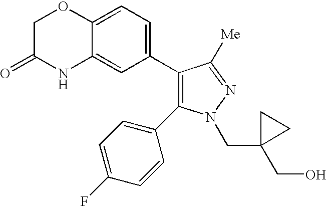

Under hydrogen atmosphere a mixture of 6-[1-({1-[(benzyloxy)methyl]cyclopropyl}methyl)-5-(4-fluorophenyl)-3-methyl-1H-pyrazol-4-yl]-2H-1,4-benzoxazin-3(4H)-one (370 mg) and 10% palladium-carbon (400 mg) in ethanol (20 mL) was stirred at 60° C. for 12 h, passed through celite pad and concentrated in vacuo. The residue was chromatographed on silica gel eluting with hexane/ethyl acetate. Crystallization from ethyl acetate/hexane gave the title compound (217 mg).

¹H-NMR (300 MHz, DMSO-d₆) δ: 0.14-0.29 (2H, m), 0.31-0.42 (2H, m), 2.19 (3H, s), 3.14 (2H, d, J=5.7 Hz), 3.96 (2H, s), 4.44 (1H, t, J=5.7 Hz), 4.53 (2H, s), 6.55 (1H, dd, J=8.3, 2.3 Hz), 6.63 (1H, d, J=2.3 Hz), 6.81 (1H, d, J=8.3 Hz), 7.16-7.37 (4H, m), 10.59 (1H, s).

LCMS (ESI⁺) M+H⁺: 408.

Anal. Calcd for C23H22N3O3F: C, 67.8; H, 5.44; N, 10.31. Found: C, 67.71; H, 5.36; N, 10.08.

### Preparation 164

### 6-[1-({1-[(Benzyloxy)methyl]cyclobutyl}methyl)-5-(4-fluorophenyl)-3-methyl-1H-pyrazol-4-yl]-2H-1,4-benzoxazin-3(4H)-one

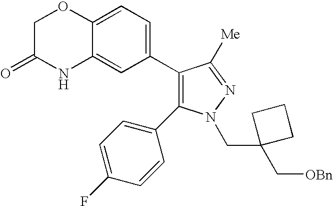

A mixture of 6-(4,4,5,5-tetramethyl-1,3,2-dioxaborolan-2-yl)-2H-1,4-benzoxazin-3(4H)-one (254 mg), 1-({1-[(benzyloxy)methyl]cyclobutyl}methyl)-4-bromo-5-(4-fluorophenyl)-3-methyl-1H-pyrazole (341 mg), [1,1′-bis(diphenylphosphino)ferrocene]dichloropalladium(II) dichloromethane adduct (126 mg), cesium carbonate (752 mg) in tetrahydrofuran/water (3/1 mL) was exposed to microwave irradiation at 150° C. for 1 h, and then filtered, treated with water, and extracted with ethyl acetate. The organic layer was washed with brine, dried over MgSO₄, and concentrated in vacuo. The residue was purified by preparative HPLC to give the title compound (150 mg).

¹H-NMR (300 MHz, DMSO-d₆) δ: 1.52-1.93 (6H, m), 2.18 (3H, s), 3.25 (2H, s), 4.05 (2H, s), 4.34 (2H, s), 4.53 (2H, s), 6.53 (1H, dd, J=8.3, 1.9 Hz), 6.64 (1H, d, J=1.9 Hz, 1H), 6.81 (1H, d, J=8.3 Hz), 7.10-7.34 (9H, m), 10.59 (1H, s).

LCMS (ESI⁺) M+H⁺: 512.

### Example 127

### 6-[5-(4-Fluorophenyl)-1-{[1-(hydroxymethyl)cyclobutyl]methyl}-3-methyl-1H-pyrazol-4-yl]-2H-1,4-benzoxazin-3(4H)-one

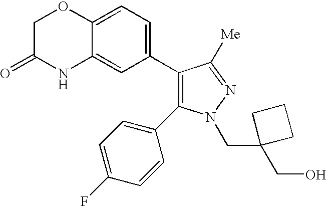

Under hydrogen atmosphere a mixture of 6-[1-({1-[(benzyloxy)methyl]cyclobutyl}methyl)-5-(4-fluorophenyl)-3-methyl-1H-pyrazol-4-yl]-2H-1,4-benzoxazin-3(4H)-one (150 mg) and 10% palladium-carbon (200 mg) in ethanol (20 mL) was stirred at 60° C. for 12 h, passed through celite pad and concentrated in vacuo. The residue was chromatographed on silica gel eluting with hexane/ethyl acetate. Crystallization from ethyl acetate/hexane gave the title compound (100 mg).

¹H-NMR (300 MHz, DMSO-d₆) δ: 1.40-1.57 (1H, m), 1.58-1.81 (5H, m), 2.19 (3H, s), 3.24 (2H, d, J=5.3 Hz), 3.98 (2H, s), 4.47-4.64 (3H, m), 6.55 (1H, dd, J=8.3, 2.1 Hz), 6.64 (1H, d, J=2.1 Hz), 6.81 (1H, d, J=8.3 Hz), 7.15-7.38 (4H, m), 10.59 (1H, s).

LCMS (ESI⁺) M+H⁺: 422.

Anal. Calcd for C24H24N3O3F: C, 68.39; H, 5.74; N, 9.97. Found: C, 68.4; H, 5.71; N, 9.82.

### Preparation 165

### 6-{1-[3-(Benzyloxy)-2,2-difluoropropyl]-5-(4-fluorophenyl)-3-methyl-1H-pyrazol-4-yl}-5-methyl-2H-1,4-benzoxazin-3(4H)-one

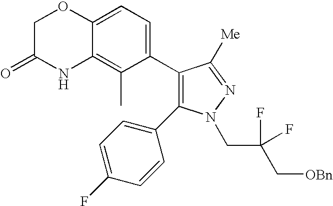

Under argon atmosphere, a mixture of 5-methyl-6-(4,4,5,5-tetramethyl-1,3,2-dioxaborolan-2-yl)-2H-1,4-benzoxazin-3(4H)-one (940 mg), 1-[3-(benzyloxy)-2,2-difluoropropyl]-4-bromo-5-(4-fluorophenyl)-3-methyl-1H-pyrazole (1.10 g), Neolyst™ CX32 (244 mg), potassium tert-buthoxide (562 mg) in 1,2-dimethoxyethane (25 mL) was stirred at 100° C. for 12 h, treated with water, and extracted with ethyl acetate. The organic layer was, dried over MgSO₄, and concentrated in vacuo. The residue was chromatographed on silica gel eluting with hexane/ethyl acetate to give the title compound (120 mg).

¹H-NMR (300 MHz, DMSO-d₆) δ: 1.83 (3H, s), 1.99 (3H, s), 3.61-3.81 (2H, m), 4.43-4.58 (6H, m), 6.61-6.71 (1H, m), 6.71-6.83 (1H, m), 7.12-7.45 (9H, m), 10.09 (1H, s).

LCMS (ESI⁺) M+H⁺: 522.

### Example 128

### 6-[1-(2,2-Difluoro-3-hydroxypropyl)-5-(4-fluorophenyl)-3-methyl-1H-pyrazol-4-yl]-5-methyl-2H-1,4-benzoxazin-3(4H)-one

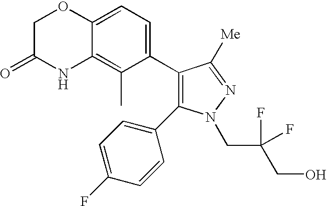

Under hydrogen atmosphere a mixture of 6-{1-[3-(benzyloxy)-2,2-difluoropropyl]-5-(4-fluorophenyl)-3-methyl-1H-pyrazol-4-yl}-5-methyl-2H-1,4-benzoxazin-3(4H)-one (120 mg) and 10 wt % palladium carbon (200 mg) in ethanol (20 mL) was stirred at 60° C. for 12 h, passed through celite pad and concentrated in vacuo. The residue was chromatographed on silica gel eluting with hexane/ethyl acetate. Crystallization from ethyl acetate/hexane gave the title compound (32 mg).

¹H-NMR (300 MHz, DMSO-d₆) δ: 1.84 (3H, s), 1.99 (3H, s), 3.60 (2H, td, J=13.9, 6.2 Hz), 4.37-4.62 (4H, m), 5.54 (1H, t, J=6.2 Hz), 6.66-6.70 (1H, m), 6.73-6.80 (1H, m), 7.14-7.26 (4H, m), 10.08 (1H, s).

LCMS (ESI⁺) M+H⁺: 432.

Anal. Calcd for C22H2ON3O3F3: C, 61.25; H, 4.67; N, 9.74. Found: C, 60.98; H, 4.75; N, 9.52.

### Example 129

### 6-[1-(2,2-Difluoropropyl)-5-(4-fluorophenyl)-3-methyl-1H-pyrazol-4-yl]-5-methyl-2H-1,4-benzoxazin-3(4H)-one

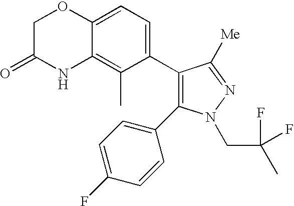

Under argon atmosphere, a mixture of 5-methyl-6-(4,4,5,5-tetramethyl-1,3,2-dioxaborolan-2-yl)-2H-1,4-benzoxazin-3(4H)-one (520 mg), 4-bromo-1-(2,2-difluoropropyl)-5-(4-fluorophenyl)-3-methyl-1H-pyrazole (500 mg), palladium acetate (3.4 mg), Xphos (14.3 mg) and cesium carbonate (1.22 g) in tetrahydrofuran/water (7.5/1.8 mL) was stirred at reflux for 16 h, treated with water, and extracted with ethyl acetate. The organic layer was, dried over MgSO₄, and concentrated in vacuo. The residue was chromatographed on silica gel eluting with hexane/ethyl acetate and then purified by preparative HPLC. Crystallization from ethyl acetate/hexane gave the title compound (30 mg).

¹H-NMR (300 MHz, DMSO-d₆) δ: 1.55 (3H, t, J=19.2 Hz) 1.85 (3H, s) 1.99 (3H, s) 4.44 (2H, t, J=12.8 Hz) 4.50 (2H, s) 6.64-6.73 (1H, m) 6.72-6.80 (1H, m) 7.15-7.32 (4H, m) 10.09 (1H, s).

LCMS (ESI⁺) M+H⁺: 416.

Anal. Calcd for C22H2ON3O2F3: C, 63.61; H, 4.85; N, 10.12. Found: C, 63.55; H, 4.82; N, 10.01.

### Preparation 166

### 5-(4-Fluorophenyl)-3-methyl-1H-pyrazole

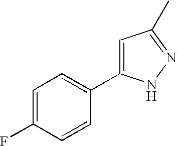

To a mixture of 1-(4-fluorophenyl)butane-1,3-dione (42.9 g), hydrochloric acid (40 mL) and methanol (500 mL) was added dropwise hydrazine monohydrate (23 mL), and stirred at 60° C. for 3 h. The reaction mixture was concentrated in vacuo, and to the residue was added ethyl acetate. The organic layer was washed with saturated aqueous sodium hydrogen carbonate, H₂O and saturated brine, dried over anhydrous magnesium sulfate, and concentrated in vacuo. The obtained solid was recrystallized from cold hexane to give the title compound (39.3 g) as colorless solid.

¹H-NMR (300 MHz, CDCl₃) δ: 2.29 (3H, s), 6.29 (1H, s), 6.95-7.13 (2H, m), 7.59-7.75 (2H, m), 10.95 (1H, br.s).

### Preparation 167

### 4-Bromo-5-(4-fluorophenyl)-3-methyl-1H-pyrazole

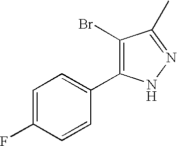

To a solution of 5-(4-fluorophenyl)-3-methyl-1H-pyrazole (39.3 g) in acetonitrile (400 mL) was added portionwise N-Bromosuccinimide (43.7 g), and stirred for 2 h. The reaction mixture was concentrated in vacuo, and to the residue was added ethyl acetate. The organic layer was washed with saturated sodium hydrogen carbonate, H₂O and saturated brine, dried over anhydrous magnesium sulfate, and concentrated in vacuo. The residue was recrystallized from cold hexane/ethyl acetate to give the title compound (56.0 g) as colorless solid.

¹H-NMR (300 MHz, CDCl₃) δ: 2.25 (3H, s), 7.01-7.17 (2H, m), 7.65-7.81 (2H, m), 8.97 (1H, br.s).

### Preparation 168

### 5-(4-Fluorophenyl)-3-methyl-1-(1-methylethyl)-1H-pyrazole

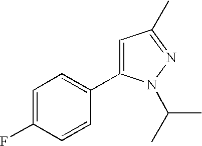

A mixture of 1-(4-fluorophenyl)butane-1,3-dione (1.15 g), (1-methylethyl)hydrazine hydrochloride (1.06 g), triethylamine (1.34 mL), trifluoroacetic acid (0.37 mL) and 2-propanol (10 mL) was stirred at 80° C. for 2 h. The reaction mixture was cooled to room temperature and concentrated in vacuo. The residue was diluted with ethyl acetate, washed with saturated aqueous sodium hydrogen carbonate (10 mL×2), H₂O (10 mL×2) and saturated brine (20 mL), dried over anhydrous magnesium sulfate, and concentrated in vacuo. The residue was purified by column chromatography on silica-gel to give the title compound (1.03 g) as amorphous.

¹H-NMR (300 MHz, CDCl₃) δ: 1.45 (6H, d, J=6.8 Hz), 2.32 (3H, s), 4.33-4.49 (1H, m), 5.99 (1H, s), 7.04-7.17 (2H, m), 7.27-7.36 (2H, m).

### Preparation 169

### 4-Bromo-5-(4-fluorophenyl)-3-methyl-1-(1-methylethyl)-1H-pyrazole

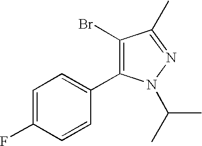

A mixture of 5-(4-fluorophenyl)-3-methyl-1-(1-methylethyl)-1H-pyrazole (1.03 g), N-Bromosuccinimide (0.88 g) and acetonitrile (10 mL) was stirred at room temperature for 1 h. The reaction mixture was concentrated in vacuo, and the residue was diluted with ethyl acetate. The solution was washed with H₂O (10 mL×2) and saturated brine (10 mL), dried over anhydrous magnesium sulfate, and concentrated in vacuo. The residue was purified by column chromatography on silica-gel to give the title compound (0.40 g) as colorless solid.

¹H-NMR (300 MHz, CDCl₃) δ: 1.41 (6H, d, J=6.8 Hz), 2.30 (3H, s), 4.27-4.42 (1H, m), 7.13-7.21 (2H, m), 7.29-7.38 (2H, m).

### Example 130

### 6-[5-(4-Fluorophenyl)-3-methyl-1-(1-methylethyl)-1H-pyrazol-4-yl]-2H-1,4-benzoxazin-3(4H)-one

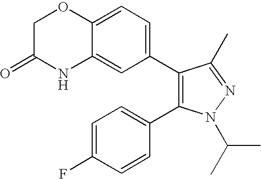

A mixture of 4-bromo-5-(4-fluorophenyl)-3-methyl-1-(1-methylethyl)-1H-pyrazole (0.40 g), 6-(4,4,5,5-tetramethyl-1,3,2-dioxaborolan-2-yl)-2H-1,4-benzoxazin-3(4H)-one (0.41 g), 2 N cesium carbonate aqueous solution (2.0 mL), [1,1′-bis(diphenylphosphino)-ferrocene]dichloropalladium(II) dichloromethane complex (0.22 g) and 1,4-dioxane (10 mL) was degassed, charged with argon, and stirred at 100° C. for 12 h. The reaction mixture was cooled to room temperature, and insoluble material was filtered off. To the filtrate was added ethyl acetate and the organic layer was separated. The organic layer was washed with H₂O (10 mL×2) and saturated brine (10 mL), dried over anhydrous magnesium sulfate, and concentrated in vacuo. The residue was purified by column chromatography on silica-gel to give the title compound (0.18 g) as colorless crystals.

¹H-NMR (300 MHz, DMSO-d₆) δ: 1.34 (6H, d, J=6.8 Hz), 2.20 (3H, s), 4.14-4.25 (1H, m), 4.53 (2H, s), 6.56 (1H, dd, J=8.3, 2.3 Hz), 6.65 (1H, d, J=1.9 Hz), 6.80 (1H, d, J=8.3 Hz), 7.25-7.33(4H, m), 10.61 (1H, s).

### Preparation 170

### 1-Benzyl-5-(4-fluorophenyl)-3-methyl-1H-pyrazole

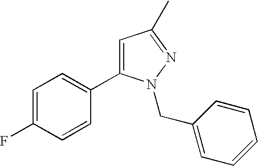

A mixture of 1-(4-fluorophenyl)butane-1,3-dione (1.15 g), benzylhydrazine dihydrochloride (1.52 g), triethylamine (1.34 mL), trifluoroacetic acid (0.37 ml) and 2-propanol (10 mL) was stirred at 80° C. for 2 h. The reaction mixture was cooled to room temperature and concentrated in vacuo. The residue was diluted with ethyl acetate and washed with saturated aqueous sodium hydrogen carbonate (10 mL×2), H₂O (10 mL×2) and saturated brine (20 mL), dried over anhydrous magnesium sulfate, and concentrated in vacuo. The residue was purified by column chromatography on silica-gel to give the title compound (1.5 g) as colorless oil.

¹H-NMR (300 MHz, CDCl₃) δ: 2.31 (3H, s), 5.22 (2H, s), 6.09 (1H, s), 6.94-7.07 (4H, m), 7.16-7.33 (5H, m).

### Preparation 171

### 1-Benzyl-4-bromo-5-(4-fluorophenyl)-3-methyl-1H-pyrazole

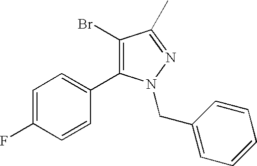

A mixture of 1-benzyl-5-(4-fluorophenyl)-3-methyl-1H-pyrazole (1.5 g), N-Bromosuccinimide (1.05 g) and acetonitrile (10 mL) was stirred at room temperature for 1 h. The reaction mixture was concentrated in vacuo, and the residue was diluted with ethyl acetate. The solution was washed with H₂O (10 mL×2) and saturated brine (10 mL), dried over anhydrous magnesium sulfate, and concentrated in vacuo. The residue was purified by column chromatography on silica-gel to give the title compound (1.56 g) as amorphous.

¹H-NMR (300 MHz, CDCl₃) δ: 2.30 (3H, s), 5.17 (2H, s), 6.97 (2H, dd, J=5.4, 1.9 Hz), 7.02-7.12 (2H, m), 7.18-7.27 (5H, m).

### Example 131

### 6-[1-Benzyl-5-(4-fluorophenyl)-3-methyl-1H-pyrazol-4-yl]-2H-1,4-benzoxazin-3(4H)-one

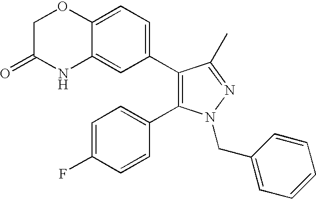

A mixture of 1-benzyl-4-bromo-5-(4-fluorophenyl)-3-methyl-1H-pyrazole (0.51 g), 6-(4,4,5,5-tetramethyl-1,3,2-dioxaborolan-2-yl)-2H-1,4-benzoxazin-3(4H)-one (0.44 g), 2 N cesium carbonate aqueous (2.2 mL), [1,1′-bis(diphenylphosphino)-ferrocene]dichloropalladium(II) dichloromethane complex (0.24 g) and 1,4-dioxane (10 mL) was degassed, charged with argon, and stirred at 100° C. for 12 h. The reaction mixture was cooled to room temperature, and insoluble material was filtered off. To the filtrate was added ethyl acetate and the organic layer was separated. The organic layer was washed with H₂O (10 mL×2) and saturated brine (10 mL), dried over anhydrous magnesium sulfate, and concentrated in vacuo. The residue was purified by column chromatography on silica-gel to give the title compound (0.23 g) as colorless crystals.

¹H-NMR (300 MHz, DMSO-d₆) δ: 2.21 (3H, s), 4.53 (2H, s), 5.15 (2H, s), 6.59 (1H, dd, J=8.3, 2.1 Hz), 6.68 (2H, d, J=2.1 Hz), 6.82 (1H, d, J=8.3 Hz), 6.97 (2H, dd, J=7.9, 1.5 Hz), 7.19-7.32 (7H, m), 10.60 (1H, s).

### Preparation 172

### 5-(4-Fluorophenyl)-3-methyl-1-(2-phenylethyl)-1H-pyrazole

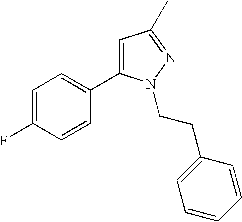

A mixture of 1-(4-fluorophenyl)butane-1,3-dione (1.15 g), (2-phenylethyl)hydrazine sulfuric acid (2.25 g), triethylamine (1.34 mL), trifluoroacetic acid (0.37 mL) and 2-propanol (10 mL) was stirred at 80° C. for 2 h. The reaction mixture was cooled to room temperature and concentrated in vacuo. The residue was diluted with ethyl acetate and washed with saturated aqueous sodium hydrogen carbonate (10 mL×2), H₂O (10 mL×2) and saturated brine (20 mL), dried over anhydrous magnesium sulfate, and concentrated in vacuo. The residue was purified by column chromatography on silica-gel to give the title compound (1.58 g) as colorless oil.

¹H-NMR (300 MHz, CDCl₃) δ: 2.45 (3H, s), 3.08 (3H, t, J=7.0 Hz), 4.39 (3H, t, J=7.0 Hz), 6.08 (1H, s), 6.83-6.94 (4H, m), 7.00-7.09 (2H, m), 7.14-7.22 (3H, m).

### Preparation 173

### 4-Bromo-5-(4-fluorophenyl)-3-methyl-1-(2-phenylethyl)-1H-pyrazole

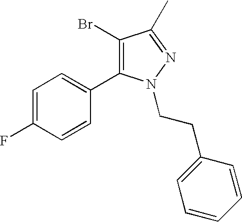

A mixture of 5-(4-fluorophenyl)-3-methyl-1-(2-phenylethyl)-1H-pyrazole (1.58 g), N-Bromosuccinimide (1.05 g) and acetonitrile (10 mL) was stirred at room temperature for 1 h. The reaction mixture was concentrated in vacuo, and the residue was diluted with ethyl acetate. The solution was washed with H₂O (10 mL×2) and saturated brine (10 mL), dried over anhydrous magnesium sulfate, and concentrated in vacuo. The residue was purified by column chromatography on silica-gel to give the title compound (1.20 g) as amorphous.

¹H-NMR (300 MHz, CDCl₃) δ: 2.33 (3H, s), 3.04 (2H, t, J=7.2 Hz), 6.85-6.96 (4H, m), 6.99-7.08 (2H, m), 7.16-7.22 (3H, m).

### Example 132

### 6-[5-(4-Fluorophenyl)-3-methyl-1-(2-phenylethyl)-1H-pyrazol-4-yl]-2H-1,4-benzoxazin-3(4H)-one

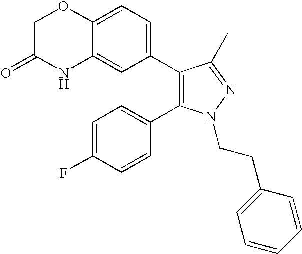

A mixture of 4-bromo-5-(4-fluorophenyl)-3-methyl-1-(2-phenylethyl)-1H-pyrazole (0.50 g), 6-(4,4,5,5-tetramethyl-1,3,2-dioxaborolan-2-yl)-2H-1,4-benzoxazin-3(4H)-one (0.42 g), 2 N cesium carbonate aqueous solution (2.1 mL), [1,1′-bis(diphenylphosphino)-ferrocene]dichloropalladium(II) dichloromethane complex (0.23 g) and 1,4-dioxane (10 mL) was degassed, charged with argon, and stirred at 100° C. for 12 h. The reaction mixture was cooled to room temperature, and insoluble material was filtered off. To the filtrate was added ethyl acetate and the organic layer was separated. The organic layer was washed with H₂O (10 mL×2) and saturated brine (10 mL), dried over anhydrous magnesium sulfate, and concentrated in vacuo. The residue was purified by column chromatography on silica-gel to give the title compound (0.18 g) as colorless crystals.

¹H-NMR (300 MHz, DMSO-d₆) δ: 2.23 (3H, s), 3.00 (2H, t, J=7.3 Hz), 4.05 (2H, t, J=7.3 Hz), 4.52 (2H, s), 6.53 (1H, dd, J=8.3, 2.1 Hz), 6.63 (1H, d, J=1.9 Hz), 6.81 (1H, d, J=8.3 Hz), 6.90-7.03 (4H, m), 7.11-7.28 (5H, m), 10.59 (1H, s).

### Preparation 174

### tert-Butyl 2-(tetrahydro-4H-thiopyran-4-ylidene)hydrazinecarboxylate

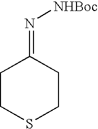

A mixture of tetrahydro-4H-thiopyran-4-one (5.0 g), tert-butyl carbazate (6.82 g) and methanol (50 mL) was stirred at room temperature for 4 h. The reaction mixture was concentrated in vacuo. The obtained white solid was extracted with diisopropyl ether to give the title compound (9.6 g) as crystals.

¹H-NMR (300 MHz, CDCl₃) δ: 1.51 (9H, s), 2.56-2.63 (2H, m), 2.69-2.79 (4H, m), 2.80-2.88 (2H, m).

### Preparation 175

### Tetrahydro-2H-thiopyran-4-ylhydrazine trifluoroacetic acid

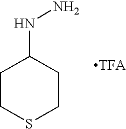

A mixture of tert-butyl 2-(tetrahydro-4H-thiopyran-4-ylidene)hydrazinecarboxylate (5.0 g), sodium cyanoborohydride (1.36 g) and acetic acid/H₂O (25/25 mL) was stirred at room temperature for 2 h. The reaction mixture was neutralized by addition of 1 N sodium hydroxide aqueous solution. To the mixture was added ethyl acetate and the organic layer was separated. The organic layer was washed with H₂O (20 mL×2) and saturated brine (30 mL), dried over anhydrous magnesium sulfate, and concentrated in vacuo. The residue was diluted with dichloromethane (50 mL) and trifluoroacetic acid was added dropwise (17 mL, 217 mmol), followed by stirring at room temperature for 2 h. The reaction mixture was concentrated in vacuo to give the title compound as a crude product as colorless oil.

### Preparation 176

### 5-(4-Fluorophenyl)-3-methyl-1-(tetrahydro-2H-thiopyran-4-yl)-1H-pyrazole

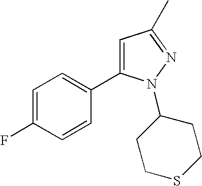

A mixture of 1-(4-fluorophenyl)butane-1,3-dione (1.5 g), tetrahydro-2H-thiopyran-4-ylhydrazine trifluoroacetic acid (2.86 g), triethylamine (1.7 mL), trifluoroacetic acid (1.0 mL) and 2-propanol (15 mL) was stirred at 80° C. for 2 h. The reaction mixture was cooled to room temperature and concentrated in vacuo. The residue was diluted with ethyl acetate and washed with saturated aqueous sodium hydrogen carbonate (10 mL×2), H₂O (10 mL×2) and saturated brine (20 mL), dried over anhydrous magnesium sulfate, and concentrated in vacuo. The residue was purified by column chromatography on silica-gel to give the title compound (0.66 g) as colorless oil.

¹H-NMR (300 MHz, CDCl₃) δ: 2.13 (2H, dd, J=12.9, 3.0 Hz), 2.30 (3H, s), 2.36-2.48 (2H, m), 2.63-2.75 (4H, m), 3.85-4.00 (1H, m), 6.00 (1H, s), 7.11-7.19 (2H, m), 7.24-7.32 (2H, m).

### Preparation 177

### 4-Bromo-5-(4-fluorophenyl)-3-methyl-1-(tetrahydro-2H-thiopyran-4-yl)-1H-pyrazole

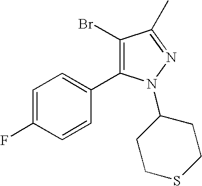

A mixture of 5-(4-fluorophenyl)-3-methyl-1-(tetrahydro-2H-thiopyran-4-yl)-1H-pyrazole (0.66 g), N-Bromosuccinimide (0.46 g) and acetonitrile (10 mL) was stirred at room temperature for 1 h. The reaction mixture was concentrated in vacuo, and the residue was diluted with ethyl acetate. The solution was washed with H₂O (10 mL×2) and saturated brine (10 mL), dried over anhydrous magnesium sulfate, and concentrated in vacuo. The residue was purified by column chromatography on silica-gel to give the title compound (0.82 g) as colorless crystals.

¹H-NMR (300 MHz, CDCl₃) δ: 2.10 (2H, dd, J=13.1, 3.1 Hz), 2.29 (3H, s), 2.31-2.43 (2H, m), 2.57-2.72 (4H, m), 3.81-3.93 (1H, m), 7.16-7.26 (2H, m), 7.27-7.35 (2H, m).

### Example 133

### 6-[5-(4-Fluorophenyl)-3-methyl-1-(tetrahydro-2H-thiopyran-4-yl)-1H-pyrazol-4-yl]-2H-1,4-benzoxazin-3(4H)-one

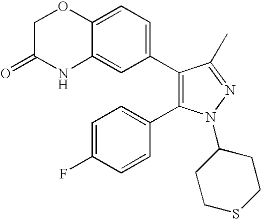

A mixture of 4-bromo-5-(4-fluorophenyl)-3-methyl-1-(tetrahydro-2H-thiopyran-4-yl)-1H-pyrazole (0.82 g), 6-(4,4,5,5-tetramethyl-1,3,2-dioxaborolan-2-yl)-2H-1,4-benzoxazin-3(4H)-one (0.70 g), 2 N cesium carbonate aqueous solution (3.5 mL), [1,1′-bis(diphenylphosphino)-ferrocene]dichloropalladium(II) dichloromethane complex (0.19 g) and 1,4-dioxane (10 mL) was degassed, charged with argon, and stirred at 100° C. for 12 h. The reaction mixture was cooled to room temperature, and insoluble material was filtered off. To the filtrate was added ethyl acetate and the organic layer was separated. The organic layer was washed with H₂O (10 mL×2) and saturated brine (10 mL), dried over anhydrous magnesium sulfate, and concentrated in vacuo. The residue was purified by column chromatography on silica-gel to give the title compound (0.34 g) as colorless crystals.

¹H-NMR (300 MHz, DMSO-d₆) δ: 2.05-2.16 (4H, m), 2.59-2.68 (4H, m), 3.32 (3H, s), 3.75-3.92 (1H, m), 4.52 (2H, s), 6.56 (1H, dd, J=8.1, 2.1 Hz), 6.64 (1H, d, J -1.9 Hz), 6.80 (1H, d, J=8.3 Hz), 10.60 (1H, s).

### Preparation 178

### tert-Butyl-2-(pyridin-2-ylmethylidene)hydrazinecarboxylate

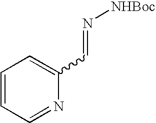

A mixture of pyridine-2-carbaldehyde (5.0 g), tert-butyl carbazate (7.4 g) and ethanol (50 mL) was stirred at room temperature for 3 h. The reaction mixture was concentrated in vacuo. The obtained white solid was extracted with diisopropyl ether to give the title compound (5.6 g) as colorless crystals.

¹H-NMR (300 MHz, CDCl₃) δ: 1.55 (9H, s), 7.21-7.28 (1H, m), 7.65-7.73 (1H, m), 7.94 (1H, s), 8.07 (1H, d, J=8.1 Hz), 8.36 (1H, s), 8.53-8.59 (1H, m).

### Preparation 179

### 2-(Hydrazinomethyl)pyridine trifluoroacetic acid

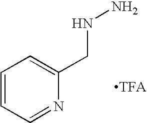

A mixture of tert-butyl-2-(pyridin-2-ylmethylidene)hydrazinecarboxylate (3.0 g), sodium cyanoborohydride (0.85 g) and acetic acid/H₂O (15/15 mL) was s stirred at room temperature for 2 h. The reaction mixture was neutralized by addition of 1 N sodium hydroxide aqueous solution. To the mixture was added ethyl acetate and the organic layer was separated. The organic layer was washed with H₂O (20 mL×2) and saturated brine (30 mL), dried over anhydrous magnesium sulfate, and concentrated in vacuo. The residue was diluted with dichloromethane (30 mL) and trifluoroacetic acid was added dropwise (10 mL, 136 mmol), followed by stirring at room temperature for 2 h. The reaction mixture was concentrated in vacuo to give the title compound as a crude product as pale yellow oil.

### Preparation 180

### 2-{[5-(4-Fluorophenyl)-3-methyl-1H-pyrazol-1-yl]methyl}pyridine

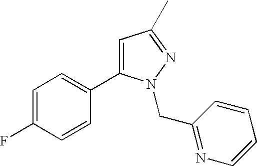

A mixture of 1-(4-fluorophenyl)butane-1,3-dione (0.6 g), 2-(hydrazinomethyl)pyridine trifluoroacetic acid (1.47 g), triethylamine (0.7 mL, 5.0 mmol), trifluoroacetic acid (0.4 mL) and 2-propanol (10 mL) was stirred at 80° C. for 2 h. The reaction mixture was cooled to room temperature and concentrated in vacuo. The residue was diluted with ethyl acetate and washed with saturated aqueous sodium hydrogen carbonate (10 mL×2), H₂O (10 mL×2) and saturated brine (20 mL), dried over anhydrous magnesium sulfate, and concentrated in vacuo. The residue was purified by column chromatography on silica-gel to give the title compound (0.27 g) as amorphous.

¹H-NMR (300 MHz, CDCl₃) δ: 2.34 (3H, s), 5.49 (2H, s), 6.19 (1H, s), 6.99-7.10 (3H, m), 7.27-7.37 (3H, m), 7.73-7.82 (1H, m), 8.61 (1H, d, J=4.9 Hz).

### Preparation 181

### 2-{[4-Bromo-5-(4-fluorophenyl)-3-methyl-1H-pyrazol-1-yl]methyl}pyridine

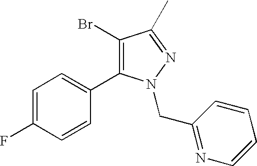

A mixture of 2-{[5-(4-fluorophenyl)-3-methyl-1H-pyrazol-1-yl]methyl}pyridine (0.27 g), N-Bromosuccinimide (0.20 g) and acetonitrile (5 mL) was stirred at room temperature for 1 h. The reaction mixture was concentrated in vacuo, and the residue was diluted with ethyl acetate. The solution was washed with H₂O (10 mL×2) and saturated brine (10 mL), dried over anhydrous magnesium sulfate, and concentrated in vacuo. The residue was purified by column chromatography on silica-gel to give the title compound (0.24 g) as colorless crystals.

¹H-NMR (300 MHz, CDCl₃) δ: 2.32 (3H, s), 5.31 (2H, s), 6.93 (1H, d, J=7.6 Hz), 7.05-7.21 (3H, m), 7.31-7.40 (2H, m), 7.58-7.68 (1H, m), 8.52 (1H, d, J=4.2 Hz).

### Example 134

### 6-[5-(4-Fluorophenyl)-3-methyl-1-(pyridin-2-ylmethyl)-1H-pyrazol-4-yl]-2H-1,4-benzoxazin-3(4H)-one

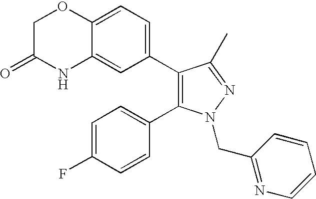

A mixture of 2-{[4-bromo-5-(4-fluorophenyl)-3-methyl-1H-pyrazol-1-yl]methyl}pyridine (0.24 g), 6-(4,4,5,5-tetramethyl-1,3,2-dioxaborolan-2-yl)-2H-1,4-benzoxazin-3(4H)-one (0.21 g), 2 N cesium carbonate aqueous solution (1.0 mL), [1,1′-bis(diphenylphosphino)-ferrocene]dichloropalladium(II) dichloromethane complex (0.08 g) and 1,4-dioxane (5 mL) was degassed, charged with argon, and stirred at 100° C. for 12 h. The reaction mixture was cooled to room temperature, and insoluble material was filtered off. To the filtrate was added ethyl acetate and the organic layer was separated. The organic layer was washed with H₂O (10 mL×2) and saturated brine (10 mL), dried over anhydrous magnesium sulfate, and concentrated in vacuo. The residue was purified by column chromatography on silica-gel to give the title compound (0.15 g) as colorless crystals.

¹H-NMR (300 MHz, DMSO-d₆) δ: 2.20 (3H, s), 4.54 (2H, s), 5.22 (2H, s), 6.61 (1H, d, J=8.1 Hz), 6.69 (1H, s), 6.83 (1H, d, J=8.1 Hz), 7.02 (1H, d, J=7.7 Hz), 7.16-7.37 (5H, m), 7.68-7.82 (1H, m), 8.49 (1H, d, J=4.5 Hz), 10.60 (1H, s).

### Preparation 182

### tert-Butyl-2-(pyridin-3-ylmethylidene)hydrazinecarboxylate

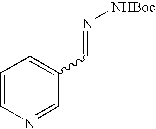

A mixture of pyridine-3-carbaldehyde (5.0 g), tert-butyl carbazate (7.4 g) and ethanol (50 mL) was stirred at room temperature for 3 h. The reaction mixture was concentrated in vacuo. The obtained white solid was extracted with diisopropyl ether to give the title compound (9.3 g) as colorless crystals.

¹H-NMR (300 MHz, CDCl₃) δ: 1.54 (9H, s), 7.27-7.34 (1H, m), 7.95 (1H, s), 8.09-8.16 (1H, m), 8.57 (1H, dd, J=4.9, 1.7 Hz), 8.74 (1H, d, J=1.9 Hz).

### Preparation 183

### 3-(Hydrazinomethyl)pyridine trifluoroacetic acid

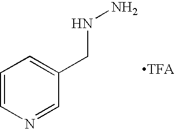

A mixture of tert-butyl-2-(pyridin-3-ylmethylidene)hydrazinecarboxylate (3.0 g), sodium cyanoborohydride (0.85 g) and acetic acid/H₂O (15/15 mL) was stirred at room temperature for 2 h. The reaction mixture was neutralized by addition of 1 N sodium hydroxide aqueous solution. To the mixture was added ethyl acetate and the organic layer was separated. The organic layer was washed with H₂O (20 mL×2) and saturated brine (30 mL), dried over anhydrous magnesium sulfate, and concentrated in vacuo. The residue was diluted with dichloromethane (30 mL) and trifluoroacetic acid was added dropwise (10 mL), followed by stirring at room temperature for 2 h. The reaction mixture was concentrated in vacuo to give the title compound as a crude product as pale yellow oil.

### Preparation 184

### 3-{[5-(4-Fluorophenyl)-3-methyl-1H-pyrazol-1-yl]methyl}pyridine

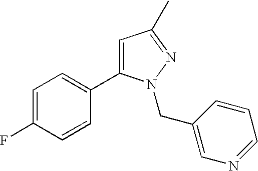

A mixture of 1-(4-fluorophenyl)butane-1,3-dione (0.6 g), 3-(hydrazinomethyl)pyridine trifluoroacetic acid (2.2 g), triethylamine (0.93 mL), trifluoroacetic acid (0.51 mL) and 2-propanol (10 mL) was stirred at 80° C. for 2 h. The reaction mixture was cooled to room temperature and concentrated in vacuo. The residue was diluted with ethyl acetate and washed with saturated aqueous sodium hydrogen carbonate (10 mL×2), H₂O (10 mL×2) and saturated brine (20 mL), dried over anhydrous magnesium sulfate, and concentrated in vacuo. The residue was purified by column chromatography on silica-gel to give the title compound (0.17 g) as colorless oil.

¹H-NMR (300 MHz, CDCl₃) δ: 2.32 (3H, s), 5.34 (2H, s), 6.16 (1H, s), 7.07-7.17 (2H, m), 7.22-7.30 (2H, m), 7.48-7.56 (1H, m), 7.72 (1H, d, J=8.1 Hz), 8.28-8.40 (1H, m), 8.63 (1H, d, J=4.0 Hz).

### Preparation 185

### 3-{[4-Bromo-5-(4-fluorophenyl)-3-methyl-1H-pyrazol-1-yl]methyl}pyridine

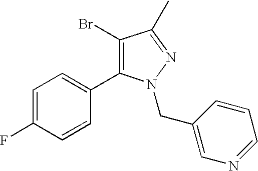

A mixture of 3-{[5-(4-fluorophenyl)-3-methyl-1H-pyrazol-1-yl]methyl}pyridine (0.17 g), N-Bromosuccinimide (0.13 g) and acetonitrile (5 mL) was stirred at room temperature for 1 h. The reaction mixture was concentrated in vacuo, and the residue was diluted with ethyl acetate. The solution was washed with H₂O (10 mL×2) and saturated brine (10 mL), dried over anhydrous magnesium sulfate, and concentrated in vacuo. The residue was purified by column chromatography on silica-gel to give the title compound (0.12 g) as colorless crystals.

¹H-NMR (300 MHz, CDCl₃) δ: 2.31 (3H, s), 5.26 (2H, s), 7.12-7.22 (2H, m), 7.22-7.31 (2H, m), 7.41-7.50 (1H, m), 7.64 (1H, d, J=7.9 Hz), 8.36 (1H, s), 8.63 (1H, s).

### Example 135

### 6-[5-(4-Fluorophenyl)-3-methyl-1-(pyridin-3-ylmethyl)-1H-pyrazol-4-yl]-2H-1,4-benzoxazin-3(4H)-one

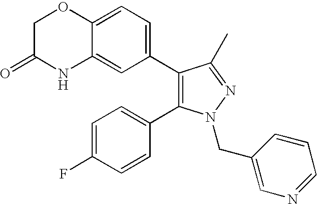

A mixture of 3-{[4-bromo-5-(4-fluorophenyl)-3-methyl-1H-pyrazol-1-yl]methyl}pyridine (0.12 g), 6-(4,4,5,5-tetramethyl-1,3,2-dioxaborolan-2-yl)-2H-1,4-benzoxazin-3(4H)-one (0.11 g), 2 N cesium carbonate aqueous solution (0.5 mL), [1,1′-bis(diphenylphosphino)ferrocene]dichloropalladium(II) dichloromethane complex (0.04 g) and 1,4-dioxane (3 mL) was degassed, charged with argon, and stirred at 100° C. for 12 h. The reaction mixture was cooled to room temperature, and insoluble material was filtered off. To the filtrate was added ethyl acetate and the organic layer was separated. The organic layer was washed with H₂O (10 mL×2) and saturated brine (10 mL), dried over magnesium sulfate, and concentrated in vacuo. The residue was purified by column chromatography on silica-gel to give the title compound (0.03 g) as colorless crystals.

¹H-NMR (300 MHz, DMSO-d₆) δ: 2.21 (3H, s), 4.53 (2H, s), 5.20 (2H, s), 6.60 (1H, dd, J=8.3, 1.9 Hz), 6.68 (1H, d, J=1.9 Hz), 6.82 (1H, d, J=8.3 Hz), 7.21-7.35 (5H, m), 7.37-7.44 (1H, m), 8.19 (1H, s), 8.45 (1H, d, J=3.8 Hz), 10.60 (1H, s).

### Preparation 186

### tert-Butyl-2-(pyridin-4-ylmethylidene)hydrazinecarboxylate

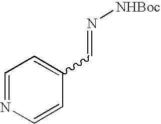

A mixture of pyridine-4-carbaldehyde (5.0 g), tert-butyl carbazate (7.4 g) and ethanol (50 mL) was stirred at room temperature for 3 h. The reaction mixture was concentrated in vacuo. The obtained white solid was extracted with diisopropyl ether to give the title compound (6.4 g) as colorless crystals.

¹H-NMR (300 MHz, CDCl₃) δ: 1.54 (9H, s), 7.51-7.57 (2H, m), 7.87 (1H, s), 8.59-8.64 (2H, m), 8.74 (1H, s).

### Preparation 187

### 4-(Hydrazinomethyl)pyridine trifluoroacetic acid

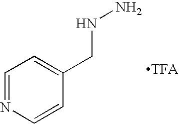

A mixture of tert-butyl-2-(pyridin-4-ylmethylidene)hydrazinecarboxylate (3.0 g), sodium cyanoborohydride (0.85 g) and acetic acid/H₂O (15/15 mL) was stirred at room temperature for 2 h. The reaction mixture was neutralized by addition of 1 N sodium hydroxide aqueous solution. To the mixture was added ethyl acetate and the organic layer was separated. The organic layer was washed with H₂O (20 mL×2) and saturated brine (30 mL), dried over anhydrous magnesium sulfate, and concentrated in vacuo. The residue was diluted with dichloromethane (30 mL) and trifluoroacetic acid was added dropwise (10 mL, 136 mmol), followed by stirring at room temperature for 2 h. The reaction mixture was concentrated in vacuo to give the title compound as a crude product as pale yellow oil.

### Preparation 188

### 4-{[5-(4-Fluorophenyl)-3-methyl-1H-pyrazol-1-yl]methyl}pyridine

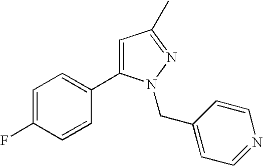

A mixture of 1-(4-fluorophenyl)butane-1,3-dione (0.6 g), 4-(hydrazinomethyl)pyridine trifluoroacetic acid (2.2 g), triethylamine (0.93 mL), trifluoroacetic acid (0.51 mL) and 2-propanol (10 mL) was stirred at 80° C. for 2 h. The reaction mixture was cooled to room temperature and concentrated in vacuo. The residue was diluted with ethyl acetate and washed with saturated aqueous sodium hydrogen carbonate (10 mL×2), H₂O (10 mL×2) and saturated brine (20 mL), dried over anhydrous magnesium sulfate, and concentrated in vacuo. The residue was purified by column chromatography on silica-gel to give the title compound (0.29 g) as pale yellow solid.

¹H-NMR (300 MHz, CDCl₃) δ: 2.34 (3H, s), 5.38 (2H, s), 6.20 (2H, s), 7.03-7.14 (2H, m), 7.19-7.34 (5H, m), 7.69-7.78 (1H, m).

### Preparation 189

### 4-{[4-Bromo-5-(4-fluorophenyl)-3-methyl-1H-pyrazol-1-yl]methyl}pyridine

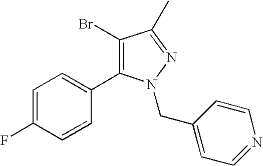

A mixture of 4-{[5-(4-fluorophenyl)-3-methyl-1H-pyrazol-1-yl]methyl}pyridine (0.29 g), N-Bromosuccinimide (1.2 g) and acetonitrile (5 mL) was stirred at room temperature for 1 h. The reaction mixture was concentrated in vacuo, and the residue was diluted with ethyl acetate. The solution was washed with H₂O (10 mL×2) and saturated brine (10 mL), dried over anhydrous magnesium sulfate, and concentrated in vacuo. The residue was purified by column chromatography on silica-gel to give the title compound (0.16 g) as colorless crystals.

¹H-NMR (300 MHz, CDCl₃) δ: 2.33 (3H, s), 5.31 (2H, s), 7.08-7.32 (6H, m), 8.66 (2H, s).

### Example 136

### 6-[5-(4-Fluorophenyl)-3-methyl-1-(pyridin-4-ylmethyl)-1H-pyrazol-4-yl]-2H-1,4-benzoxazin-3(4H)-one

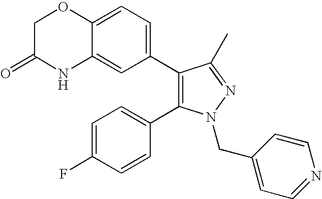

A mixture of 4-{[4-bromo-5-(4-fluorophenyl)-3-methyl-1H-pyrazol-1-yl]methyl}pyridine (0.16 g), 6-(4,4,5,5-tetramethyl-1,3,2-dioxaborolan-2-yl)-2H-1,4-benzoxazin-3(4H)-one (0.14 g), 2 N cesium carbonate aqueous solution (0.7 mL), [1,1′-bis(diphenylphosphino)-ferrocene]dichloropalladium(II) dichloromethane complex (0.04 g) and 1,4-dioxane (4 mL) was degassed, charged with argon, and stirred at 100° C. for 12 h. The reaction mixture was cooled to room temperature, and insoluble material was filtered off. To the filtrate was added ethyl acetate and the organic layer was separated. The organic layer was washed with H₂O (10 mL×2) and saturated brine (10 mL), dried over anhydrous magnesium sulfate, and concentrated in vacuo. The residue was purified by column chromatography on silica-gel to give the title compound (0.01 g) as colorless crystals.

¹H-NMR (300 MHz, DMSO-d₆) δ: 2.22 (3H, s), 4.54 (2H, s), 5.20 (2H, s), 6.62 (1H, dd, J=8.3, 2.3H), 6.69 (1H, d, J=1.9 Hz), 6.83 (1H, d, J=8.0 Hz), 6.96 (2H, d, J=6.1 Hz), 7.20-7.25 (4H, m), 8.47 (2H, d, J=6.1 Hz), 10.60 (1H, s).

### Preparation 190

### tert-Butyl-2-[(4-methylphenyl)methylidene]hydrazinecarboxylate

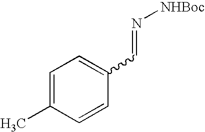

A mixture of 4-methylbenzaldehyde (3.0 g), tert-butyl carbazate (4.0 g) and ethanol (30 mL) was stirred at room temperature for 3 h. The reaction mixture was concentrated in vacuo. The obtained white solid was extracted with diisopropyl ether to give the title compound (4.4 g) as colorless crystals.

¹H-NMR (300 MHz, CDCl₃) δ: 1.53 (9H, s), 2.36 (3H, s), 7.17 (2H, d, J=8.0 Hz), 7.57 (2H, d, J=8.0 Hz), 7.75-7.87 (2H, m).

### Preparation 191

### (4-Methylbenzyl)hydrazine trifluoroacetic acid

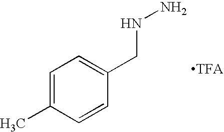

A mixture of tert-butyl-2-[(4-methylphenyl)methylidene]hydrazinecarboxylate (2.5 g), sodium cyanoborohydride (0.84 g) and acetic acid/H₂O (15/15 mL) was stirred at room temperature for 2 h. The reaction mixture was neutralized by addition of aqueous 1 N sodium hydroxide solution. To the mixture was added ethyl acetate and the organic layer was separated. The organic layer was washed with H₂O (20 mL×2) and saturated brine (30 mL), dried over anhydrous magnesium sulfate, and concentrated in vacuo. The residue was diluted with dichloromethane (30 mL) and trifluoroacetic acid was added dropwise (8.2 mL), followed by stirring at room temperature for 2 h. The reaction mixture was concentrated in vacuo to give the title compound as a crude product as colorless oil.

### Preparation 192

### 5-(4-Fluorophenyl)-3-methyl-1-(4-methylbenzyl)-1H-pyrazole

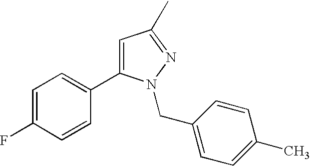

A mixture of 1-(4-fluorophenyl)butane-1,3-dione (0.6 g), (4-methylbenzyl)hydrazine trifluoroacetic acid (2.3 g), triethylamine (0.93 mL), trifluoroacetic acid (0.51 mL) and 2-propanol (10 mL) was stirred at 80° C. for 2 h. The reaction mixture was cooled to room temperature and concentrated in vacuo. The residue was diluted with ethyl acetate and washed with saturated aqueous sodium hydrogen carbonate (10 mL×2), H₂O (10 mL×2) and saturated brine (20 mL), dried over anhydrous magnesium sulfate, and concentrated in vacuo. The residue was purified by column chromatography on silica-gel to give the title compound (0.9 g) as colorless oil.

¹H-NMR (300 MHz, CDCl₃) δ: 2.28 (3H, s), 2.42 (3H, s), 5.39 (2H, s), 6.28 (1H, s), 6.88 (2H, d, J=8.0 Hz), 7.08 (2H, d, J=7.6 Hz), 7.12-7.21 (2H, m), 7.31-7.38 (2H, m).

### Example 137

### 6-[5-(4-Fluorophenyl)-3-methyl-1-(4-methylbenzyl)-1H-pyrazol-4-yl]-2H-1,4-benzoxazin-3(4H)-one

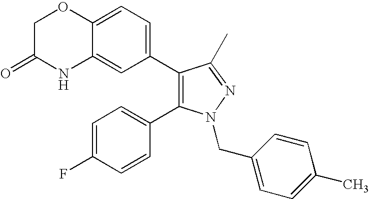

A mixture of 5-(4-fluorophenyl)-3-methyl-1-(4-methylbenzyl)-1H-pyrazole (0.90 g), N-Bromosuccinimide (0.63 g) and acetonitrile (10 mL) was stirred at room temperature for 1 h. The reaction mixture was concentrated in vacuo, and the residue was diluted with ethyl acetate. The solution was washed with H₂O (10 mL×2) and saturated brine (10 mL), dried over anhydrous magnesium sulfate, and concentrated in vacuo. The residue was purified by column chromatography on silica-gel to give 4-bromo-5-(4-fluorophenyl)-3-methyl-1-(4-methylbenzyl)-1H-pyrazole as crude product. The product was diluted with 1,4-dioxane (10 mL) and added to 6-(4,4,5,5-tetramethyl-1,3,2-dioxaborolan-2-yl)-2H-1,4-benzoxazin-3(4H)-one (0.97 g), 2 N cesium carbonate aqueous solution (4.8 mL), [1,1′-bis(diphenylphosphino)ferrocene]dichloropalladium(II) dichloromethane complex (0.39 g) and 1,4-dioxane (10 mL) was degassed, charged with argon, and stirred at 100° C. for 12 h. The reaction mixture was cooled to room temperature, and insoluble material was filtered off. To the filtrate was added ethyl acetate and the organic layer was separated. The organic layer was washed with H₂O (10 mL×2) and saturated brine (10 mL), dried over anhydrous magnesium sulfate, and concentrated in vacuo. The residue was purified by column chromatography on silica-gel to give the title compound (0.27 g) as colorless crystals.

¹H-NMR (300 MHz, DMSO-d₆) δ: 2.20 (3H, s), 2.24 (3H, s), 4.53 (2H, s), 5.09 (2H, s), 6.58 (1H, dd, J=8.3, 1.9 Hz), 6.67 (1H, d, J=1.9 Hz), 6.78-6.89 (3H, m), 7.08 (2H, d, J=7.6 Hz), 7.20-7.26 (4H, m), 10.59 (1H, s).

### Preparation 193

### tert-Butyl-2-[(4-fluorophenyl)methylidene]hydrazinecarboxylate

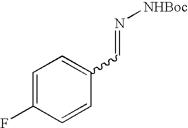

A mixture of 4-fluorobenzaldehyde (3.0 g), tert-butyl carbazate (4.0 g) and ethanol (30 mL) was stirred at room temperature for 3 h. The reaction mixture was concentrated in vacuo. The obtained white solid was extracted with diisopropyl ether to give the title compound (4.4 g) as colorless crystals.

¹H-NMR (300 MHz, CDCl₃) δ: 1.53 (9H, s), 2.36 (3H, s), 7.17 (2H, d, J=8.0 Hz), 7.57 (2H, d, J=8.0 Hz), 7.75-7.87 (2H, m).

### Preparation 194

### (4-Fluorobenzyl)hydrazine trifluoroacetic acid

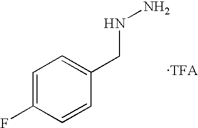

A mixture of tert-butyl-2-[(4-methylphenyl)methylidene]hydrazinecarboxylate (2.5 g), sodium cyanoborohydride (0.79 g) and acetic acid/H₂O (15/15 mL) was stirred at room temperature for 2 h. The reaction mixture was neutralized by addition of aqueous 1 N sodium hydroxide solution. To the mixture was added ethyl acetate and the organic layer was separated. The organic layer was washed with H₂O (20 mL×2) and saturated brine (30 mL), dried over anhydrous magnesium sulfate, and concentrated in vacuo. The residue was diluted with dichloromethane (30 mL) and trifluoroacetic acid was added dropwise (8.0 mL), followed by stirring at room temperature for 2 h. The reaction mixture was concentrated in vacuo to give the title compound as a crude product as colorless oil.

### Preparation 195

### 1-(4-Fluorobenzyl)-5-(4-fluorophenyl)-3-methyl-1H-pyrazole

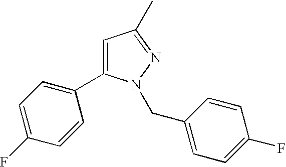

A mixture of 1-(4-fluorophenyl)butane-1,3-dione (0.6 g), (4-methylbenzyl)hydrazine trifluoroacetic acid (2.4 g), triethylamine (0.93 mL, 6.6 mmol), trifluoroacetic acid (0.51 mL) and 2-propanol (10 mL) was stirred at 80° C. for 2 h. The reaction mixture was cooled to room temperature and concentrated in vacuo. The residue was diluted with ethyl acetate and washed with saturated aqueous sodium hydrogen carbonate (10 mL×2), H₂O (10 mL×2) and saturated brine (20 mL), dried over anhydrous magnesium sulfate, and concentrated in vacuo. The residue was purified by column chromatography on silica-gel to give the title compound (0.89 g) as oil.

¹H-NMR (300 MHz, CDCl₃) δ: 2.41 (3H, s), 5.39 (2H, s), 6.25 (1H, s), 6.93-7.00 (4H, m), 7.12-7.20 (2H, m), 7.28-7.35 (2H, m).

### Example 138

### 6-[1-(4-Fluorobenzyl)-5-(4-fluorophenyl)-3-methyl-1H-pyrazol-4-yl]-2H-1,4-benzoxazin-3(4H)-one

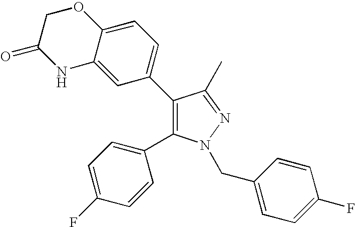

A mixture of 1-(4-fluorobenzyl)-5-(4-fluorophenyl)-3-methyl-1H-pyrazole (0.89 g), N-Bromosuccinimide (0.62 g) and acetonitrile (10 mL) was stirred at room temperature for 1 h. The reaction mixture was concentrated in vacuo, and the residue was diluted with ethyl acetate. The solution was washed with H₂O (10 mL×2) and saturated brine (10 mL), dried over anhydrous magnesium sulfate, and concentrated in vacuo. The residue was purified by column chromatography on silica-gel to give 4-bromo-1-(4-fluorobenzyl)-5-(4-fluorophenyl)-3-methyl-1H-pyrazole as crude product. The product was diluted with 1,4-dioxane (10 mL) and added to 6-(4,4,5,5-tetramethyl-1,3,2-dioxaborolan-2-yl)-2H-1,4-benzoxazin-3(4H)-one (0.95 g, 2 N cesium carbonate aqueous solution (4.7 mL), [1,1′-bis(diphenylphosphino)ferrocene]dichloropalladium(II) dichloromethane complex (0.39 g) and 1,4-dioxane (10 mL) was degassed, charged with argon, and stirred at 100° C. for 12 h. The reaction mixture was cooled to room temperature, and insoluble material was filtered off. To the filtrate was added ethyl acetate and the organic layer was separated. The organic layer was washed with H₂O (10 mL×2) and saturated brine (10 mL), dried over anhydrous magnesium sulfate, and concentrated in vacuo. The residue was purified by column chromatography on silica-gel to give the title compound (0.26 g) as colorless crystals.

¹H-NMR (300 MHz, DMSO-d₆) δ: 2.21 (3H, s), 4.53 (2H, s), 5.14 (2H, s), 6.59 (1H, dd, J=8.3, 1.9 Hz), 6.68 (1H, d, J=2.3 Hz), 6.82 (1H, d, J=8.3 Hz), 6.98-7.05 (2H, m), 7.07-7.15 (2H, m), 7.20-7.28 (4H, m), 10.60 (1H, s).

### Preparation 196

### (Tetrahydro-2H-pyran-4-ylmethyl)hydrazine trifluoroacetic acid

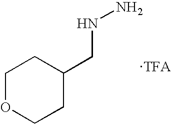

A mixture of tetrahydro-2H-pyran-4-carbaldehyde (1.5 g), tert-butyl carbazate (2.1 g) and ethanol (20 mL) was stirred at room temperature for 3 h. The reaction mixture was concentrated in vacuo. The obtained white solid was extracted with diisopropyl ether, and the extracted solid was diluted with acetic acid/H₂O (15/15 mL). To the mixture was added to sodium cyanoborohydride (0.82 g) and stirred at room temperature for 2 h. The reaction mixture was neutralized by addition of 1 N sodium hydroxide aqueous solution. To the mixture was added ethyl acetate and the organic layer was separated. The organic layer was washed with H₂O (20 mL×2) and saturated brine (30 mL), dried over anhydrous magnesium sulfate, and concentrated in vacuo. The residue was diluted with dichloromethane (30 mL) and trifluoroacetic acid was added dropwise (10 mL), followed by stirring at room temperature for 2 h. The reaction mixture was concentrated in vacuo to give the title compound as a crude product as colorless oil.

### Preparation 197

### 4-Bromo-5-(4-fluorophenyl)-3-methyl-1-(tetrahydro-2H-pyran-4-ylmethyl)-1H-pyrazole

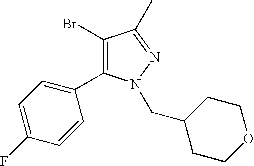

A mixture of 1-(4-fluorophenyl)butane-1,3-dione (0.6 g), (tetrahydro-2H-pyran-4-ylmethyl)hydrazine trifluoroacetic acid (2.27 g), triethylamine (0.93 mL), trifluoroacetic acid (0.51 mL) and 2-propanol (10 mL) was stirred at 80° C. for 2 h. The reaction mixture was cooled to room temperature and concentrated in vacuo. The residue was diluted with ethyl acetate and washed with saturated aqueous sodium hydrogen carbonate (10 mL×2), H₂O (10 mL×2) and saturated brine (20 mL), dried over anhydrous magnesium sulfate, and concentrated in vacuo. To the residue was added the mixture of N-Bromosuccinimide (0.62 g) and acetonitrile (10 mL), the resulting mixture was stirred at room temperature for 1 h. The reaction mixture was concentrated in vacuo, and the residue was diluted with ethyl acetate. The solution was washed with H₂O (10 mL×2) and saturated brine (10 mL), dried over anhydrous magnesium sulfate, and concentrated in vacuo. The residue was purified by column chromatography on silica-gel to give the title compound (0.76 g) as amorphous.

¹H-NMR (300 MHz, CDCl₃) δ: 0.97-1.20 (2H, m), 1.22-1.59 (2H, m), 1.99-2.17 (1H, m), 2.00-2.19 (2H, m), 2.29 (3H, s), 3.22-3.43 (2H, m), 3.86 (2H, d, J=7.19 Hz), 7.05-7.38 (4H, m).

### Example 139

### 6-[5-(4-Fluorophenyl)-3-methyl-1-(tetrahydro-2H-pyran-4-ylmethyl)-1H-pyrazol-4-yl]-2H-1,4-benzoxazin-3(4H)-one

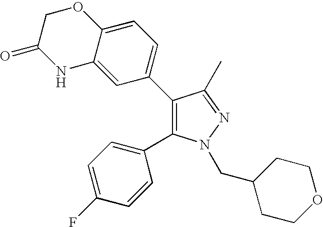

A mixture of 4-bromo-5-(4-fluorophenyl)-3-methyl-1-(tetrahydro-2H-pyran-4-ylmethyl)-1H-pyrazole (0.76 g), 6-(4,4,5,5-tetramethyl-1,3,2-dioxaborolan-2-yl)-2H-1,4-benzoxazin-3(4H)-one (0.71 g), 2 N cesium carbonate aqueous solution (3.2 mL), [1,1′-bis(diphenylphosphino)ferrocene]dichloropalladium(II) dichloromethane complex (0.17 g) and 1,4-dioxane (10 mL) was degassed, charged with argon, and stirred at 100° C. for 12 h. The reaction mixture was cooled to room temperature, and insoluble material was filtered off. To the filtrate was added ethyl acetate and the organic layer was separated. The organic layer was washed with H₂O (10 mL×2) and saturated brine (10 mL), dried over anhydrous magnesium sulfate, and concentrated in vacuo. The residue was purified by column chromatography on silica-gel to give the title compound (0.28 g) as colorless crystals.

¹H-NMR (300 MHz, DMSO-d₆) δ: 0.93-1.10 (2H, m), 1.28-1.39 (2H, m), 1.89-2.07 (1H, m), 2.19 (3H, s), 3.12-3.23 (2H, m), 3.68-3.81 (4H, m), 4.53 (2H, s), 6.56 (1H, dd, J=8.3, 1.9 Hz), 6.65 (1H, d, J=1.9 Hz), 6.81 (1H, d, J=8.3 Hz), 7.22-7.37 (4H, m), 10.59 (1H, s).

### Example 140

### 6-[1-(2,2-Difluoropropyl)-5-(4-fluorophenyl)-3-methyl-1H-pyrazol-4-yl]-8-methyl-2H-1,4-benzoxazin-3(4H)-one

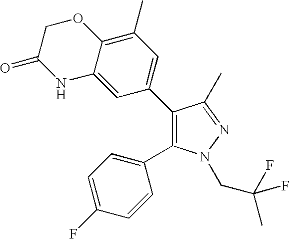

A mixture of 4-bromo-1-(2,2-difluoropropyl)-5-(4-fluorophenyl)-3-methyl-1H-pyrazole (0.21 g), 8-methyl-6-(4,4,5,5-tetramethyl-1,3,2-dioxaborolan-2-yl)-2H-1,4-benzoxazin-3(4H)-one (0.27 g), 2 N cesium carbonate aqueous solution (1.0 mL), [1,1′-bis(diphenylphosphino)ferrocene]dichloropalladium(II) dichloromethane complex (0.08 g) and 1,4-dioxane (5 mL) was degassed, charged with argon, and stirred at 100° C. for 12 h. The reaction mixture was cooled to room temperature, and insoluble material was filtered off. To the filtrate was added ethyl acetate and the organic layer was separated. The organic layer was washed with H₂O (10 mL×2) and saturated brine (10 mL), dried over anhydrous magnesium sulfate, and concentrated in vacuo. The residue was purified by column chromatography on silica-gel to give the title compound (0.11 g) as colorless crystals.

¹H-NMR (300 MHz, DMSO-d₆) δ: 1.56 (3H, t, J=19.3 Hz), 2.05 (3H, s), 2.20 (3H, s), 4.38 (2H, t, J=12.9 Hz), 4.54 (2H, s), 6.45 (1H, d, J=1.7 Hz), 6.53 (1H, d, J=1.3 Hz), 7.24-7.32 (4H, m), 10.52 (1H, s).

### Example 141

### 8-Chloro-6-[1-(2,2-difluoropropyl)-5-(4-fluorophenyl)-3-methyl-1H-pyrazol-4-yl]-2H-1,4-benzoxazin-3(4H)-one

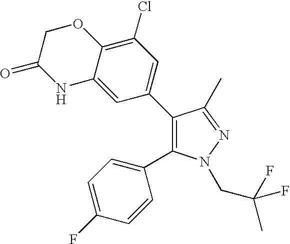

A mixture of 4-bromo-1-(2,2-difluoropropyl)-5-(4-fluorophenyl)-3-methyl-1H-pyrazole (0.21 g), 8-chloro-6-(4,4,5,5-tetramethyl-1,3,2-dioxaborolan-2-yl)-2H-1,4-benzoxazin-3(4H)-one (0.29 g), 2 N cesium carbonate aqueous solution (1.0 mL), [1,1′-bis(diphenylphosphino)ferrocene]dichloropalladium(II) dichloromethane complex (0.08 g) and 1,4-dioxane (5 mL) was degassed, charged with argon, and stirred at 100° C. for 12 h. The reaction mixture was cooled to room temperature, and insoluble material was filtered off. To the filtrate was added ethyl acetate and the organic layer was separated. The organic layer was washed with H₂O (10 mL×2) and saturated brine (10 mL), dried over anhydrous magnesium sulfate, and concentrated in vacuo. The residue was purified by column chromatography on silica-gel to give the title compound (0.08 g) as colorless crystals.

¹H-NMR (300 MHz, DMSO-d₆) δ: 1.57 (3H, t, J=19.3 Hz), 2.22 (3H, s), 4.39 (2H, t, J=13.0 Hz), 4.67 (2H, s), 6.59 (1H, d, J=1.9 Hz), 6.70 (1H, d, J=1.9 Hz), 7.27-7.35 (4H, m), 10.80 (1H, s).

### Preparation 198

### 2-Fluoro-3-(hydrazinomethyl)pyridine trifluoroacetic acid

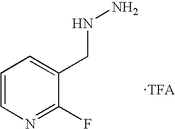

A mixture of 2-fluoropyridine-3-carbaldehyde (3.5 g), tert-butyl carbazate (4.4 g) and ethanol (35 mL) was stirred at room temperature for 3 h. The reaction mixture was concentrated in vacuo. The obtained white solid was extracted with diisopropyl ether, and the extracted solid was diluted with acetic acid/H₂O (15/15 mL). To the mixture was added to sodium cyanoborohydride (1.76 g) and stirred at room temperature for 2 h. The reaction mixture was neutralized by addition of 1 N sodium hydroxide aqueous solution. To the mixture was added ethyl acetate and the organic layer was separated. The organic layer was washed with H₂O (20 mL×2) and saturated brine (30 mL), dried over anhydrous magnesium sulfate, and concentrated in vacuo. The residue was diluted with dichloromethane (30 mL) and trifluoroacetic acid was added dropwise (21 mL), followed by stirring at room temperature for 2 h. The reaction mixture was concentrated in vacuo to give the title compound as a crude product as colorless oil.

### Preparation 199

### 2-Fluoro-3-{[5-(4-fluorophenyl)-3-methyl-1H-pyrazol-1-yl]methyl}pyridine

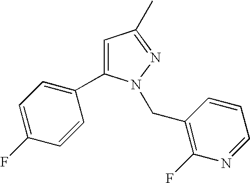

A mixture of 1-(4-fluorophenyl)butane-1,3-dione (0.60 g), 2-fluoro-3-(hydrazinomethyl)pyridine trifluoroacetic acid (2.38 g), triethylamine (0.93 mL), trifluoroacetic acid (0.51 mL) and 2-propanol (10 mL) was stirred at 80° C. for 2 h. The reaction mixture was cooled to room temperature and concentrated in vacuo. The residue was diluted with ethyl acetate and washed with saturated aqueous sodium hydrogen carbonate (10 mL×2), H₂O (10 mL×2) and saturated brine (20 mL), dried over anhydrous magnesium sulfate, and concentrated in vacuo. The residue was purified by column chromatography on silica-gel to give the title compound (0.34 g) as amorphous.

¹H-NMR (300 MHz, CDCl₃) δ: 2.33 (3H, s), 5.29 (2H, s), 6.16 (1H, s), 7.05-7.16 (3H, m), 7.22-7.30 (2H, m), 7.31-7.40 (1H, m), 8.10 (1H, d, J=4.9 Hz).

### Example 142

### 6-{5-(4-Fluorophenyl)-1-[(2-fluoropyridin-3-yl)methyl]-3-methyl-1H-pyrazol-4-yl}-2H-1,4-benzoxazin-3(4H)-one

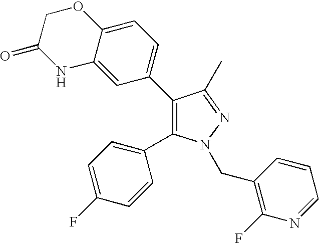

A mixture of 2-fluoro-3-{[5-(4-fluorophenyl)-3-methyl-1H-pyrazol-1-yl]methyl}pyridine (0.34 g), N-Bromosuccinimide (0.22 g) and acetonitrile (8 mL) was stirred at room temperature for 1 h. The reaction mixture was concentrated in vacuo, and the residue was diluted with ethyl acetate. The solution was washed with H₂O (10 mL×2) and saturated brine (10 mL), dried over anhydrous magnesium sulfate, and concentrated in vacuo. The residue was diluted with 1,4-dioxane (10 mL) and added to 6-(4,4,5,5-tetramethyl-1,3,2-dioxaborolan-2-yl)-2H-1,4-benzoxazin-3(4H)-one (0.49 g), 2 N cesium carbonate aqueous solution (1.8 mL) and [1,1′-bis(diphenylphosphino)ferrocene]dichloropalladium(II) dichloromethane complex (0.10 g). The mixture was degassed, charged with argon, and stirred at 100° C. for 12 h. The reaction mixture was cooled to room temperature, and insoluble material was filtered off. To the filtrate was added ethyl acetate and the organic layer was separated. The organic layer was washed with H₂O (10 mL×2) and saturated brine (10 mL), dried over anhydrous magnesium sulfate, and concentrated in vacuo. The residue was purified by column chromatography on silica-gel to give the title compound as colorless (0.24 g) crystals.

¹H-NMR (300 MHz, DMSO-d₆) δ: 2.19 (3H, s), 4.54 (2H, s), 5.20 (2H, s), 6.60 (1H, dd, J=8.3, 2.1 Hz), 6.68 (1H, d, J=2.1 Hz), 6.83 (1H, d, J=8.3 Hz), 7.19-7.36 (5H, m), 7.50-7.61 (1H, m), 8.14 (1H, d, J=4.9 Hz), 10.61 (1H, s).

### Preparation 200

### (Tetrahydro-2H-thiopyran-4-ylmethyl)hydrazine trifluoroacetic acid

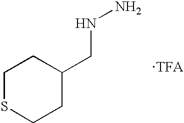

A mixture of tetrahydro-2H-thiopyran-4-carbaldehyde (2.0 g), tert-butyl carbazate (4.4 g) and ethanol (35 mL) was stirred at room temperature for 3 h. The reaction mixture was concentrated in vacuo. The obtained white solid was extracted with diisopropyl ether, and the extracted solid was diluted with acetic acid/H₂O (15/15 mL). To the mixture was added to sodium cyanoborohydride (1.76 g) and stirred at room temperature for 2 h. The reaction mixture was neutralized by addition of 1 N sodium hydroxide aqueous solution. To the mixture was added ethyl acetate and the organic layer was separated. The organic layer was washed with H₂O (20 mL×2) and saturated brine (30 mL), dried over anhydrous magnesium sulfate, and concentrated in vacuo. The residue was diluted with dichloromethane (30 mL) and trifluoroacetic acid was added dropwise (21 mL), followed by stirring at room temperature for 2 h. The reaction mixture was concentrated in vacuo to give the title compound as a crude product as colorless oil.

### Preparation 201

### 4-Bromo-5-(4-fluorophenyl)-3-methyl-1-(tetrahydro-2H-thiopyran-4-ylmethyl)-1H-pyrazole

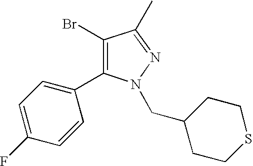

A mixture of 1-(4-fluorophenyl)butane-1,3-dione (0.6 g), (tetrahydro-2H-thiopyran-4-ylmethyl)hydrazine trifluoroacetic acid (2.43 g), triethylamine (0.93 mL), trifluoroacetic acid (0.51 mL) and 2-propanol (10 mL) was stirred at 80° C. for 2 h. The reaction mixture was cooled to room temperature and concentrated in vacuo. The residue was diluted with ethyl acetate and washed with saturated aqueous sodium hydrogen carbonate (10 mL×2), H₂O (10 mL×2) and saturated brine (20 mL), dried over anhydrous magnesium sulfate, and concentrated in vacuo. The residue was added to the mixture of N-Bromosuccinimide (0.62 g) and acetonitrile (10 mL) and the mixture was stirred at room temperature for 1 h. The reaction mixture was concentrated in vacuo, and the residue was diluted with ethyl acetate. The solution was washed with H₂O (10 mL×2) and saturated brine (10 mL), dried over anhydrous magnesium sulfate, and concentrated in vacuo. The residue was purified by column chromatography on silica-gel to give the title compound (0.44 g) as amorphous.

¹H-NMR (300 MHz, CDCl₃) δ: 1.06-1.56 (4H, m), 1.74 (1H, dd, J=13.2, 2.8 Hz), 1.85-2.09 (2H, m), 2.28 (3H, s), 2.43-2.72 (2H, m), 3.78-3.95 (2H, m), 7.05-7.22 (2H, m), 7.27-7.37 (1H, m), 7.79-7.88 (1H, m).

### Example 143

### 6-[5-(4-Fluorophenyl)-3-methyl-1-(tetrahydro-2H-thiopyran-4-ylmethyl)-1H-pyrazol-4-yl]-2H-1,4-benzoxazin-3(4H)-one

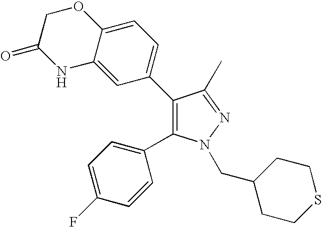

A mixture of 4-bromo-5-(4-fluorophenyl)-3-methyl-1-(tetrahydro-2H-thiopyran-4-ylmethyl)-1H-pyrazole (0.44 g), 6-(4,4,5,5-tetramethyl-1,3,2-dioxaborolan-2-yl)-2H-1,4-benzoxazin-3(4H)-one (0.50 g), 2 N cesium carbonate aqueous solution (1.8 mL), [1,1′-bis(diphenylphosphino)ferrocene]dichloropalladium(II) dichloromethane complex (0.10 g) and 1,4-dioxane (8 mL) was degassed, charged with argon, and stirred at 100° C. for 12 h. The reaction mixture was cooled to room temperature, and insoluble material was filtered off. To the filtrate was added ethyl acetate and the organic layer was separated. The organic layer was washed with H₂O (10 mL×2) and saturated brine (10 mL), dried over anhydrous magnesium sulfate, and concentrated in vacuo. The residue was purified by column chromatography on silica-gel to give the title compound (0.10 g) as colorless crystals.

¹H-NMR (300 MHz, DMSO-d₆) δ: 0.97-1.18 (3H, m), 1.65-1.91 (3H, m), 2.19 (3H, s), 2.45-2.56 (2H, m), 3.75 (2H, d, J=7.0 Hz), 4.53 (2H, s), 6.56 (1H, dd, J=8.3, 1.9 Hz), 6.64 (1H, d, J=1.9 Hz), 6.81 (1H, d, J=8.3 Hz), 7.28 (4H, d, J=7.2 Hz), 10.59 (1H, s).

### Preparation 202

### 3-(Hydrazinomethyl)benzonitrile trifluoroacetic acid

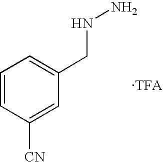

A mixture of 3-formylbenzonitrile (3.0 g), tert-butyl carbazate (3.63 g) and ethanol (30 mL) was stirred at room temperature for 3 h. The reaction mixture was concentrated in vacuo. The obtained white solid was extracted with diisopropyl ether, and the extracted solid was diluted with acetic acid/H₂O (15/15 mL). To the mixture was added to sodium cyanoborohydride (1.44 g) and stirred at room temperature for 2 h. The reaction mixture was neutralized by addition of 1 N sodium hydroxide aqueous solution. To the mixture was added ethyl acetate and the organic layer was separated. The organic layer was washed with H₂O (20 mL×2) and saturated brine (30 mL), dried over anhydrous magnesium sulfate, and concentrated in vacuo. The residue was diluted with dichloromethane (30 mL) and trifluoroacetic acid was added dropwise (18 mL), followed by stirring at room temperature for 2 h. The reaction mixture was concentrated in vacuo to give the title compound as a crude product as colorless oil.

### Preparation 203

### 3-{[5-(4-Fluorophenyl)-3-methyl-1H-pyrazol-1-yl]methyl}benzonitrile

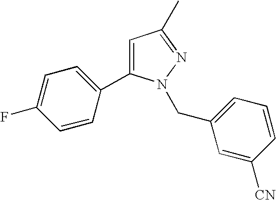

A mixture of 1-(4-fluorophenyl)butane-1,3-dione (0.60 g), 3-(hydrazinomethyl)benzonitrile trifluoroacetic acid (2.44 g), triethylamine (0.93 mL), trifluoroacetic acid (0.51 mL) and 2-propanol (10 mL) was stirred at 80° C. for 2 h. The reaction mixture was cooled to room temperature and concentrated in vacuo. The residue was diluted with ethyl acetate and washed with saturated aqueous sodium hydrogen carbonate (10 mL×2), H₂O (10 mL×2) and saturated brine (20 mL), dried over anhydrous magnesium sulfate, and concentrated in vacuo. The residue was purified by column chromatography on silica-gel to give the title compound (0.82 g) as amorphous.

¹H-NMR (300 MHz, CDCl₃) δ: 2.35 (3H, s), 5.34 (2H, s), 6.19 (1H, s), 7.01-7.15 (2H, m), 7.21-7.31 (2H, m), 7.35-7.49 (2H, m), 7.50-7.62 (1H, m), 7.74 (1H, dd, J=9.1, 5.3).

### Preparation 204

### 3-{[4-Bromo-5-(4-fluorophenyl)-3-methyl-1H-pyrazol-1-yl]methyl}benzonitrile

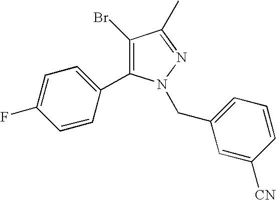

A mixture of 3-{[5-(4-fluorophenyl)-3-methyl-1H-pyrazol-1-yl]methyl}benzonitrile (0.82 g), N-Bromosuccinimide (0.52 g) and acetonitrile (10 mL) was stirred at room temperature for 1 h. The reaction mixture was concentrated in vacuo, and the residue was diluted with ethyl acetate. The solution was washed with H₂O (10 mL×2) and saturated brine (10 mL), dried over anhydrous magnesium sulfate, and concentrated in vacuo. The residue was purified by column chromatography on silica-gel to give the title compound (0.49 g) as colorless crystals.

¹H-NMR (300 MHz, CDCl₃) δ: 2.32 (3H, s), 5.21 (2H, s), 7.05-7.19 (2H, m), 7.20-7.30 (2H, m), 7.34-7.48 (2H, m), 7.51-7.64 (1H, m), 7.81-7.98 (1H, m).

### Example 144

### 3-{[5-(4-Fluorophenyl)-3-methyl-4-(3-oxo-3,4-dihydro-2H-1,4-benzoxazin-6-yl)-1H-pyrazol-1-yl]methyl}benzonitrile

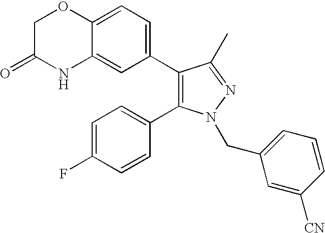

A mixture of 3-{[4-bromo-5-(4-fluorophenyl)-3-methyl-1H-pyrazol-1-yl]methyl}benzonitrile (0.49 g), 6-(4,4,5,5-tetramethyl-1,3,2-dioxaborolan-2-yl)-2H-1,4-benzoxazin-3(4H)-one (0.55 g), 2 N cesium carbonate aqueous solution (2.0 mL), [1,1′-bis(diphenylphosphino)-ferrocene]dichloropalladium(II) dichloromethane complex (0.11 g) and 1,4-dioxane (8 mL) was degassed, charged with argon, and stirred at 100° C. for 12 h. The reaction mixture was cooled to room temperature, and insoluble material was filtered off. To the filtrate was added ethyl acetate and the organic layer was separated. The organic layer was washed with H₂O (10 mL×2) and saturated brine (10 mL), dried over anhydrous magnesium sulfate, and concentrated in vacuo. The residue was purified by column chromatography on silica-gel to give the title compound (0.13 g) as colorless crystals.

¹H-NMR (300 MHz, DMSO-d₆) δ: 2.22 (3H, s), 4.53 (2H, s), 5.21 (2H, s), 6.61 (1H, dd, J=8.3, 2.1 Hz), 6.69 (1H, d, J=2.1 Hz), 6.82 (1H, d, J=8.1 Hz), 7.21-7.32 (5H, m), 7.41-7.45 (1H, m), 7.48-7.55 (1H, m), 7.73 (1H, d, J=7.7 Hz), 10.60 (1H, s).

### Preparation 205

### 5-(Hydrazinomethyl)-2-methoxypyridine trifluoroacetic acid

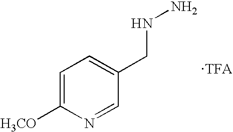

A mixture of 6-methoxypyridine-3-carbaldehyde (2.5 g), tert-butyl carbazate (2.89 g) and ethanol (30 mL) was stirred at room temperature for 3 h. The reaction mixture was concentrated in vacuo. The obtained white solid was extracted with diisopropyl ether, and the extracted solid was diluted with acetic acid/H₂O (15/15 mL). To the mixture was added to sodium cyanoborohydride (1.14 g) and stirred at room temperature for 2 h. The reaction mixture was neutralized by addition of 1 N sodium hydroxide aqueous solution. To the mixture was added ethyl acetate and the organic layer was separated. The organic layer was washed with H₂O (20 mL×2) and saturated brine (30 mL), dried over anhydrous magnesium sulfate, and concentrated in vacuo. The residue was diluted with dichloromethane (30 mL) and trifluoroacetic acid was added dropwise (14 mL), followed by stirring at room temperature for 2 h. The reaction mixture was concentrated in vacuo to give the title compound as a crude product as colorless oil.

### Preparation 206

### 5-{[4-Bromo-5-(4-fluorophenyl)-3-methyl-1H-pyrazol-1-yl]methyl}-2-methoxypyridine

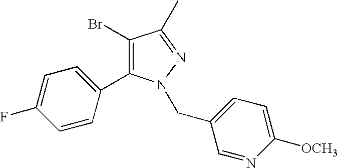

A mixture of 1-(4-fluorophenyl)butane-1,3-dione (0.6 g), 5-(hydrazinomethyl)-2-methoxypyridine trifluoroacetic acid (2.50 g), triethylamine (0.93 mL), trifluoroacetic acid (0.51 mL) and 2-propanol (10 mL) was stirred at 80° C. for 2 h. The reaction mixture was cooled to room temperature and concentrated in vacuo. The residue was diluted with ethyl acetate and washed with saturated aqueous sodium hydrogen carbonate (10 mL×2), H₂O (10 mL×2) and saturated brine (20 mL), dried over anhydrous magnesium sulfate, and concentrated in vacuo. The residue was added to the mixture of N-Bromosuccinimide (0.62 g) and acetonitrile (10 mL) and the mixture was stirred at room temperature for 1 h. The reaction mixture was concentrated in vacuo, and the residue was diluted with ethyl acetate. The solution was washed with H₂O (10 mL×2) and saturated brine (10 mL), dried over anhydrous magnesium sulfate, and concentrated in vacuo. The residue was purified by column chromatography on silica-gel to give the title compound (0.40 g) as amorphous.

¹H-NMR (300 MHz, CDCl₃) δ: 3.87 (3H, s), 5.10 (2H, s), 6.63 (1H, d, J=8.7 Hz), 7.04-7.21 (2H, m), 7.23-7.36 (3H, m), 7.76 (1H, d, J=2.1 Hz).

### Example 145

### 6-{5-(4-Fluorophenyl)-1-[(6-methoxypyridin-3-yl)methyl]-3-methyl-1H-pyrazol-4-yl}-2H-1,4-benzoxazin-3(4H)-one

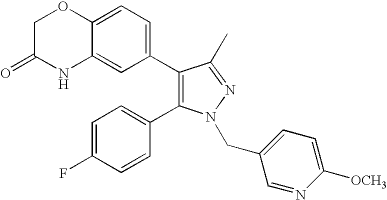

A mixture of 5-{[4-bromo-5-(4-fluorophenyl)-3-methyl-1H-pyrazol-1-yl]methyl}-2-methoxypyridine (0.40 g), 6-(4,4,5,5-tetramethyl-1,3,2-dioxaborolan-2-yl)-2H-1,4-benzoxazin-3(4H)-one (0.44 g), 2 N cesium carbonate aqueous solution (1.6 mL), [1,1′-bis(diphenylphosphino)-ferrocene]dichloropalladium(II) dichloromethane complex (0.09 g) and tetrahydrofuran (5 mL) was degassed, charged with argon, and stirred at 100° C. for 12 h. The reaction mixture was cooled to room temperature, and insoluble material was filtered off. To the filtrate was added ethyl acetate and the organic layer was separated. The organic layer was washed with H₂O (10 mL×2) and saturated brine (10 mL), dried over anhydrous magnesium sulfate, and concentrated in vacuo. The residue was purified by column chromatography on silica-gel to give the title compound (0.25 g) as colorless crystals.

¹H-NMR (300 MHz, DMSO-d₆) δ: 2.19 (3H, s), 3.31 (2H, s), 4.53 (2H, s), 5.09 (2H, s), 6.58 (1H, dd, J=8.1, 2.1 Hz), 6.66 (1H, d, J=2.3 Hz), 6.74 (1H, d, J=8.7 Hz), 6.81 (1H, d, J=8.3 Hz), 7.27 (4H, d, J=7.2 Hz), 7.36 (1H, dd, J=8.5, 2.5 Hz), 7.76 (1H, d, J=1.9 Hz), 10.60 (1H, s).

### Example 146

### 6-[5-(4-Fluorophenyl)-3-methyl-1-(tetrahydrofuran-3-ylmethyl)-1H-pyrazol-4-yl]-2H-1,4-benzoxazin-3(4H)-one

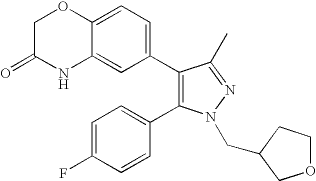

To a mixture of 4-bromo-5-(4-fluorophenyl)-3-methyl-1H-pyrazole (1.50 g), tetrahydrofuran-3-ylmethanol (1.21 g), triphenylphosphine (3.86 g) and tetrahydrofuran (20 mL) was added diisopropyl azodicarboxylate (40% in toluene, 1.9 mol/L, 7.7 mL) and stirred at 60° C. for 4 h. The reaction mixture was concentrated in vacuo, and the residue was added to H₂O and ethyl acetate, and the organic layer was separated. The organic layer was washed with H₂O and saturated brine, dried over anhydrous magnesium sulfate, and concentrated in vacuo. The residue was subjected to short-column chromatography on silica-gel to give crude compound. The residue was diluted with tetrahydrofuran (10 mL) and added to 6-(4,4,5,5-tetramethyl-1,3,2-dioxaborolan-2-yl)-2H-1,4-benzoxazin-3(4H)-one (1.22 g), 2 N cesium _to carbonate aqueous solution (3 mL), [1,1′-bis(diphenylphosphino)-ferrocene]dichloropalladium(II) dichloromethane complex (0.48 g). The mixture was degassed, charged with argon, and stirred at 100° C. for 12 h. The reaction mixture was cooled to room temperature, and insoluble material was filtered off. To the filtrate was added ethyl acetate and the organic layer was separated. The organic layer was washed with H₂O (10 mL×2) and saturated brine (10 mL), dried over anhydrous magnesium sulfate, and concentrated in vacuo. The residue was purified by column chromatography on silica-gel to give the title compound (0.05 g) as colorless crystals.

¹H-NMR (300 MHz, DMSO-d₆) δ: 1.37-1.49 (1H, m), 1.76-1.91 (1H, m), 2.19 (3H, s), 2.55-2.66 (1H, m), 3.33-3.37 (1H, m), 3.48-3.61 (3H, m), 3.88 (2H, d, J=7.6 Hz), 4.53 (2H, s), 6.57 (1H, dd, J=8.1, 2.1 Hz), 6.65 (1H, d, J=1.9 Hz), 6.81 (1H, d, J=8.3 Hz), 7.22-7.36 (4H, m), 10.60 (1H, s).

### Preparation 207

### 4-(Hydrazinomethyl)thiophene-2-carbonitrile trifluoroacetic acid

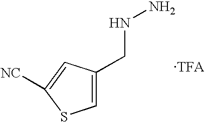

A mixture of 5-bromothiophene-3-carbaldehyde (3.0 g), tert-butyl carbazate (2.49 g) and ethanol (30 mL) was stirred at room temperature for 3 h. The reaction mixture was concentrated in vacuo. The obtained white solid was extracted with diisopropyl ether, and the extracted was diluted with acetic acid/H₂O (15/15 mL). To the mixture was added to sodium cyanoborohydride (0.99 g) and stirred at room temperature for 2 h. The reaction mixture was neutralized by addition of 1 N sodium hydroxide aqueous solution. To the mixture was added ethyl acetate and the organic layer was separated. The organic layer was washed with H₂O (20 mL×2) and saturated brine (30 mL), dried over anhydrous magnesium sulfate, and concentrated in vacuo. The residue was diluted with dichloromethane (30 mL) and trifluoroacetic acid (12 mL) was added dropwise, followed by stirring at room temperature for 2 h. The reaction mixture was concentrated in vacuo to give the title compound as a crude product as colorless oil.

### Example 147

### 4-{[5-(4-Fluorophenyl)-3-methyl-4-(3-oxo-3,4-dihydro-2H-1,4-benzoxazin-6-yl)-1H-pyrazol-1-yl]methyl}thiophene-2-carbonitrile

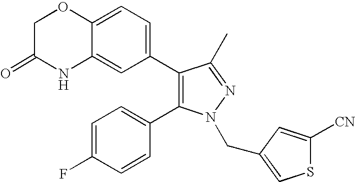

A mixture of 1-(4-fluorophenyl)butane-1,3-dione (1.89 g), 4-(hydrazinomethyl)thiophene-2-carbonitrile trifluoroacetic acid (4.77 g), triethylamine (2.93 mL), trifluoroacetic acid (1.61 mL) and 2-propanol (20 mL) was stirred at 80° C. for 2 h. The reaction mixture was cooled to room temperature and concentrated in vacuo. The residue was diluted with ethyl acetate and washed with saturated aqueous sodium hydrogen carbonate (10 mL×2), H₂O (10 mL×2) and saturated brine (20 mL), dried over anhydrous magnesium sulfate, and concentrated in vacuo. The residue was added to a mixture of copper (I) cyanide (1.32 g) and N,N-dimethylformamide (30 mL), followed by stirring at 170° C. for 15 h. The reaction mixture was cooled to room temperature, and to the mixture was added ethyl acetate/H₂O and the organic layer was separated. The organic layer was washed with H₂O and saturated brine, dried over anhydrous magnesium sulfate, and concentrated in vacuo. The residue was through by short-column chromatography on silica-gel to give crude compound. The residue was added to a mixture of N-Bromosuccinimide (0.28 g) and acetonitrile (10 mL) and the mixture was stirred at room temperature for 1 h. The reaction mixture was concentrated in vacuo, and the residue was diluted with ethyl acetate. The solution was washed with H₂O (10 mL×2) and saturated brine (10 mL), dried over anhydrous magnesium sulfate, and concentrated in vacuo. The residue was through by short-column chromatography on silica-gel to give crude compound. To the residue was added to tetrahydrofuran (10 mL), 6-(4,4,5,5-tetramethyl-1,3,2-dioxaborolan-2-yl)-2H-1,4-benzoxazin-3(4H)-one (0.64 g), 2 N cesium carbonate aqueous solution (1.5 mL) and [1,1′-bis(diphenylphosphino)-ferrocene]dichloropalladium(II) dichloromethane complex (0.25 g). The mixture was degassed, charged with argon, and stirred at 100° C. for 12 h. The reaction mixture was cooled to room temperature, and insoluble material was filtered off. To the filtrate was added ethyl acetate and the organic layer was separated. The organic layer was washed with H₂O (10 mL×2) and saturated brine (10 mL), dried over anhydrous magnesium sulfate, and concentrated in vacuo. The residue was purified by column chromatography on silica-gel to give the title compound (0.05 g) as colorless crystals.

¹H-NMR (300 MHz, DMSO-d₆) δ: 2.21 (3H, s), 4.54 (2H, s), 5.41 (2H, s), 6.55-6.63 (1H, m), 6.66 (1H, d, J=1.9 Hz), 6.83 (1H, d, J=8.3 Hz), 6.96 (1H, d, J=3.8 Hz), 7.24-7.33 (4H, m, J=7.2 Hz), 7.78 (1H, d, J=3.8 Hz), 10.61 (1H, s, 1H).

### Preparation 208

### [4-(Tetrahydro-2H-pyran-2-yloxy)cyclohexyl]methanol

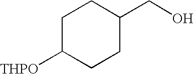

To a cold (0° C.) mixture of ethyl 4-hydroxycyclohexanecarboxylate (5.0 g), p-toluenesulfonic acid (1.65 g) and ether (50 mL) was added dropwise 3,4-dihydro-2H-pyran (4.0 mL) over a period of 15 min, and the mixture was allowed to warm to room temperature, followed by stirring for 4 h. The reaction mixture was added to saturated aqueous sodium hydrogen carbonate and the organic layer was separated. The organic layer was washed with H₂O and saturated brine, dried over anhydrous magnesium sulfate, and concentrated in vacuo, the residue was diluted with tetrahydrofuran (50 mL) and the mixture was cooled to 0° C. To the mixture was added lithium aluminum hydride (1.10 g) over a period of 10 min, and the mixture was allowed to warm to room temperature. The mixture was stirred for 2 h. The reaction mixture was cooled to 0° C., and quenched by addition of H₂O. The separated organic layer was washed with H₂O and saturated brine, dried over anhydrous magnesium sulfate, and concentrated in vacuo, to give the title compound as crude product as colorless oil.

### Preparation 209

### 4-{[4-Bromo-5-(4-fluorophenyl)-3-methyl-1H-pyrazol-1-yl]methyl}cyclohexanol

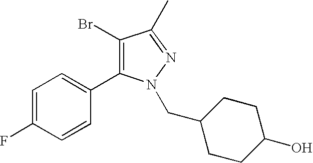

To a mixture of 4-bromo-5-(4-fluorophenyl)-3-methyl-1H-pyrazole (2.0 g), [4-(tetrahydro-2H-pyran-2-yloxy)cyclohexyl]methanol (3.36 g), triphenylphosphine (5.14 g) and tetrahydrofuran (20 mL) was added diisopropyl azodicarboxylate (40% in toluene, 1.9 mol/L, 10.3 mL) and stirred at 60° C. for 4 h. The reaction mixture was concentrated in vacuo, and the residue was added to H₂O and ethyl acetate, and the organic layer was separated. The organic layer was washed with H₂O and saturated brine, dried over anhydrous magnesium sulfate, and concentrated in vacuo. The residue was through by short-column chromatography on silica-gel to give crude product. The residue was diluted with methanol (50 mL) and added to p-toluenesulfonic acid monohydrate (0.07 g), and stirred at room temperature for 4 h. The reaction mixture was quenched by addition of saturated aqueous sodium hydrogen carbonate and concentrated in vacuo. The residue was added to ethyl acetate and the organic layer was separated. The organic layer was washed with H₂O and saturated brine, dried over anhydrous magnesium sulfate, and concentrated in vacuo to give the title compound as crude as amorphous.

### Example 148

### 6-{5-(4-Fluorophenyl)-1-[(cis-4-hydroxycyclohexyl)methyl]-3-methyl-1H-pyrazol-4-yl}-2H-1,4-benzoxazin-3(4H)-one

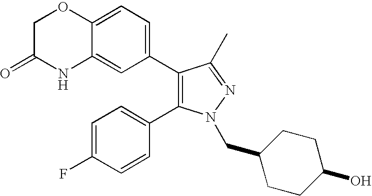

To a mixture of 4-{[4-bromo-5-(4-fluorophenyl)-3-methyl-1H-pyrazol-1-yl]methyl}cyclohexanol (0.5 g), 6-(4,4,5,5-tetramethyl-1,3,2-dioxaborolan-2-yl)-2H-1,4-benzoxazin-3(4H)-one (0.56 g), 2 N cesium carbonate aqueous solution (1.4 mL), [1,1′-bis(diphenylphosphino)-ferrocene]dichloropalladium(II) dichloromethane complex (0.22 g) and tetrahydrofuran (10 mL) was degassed, charged with argon, and stirred at 100° C. for 12 h. The reaction mixture was cooled to room temperature, and insoluble material was filtered off. To the filtrate was added ethyl acetate and the organic layer was separated. The organic layer was washed with H₂O (10 mL×2) and saturated brine (10 mL), dried over anhydrous magnesium sulfate, and concentrated in vacuo. The residue was purified by column is chromatography on silica-gel to give the title compound (0.06 g) as colorless crystals.

¹H-NMR (300 MHz, DMSO-d₆) δ: 1.08-1.45 (8H, m), 1.73-1.86 (1H, m), 2.9 (3H, s), 3.61-3.68 (1H, m), 3.76 (2H, d, J=7.2 Hz), 4.22 (1H, d, J=3.4 Hz), 4.53 (2H, s), 6.56 (1H, dd, J=8.2, 2.0 Hz), 6.64 (1H, d, J=1.9 Hz), 6.80 (1H, d, J=8.3 Hz), 7.28 (4H, d, J=7.2 Hz), 10.59 (1H, s).

### Preparation 210

### 4-{[4-Bromo-5-(4-fluorophenyl)-3-methyl-1H-pyrazol-1-yl]methyl}cyclohexanone

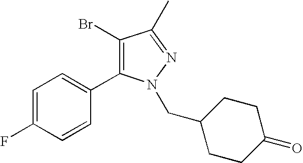

To a solution of 4-{[4-bromo-5-(4-fluorophenyl)-3-methyl-1H-pyrazol-1-yl]methyl}cyclohexanol (2.78 g) in acetonitrile (40 mL) was added 1,1,1-Tris(acetyloxy)-1,1-dihydro-1,2-benziodoxol-3-(1H)-one (3.53 g), and the mixture was stirred at room temperature for 2 h. The reaction mixture was quenched by addition of saturated sodium hydrogen carbonate, and the organic layer was separated. The organic layer was washed with H₂O and saturated brine, dried over anhydrous magnesium sulfate, and concentrated in vacuo. The residue was through short-column chromatography on silica-gel to give the title compound as crude as amorphous.

### Example 149

### 6-{5-(4-Fluorophenyl)-3-methyl-1-[(4-oxocyclohexyl)methyl]-1H-pyrazol-4-yl}-2H-1,4-benzoxazin-3(4H)-one

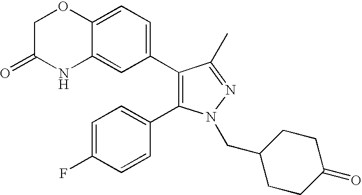

To a mixture of 4-{[4-bromo-5-(4-fluorophenyl)-3-methyl-1H-pyrazol-1-yl]methyl}cyclohexanone (0.6 g), 6-(4,4,5,5-tetramethyl-1,3,2-dioxaborolan-2-yl)-2H-1,4-benzoxazin-3(4H)-one (0.68 g), 2 N cesium carbonate aqueous solution (1.6 mL), [1,1′-bis(diphenylphosphino)-ferrocene]dichloropalladium(II) dichloromethane complex (0.27 g) and tetrahydrofuran (10 mL) was degassed, charged with argon, and stirred at 100° C. for 12 h. The reaction mixture was cooled to room temperature, and insoluble material was filtered off. To the filtrate was added ethyl acetate and the organic layer was separated. The organic layer was washed with H₂O (10 mL×2) and saturated brine (10 mL), dried over anhydrous magnesium sulfate, and concentrated in vacuo. The residue was purified by column chromatography on silica-gel to give the title compound (0.08 g) as colorless crystals.

¹H-NMR (300 MHz, DMSO-d₆) δ: 1.12-1.31 (2H, m), 1.64-1.83 (2H, m), 2.01-2.17 (2H, m), 2.21 (3H, s), 2.23-2.39 (3H, m), 3.86 (2H, d, J=7.2 Hz), 4.53 (2H, s), 6.57 (1H, dd, J=8.3, 2.1 Hz), 6.65 (1H, d, J=2.1 Hz), 6.81 (1H, d, J=8.3 Hz), 7.21-7.36 (4H, m), 10.60 (1H, s).

### Example 150

### 6-{1-[(4,4-Difluorocyclohexyl)methyl]-5-(4-fluorophenyl)-3-methyl-1H-pyrazol-4-yl}-2H-1,4-benzoxazin-3(4H)-one

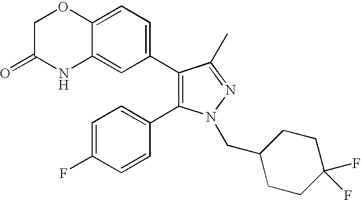

To a cold (0° C.) solution of 4-{[4-bromo-5-(4-fluorophenyl)-3-methyl-1H-pyrazol-1-yl]methyl}cyclohexanone (0.6 g) in toluene (10 mL) was added dropwise (diethylamino)sulfur trifluoride (0.33 mL) over a period of 5 min. The resulting mixture was allowed to warm to room temperature and stirred for 2 h. The reaction mixture was quenched by addition of saturated aqueous sodium hydrogen carbonate and the organic layer was separated. The organic layer was washed with H₂O and saturated brine, dried over anhydrous magnesium sulfate, and concentrated in vacuo. The residue was purified by column chromatography on silica-gel. The residue was added to 6-(4,4,5,5-tetramethyl-1,3,2-dioxaborolan-2-yl)-2H-1,4-benzoxazin-3(4H)-one (0.64 g), 2 N cesium carbonate aqueous solution (1.5 mL), [1,1′-bis(diphenylphosphino)-ferrocene]dichloropalladium(II) dichloromethane complex (0.25 g) and tetrahydrofuran/H₂O (6/2 mL). The mixture was degassed, charged with argon, and stirred at 100° C. for 12 h. The reaction mixture was cooled to room temperature, and insoluble material was filtered off. To the filtrate was added ethyl acetate and the organic layer was separated. The organic layer was washed with H₂O (10 mL×2) and saturated brine (10 mL), dried over anhydrous magnesium sulfate, and concentrated in vacuo. The residue was purified by column chromatography on silica-gel to give the title compound (0.05 g) as colorless crystals.

¹H-NMR (300 MHz, DMSO-d₆) δ: 1.15-1.57 (2H, m), 1.60-2.14 (7H, m), 2.19 (3H, s), 4.03 (2H, d, J=7.4 Hz), 4.60 (2H, s), 6.49-6.79 (2H, m), 6.95 (1H, d, J=7.9 Hz), 7.05-7.20 (2H, m), 7.28-7.45 (2H, m), 10.63 (1H, s).

### Preparation 211

### 4-Bromo-1-(cyclobutylmethyl)-5-(4-fluorophenyl)-3-methyl-1H-pyrazole

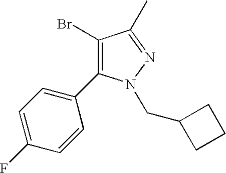

To a mixture of 4-bromo-5-(4-fluorophenyl)-3-methyl-1H-pyrazole (1.50 g), cyclobutanemethanol (1.02 g), triphenylphosphine (3.86 g) and tetrahydrofuran (20 mL) was added diisopropyl azodicarboxylate (40% in toluene, 1.9 mol/L, 7.7 mL) and stirred at 60° C. for 4 h. The reaction mixture was concentrated in vacuo, and the residue was added to H₂O and ethyl acetate, and the organic layer was separated. The organic layer was washed with H₂O and saturated brine, dried over anhydrous magnesium sulfate, and concentrated in vacuo. The residue was purified by column chromatography on silica-gel to give the title compound (0.61 g) as amorphous.

¹H-NMR (300 MHz, CDCl₃) δ: 1.49-1.98 (6H, m), 2.28 (3H, s), 2.58-2.74 (1H, m), 3.99 (2H, d, J=7.4 Hz), 7.14-7.23 (2H, m), 7.30-7.38 (2H, m).

### Example 151

### 6-[1-(Cyclobutylmethyl)-5-(4-fluorophenyl)-3-methyl-1H-pyrazol-4-yl]-2H-1,4-benzoxazin-3(4H)-one

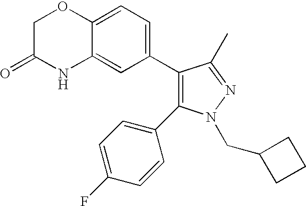

A mixture of 4-bromo-1-(cyclobutylmethyl)-5-(4-fluorophenyl)-3-methyl-1H-pyrazole (0.61 g), 6-(4,4,5,5-tetramethyl-1,3,2-dioxaborolan-2-yl)-2H-1,4-benzoxazin-3(4H)-one (0.78 g), 2 N cesium carbonate aqueous solution (1.9 mL), [1,1′-bis(diphenylphosphino)-ferrocene]dichloropalladium(II) dichloromethane complex (0.31 g) and tetrahydrofuran/H₂O (10/2 mL) was degassed, charged with argon, and stirred at 100° C. for 12 h. The reaction mixture was cooled to room temperature, and insoluble material was filtered off. To the filtrate was added ethyl acetate and the organic layer was separated. The organic layer was washed with H₂O (10 mL×2) and saturated brine (10 mL), dried over anhydrous magnesium sulfate, and concentrated in vacuo. The residue was purified by column chromatography on silica-gel to give the title compound (0.27 g) as colorless crystals.

¹H-NMR (300 MHz, DMSO-d₆) δ: 1.45-1.93 (6H, m), 2.18 (3H, s), 2.54-2.67 (1H, m), 3.92 (2H, d, J=7.4 Hz), 4.53 (2H, s), 6.55 (1H, dd, J=8.2, 2.0 Hz), 6.64 (1H, d, J=2.1 Hz), 6.80 (1H, d, J=8.3 Hz), 7.20-7.33 (4H, m), 10.60 (1H, s).

### Preparation 212

### [3-(Tetrahydro-2H-pyran-2-yloxy)cyclopentyl]methanol

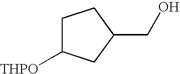

To a cold (0° C.) solution of 3-oxocyclopentanecarboxylic acid (5.0 g) in methanol was added dropwise thionyl chloride (3.1 mL) over a period of 15 min, and the mixture was allowed to warm to room temperature. The resulting mixture was stirred for 5 h. The reaction mixture was concentrated in vacuo, to the residue was added ethyl acetate. The organic layer was washed with saturated aqueous sodium hydrogen carbonate, H₂O and saturated brine, dried over anhydrous magnesium sulfate, and concentrated in vacuo. The residue was added to tetrahydrofuran (50 mL) and cooled to 0° C. To the mixture was added sodium tetrahydroborate (2.21 g) over a period of 10 min, and the mixture was allowed to warm to room temperature. The mixture was stirred for 12 h. The reaction mixture was quenched by addition of H₂O, and the organic layer was separated. The organic layer was washed with H₂O and saturated brine, dried over anhydrous magnesium sulfate, and concentrated in vacuo. The residue was added to diethyl ether (50 mL) and cooled to 0° C. The mixture was added to a mixture of p-toluenesulfonic acid (2.23 g) and 3,4-dihydro-2H-pyran (5.3 mL), and the mixture was allowed to warm to room temperature. The mixture was stirred for 12 h. The reaction mixture was concentrated in vacuo, the residue was diluted with ethyl acetate. The organic layer was washed with saturated aqueous sodium hydrogen carbonate, H₂O and saturated brine, dried over anhydrous magnesium sulfate, and concentrated in vacuo. The residue was added to tetrahydrofuran (50 mL) and cooled to 0° C. To the mixture was added by portionwise lithium aluminum hydride (1.48 g) over a period of 10 min. The resulting mixture was allowed to warm to room temperature and stirred for 3 h. The reaction mixture was quenched by addition of H₂O and the organic layer was separated. The organic layer was washed with H₂O and saturated brine, dried over anhydrous magnesium sulfate, and concentrated in vacuo to give the title compound as colorless oil.

¹H-NMR (300 MHz, CDCl₃) δ: 1.42-1.90 (12H, m), 2.15-2.42 (1H, m), 3.43-3.56 (2H, m), 3.80-3.95 (2H, m), 4.19-4.35 (1H, m), 4.56-4.62 (1H, m).

### Preparation 213

### 3-{[4-Bromo-5-(4-fluorophenyl)-3-methyl-1H-pyrazol-1-yl]methyl}cyclopentanol

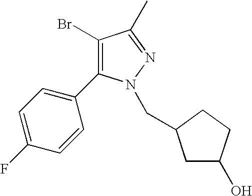

To a mixture of 4-bromo-5-(4-fluorophenyl)-3-methyl-1H-pyrazole (2.5 g), [3-(tetrahydro-2H-pyran-2-yloxy)cyclopentyl]methanol (3.9 g), triphenylphosphine (6.4 g) and tetrahydrofuran (30 mL) was added diisopropyl azodicarboxylate (40% in toluene, 1.9 mol/L, 12.9 mL) and stirred at 60° C. for 4 h. The reaction mixture was concentrated in vacuo, and the residue was added to H₂O and ethyl acetate, and the organic layer was separated. The organic layer was washed with H₂O and saturated brine, dried over anhydrous magnesium sulfate, and concentrated in vacuo. The residue was through by short-column chromatography on silica-gel to give crude product. The residue was diluted with methanol (30 mL). To the mixture was added p-toluenesulfonic acid monohydrate (0.06 g), and stirred at room temperature for 4 h. The reaction mixture was quenched by addition of saturated aqueous sodium hydrogen carbonate and concentrated in vacuo. To the residue was added ethyl acetate and the organic layer was separated. The organic layer was washed with H₂O and saturated brine, dried over anhydrous magnesium sulfate, and concentrated in vacuo to give the title compound as crude as amorphous.

### Example 152

### 6-{5-(4-Fluorophenyl)-1-[(3-hydroxycyclopentyl)methyl]-3-methyl-1H-pyrazol-4-yl}-2H-1,4-benzoxazin-3(4H)-one

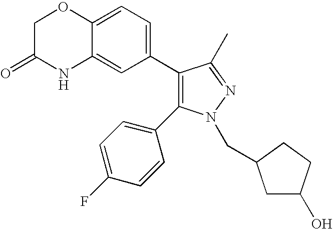

A mixture of 4-bromo-5-(4-fluorophenyl)-3-methyl-1-{[3-(tetrahydro-2H-pyran-2-yloxy)cyclopentyl]methyl}-1H-pyrazole (0.70 g), 6-(4,4,5,5-tetramethyl-1,3,2-dioxaborolan-2-yl)-2H-1,4-benzoxazin-3(4H)-one (0.82 g), 2 N cesium carbonate aqueous solution (2 mL), [1,1′-bis(diphenylphosphino)-ferrocene]dichloropalladium(II) dichloromethane complex (0.32 g) and tetrahydrofuran/H₂O (10/2 mL) was degassed, charged with argon, and stirred at 100° C. for 12 h. The reaction mixture was cooled to room temperature, and insoluble material was filtered off. To the filtrate was added ethyl acetate and the organic layer was separated. The organic layer was washed with H₂O (10 mL×2) and saturated brine (10 mL), dried over anhydrous magnesium sulfate, and concentrated in vacuo. The residue was purified by column chromatography on silica-gel to give the title compound (0.06 g) as colorless crystals.

¹H-NMR (300 MHz, DMSO-d₆) δ: 0.98-1.68 (6H, m), 1.71-1.82 (1H, m), 2.19 (3H, s), 2.21-2.32 (1H, m), 3.80-3.92 (2H, m), 3.97-4.04 (1H, m), 4.53 (2H, s), 6.56 (1H, dd, J=8.3, 2.1 Hz), 6.65 (1H, d, J=1.9 Hz), 6.81 (1H, d, J=8.3 Hz), 7.21-7.34 (4H, m), 10.59 (1H, s).

### Preparation 214

### 4-Bromo-5-(4-fluorophenyl)-3-methyl-1-(tetrahydrofuran-2-ylmethyl)-1H-pyrazole

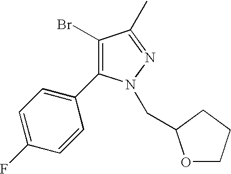

To a mixture of 4-bromo-5-(4-fluorophenyl)-3-methyl-1H-pyrazole (1.50 g), tetrahydrofuran-3-yl methanol (1.21 g), triphenylphosphine (3.86 g) and tetrahydrofuran (20 mL) was added diisopropyl azodicarboxylate (40% in toluene, 1.9 mol/L, 7.7 mL) and stirred at 60° C. for 4 h. The reaction mixture was concentrated in vacuo, and the residue was added to H₂O and ethyl acetate, and the organic layer was separated. The organic layer was washed with H₂O and saturated brine, dried over anhydrous magnesium sulfate, and concentrated in vacuo to give the title compound as crude as amorphous.

### Example 153

### 6-[5-(4-Fluorophenyl)-3-methyl-1-(tetrahydrofuran-2-ylmethyl)-1H-pyrazol-4-yl]-2H-1,4-benzoxazin-3(4H)-one

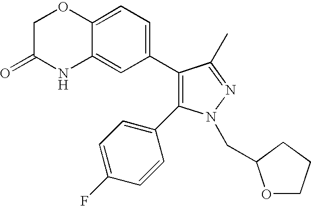

A mixture of 4-bromo-5-(4-fluorophenyl)-3-methyl-1-(tetrahydrofuran-2-ylmethyl)-1H-pyrazole (0.80 g), 6-(4,4,5,5-tetramethyl-1,3,2-dioxaborolan-2-yl)-2H-1,4-benzoxazin-3(4H)-one (0.97 g), 2 N cesium carbonate aqueous solution (2.4 mL), [1,1′-bis(diphenylphosphino)-ferrocene]dichloropalladium(II) dichloromethane complex (0.39 g) and tetrahydrofuran/H₂O (10/2 mL) was degassed, charged with argon, and stirred at 100° C. for 12 h. The reaction mixture was cooled to room temperature, and insoluble material was filtered off. To the filtrate was added ethyl acetate and the organic layer was separated. The organic layer was washed with H₂O (10 mL×2) and saturated brine (10 mL), dried over anhydrous magnesium sulfate, and concentrated in vacuo. The residue was purified by column chromatography on silica-gel to give the title compound (0.21 g) as colorless crystals.

¹H-NMR (300 MHz, DMSO-d₆) δ: 1.46 (4H, m), 2.19 (3H, s), 3.49-3.61 (2H, m), 3.79-3.96 (2H, m), 4.12-4.22 (1H, m), 4.53 (2H, s), 6.56 (1H, dd, J=8.3, 2.1 Hz), 6.65 (1H, d, J=1.9 Hz), 6.81 (1H, d, J=8.3 Hz), 7.21-7.37 (4H, m), 10.60 (1H, s).

### Example 154

### 6-{5-(4-Fluorophenyl)-3-methyl-1-[(3-oxocyclopentyl)methyl]-1H-pyrazol-4-yl}-2H-1,4-benzoxazin-3(4H)-one

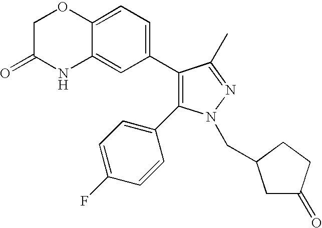

To a solution of 3-{[4-bromo-5-(4-fluorophenyl)-3-methyl-1H-pyrazol-1-yl]methyl}cyclopentanol (1.44 g) in acetonitrile (20 mL) was added 1,1,1-Tris(acetyloxy)-1,1-dihydro-1,2-benziodoxol-3-(1H)-one (1.90 g), and the mixture was stirred at room temperature for 2 h. The reaction mixture was quenched by addition of saturated sodium hydrogen carbonate, and the organic layer was separated. The organic layer was washed with H₂O and saturated brine, dried over anhydrous magnesium sulfate, and concentrated in vacuo. The residue was added to a mixture of 6-(4,4,5,5-tetramethyl-1,3,2-dioxaborolan-2-yl)-2H-1,4-benzoxazin-3(4H)-one (0.48 g), 2 N cesium carbonate aqueous solution (1.6 mL), [1,1′-bis(diphenylphosphino)-ferrocene]dichloropalladium(II) dichloromethane complex (0.19 g) and tetrahydrofuran/H₂O (10/2 mL). The mixture was degassed, charged with argon, and stirred at 100° C. for 12 h. The reaction mixture was cooled to room temperature, and insoluble material was filtered off. To the filtrate was added ethyl acetate and the organic layer was separated. The organic layer was washed with H₂O (10 mL×2) and saturated brine (10 mL), dried over anhydrous magnesium sulfate, and concentrated in vacuo. The residue was purified by column chromatography on silica-gel to give the title compound (0.09 g) as colorless crystals.

¹H-NMR (300 MHz, DMSO-d₆) δ: 1.31-1.51 (1H, m), 1.75-1.93 (2H, m), 1.95-2.16 (3H, m), 2.20 (3H, s), 2.54-2.69 (1H, m), 3.97 (2H, dd, J=7.0, 2.1 Hz), 4.53 (2H, s), 6.57 (1H, dd, J=8.3, 1.9 Hz), 6.65 (1H, d, J=1.9 Hz), 6.81 (1H, d, J=8.3 Hz), 6.95-7.13 (1H, m), 7.21-7.42 (3H, m), 10.61 (1H, s).

### Preparation 215

### 4-Bromo-5-(4-fluorophenyl)-1-(furan-3-ylmethyl)-3-methyl-1H-pyrazole

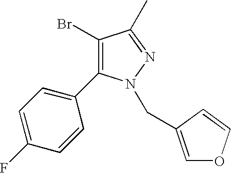

To a solution of 4-bromo-5-(4-fluorophenyl)-3-methyl-1H-pyrazole (1.00 g), furan-3-ylmethanol (0.73 ml) and triphenylphosphine (2.53 g) in tetrahydrofuran (20 ml) was added slowly diisopropyl azodicarboxylate (40% in toluene, 5.2 ml) at 60° C. and the reaction mixture was stirred for 16 h at 60° C. The mixture was treated with water and extracted with ethyl acetate. Organic layer was washed with water and saturated brine, dried over anhydrous magnesium sulfate and concentrated in vacuo. The residue was washed with diisopropyl ether and filtered. The filtrate was concentrated and purified by chromatography on silica gel using hexane/ethyl acetate as an eluent to give the title compound as gray oil (0.34 g).

¹H-NMR (300 MHz, CDCl₃) δ: 2.30 (3H, s), 5.02 (2H, s), 6.20 (1H, d, J=0.1 Hz,), 7.14-7.21 (3H, m), 7.29-7.35 (3H, m).

### Example 155

### 6-[5-(4-Fluorophenyl)-1-(furan-3-ylmethyl)-3-methyl-1H-pyrazol-4-yl]-2H-1,4-benzoxazin-3(4H)-one

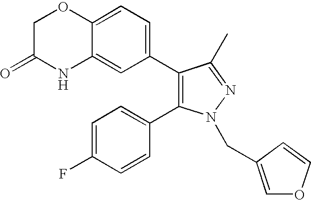

To a mixture of 4-bromo-5-(4-fluorophenyl)-1-(furan-3-ylmethyl)-3-methyl-1H-pyrazole (340 mg) and 6-(4,4,5,5-tetramethyl-1,3,2-dioxaborolan-2-yl)-2H-1,4-benzoxazin-3(4H)-one (419 mg) in tetrahydrofuran (8 ml) were added 2M cesium carbonate solution (2.0 ml) and [1,1′-bis(diphenylphosphino)ferrocene]dichloropalladium(II) dichloromethane complex (163 mg). The mixture was refluxed for 19 h under argon atmosphere. The mixture was treated with water and filtered. The filtrate was extracted with ethyl acetate. Organic layer was washed with water and saturated brine, dried over anhydrous magnesium sulfate and concentrated in vacuo. The residue was purified by chromatography on silica gel using n-hexane/ethyl acetate as an eluent to give the title compound as a white solid (173 mg).

¹H-NMR (300 MHz, CDCl₃) δ: 2.32 (3H, s), 4.59 (2H, s), 5.02 (2H, s), 6.24 (1H, m), 6.45 (1H, d, J=2.0 Hz), 6.68 (1H, dd, J=8.3 Hz, 2.0 Hz), 6.86 (1H, d, J=8.3 Hz), 7.02-7.11 (2H, m), 7.12-7.20 (3H, m), 7.33 (1H, t, J=1.7 Hz), 7.84 (1H, s).

### Preparation 216

### 4-Bromo-5-(4-fluorophenyl)-3-methyl-1-[(2-methylfuran-3-yl)methyl]-1H-pyrazole

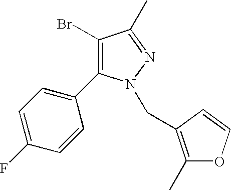

To a solution of 4-bromo-5-(4-fluorophenyl)-3-methyl-1H-pyrazole (0.60 g), (2-methylfuran-3-yl)methanol (0.53 g) and triphenylphosphine (1.54 g) in tetrahydrofuran (12 ml) was added slowly diisopropyl azodicarboxylate (40% in toluene, 3.1 ml) at 60° C. and the reaction mixture was stirred at 60° C. for 2 h. The mixture was treated with water and extracted with ethyl acetate. Organic layer was washed with water and saturated brine, dried over anhydrous magnesium sulfate and concentrated in vacuo. The residue was washed with diisopropyl ether and filtered. The filtrate was concentrated and purified by chromatography on silica gel using hexane/ethyl acetate as an eluent to give the title compound as yellow oil (0.33 g).

¹H-NMR (300 MHz, CDCl₃) δ: 1.96 (3H, s), 2.29 (3H, s), 4.94 (2H, s), 6.07 (1H, d, J=1.9 Hz), 7.15 (1H, d, J=2.1 Hz), 7.16-7.21 (2H, m), 7.26-7.33 (2H, m).

### Example 156

### 6-{5-(4-Fluorophenyl)-3-methyl-1-[(2-methylfuran-3-yl)methyl]-1H-pyrazol-4-yl}-2H-1,4-benzoxazin-3(4H)-one

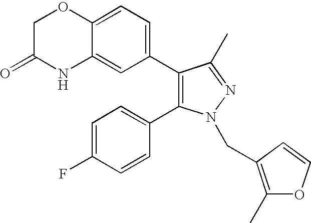

To a mixture of 4-bromo-5-(4-fluorophenyl)-3-methyl-1-[(2-methylfuran-3-yl)methyl]-1H-pyrazole (333 mg) and 6-(4,4,5,5-tetramethyl-1,3,2-dioxaborolan-2-yl)-2H-1,4-benzoxazin-3(4H)-one (392 mg) in tetrahydrofuran (6 ml) were added 2M cesium carbonate solution (1.0 ml) and [1,1′-bis(diphenylphosphino)ferrocene]dichloropalladium(II) dichloromethane complex (155 mg). The reaction mixture was stirred for 20 minutes at 150° C. with microwave irradiation. The mixture was treated with water and filtered. The filtrate was extracted with ethyl acetate and water. Organic layer was washed with water and saturated brine, dried over anhydrous magnesium sulfate and concentrated in vacuo. The residue was purified by chromatography on silica is gel using hexane/ethyl acetate as an eluent. Obtained solid was washed with diisopropyl ether to give the title compound as a white solid (35 mg).

¹H-NMR (300 MHz, DMSO-d₆) δ: 1.94 (3H, s), 2.19 (3H, s), 4.53 (2H, s), 4.92 (2H, s), 6.04 (1H, d, J=1.9 Hz), 6.52-6.59 (1H, dd, J=8.3, 2.1 Hz), 6.65 (1H, d, J=2.1 Hz), 6.81 (1H, d, J=8.3 Hz), 7.24 (2H, s), 7.29 (2H, d, J=2.6 Hz), 7.36 (1H, d, J=1.9 Hz), 10.60 (1H, s).

### Preparation 217

### 3-{[4-Bromo-5-(4-fluorophenyl)-3-methyl-1H-pyrazol-1-yl]methyl}-5-methylisoxazole

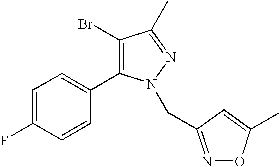

To a solution of 4-bromo-5-(4-fluorophenyl)-3-methyl-1H-pyrazole (0.60 g), (5-methylisoxazol-3-yl)methanol (0.53 g) and triphenylphosphine (1.54 g) in tetrahydrofuran (12 ml) was added slowly diisopropyl azodicarboxylate (40% in toluene, 3.1 ml) at 60° C. and the reaction mixture was stirred for 6 h at 60° C. The mixture was treated with water and extracted with ethyl acetate. Organic layer was washed with water and saturated brine, dried over anhydrous magnesium sulfate and concentrated in vacuo. The residue was to washed with diisopropyl ether and filtered. The filtrate was concentrated and purified by chromatography on silica gel using hexane/ethyl acetate as an eluent to give the title compound as yellow oil (0.17 g).

¹H-NMR (300 MHz, CDCl₃) δ: 2.30 (3H, s), 2.39 (3H, s), 5.17 (2H, s), 5.93 (1H, s), 7.11-7.24 (2H, m), 7.31-7.44 (2H, m).

### Example 157

### 6-{5-(4-Fluorophenyl)-3-methyl-1-[(5-methylisoxazol-3-yl)methyl]-1H-pyrazol-4-yl}-2H-1,4-benzoxazin-3(4H)-one

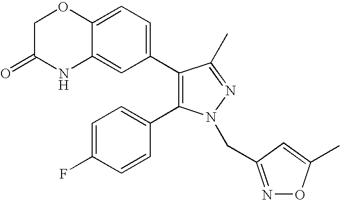

To a mixture of 3-{[4-bromo-5-(4-fluorophenyl)-3-methyl-1H-pyrazol-1-yl]methyl}-5-methylisoxazole (167 mg) and 6-(4,4,5,5-tetramethyl-1,3,2-dioxaborolan-2-yl)-2H-1,4-benzoxazin-3(4H)-one (198 mg) in tetrahydrofuran (3 ml) were added 2M cesium carbonate solution (0.5 ml) and [1,1′-bis(diphenylphosphino)ferrocene]dichloropalladium(II) dichloromethane complex (78 mg). The reaction mixture was refluxed for 18 h under argon atmosphere. The mixture was treated with water and filtered. The filtrate was extracted with ethyl acetate. Organic layer was washed with water and saturated brine, dried over anhydrous magnesium sulfate and concentrated in vacuo. The residue was purified by chromatography on silica gel using hexane/ethyl acetate as an eluent. Obtained solid was washed with diisopropyl ether to give the title compound as a brown solid (7 mg).

¹H-NMR (300 MHz, DMSO-d₆) δ: 2.31 (3H, s), 2.40 (3H, s), 4.59 (2H, s), 5.17 (2H, s), 6.00 (1H, s), 6.51 (1H, s), 6.62-6.74 (1H, m), 6.85 (1H, d, J=8.1 Hz), 7.06 (2H, t, J=8.4 Hz), 7.18 (1H, d, J=5.3 Hz), 7.26 (1H, s), 8.64 (1H, s).

### Example 158

### 6-[5-(4-Fluorophenyl)-3-methyl-1-{[(2R)-5-oxotetrahydrofuran-2-yl]methyl}-1H-pyrazol-4-yl]-2H-1,4-benzoxazin-3(4H)-one

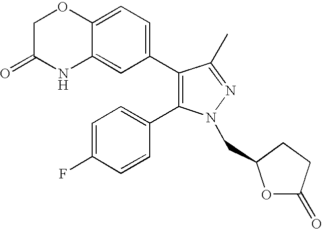

To a solution of 4-bromo-5-(4-fluorophenyl)-3-methyl-1H-pyrazole (1.00 g), (5R)-5-(hydroxymethyl)dihydrofuran-2(3H)-one (0.74 ml) and triphenylphosphine (2.57 g) in tetrahydrofuran (20 ml) was added slowly diisopropyl azodicarboxylate (40% in toluene, 5.2 ml) at 60° C. and the reaction mixture was stirred for 24 h at 60° C. The mixture was treated with water and extracted with ethyl acetate. The organic layer was washed with water and saturated brine, dried over anhydrous magnesium sulfate and concentrated in vacuo. The residue was washed with diisopropyl ether and filtered. The filtrate was concentrated in vacuo and the residue was through short-column on silica gel using n-hexane/ethyl acetate as an eluent to give crude (5R)-5-{[4-bromo-5-(4-fluorophenyl)-3-methyl-1H-pyrazol-1-yl]methyl}dihydrofuran-2(3H)-one (490 mg). To a mixture of crude (5R)-5-{[4-bromo-5-(4-fluorophenyl)-3-methyl-1H-pyrazol-1-yl]methyl}dihydrofuran-2(3H)-one (490 mg) and 6-(4,4,5,5-tetramethyl-1,3,2-dioxaborolan-2-yl)-2H-1,4-benzoxazin-3(4H)-one (573 mg) in tetrahydrofuran (2 ml) were added 2M cesium carbonate aqueous solution (1.4 ml) and [1,1′-bis(diphenylphosphino)ferrocene]dichloropalladium(II) dichloromethane complex (227 mg). The reaction mixture was to refluxed for 20 minutes with microwave irradiation. The mixture was treated with water and filtered. The filtrate was extracted with ethyl acetate. The organic layer was washed with water and saturated brine, dried over anhydrous magnesium sulfate and concentrated in vacuo. The residue was purified by chromatography on silica gel using n-hexane/ethyl acetate as an eluent, and further purified by reverse phase HPLC eluted with water and acetonitrile to give the title compound as a white solid (3 mg).

¹H-NMR (300 MHz, CDCl₃) δ: 2.27 (3H, s), 2.27-2.54 (2H, m), 4.23-4.38 (2H, m), 4.39 (2H, m), 4.66 (2H, s), 4.89-5.05 (1H, m), 6.57 (1H, d, J=1.9 Hz), 6.76 (1H, m), 6.89-7.04 (3H, m), 7.29-7.40 (2H, m), 7.97 (1H, s).

### Example 159

### 6-[5-(4-Fluorophenyl)-3-methyl-1-{[(2S)-5-oxotetrahydrofuran-2-yl]methyl}-1H-pyrazol-4-yl]-2H-1,4-benzoxazin-3(4H)-one

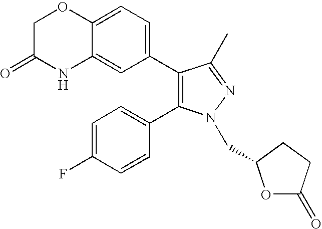

To a solution of 4-bromo-5-(4-fluorophenyl)-3-methyl-1H-pyrazole (1.00 g), (5S)-5-(hydroxymethyl)dihydrofuran-2(3H)-one (0.74 ml) and triphenylphosphine (2.57 g) in tetrahydrofuran (20 ml) was added slowly diisopropyl azodicarboxylate (40% in toluene, 5.2 ml) at 60° C. and the reaction mixture was stirred for 24 h at 60° C. The mixture was treated with water and extracted with ethyl acetate. Organic layer was washed with water and saturated brine, dried over anhydrous magnesium sulfate and concentrated in vacuo. The residue was washed with diisopropyl ether and filtered. The filtrate was concentrated, purified by chromatography on silica gel using hexane/ethyl acetate as an eluent, and further purified by reverse phase HPLC to give crude (5S)-5-{[4-bromo-5-(4-fluorophenyl)-3-methyl-1H-pyrazol-1-yl]methyl}dihydrofuran-2(3H)-one (130 mg). To a mixture of crude (5S)-5-{[4-bromo-5-(4-fluorophenyl)-3-methyl-1H-pyrazol-1-yl]methyl}dihydrofuran-2(3H)-one (130 mg) and 6-(4,4,5,5-tetramethyl-1,3,2-dioxaborolan-2-yl)-2H-1,4-benzoxazin-3(4H)-one (152 mg) in tetrahydrofuran (3 ml) were added 2M cesium carbonate solution (0.4 ml) and [1,1′-bis(diphenylphosphino)ferrocene]dichloropalladium(II) dichloromethane complex (80 mg). The reaction mixture was refluxed for 20 minutes with microwave irradiation, and further refluxed overnight under argon atmosphere. The mixture was treated with water and filtered. The filtrate was extracted with ethyl acetate and water. Organic layer was washed with water and saturated brine, dried over anhydrous magnesium sulfate and concentrated in vacuo. The residue was purified by chromatography on silica gel using n-hexane/ethyl acetate as an eluent to give the title compound as a white solid (25 mg).

¹H-NMR (300 MHz, CDCl₃) δ: 2.30(3H, s), 2.32-2.58 (3H, m), 4.08-4.22 (2H, m), 4.58 (2H, s), 4.89-5.03 (1H, m), 6.50 (1H, d, J=1.9 Hz) 6.67 (1H, dd, J=8.3, 1.9 Hz), 6.85 (1H, d, J=8.3 Hz), 7.02-7.13 (2H, m), 7.21-7.29 (3H, m), 8.43 (1H, s).

### Preparation 218

### (3,3-Difluorocyclopentyl)methanol

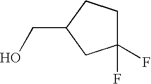

To a solution of 3-oxocyclopentanecarboxylic acid (5.00 g) in methanol (50 ml) was added dropwise thionyl chloride (3.1 ml) at 0° C., and the reaction mixture was stirred at 0° C. for 2 h. The mixture was treated with saturated sodium carbonate solution and extracted with ethyl acetate. Organic layer was washed with water and saturated brine, dried over anhydrous magnesium sulfate and concentrated in vacuo. The residue was dissolved in toluene (30 ml) and (diethylamino)sulfur trifluoride (6.2 ml) was added to the solution. The reaction mixture was stirred at room temperature for 2 h, treated with water and extracted with ethyl acetate. Organic layer was washed with water and saturated brine, dried over anhydrous magnesium sulfate and concentrated in vacuo. The residue was dissolved in tetrahydrofuran (40 ml) and added dropwise to a mixture of lithium aluminum hydride (1.48 g) in tetrahydrofuran (80 ml) at −78° C. The mixture was stirred at room temperature for 25 h, treated with water and extracted with ethyl acetate. Organic layer was washed with water and saturated brine, dried over anhydrous magnesium sulfate and concentrated in vacuo to give the title compound as yellow oil (1.23 g).

¹H-NMR (300 MHz, CDCl₃) δ: 1.43-1.73 (1H, m), 1.75-2.53 (6H, m), 3.60 (2H, d, J=6.4 Hz), 1H unconfirmed.

### Example 160

### 6-{1-[(3,3-Difluorocyclopentyl)methyl]-5-(4-fluorophenyl)-3-methyl-1H-pyrazol-4-yl}-2H-1,4-benzoxazin-3(4H)-one

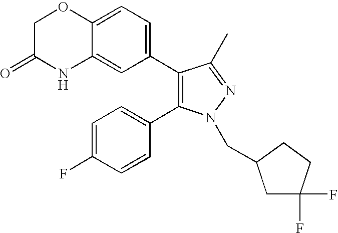

To a solution of 4-bromo-5-(4-fluorophenyl)-3-methyl-1H-pyrazole (1.54 g), (3,3-difluorocyclopentyl)methanol (1.23 g) and triphenylphosphine (3.95 g) in tetrahydrofuran (30 ml) was added slowly diisopropyl azodicarboxylate (40% in toluene, 7.9 ml) at 60° C. and the reaction mixture was stirred overnight at 60° C. The mixture was treated with water and extracted with ethyl acetate. Organic layer was washed with water and saturated brine, dried over anhydrous magnesium sulfate and concentrated in vacuo. The residue was treated with diisopropyl ether and filtered. The filtrate was concentrated and purified by chromatography on silica gel using hexane/ethyl acetate as an eluent to give crude 4-Bromo-1-[(3,3-difluorocyclopentyl)methyl]-5-(4-fluorophenyl)-3-methyl-1H-pyrazole (510 mg). To a mixture of crude 4-Bromo-1-[(3,3-difluorocyclopentyl)methyl]-5-(4-fluorophenyl)-3-methyl-1H-pyrazole (510 mg) and 6-(4,4,5,5-tetramethyl-1,3,2-dioxaborolan-2-yl)-2H-1,4-benzoxazin-3(4H)-one (564 mg) in tetrahydrofuran (3 ml) were added 2M cesium carbonate solution (1.4 ml) and [1,1′-bis(diphenylphosphino)ferrocene]dichloropalladium(II) dichloromethane complex (224 mg). The reaction mixture was refluxed for 18 h under argon atmosphere. The mixture was treated with water and filtered. The filtrate was extracted with ethyl acetate. Organic layer was washed with water and saturated brine, dried over anhydrous magnesium sulfate and concentrated in vacuo. The residue was purified by chromatography on silica gel using hexane/ethyl acetate as an eluent to give the title compound as a white solid (223 mg).

¹H-NMR (300 MHz, DMSO-d₆) δ: 1.28-1.42 (1H, m), 1.65-1.83 (2H, m), 1.90-2.13 (3H, m), 2.18 (3H, s), 2.53-2.58 (1H, m), 3.92 (2H, J=7.2 Hz), 4.53 (2H, s), 6.56 (1H, dd, J=8.3, 1.9 Hz), 6.65 (1H, d, J=1.9 Hz), 6.81 (1H, d, J=8.3 Hz), 7.20-7.45 (4H, m), 10.59 (1H, s).

### Example 161

### 1,1,1-Trifluoro-N-{2-[5-(4-fluorophenyl)-3-methyl-4-(3-oxo-3,4-dihydro-2H-1,4-benzoxazin-6-yl)-1H-pyrazol-1-yl]ethyl}methanesulfonamide

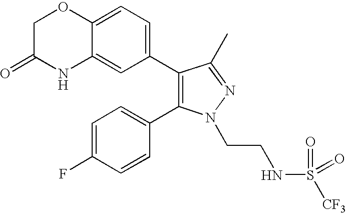

### Preparation 219

### 1-(4-Chlorophenyl)butane-1,3-dione

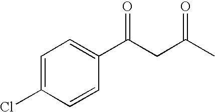

To a stirred suspension of 60% sodium hydride (6.2 g) in THF (150 mL) was added dropwise 4-chlorophenylacetone (20 g) with ice-cooling. The mixture was stirred for 1 h, and then ethyl acetate (38.4 mL) was added dropwise. The mixture was stirred at room temperature for 12 h and 6N HCl (30 mL) was added with ice-cooling. The mixture was stirred for 5 min, evaporated, treated with water, and extracted with ethyl acetate. The organic layer was dried over MgSO₄ and concentrated in vacuo. The residue was crystallized from hexane to give the title compound (22 g). This product was used for the next reaction without further purification.

### Preparation 220

### 5-(4-Chlorophenyl)-1,3-dimethyl-1H-pyrazole

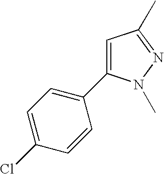

To a stirred mixture of 1-(4-chlorophenyl)butane-1,3-dione (15 g), 13N HCl solution (20 mL) and methanol (130 mL) was added dropwise methyl hydrazine (5.3 g) with ice-cooling. The mixture was stirred for 3 days, evaporated, treated with aqueous NaHCO₃ solution and extracted with ethyl acetate. The organic layer was dried over MgSO₄ and concentrated in vacuo. The residue was chromatographed on silica gel eluting with hexane/ethyl acetate to give the title compound (3.6 g).

¹H-NMR (300 MHz, DMSO-d₆) δ: 2.16 (3H, s), 3.75 (3H, s), 6.20 (1H, s), 7.48-7.61 (4H, m)

### Preparation 221

### 4-Bromo-5-(4-chlorophenyl)-1,3-dimethyl-1H-pyrazole

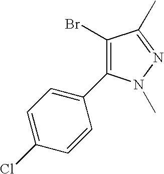

To a stirred solution of 5-(4-chlorophenyl)-1,3-dimethyl-1H-pyrazole (3.6 g) in acetonitrile (40 mL) was added N-bromosuccinimide (NBS) (4.7 g) with ice-cooling. The mixture was stirred for 1 h, treated with water and extracted with ethyl acetate. The organic layer was dried over MgSO₄ and concentrated in vacuo. The residue was chromatographed on silica gel eluting with hexane/ethyl acetate to give the title compound (4.0 g).

¹H-NMR (300 MHz, DMSO-d₆) δ: 2.17 (3H, s), 3.70 (3H, s), 7.44-7.56 (2H, m), 7.57-7.68 (2H, m).

### Preparation 222

### 1-(4-Methylphenyl)butane-1,3-dione

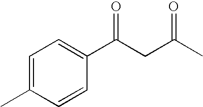

To a stirred suspension of 60% sodium hydride (3.6 g) in THF (75 mL) was added dropwise 1-(4-methylphenyl)ethanone (10 g) with ice-cooling. The mixture was stirred for 1 h, and then ethyl acetate (22 mL) was added dropwise. The mixture was stirred at room temperature for 12 h and 6N HCl (15 mL) was added with ice-cooling. The mixture was stirred for 5 min, evaporated, treated with water, and extracted with ethyl acetate. The organic layer was dried over MgSO₄ and concentrated in vacuo to give the title compound (13 g). This product was used for the next reaction without further purification.

### Preparation 223

### 1,3-Dimethyl-5-(4-methylphenyl)-1H-pyrazole

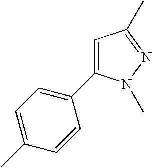

To a stirred mixture of 1-(4-methylphenyl)butane-1,3-dione (13 g), 13N HCl solution (20 mL) and methanol (120 mL) was added dropwise methyl hydrazine (5.2 g) with ice-cooling. The mixture was stirred for 16 h, evaporated, treated with aqueous NaHCO₃ solution and extracted with ethyl acetate. The organic layer was dried over MgSO₄ and concentrated in vacuo. The residue was chromatographed on silica gel eluting with hexane/ethyl acetate to give the title compound (5.9 g).

¹H-NMR (300 MHz, DMSO-d₆) δ: 2.15 (3H, s), 2.35 (3H, s), 3.73 (3H, s), 6.12 (1H, s), 7.24-7.32 (2H, m), 7.32-7.42 (2H, m). LCMS (ESI⁺) M+H⁺: 187.

### Preparation 224

### 4-Bromo-1,3-dimethyl-5-(4-methylphenyl)-1H-pyrazole

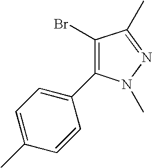

To a stirred solution of 1,3-dimethyl-5-(4-methylphenyl)-1H-pyrazole (500 mg) in acetonitrile (5 mL) was added NBS (5.0 g) with ice-cooling. The mixture was stirred for 1 h, treated with water and extracted with ethyl acetate. The organic layer was dried over MgSO₄ and concentrated in vacuo. The residue was chromatographed on silica gel eluting with hexane/ethyl acetate to give the title compound (544 mg).

¹H-NMR (300 MHz, DMSO-d₆) δ: 2.16 (3H, s), 2.38 (3H, s), 3.68 (3H, s), 7.35 (4H, s). LCMS (ESI⁺) M+H⁺: 265, M+3H⁺: 267.

### Preparation 225

### 5-(4-fluorophenyl)-3-methyl-1-[(2-methyloxiran-2-yl)methyl]-1H-pyrazole

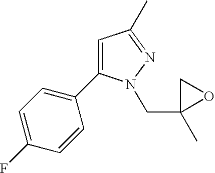

To a stirred suspension of trimethylsulfonyl iodide (3.38 g) in DMSO (30 mL) was added portionwise 60% sodium hydride (563 mg) at room temperature. After stirring for 30 min, a solution of 1-[5-(4-fluorophenyl)-3-methyl-1H-pyrazol-1-yl]propan-2-one (3 g) in DMSO (30 mL) was added. The mixture was stirred for 2 h, treated with aqueous NH₄Cl solution and extracted with ethyl acetate. The organic layer was washed with water, dried over MgSO₄ and concentrated in vacuo. The residue was chromatographed on silica gel eluting with hexane/ethyl acetate to give the title compound (2.2 g).

¹H-NMR (300 MHz, DMSO-d₆) δ: 1.12 (3H, s), 2.19 (3H, s), 2.44 (1H, d, J=5.3 Hz), 2.55 (1H, d, J=5.3 Hz), 4.08-4.18 (2H, m), 6.16 (1H, s), 7.27-7.38 (2H, m), 7.45-7.64 (2H, m). LCMS (ESI⁺) M+H⁺: 247.

### Preparation 226

### 2-Fluoro-3-[5-(4-fluorophenyl)-3-methyl-1H-pyrazol-1-yl]-2-methylpropan-1-ol

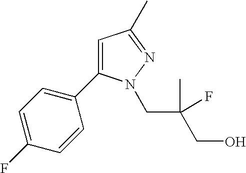

To a dry polypropylene flask was added Olah's reagent (10 mL). A solution of 5-(4-fluorophenyl)-3-methyl-1-[(2-methyloxiran-2-yl)methyl]-1H-pyrazole (2.2 g) in toluene (15 mL) was added with ice-cooling. The mixture was stirred at room temperature for 12 h, poured into cold aqueous NaHCO₃ solution, and extracted with ethyl acetate. The organic layer was dried over MgSO₄ and concentrated in vacuo. The residue was chromatographed on silica gel eluting with hexane/ethyl acetate to give the title compound (2.0 g).

¹H-NMR (300 MHz, DMSO-d₆) δ: 1.11 (3H, d, J=21.0 Hz), 2.19 (3H, s), 3.32-3.39 (1H, m), 3.39-3.46 (1H, m), 4.08-4.40 (2H, m), 5.04 (1H, t, J=5.9 Hz), 6.16 (1H, s), 7.24-7.35 (2H, m), 7.45-7.54 (2H, m). LCMS (ESI⁺) M+H⁺: 267.

### Preparation 227

### 3-[4-Bromo-5-(4-fluorophenyl)-3-methyl-1H-pyrazol-1-yl]-2-fluoro-2-methylpropan-1-ol

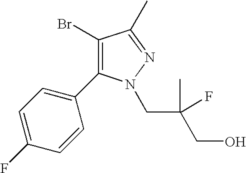

To a stirred solution of 2-fluoro-3-[5-(4-fluorophenyl)-3-methyl-1H-pyrazol-1-yl]-2-methylpropan-1-ol (2 g) in acetonitrile (20 mL) was added NBS (1.35 g) with ice-cooling. The mixture was stirred for 1 h, treated with water and extracted with ethyl acetate. The organic layer was dried over MgSO₄ and concentrated in vacuo. The residue was chromatographed on silica gel eluting with hexane/ethyl acetate to give the title compound (2.59 g).

¹H-NMR (300 MHz, DMSO-d₆) δ: 1.08 (3H, d, J=22.0 Hz), 2.20 (3H, s), 3.25-3.42 (2H, m), 4.06-4.38 (2H, m), 5.03 (1H, t, J=5.9 Hz), 7.31-7.41 (2H, m), 7.43-7.51 (2H, m).

LCMS (ESI⁺) M+H⁺: 345, M+3H⁺: 347.

### Preparation 228

### 5-(4-Fluorophenyl)-3-methyl-1-[4-(methylsulfanyl)butyl]-1H-pyrazole

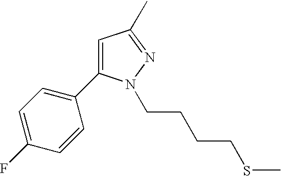

To a stirred solution of 1-[4-(benzyloxy)butyl]-5-(4-fluorophenyl)-3-methyl-1H-pyrazole (1.8 g) and 20% palladium hydroxide on carbon (0.54 g) in ethanol/acetic acid (18 mL/3 mL) was added portionwise ammonium formate (1.3 g) at 80° C. The mixture was stirred at 80° C. for 1 h, filtered, evaporated to give 4-[5-(4-fluorophenyl)-3-methyl-1H-pyrazol-1-yl]butan-1-ol.

The alcohol obtained was dissolved in pyridine (10 mL) and then, 4-methylbenzenesulfonyl chloride (1.1 g) was added at room temperature. The mixture was stirred for 5 h, treated with 1N HCl solution, and extracted with ethyl acetate. The organic layer was dried over MgSO₄ and concentrated in vacuo. The residue was dissolved in ethanol (10 mL) and sodium methanethiolate (559 mg) was added. The mixture was stirred at room temperature for 3 days, treated with water and extracted with ethyl acetate. The organic layer was dried over MgSO₄ and concentrated in vacuo. The residue was chromatographed on silica gel eluting with hexane/ethyl acetate to give the title compound (315 mg).

¹H-NMR (300 MHz, DMSO-d₆) δ: 1.30-1.46 (2H, m), 1.64-1.81 (2H, m), 1.95 (3H, s), 2.17 (3H, s), 2.33 (2H, t, J=7.4 Hz), 4.00 (2H, t, J=7.0 Hz), 6.12 (1H, s), 7.28-7.38 (2H, m), 7.40-7.55 (2H, m). LCMS (ESI⁺) M+H⁺: 279.

### Preparation 229

### 4-Bromo-5-(4-fluorophenyl)-3-methyl-1-[4-(methylsulfonyl)butyl]-1H-pyrazole

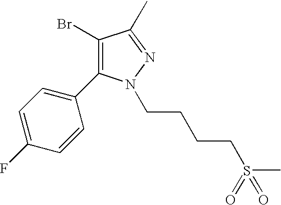

To a stirred solution of 5-(4-fluorophenyl)-3-methyl-1-[4-(methylsulfanyl)butyl]-1H-pyrazole (315 mg) in acetonitrile (5 mL) was added NBS (204 mg) with ice-cooling. After stirring for 1 h, NBS (204 mg) was added again. The mixture was stirred for 30 min and evaporated. The residue was dissolved in DMF (5 mL) and m-chlorobenzoic acid (mCPBA) (214 mg) was added. After stirring for 1 h, mCPBA (214 mg) was added again. The mixture was stirred for 30 min, treated with water and extracted with ethyl acetate. The organic layer was dried over MgSO₄ and concentrated in vacuo. The residue was chromatographed on silica gel eluting with hexane/ethyl acetate to give the title compound (385 mg).

The product obtained was used for the next reaction without further purification.

### Preparation 230

### N,N-Dibenzyl-2-[4-bromo-5-(4-fluorophenyl)-3-methyl-1H-pyrazol-1-yl]ethanesulfonamide

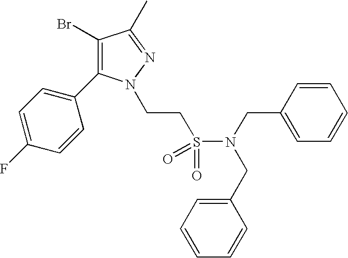

A mixture of 2-[4-bromo-5-(4-fluorophenyl)-3-methyl-1H-pyrazol-1-yl]ethanesulfonic acid (3 g) and thionyl chloride (30 mL) was stirred at reflux for 16 h and then concentrated in vacuo. The residue was dissolved in THF (40 mL). This sulfonyl chloride solution was added dropwise to a stirred solution of dibenzylamine (16 g) in THF (60 mL) with ice-cooling. The mixture was stirred at room temperature for 3 h. The white precipitate was filtered off and washed with ethyl acetate. The filtrate was concentrated in vacuo, treated with 1N HCl solution and extracted with ethyl acetate. The organic layer was washed with 1N HCl solution and brine, dried over MgSO₄ and concentrated in vacuo. The residue was purified by preparative HPLC to give the title compound (2.3 g).

¹H-NMR (300 MHz, DMSO-d₆) δ: 2.21 (3H, s), 3.62 (2H, t, J=7.0 Hz), 4.21 (4H, s), 4.24-4.37 (2H, m), 7.07-7.20 (4H, m), 7.19-7.33 (6H, m), 7.36-7.47 (2H, m), 7.47-7.60 (2H, m).

### Preparation 231

### 2-[4-Bromo-5-(4-fluorophenyl)-3-methyl-1H-pyrazol-1-yl]ethanesulfonamide

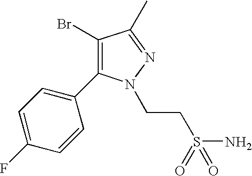

A mixture of N,N-dibenzyl-2-[4-bromo-5-(4-fluorophenyl)-3-methyl-1H-pyrazol-1-yl]ethanesulfonamide (700 mg) and H₂SO₄ (7 mL) was stirred at room temperature for 5 min, and ethanol (5 mL) was added. The mixture was stirred for 1 h, diluted with ethyl acetate and treated with 1N NaOH solution. The organic layer was separated and the aqueous layer was extracted with ethyl acetate. The combined organic layer was dried over MgSO₄ and concentrated in vacuo. The residue was chromatographed on silica gel eluting with hexane/ethyl acetate to give the title compound (425 mg).

¹H-NMR (300 MHz, DMSO-d₆) δ: 2.20 (3H, s), 3.39-3.54 (2H, m), 4.22-4.35 (2H, m), 6.99 (2H, s), 7.34-7.48 (2H, m), 7.50-7.65 (2H, m).

### Preparation 232

### 1-(3-Azidopropyl)-4-bromo-5-(4-fluorophenyl)-3-methyl-1H-pyrazole

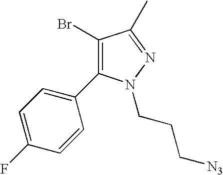

To a stirred mixture of 3-[4-bromo-5-(4-fluorophenyl)-3-methyl-1H-pyrazol-1-yl]propan-1-ol (2.0 g) and triethylamine (1.8 mL) in THF (20 mL) was added methanesulfonyl chloride (0.77 g) with ice-cooling. The mixture was stirred for 1 h, evaporated, treated with water and extracted with ethyl acetate. The organic layer was dried over MgSO₄ and concentrated in vacuo. The residue was dissolved in DMF (20 mL) and sodium azido (0.54 g) was added. The mixture was stirred at 80° C. for 3 h, treated with water and extracted with ethyl acetate. The organic layer was dried over MgSO₄ and concentrated in vacuo. The residue was chromatographed on silica gel eluting with ethyl acetate/hexane to give the titled compound (1.48 g).

¹H-NMR (300 MHz, DMSO-d₆) δ: 1.78-1.93 (2H, m), 2.19 (3H, s), 3.26 (2H, t, J=6.4 Hz), 4.00 (2H, t, J=6.8 Hz), 7.34-7.45 (2H, m), 7.46-7.57 (2H, m).

LCMS (ESI⁺) M+H⁺: 338, M+3H⁺: 340.

### Preparation 233

### tert-Butyl{3-[4-bromo-5-(4-fluorophenyl)-3-methyl-1H-pyrazol-1-yl]propyl}carbamate

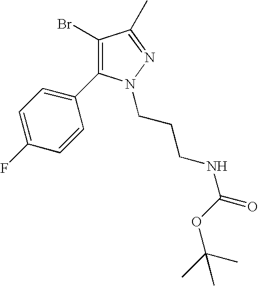

To a stirred solution of 1-(3-azidopropyl)-4-bromo-5-(4-fluorophenyl)-3-methyl-1H-pyrazole (1.40 g) in THF (15 mL) was added triphenylphosphine (1.63 g) at room temperature. The mixture was stirred for 12 h. After addition of water (1 mL), the mixture was stirred at 50° C. for 12 h, treated with water and extracted with ethyl acetate. The organic layer was dried over MgSO₄ and concentrated in vacuo. The residue was dissolved in THF (15 mL) and di-tert-butyl dicarbonate (1.36 g) was added. The mixture was stirred for 12 h, treated with water and extracted with ethyl acetate. The organic layer was dried over MgSO₄ and concentrated in vacuo. The residue was chromatographed on silica gel eluting with hexane/ethyl acetate to give the title compound (1.33 g).

¹H-NMR (300 MHz, DMSO-d₆) δ: 1.32 (9H, s), 1.68-1.81 (2H, m), 2.18 (3H, s), 2.75-2.85 (2H, m), 3.91 (2H, t, J=7.4 Hz), 6.73 (1H, t, J=5.3 Hz), 7.30-7.41 (2H, m), 7.42-7.53 (2H, m). LCMS (ESI⁺) M+H⁺: 412, M+3H⁺: 414.

### Example 162

### 6-[5-(4-Chlorophenyl)-1,3-dimethyl-1H-pyrazol-4-yl]-2H-1,4-benzoxazin-3 (4H) -one

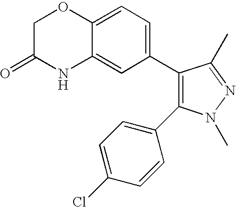

To a mixture of 6-(4,4,5,5-tetramethyl-1,3,2-dioxaborolan-2-yl)-2H-1,4-benzoxazin-3(4H)-one (626 mg), 4-bromo-5-(4-chlorophenyl)-1,3-dimethyl-1H-pyrazole (500 mg), and [1,1-bis(diphenylphosphino)ferrocene]dichloro palladium(II) dichloromethane adduct (286 mg) in THF (10 mL) was added a solution of cesium carbonate (1.4 g) in water (2.5 mL). The mixture was stirred at reflux for 16 h, treated with water and extracted with ethyl acetate. The organic layer was dried over MgSO₄, and concentrated in vacuo. The residue was chromatographed on silica gel eluting with ethyl acetate/hexane. Crystallization from ethyl acetate/hexane gave the titled compound (225 mg).

¹H-NMR (300 MHz, DMSO-d₆) δ: 2.17 (3H, s), 3.67 (3H, s), 4.54 (2H, s), 6.58 (1H, dd, J=8.0, 1.9 Hz), 6.65 (1H, d, J=1.9 Hz), 6.84 (1H, d, J=8.0 Hz), 7.20-7.38 (2H, m), 7.44-7.51 (2H, m), 10.59 (1H, s).

LCMS (ESI⁺) M+H⁺: 354, M+3H⁺: 356.

Anal. Calcd for C19H16N3O2C1: C, 64.5; H, 4.56; N, 11.88. Found: C, 64.48; H, 4.65; N, 11.75.

### Example 163

### 6-(1,3-Dimethyl-5-phenyl-1H-pyrazol-4-yl)-2H-1,4-benzoxazin-3(4H)-one

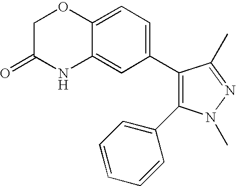

A mixture of 6-[5-(4-chlorophenyl)-1,3-dimethyl-1H-pyrazol-4-yl]-2H-1,4-benzoxazin-3(4H)-one (125 mg), 20 wt % palladium hydroxide on carbon (37.5 mg) and ammonium formate (89 mg) in ethanol/acetic acid (3 mL/0.5 mL) was stirred at 80° C. for 1 h, filtered, treated with water and extracted with ethyl acetate. The organic layer was dried over MgSO₄ and concentrated in vacuo. Crystallization from ethyl acetate/hexane gave the titled compound (85 mg).

¹H-NMR (300 MHz, DMSO-d₆) δ: 2.16 (3H, s), 3.64 (3H, s), 4.53 (2H, s), 6.55 (1H, dd, J=8.3, 2.0 Hz), 6.68 (1H, d, J=2.0 Hz), 6.81 (1H, d, J=8.3 Hz), 7.21-7.31 (2H, m), 7.34-7.49 (3H, m), 10.58 (1H, s).

LCMS (ESI⁺) M+H⁺: 320.

Anal. Calcd for C19H17N3O2: C, 71.46; H, 5.37; N, 13.16. Found: C, 71.39; H, 5.4; N, 13.13.

### Example 164

### 6-[1,3-Dimethyl-5-(4-methylphenyl)-1H-pyrazol-4-yl]-2H-1,4-benzoxazin-3(4H)-one

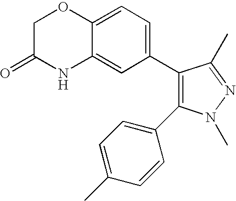

A mixture of 6-(4,4,5,5-tetramethyl-1,3,2-dioxaborolan-2-yl)-2H-1,4-benzoxazin-3(4H)-one (677 mg), 4-bromo-1,3-dimethyl-5-(4-methylphenyl)-1H-pyrazole (544 mg), [1,1-bis(diphenylphosphino)ferrocene]dichloro palladium(II) dichloromethane adduct (335 mg), and cesium carbonate (1.67 g) in THF/water (10/3 mL) was exposed to microwave irradiation at 150° C. for 1 h, treated with water and extracted with ethyl acetate. The organic layer was dried over MgSO₄, and concentrated in vacuo. The residue was chromatographed on silica gel eluting with ethyl acetate/hexane. Crystallization from ethyl acetate/hexane gave the titled compound (130 mg).

¹H-NMR (300 MHz, DMSO-d₆) δ: 2.15 (3H, s), 2.33 (3H, s), 3.63 (3H, s), 4.53 (2H, s), 6.56 (1H, dd, J=8.1, 2.1 Hz), 6.68 (1H, d, J=2.1 Hz), 6.81 (1H, d, J=8.1 Hz), 7.10-7.19 (2H, m), 7.19-7.28 (2H, m), 10.58 (1H, s).

LCMS (ESI⁺) M+H⁺: 334.

Anal. Calcd for C20H19N3O2: C, 72.05; H, 5.74; N, 12.6. Found: C, 71.77; H, 5.8; N, 12.5.

### Example 165

### 6-[1-(2-Fluoro-3-hydroxy-2-methylpropyl)-5-(4-fluorophenyl)-3-methyl-1H-pyrazol-4-yl]-2H-1,4-benzoxazin-3(4H)-one

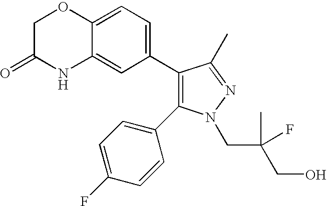

A mixture of 6-(4,4,5,5-tetramethyl-1,3,2-dioxaborolan-2-yl)-2H-1,4-benzoxazin-3(4H)-one (574 mg), 3-[4-bromo-5-(4-fluorophenyl)-3-methyl-1H-pyrazol-1-yl]-2-fluoro-2-methylpropan-1-ol (600 mg), palladium acetate (11.7 mg), 30 Xphos (49.6 mg) and cesium carbonate (1.67 g) in THF/water (8.8/2.2 mL) was exposed to microwave irradiation at 150° C. for 30 min., treated with water and extracted with ethyl acetate. The organic layer was dried over MgSO₄, and concentrated in vacuo. The residue was chromatographed on silica gel eluting with ethyl acetate/hexane. Crystallization from ethyl acetate/hexane gave the titled compound (341 mg).

¹H-NMR (300 MHz, DMSO-d₆) δ: 1.13 (3H, d, J=22.2 Hz), 2.20 (3H, s), 3.34-3.47 (2H, m), 4.00-4.34 (2H, m), 4.53 (2H, s) 5.02 (1H, t, J=5.8 Hz), 6.56 (1H, dd, J=8.3, 1.9 Hz), 6.64 (1H, d, J=1.9 Hz), 6.81 (1H, d, J=8.3 Hz), 7.19-7.35 (4H, m), 10.59 (1H, s).

LCMS (ESI⁺) M+H⁺: 414.

Anal. Calcd for C22H21N3O3F2: C, 63.91; H, 5.12; N, 10.16. Found: C, 63.86; H, 5.03; N, 10.18

### Example 166

### 6-{5-(4-Fluorophenyl)-3-methyl-1-[4-(methylsulfonyl)butyl]-1H-pyrazol-4-yl}-2H-1,4-benzoxazin-3(4H)-one

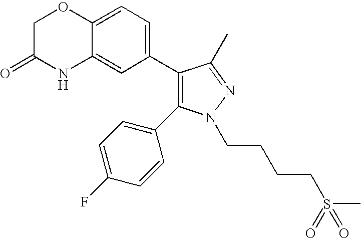

A mixture of 6-(4,4,5,5-tetramethyl-1,3,2-dioxaborolan-2-yl)-2H-1,4-benzoxazin-3(4H)-one (354 mg), 4-bromo-5-(4-fluorophenyl)-3-methyl-1-[4-(methylsulfonyl)butyl]-1H-pyrazole (385 mg), palladium acetate (44.4 mg), Xphos (189 mg) and cesium carbonate (0.81 g) in THF/water (12/3 mL) was exposed to microwave irradiation at 150° C. for 30 min., treated with water and extracted with ethyl acetate. The organic layer was dried over MgSO₄, and concentrated in vacuo. The residue was chromatographed on silica gel eluting with ethyl acetate/hexane. Crystallization from ethyl acetate/hexane gave the titled compound (280 mg).

¹H-NMR (300 MHz, DMSO-d₆) δ: 1.50-1.67 (2H, m), 1.69-1.84 (2H, m), 2.19 (3H, s), 2.90 (3H, s), 2.96-3.09 (2H, m), 3.92 (2H, t, J=7.0 Hz), 4.53 (2H, s), 6.57 (1H, dd, J=8.3, 2.0 Hz), 6.65 (1H, d, J=2.0 Hz), 6.81 (1H, d, J=8.3 Hz), 7.23-7.40 (4H, m), 10.60 (1H, s).

LCMS (ESI⁺) M+H⁺: 458.

Anal. Calcd for C23H24N3O4SF: C, 60.38; H, 5.29; N, 9.18. Found: C, 60.27; H, 5.29; N, 9.06.

### Example 167

### 2-[5-(4-Fluorophenyl)-3-methyl-4-(3-oxo-3,4-dihydro-2H-1,4-benzoxazin-6-yl)-1H-pyrazol-1-yl]ethanesulfonamide

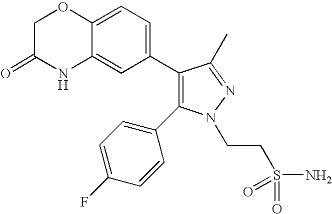

To a mixture of 6-(4,4,5,5-tetramethyl-1,3,2-dioxaborolan-2-yl)-2H-1,4-benzoxazin-3(4H)-one (273 mg), 2-[4-bromo-5-(4-fluorophenyl)-3-methyl-1H-pyrazol-1-yl]ethanesulfonamide (300 mg), palladium acetate (18.6 mg) and Xphos (78.9 mg) in THF (6 mL) was added a solution of cesium carbonate (0.68 g) in water (1.5 mL). The mixture was stirred at reflux for 3 days, treated with water and extracted with ethyl acetate. The organic layer was dried over MgSO₄, and concentrated in vacuo. The residue was chromatographed on silica gel eluting with ethyl acetate/hexane. Crystallization from ethyl acetate/hexane gave the titled compound (264 mg).

¹H-NMR (300 MHz, DMSO-d₆) δ: 2.20 (3H, s), 3.41-3.55 (2H, m), 4.13-4.31 (2H, m), 4.53 (2H, s), 6.58 (1H, dd, J=8.1, 2.0 Hz), 6.65 (1H, d, J=2.0 Hz), 6.83 (1H, d, J=8.1 Hz), 6.99 (2H, br. s.), 7.21-7.48 (4H, m), 10.61 (1H, br. s.).

LCMS (ESI⁺) M+H⁺: 431.

Anal. Calcd for C20H19N4O4SF.0.2AcOEt: C, 55.76; H, 4.63; N, 12.50. Found: C, 55.77; H, 4.64; N, 12.50.

### Example 168

### 2,2-Difluoro-3-[5-(4-fluorophenyl)-3-methyl-4-(3-oxo-3,4-dihydro-2H-1,4-benzoxazin-6-yl)-1H-pyrazol-1-yl]propyl ethylcarbamate

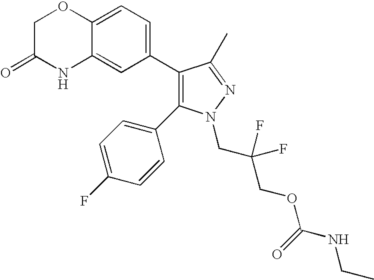

To a stirred suspension of triphosgene (2.3 g) in acetonitrile (10 mL) was added dropwise triethylamine (1.1 mL) with ice-cooling. After stirring for 5 min, 6-[1-(2,2-difluoro-3-hydroxypropyl)-5-(4-fluorophenyl)-3-methyl-1H-pyrazol-4-yl]-2H-1,4-benzoxazin-3(4H)-one (328 mg) was added portionwise to the mixture. After stirring for 1 h, ethylamine in THF (2M, 15 mL) was added to the mixture. The mixture was stirred for 3 days, treated with water, and extracted with ethyl acetate. The organic layer was washed with brine, dried over MgSO₄, and concentrated in vacuo. The residue was purified by preparative HPLC. Crystallization from ethyl acetate/hexane gave the titled compound (52 mg).

¹H-NMR (300 MHz, DMSO-d₆) δ: 1.00 (3H, t, J=7.2 Hz), 2.20 (3H, s), 2.93-3.06 (2H, m), 4.23-4.36 (2H, m), 4.39-4.52 (2H, m), 4.54 (2H, s), 6.58 (1H, dd, J=8.1, 2.0 Hz), 6.66 (1H, d, J=2.0 Hz), 6.82 (1H, d, J=8.1 Hz), 7.24-7.31 (4H, m), 7.39 (1H, t, J=5.7 Hz), 10.61 (1H, s).

LCMS (ESI⁺) M+H⁺: 489.

Anal. Calcd for C24H23N4O4F3: C, 59.01; H, 4.75; N, 11.47. Found: C, 58.83; H, 4.88; N, 11.44.

### Example 169

### 2,2-Difluoro-3-[5-(4-fluorophenyl)-3-methyl-4-(3-oxo-3,4-dihydro-2H-1,4-benzoxazin-6-yl)-1H-pyrazol-1-yl]propyl (2-hydroxyethyl)carbamate

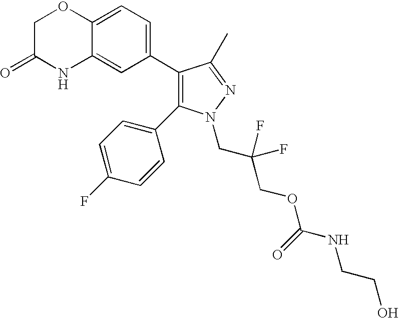

To a stirred suspension of triphosgene (2.84 g) in acetonitrile (16 mL) was added dropwise triethylamine (1.33 mL) with ice-cooling. After stirring for 5 min, 6-[1-(2,2-difluoro-3-hydroxypropyl)-5-(4-fluorophenyl)-3-methyl-1H-pyrazol-4-yl]-2H-1,4-benzoxazin-3(4H)-one (400 mg) was added portionwise to the mixture. After stirring for 1 h, hydoxyethylamine (2.9 g) was added to the mixture. The mixture was stirred for 3 days, treated with water, and extracted with ethyl acetate. The organic layer was washed with brine, dried over MgSO₄, and concentrated in vacuo. The residue was purified by preparative HPLC. Crystallization from ethyl acetate/hexane gave the titled compound (80 mg).

¹H-NMR (300 MHz, DMSO-d₆) δ: 2.20 (3H, s), 2.97-3.07 (2H, m), 3.34-3.42 (2H, m), 4.30 (2H, t, J=14.4 Hz), 4.47 (2H, t, J=13.4 Hz), 4.53 (2H, s), 4.63 (1H, t, J=5.5 Hz), 6.59 (1H, dd, J=8.1, 1.9 Hz), 6.66 (1H, d, J=1.9 Hz), 6.82 (1H, d, J=8.1 Hz), 7.24-7.32 (4H, m), 7.34 (1H, t, J=5.7 Hz), 10.60 (1H, s).

LCMS (ESI⁺) M+H⁺: 505.

Anal. Calcd for C24H23N4O5F3.0.1H2O.0.2acetone.0.1AcOEt): C, 57.01; H, 4.82; N, 10.64. Found: C, 57.06; H, 5.01; N, 10.53.

### Example 170

### tert-Butyl{3-[5-(4-fluorophenyl)-3-methyl-4-(3-oxo-3,4-dihydro-2H-1,4-benzoxazin-6-yl)-1H-pyrazol-1-yl]propyl}carbamate

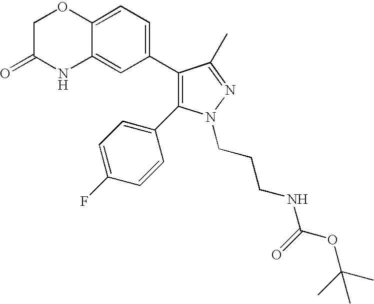

To a mixture of 6-(4,4,5,5-tetramethyl-1,3,2-dioxaborolan-2-yl)-2H-1,4-benzoxazin-3(4H)-one (1.11 g), tert-Butyl {3-[4-bromo-5-(4-fluorophenyl)-3-methyl-1H-pyrazol-1-yl]propyl}carbamate (1.33 g), palladium acetate (36.2 mg) and Xphos (153.8 mg) in THF (16 mL) was added a solution of cesium carbonate (2.63 g) in water (4 mL). The mixture was stirred at reflux for 24 h, treated with water and extracted with ethyl acetate. The organic layer was dried over MgSO₄, and concentrated in vacuo. The residue was chromatographed on silica gel eluting with ethyl acetate/hexane to give the titled compound (1.16 g) as an amorphous solid.

¹H-NMR (300 MHz, DMSO-d₆) δ: 1.33 (9H, s), 1.67-1.87 (2H, m), 2.19 (3H, s), 2.76-2.93 (2H, m), 3.86 (2H, t, J=7.2 Hz), 4.53 (2H, s), 6.56 (1H, dd, J=8.0, 1.9 Hz), 6.65 (1H, d, J=1.9 Hz), 6.75 (1H, t, J=5.3 Hz), 6.81 (1H, d, J=8.0 Hz), 7.19-7.37 (4H, m), 10.60 (1H, s).

LCMS (ESI⁺) M+H⁺: 481.

Anal. Calcd for C26H29N4O4F: C, 64.99; H, 6.08; N, 11.66. Found: C, 64.96; H, 6.07; N, 11.5.

### Example 171

### 6-[1-(3-Aminopropyl)-5-(4-fluorophenyl)-3-methyl-1H-pyrazol-4-yl]-2H-1,4-benzoxazin-3(4H)-one

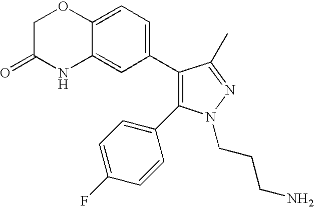

To a mixture of tert-butyl {3-[5-(4-fluorophenyl)-3-methyl-4-(3-oxo-3,4-dihydro-2H-1,4-benzoxazin-6-yl)-1H-pyrazol-1-yl]propyl}carbamate (1 g) in ethanol (8 mL) was added dropwise 4N HCl in ethyl acetate (2.1 mL) at room temperature. The mixture was stirred for 12 h, evaporated, treated with aqueous NaHCO₃ solution, and extracted with ethyl acetate. The organic layer was washed with brine, dried over MgSO₄, and concentrated in vacuo. The residue was purified by preparative HPLC and the crystals were suspended in ethyl acetate/diisopropyl ether and collected by filtration to give the titled compound (500 mg).

¹H-NMR (300 MHz, DMSO-d₆) δ: 1.69-1.87 (m, 2H), 2.19 (s, 3H), 2.53-2.63 (m, 2H), 3.95 (t, J=7.0 Hz, 2H), 4.53 (s, 2H), 6.56 (dd, J=8.3, 1.9 Hz, 1H), 6.65 (d, J=1.9 Hz, 1H), 6.82 (d, J=8.3 Hz, 1H), 7.20-7.36 (m, 4H), 3H was unconfirmed. LCMS (ESI⁺) M+H⁺: 381.

### Example 172

### N-{3-[5-(4-Fluorophenyl)-3-methyl-4-(3-oxo-3,4-dihydro-2H-1,4-benzoxazin-6-yl)-1H-pyrazol-1-yl]propyl}acetamide

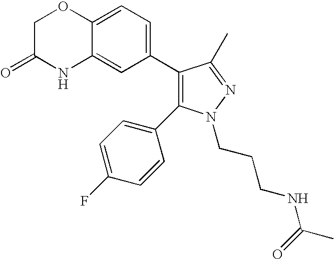

To a stirred mixture of 6-[1-(3-aminopropyl)-5-(4-fluorophenyl)-3-methyl-1H-pyrazol-4-yl]-2H-1,4-benzoxazin-3(4H)-one (200 mg) and triethylamine (0.14 mL) in ethyl acetate/THF/H₂O (4 mL/1 mL/4 mL) was added acetyl chloride (41.3 mg) with ice-cooling. The mixture was stirred for 12 h, treated with water, and extracted with ethyl acetate. The organic layer was washed with brine, dried over MgSO₄, and concentrated in vacuo. The residue was purified by preparative HPLC and crystallization from ethyl acetate/hexane gave the titled compound (136 mg).

¹H-NMR (300 MHz, DMSO-d₆) δ: 1.71 (3H, s), 1.74-1.87 (2H, m), 2.19 (3H, s), 2.81-2.99 (2H, m), 3.87 (2H, t, J=7.2 Hz), 4.53 (2H, s), 6.56 (1H, dd, J=8.3, 1.9 Hz), 6.65 (1H, d, J=1.9 Hz), 6.81 (1H, d, J=8.3 Hz), 7.21-7.36 (4H, m), 7.75 (1H, t, J=5.5 Hz), 10.60 (1H, s).

LCMS (ESI⁺) M+H⁺: 423.

Anal. Calcd for C23H23N4O3F: C, 65.39; H, 5.49; N, 13.26. Found: C, 65.34; H, 5.51; N, 13.23.

### Example 173

### N-{3-[5-(4-Fluorophenyl)-3-methyl-4-(3-oxo-3,4-dihydro-2H-1,4-benzoxazin-6-yl)-1H-pyrazol-1-yl]propyl}methanesulfonamide

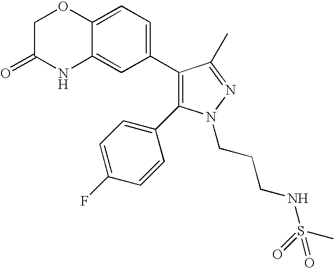

To a stirred mixture of 6-[1-(3-aminopropyl)-5-(4-fluorophenyl)-3-methyl-1H-pyrazol-4-yl]-2H-1,4-benzoxazin-3(4H)-one (200 mg) and triethylamine (0.14 mL) in DMF (2 mL) was added methanesulfonyl chloride (60.2 mg) with ice-cooling. The mixture was stirred for 12 h, treated with water, and extracted with ethyl acetate. The organic layer was washed with brine, dried over MgSO₄, and concentrated in vacuo. The residue was purified by preparative HPLC and crystallization from ethyl acetate/hexane gave the titled compound (135 mg).

¹H-NMR (300 MHz, DMSO-d₆) δ: 1.79-1.96 (2H, m), 2.19 (3H, s), 2.85 (3H, s), 2.86-2.96 (2H, m), 3.93 (2H, t, J=7.0 Hz), 4.53 (2H, s), 6.57 (1H, dd, J=8.0, 1.9 Hz), 6.65 (1H, d, J=1.9 Hz), 6.82 (1H, d, J=8.0 Hz), 6.97 (1H, t, J=5.9 Hz), 7.20-7.41 (4H, m), 10.60 (1H, s).

LCMS (ESI⁺) M+H⁺: 459.

Anal. Calcd for C22H23N4O4SF: C, 57.63; H, 5.06; N, 12.22. Found: C, 57.63; H, 5.08; N, 12.14.

### Example 174

### 6-[1-(2,2-Difluoropropyl)-5-(4-fluorophenyl)-3-methyl-1H-pyrazol-4-yl]-2H-1,4-(2,2-dideuterobenzoxazin-3(4H)-one

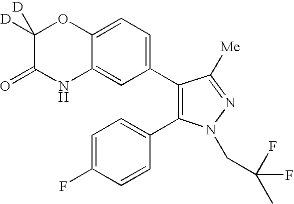

To a solution of 6-[1-(2,2-difluoropropyl)-5-(4-fluorophenyl)-3-methyl-1H-pyrazol-4-yl]-2H-1,4-benzoxazin-3(4H)-one (0.5 g) in CD₃OD (5 mL) was added 40% NaOD in D₂O (0.05 mL) and the mixture was stirred at 50° C. for 30 h. To the mixture was added 1N HCl and the pH was adjusted to about 7. The solvent was removed under reduced pressure and the residue was dissolved with EtOAc and washed with aq. NaHCO₃, water and brine, dried and evaporated. The residue was recrystallized from EtOAc-hexane to afford the title compound as colorless crystals (0.4 g)

Mp 205-206° C.

¹H-NMR (DMSO-d₆) δ: 1.57 (3H, t, J=19.2 Hz), 2.20 (3H, s), 4.38 (2H, t, J=12.9 Hz), 6.57 (1H, dd, J=8.7, 2.4 Hz), 6.64 (1H, d, J=2.4 Hz), 6.81 (1H, d, J=8.7 Hz), 7.2-7.36 (4H, m), 10.60 (1H, s).

Anal. Calcd. for C21H16D2F3N3O2: C, 62.53; H, 4.00; N, 10.42. Found: C, 62.31; H, 4.07; N, 10.29

### Example 175

### 6-[1-(2,2-Difluoro-3-hydroxypropyl)-5-(4-fluorophenyl)-3-methyl-1H-pyrazol-4-yl]-2H-1,4-(2,2-dideuterobenzoxazin-3(4H)-one

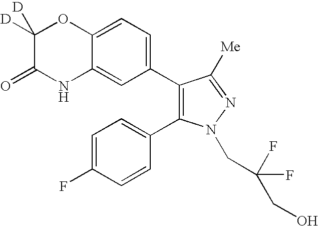

To a solution of 6-[1-(2,2-difluoro-3-hydroxypropyl)-5-(4-fluorophenyl)-3-methyl-1H-pyrazol-4-yl]-2H-1,4-benzoxazin-3(4H)-one (0.5 g) in CD3OD (5 mL) was added 40% NaOD in D2O (0.05 mL) and the mixture was stirred at 50° C. for 80 h. To the mixture 1N HCl was added and the pH was adjusted to about 7. The solvent was removed under reduced pressure and the residue was dissolved with EtOAc and washed with aq. NaHCO₃, water and brine successively, dried and evaporated. The residue was recrystallized from EtOH to afford the title compound as colorless crystals (0.44 g)

Mp 194-195° C.

¹H-NMR (DMSO-d₆) δ: 2.2 (3H, s), 3.52-3.68 (2H, m), 4.35-4.5 (2H, m), 5.54 (1H, t, J=6.3 Hz), 6.54-6.64 (2H, m), 6.8-6.86 (1H, m), 7.21-7.36 (4H, m), 10.60 (1H, s).

Anal. Calcd. for C21H16D2F3N3O3: C, 60.14; H, 3.85; N, 10.02. Found: C, 59.96; H, 3.99; N, 9.92.

### Example 176

### 2,2-Difluoro-3-[5-(4-fluorophenyl)-3-methyl-4-(3-oxo-3,4-dihydro-2H-1,4-benzoxazin-6-yl)-1H-pyrazol-1-yl]propyl N,N-dimethylglycinate hydrochloride

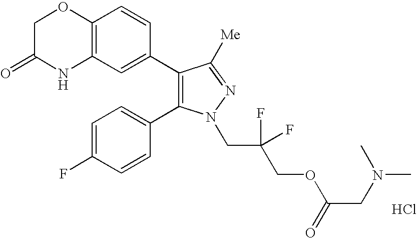

To a solution of 6-[1-(2,2-difluoro-3-hydroxypropyl)-5-(4-fluorophenyl)-3-methyl-1H-pyrazol-4-yl]-2H-1,4-benzoxazin-3(4H)-one (300 mg) in pyridine (6 mL) were added N,N-dimethylglycine hydrochloride (120 mg) and N-(3-dimethylaminopropyl)-N′-ethylcarbodiimide hydrochloride (170 mg) at room temperature. After stirring the mixture for 12 h at room temperature, N,N-dimethylglycine hydrochloride (30 mg) and N-(3-dimethylaminopropyl)-N′-ethylcarbodiimide hydrochloride (43 mg) were added to the mixture and the mixture was stirred for 6 h at room temperature. The mixture was concentrated in vacuo and saturated aqueous sodium bicarbonate solution and water were added to the residue. The mixture was extracted with ethyl acetate. The organic layer was washed with water and brine, dried over Na₂SO₄ and concentrated in vacuo. The residue was dissolved in ethanol (6 mL) and 4N hydrogen chloride in ethyl acetate (2 mL) was added to the solution. The mixture was concentrated in vacuo and the residue was crystallized from ethanol/ethyl acetate to give the title compound as crystals (270 mg).

Mp 198-202° C.

¹H-NMR (300 MHz, DMSO-d₆) δ: 2.21 (3H, s), 2.84 (6H, s), 4.27 (2H, s), 4.49-4.66 (6H, m), 6.55-6.61 (1H, m), 6.69 (1H, d, J=2.3 Hz), 6.83 (1H, d, J=8.3 Hz), 7.27-7.34 (4H, m), 10.62 (1H, s), 10.65 (1H, s).

### Example 177

### 2,2-Difluoro-3-[5-(4-fluorophenyl)-3-methyl-4-(3-oxo-3,4-dihydro-2H-1,4-benzoxazin-6-yl)-1H-pyrazol-1-yl]propyl N,N-dimethylglycinate

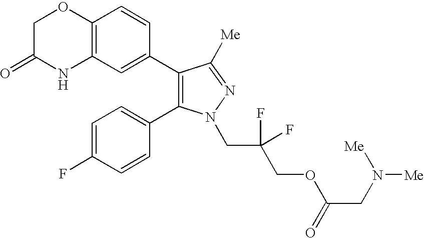

To a solution of 6-[1-(2,2-difluoro-3-hydroxypropyl)-5-(4-fluorophenyl)-3-methyl-1H-pyrazol-4-yl]-2H-1,4-benzoxazin-3(4H)-one (300 mg) in pyridine (6 mL) were added N,N-dimethylglycine hydrochloride (350 mg) and N-(3-dimethylaminopropyl)-N′-ethylcarbodiimide hydrochloride (210 mg) at room temperature. After stirring the mixture for 12 h at room temperature, the mixture was concentrated in vacuo and saturated aqueous sodium bicarbonate solution and water were added to the residue. The mixture was extracted with ethyl acetate. The organic layer was washed with water and brine, dried over Na₂SO₄ and concentrated in vacuo. The residue was dissolved in methanol and the solution was passed through charcoal filter. The filtrate was concentrated in vacuo and the residue was crystallized from ethyl acetate/hexane to give the title compound as crystals (300 mg).

Mp 153-155° C.

¹H-NMR (300 MHz, DMSO-d₆) δ: 2.20 (3H, s), 2.22 (6H, s), 3.17 (2H, s), 4.34-4.58 (6H, m), 6.56-6.62 (1H, m), 6.66 (1H, d, J=2.3 Hz), 6.83 (1H, d, J=8.3 Hz), 7.24-7.34 (4H, m), 10.61 (1H, s).

### Preparation 234

### 2,2-Difluoro-3-[5-(4-fluorophenyl)-3-methyl-4-(3-oxo-3,4-dihydro-2H-1,4-benzoxazin-6-yl)-1H-pyrazol-1-yl]propyl N-(tert-butoxycarbonyl)glycinate

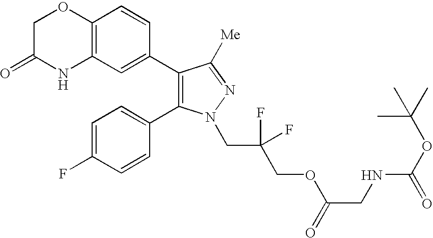

To a mixture of 6-[1-(2,2-difluoro-3-hydroxypropyl)-5-(4-fluorophenyl)-3-methyl-1H-pyrazol-4-yl]-2H-1,4-benzoxazin-3(4H)-one (500 mg), N-(tert-butoxycarbonyl)glycine (320 mg) and pyridine (10 mL) was added N-(3-dimethylaminopropyl)-N′-ethylcarbodiimide hydrochloride (350 mg) at room temperature. After stirring for 12 h at room temperature, N-(3-dimethylaminopropyl)-N′-ethylcarbodiimide hydrochloride (350 mg) was added to the mixture. After stirring for 12 h, the mixture was concentrated in vacuo and saturated aqueous sodium bicarbonate solution and water were added to the residue. The mixture was extracted with ethyl acetate. The organic layer was washed with water and brine, dried over Na₂SO₄ and concentrated in vacuo. The residue was purified by chromatography on silica gel (hexane hexane:ethyl acetate=1:2) followed by crystallization from ethyl acetate/hexane to give the title compound as crystals (670 mg).

¹H-NMR (300 MHz, CDCl₃) δ: 1.46 (9H, s), 2.28 (3H, s), 3.96 (2H, d, J=5.7 Hz), 4.35-4.51 (4H, m), 4.59 (2H, s), 4.98-5.07 (1H, m), 6.49-6.54 (1H, m), 6.65 (1H, dd, J=8.3, 1.9 Hz), 6.85 (1H, d, J=8.3 Hz), 7.05-7.14 (2H, m), 7.15-7.23 (2H, m), 7.84 (1H, s).

### Example 178

### 2,2-Difluoro-3-[5-(4-fluorophenyl)-3-methyl-4-(3-oxo-3,4-dihydro-2H-1,4-benzoxazin-6-yl)-1H-pyrazol-1-yl]propyl glycinate hydrochloride

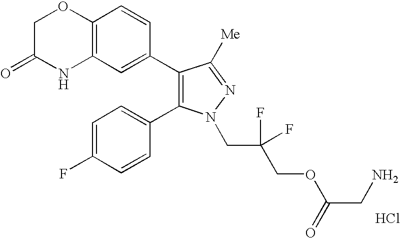

2,2-Difluoro-3-[5-(4-fluorophenyl)-3-methyl-4-(3-oxo-3,4-dihydro-2H-1,4-benzoxazin-6-yl)-1H-pyrazol-1-yl]propyl N-(tert-butoxycarbonyl)glycinate (650 mg) was added to 4N hydrogen chloride in ethyl acetate (13 mL) at room temperature and the mixture was stirred for 1 h. The mixture was concentrated in vacuo and the residue was crystallized from ethanol/ethyl acetate to give the title compound as crystals (175 mg).

Mp 169-171° C.

¹H-NMR (300 MHz, DMSO-d₆) δ: 2.21 (3H, s), 3.87 (2H, s), 4.48-4.68 (6H, m), 6.55-6.63 (1H, m), 6.69 (1H, d, J=1.9 Hz), 6.83 (1H, d, J=8.0 Hz), 7.24-7.37 (4H, m), 8.42 (3H, s), 10.65 (1H, s).

### Example 179

### 2,2-Difluoro-3-[5-(4-fluorophenyl)-3-methyl-4-(3-oxo-3,4-dihydro-2H-1,4-benzoxazin-6-yl)-1H-pyrazol-1-yl]propyl glycinate

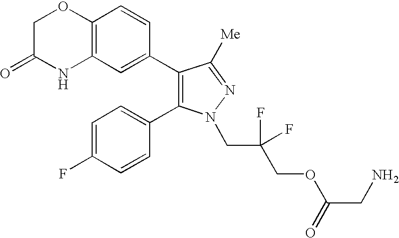

2,2-Difluoro-3-[5-(4-fluorophenyl)-3-methyl-4-(3-oxo-3,4-dihydro-2H-1,4-benzoxazin-6-yl)-1H-pyrazol-1-yl]propyl N-(tert-butoxycarbonyl)glycinate (400 mg) was added to 4N hydrogen chloride in ethyl acetate (8 mL) at room temperature and the mixture was stirred for 0.5 h. The mixture was concentrated in vacuo and saturated aqueous sodium bicarbonate solution was added to the residue. The mixture was extracted with ethyl acetate. The organic layer was washed with brine, dried over Na₂SO₄ and concentrated in vacuo. The residue was crystallized from ethyl acetate/hexane to give the title compound as crystals (260 mg).

Mp 142-146° C.

¹H-NMR (300 MHz, DMSO-d₆) δ: 1.76 (2H, s), 2.20 (3H, s), 3.25 (2H, s), 4.32-4.58 (6H, m), 6.55-6.62 (1H, m), 6.66 (1H, d, J=2.3 Hz), 6.83 (1H, d, J=8.3 Hz), 7.23-7.33 (4H, m), 10.60 (1H, s).

### Example 180

### 2,2-Difluoro-3-[5-(4-fluorophenyl)-3-methyl-4-(3-oxo-3,4-dihydro-2H-1,4-benzoxazin-6-yl)-1H-pyrazol-1-yl]propyl pyridine-3-carboxylate

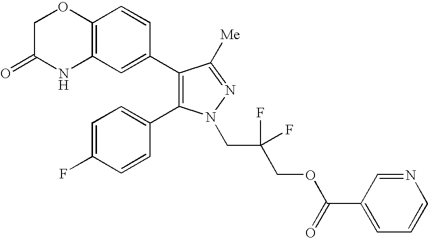

To a solution of 6-[1-(2,2-difluoro-3-hydroxypropyl)-5-(4-fluorophenyl)-3-methyl-1H-pyrazol-4-yl]-2H-1,4-benzoxazin-3(4H)-one (400 mg) in pyridine (4 mL) were added pyridine-3-carboxylic acid (180 mg) and N-(3-dimethylaminopropyl)-N′-ethylcarbodiimide hydrochloride (290 mg) at room temperature. After stirring the mixture for 12 h at room temperature, the mixture was concentrated in vacuo and saturated aqueous sodium bicarbonate solution and water were added to the residue. The mixture was extracted with ethyl acetate. The organic layer was washed with water and brine, dried over Na₂SO₄ and concentrated in vacuo. The residue was crystallized from diethyl ether to give the title compound as crystals (465 mg).

Mp 169-172° C.

¹H-NMR (300 MHz, DMSO-d₆) δ: 2.18 (3H, s), 4.53 (2H, s), 4.58-4.73 (4H, m), 6.57 (1H, dd, J=8.3, 1.9 Hz), 6.65 (1H, d, J=1.9 Hz), 6.82 (1H, d, J=8.3 Hz), 7.16-7.36 (4H, m), 7.55-7.62 (1H, m), 8.14-8.21. (1H, m), 8.87 (1H, dd, J=4.7, 1.7 Hz), 8.98-9.02 (1H, m), 10.60 (1H, s).

### Example 181

### 4-{2,2-Difluoro-3-[5-(4-fluorophenyl)-3-methyl-4-(3-oxo-3,4-dihydro-2H-1,4-benzoxazin-6-yl)-1H-pyrazol-1-yl]propoxy}-4-oxobutanoic acid

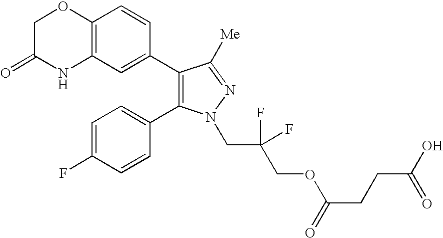

To a solution of 6-[1-(2,2-difluoro-3-hydroxypropyl)-5-(4-fluorophenyl)-3-methyl-1H-pyrazol-4-yl]-2H-1,4-benzoxazin-3(4H)-one (600 mg) in pyridine (6 ml) was added succinic anhydride (170 mg) at room temperature and the mixture was stirred for 24 h at room temperature. Then succinic anhydride (90 mg) was added to the mixture and the mixture was stirred for 24 h at room temperature. The mixture was concentrated in vacuo. Water and ethyl acetate were added to the residue and the aqueous layer was adjusted to pH 4 with 10% hydrochloric acid. The organic layer was separated, washed with water and brine, dried over Na₂SO₄ and concentrated in vacuo. The residue was crystallized from ethyl acetate/hexane to give the title compound as crystals (720 mg).

Mp 186-188° C.

¹H-NMR (300 MHz, DMSO-d₆) δ: 2.20 (3H, s), 2.40-2.57 (4H, m), 4.30-4.58 (6H, m), 6.59 (1H, dd, J=8.3, 1.9 Hz), 6.66 (1H, d, J=1.9 Hz), 6.83 (1H, d, J=8.3 Hz), 7.23-7.34 (4H, m), 10.61 (1H, s), 12.27 (1H, s).

### Example 182

### Sodium 4-{2,2-difluoro-3-[5-(4-fluorophenyl)-3-methyl-4-(3-oxo-3,4-dihydro-2H-1,4-benzoxazin-6-yl)-1H-pyrazol-1-yl]propoxy}-4-oxobutanoate

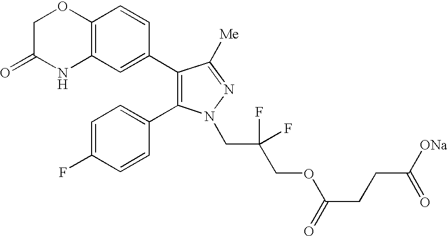

To a solution of 4-{2,2-Difluoro-3-[5-(4-fluorophenyl)-3-methyl-4-(3-oxo-3,4-dihydro-2H-1,4-benzoxazin-6-yl)-1H-pyrazol-1-yl]propoxy}-4-oxobutanoic acid (300 mg) in THF (6 mL) was added ethanol (3 mL) and then 1N sodium hydroxide solution (0.58 mL) was added to the mixture dropwise below 10° C. The mixture was allowed to warm to room temperature and stirred for 0.5 h. The mixture was concentrated in vacuo and the residue was crystallized from ethyl acetate to give the title compound as crystals (280 mg).

Mp 153-158° C.

¹H-NMR (300 MHz, DMSO-d₆) δ: 2.03-2.12 (2H, m), 2.19 (3H, s), 2.30-2.38 (2H, m), 4.29 (2H, t, J=13.6 Hz), 4.45-4.59 (4H, m), 6.53-6.60 (1H, m), 6.74 (1H, d, J=2.3 Hz), 6.81 (1H, d, J=8.3 Hz), 7.22-7.34 (4H, m), 10.76 (1H, s).

### Example 183

### 5-{12,2-Difluoro-3-[5-(4-fluorophenyl)-3-methyl-4-(3-oxo-3,4-dihydro-2H-1,4-benzoxazin-6-yl)-1H-pyrazol-1-yl]propoxy}-5-oxopentanoic acid

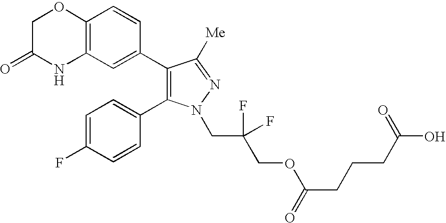

To a solution of 6-[1-(2,2-difluoro-3-hydroxypropyl)-5-(4-fluorophenyl)-3-methyl-1H-pyrazol-4-yl]-2H-1,4-benzoxazin-3(4H)-one (300 mg) in pyridine (3 mL) was added glutaric anhydride (130 mg) at room temperature and the mixture was stirred for 24 h at room temperature. Then glutaric anhydride (130 mg) was added to the mixture and the mixture was stirred for 24 h at room temperature. The mixture was concentrated in vacuo. Water and ethyl acetate were added to the residue and the aqueous layer was adjusted to pH 4 with 10% hydrochloric acid. The organic layer was separated, washed with water and brine, dried over Na₂SO₄ and concentrated in vacuo. The residue was crystallized from ethyl acetate/hexane and washed with diethyl ether to give the title compound as crystals (315 mg).

Mp 152-155° C.

¹H-NMR (300 MHz, DMSO-d₆) δ: 1.70 (2H, quin, J=7.3 Hz) 2.15-2.39 (7H, m) 4.29-4.58 (6H, m) 6.59 (1H, dd, J=8.3, 1.9 Hz) 6.66 (1H, d, J=1.9 Hz) 6.82 (1H, d, J=8.3 Hz) 7.22-7.35 (4H, m) 10.60 (1H, s) 12.11 (1H, s).

### Preparation 235

### Dibenzyl 2,2-difluoro-3-[5-(4-fluorophenyl)-3-methyl-4-(3-oxo-3,4-dihydro-2H-1,4-benzoxazin-6-yl)-1H-pyrazol-1-yl]propyl phosphate

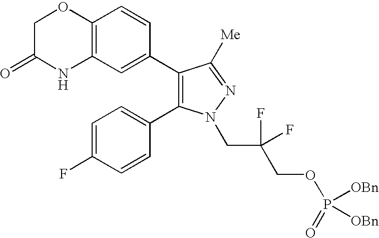

To a solution of 6-[1-(2,2-difluoro-3-hydroxypropyl)-5-(4-fluorophenyl)-3-methyl-1H-pyrazol-4-yl]-2H-1,4-benzoxazin-3(4H)-one (300 mg) and 1H-tetrazole (140 mg) in acetonitrile (4 mL) was added dibenzyl N,N-diisopropylphosphoramidite (500 mg) at room temperature and the mixture was stirred for 3 h at room temperature. Then m-chloroperbenzoic acid (69-75%, 620 mg) was added to the mixture at room temperature and the mixture was stirred for 0.5 h at room temperature. The mixture was diluted with ethyl acetate, washed with aqueous sodium sulfite solution, water and brine, dried over Na₂SO₄ and concentrated in vacuo. The residue was purified by chromatography on silica gel (hexane:ethyl acetate=10:1→1:2) to give the title compound as an oil (460 mg).

¹H-NMR (300 MHz, CDCl₃) δ: 2.26 (3H, s), 4.22-4.40 (4H, m), 4.58 (2H, s), 5.05 (2H, s), 5.08 (2H, s), 6.45 (1H, d, J=1.9 Hz), 6.66 (1H, dd, J=8.3, 1.9 Hz), 6.85 (1H, d, J=8.3 Hz), 7.00-7.10 (2H, m), 7.13-7.22 (2H, m), 7.29-7.40 (10H, m), 7.96 (1H, s).

### Example 184

### 2,2-Difluoro-3-[5-(4-fluorophenyl)-3-methyl-4-(3-oxo-3,4-dihydro-2H-1,4-benzoxazin-6-yl)-1H-pyrazol-1-yl]propyl dihydrogen phosphate

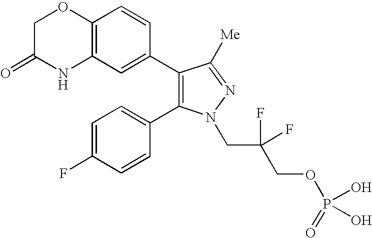

A mixture of dibenzyl 2,2-difluoro-3-[5-(4-fluorophenyl)-3-methyl-4-(3-oxo-3,4-dihydro-2H-1,4-benzoxazin-6-yl)-1H-pyrazol-1-yl]propyl phosphate (430 mg), 10% palladium-carbon (200 mg), methanol (8 mL) and THF (4 mL) was stirred under hydrogen atmosphere (1 atm) at room temperature for 12 h. The catalyst was filtered off and the filtrate was concentrated in vacuo. The residue was crystallized from ethyl acetate to give the title compound as crystals (290 mg).

Mp 224-226° C.

¹H-NMR (300 MHz, DMSO-d₆) δ: 2.21 (3H, s) 4.10 (2H, td, J=14.0, 6.1 Hz) 4.47 (2H, t, J=13.8 Hz) 4.54 (2H, s) 6.56-6.63 (1H, m) 6.66 (1H, d, J=2.3 Hz) 6.82 (1H, d, J=8.3 Hz) 7.21-7.34 (4H, m) 10.61 (1H, s), 2H unconfirmed.

### Example 185

### 2,2-Difluoro-3-[5-(4-fluorophenyl)-3-methyl-4-(3-oxo-3,4-dihydro-2H-1,4-benzoxazin-6-yl)-1H-pyrazol-1-yl]propyl ethyl butanedioate

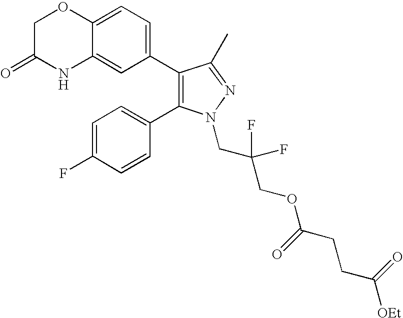

To a mixture of 6-[1-(2,2-difluoro-3-hydroxypropyl)-5-(4-fluorophenyl)-3-methyl-1H-pyrazol-4-yl]-2H-1,4-benzoxazin-3(4H)-one (0.40 g) and 4-ethoxy-4-oxobutanoic acid (0.21 g) in pyridine (8 ml) was added WSC (0.28 g) at room temperature, and the reaction mixture was stirred for 48 h at the same temperature. 4-ethoxy-4-oxobutanoic acid (1.05 g) and WSC (1.40 g) was added to the mixture and the mixture was stirred for 28 h at 60° C. The mixture was treated with saturated aqueous NaHCO₃ and extracted with ethyl acetate. The organic layer was washed with brine, dried over anhydrous magnesium sulfate and concentrated in vacuo. The residue was purified by chromatography on silica gel using n-hexane/ethyl acetate as an eluent and recrystallized from diisopropyl ether and n-hexane to give the title compound as white crystals (0.24 g).

¹H-NMR (300 MHz, DMSO-d₆) δ: 1.16 (3H, t, J=7.0 Hz), 2.20 (3H, s), 2.54-2.63 (4H, m), 4.05 (2H, q, J=7.0 Hz), 4.38 (2H, t, J=14.0 Hz), 4.49-4.60 (4H, m), 6.59 (1H, dd, J=8.1, 2.1 Hz), 6.66 (1H, d, J=2.1 Hz), 6.82 (1H, d, J=8.1 Hz), 7.22-7.35 (4H, m), 10.60 (1H, s).

Mp 100° C.

LCMS (ESI⁺) M+H⁺: 546.

### Example 186

### 2,2-Difluoro-3-[5-(4-fluorophenyl)-3-methyl-4-(3-oxo-3,4-dihydro-2H-1,4-benzoxazin-6-yl)-1H-pyrazol-1-yl]propyl hexadecanoate

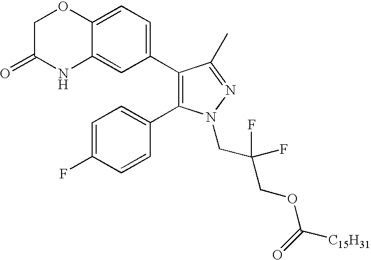

To a mixture of 6-[1-(2,2-difluoro-3-hydroxypropyl)-5-(4-fluorophenyl)-3-methyl-1H-pyrazol-4-yl]-2H-1,4-benzoxazin-3(4H)-one (0.30 g) and hexadecanoic acid (0.28 g) in pyridine (6 ml) was added WSC (0.21 g) at room temperature and the reaction mixture was stirred for 8 days at 60° C. The mixture was treated with saturated aqueous NaHCO₃ and extracted with ethyl acetate. The organic layer was washed with brine, dried over anhydrous magnesium sulfate and concentrated in vacuo. The residue was purified by chromatography on silica gel using n-hexane/ethyl acetate as an eluent and recrystallized from ethyl acetate and n-hexane to give the title compound as white crystals (0.07 g).

¹H-NMR (300 MHz, DMSO-d₆) δ: 0.79-0.90 (3H, m), 1.18-1.31, (26H, m), 2.20 (3H, s), 2.26 (2H, t, J=7.4 Hz), 4.35 (2H, t, J=13.8 Hz), 4.43-4.59 (4H, m), 6.58 (1H, dd, J=8.3, 1.9 Hz), 6.66 (1H, d, J=1.9 Hz), 6.82 (1H, d, J=8.0 Hz), 7.24-7.34 (4H, m), 10.61 (1H, s).

Mp 106° C.

LCMS (ESI⁺) M+H⁺: 656.

### Example 187

### 2,2-Difluoro-3-[5-(4-fluorophenyl)-3-methyl-4-(3-oxo-3,4-dihydro-2H-1,4-benzoxazin-6-yl)-1H-pyrazol-1-yl]propyl ethyl carbonate

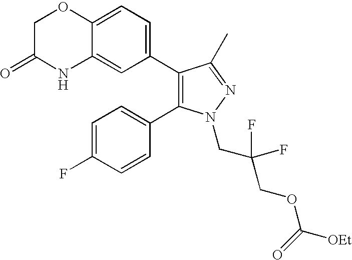

To a solution of 6-[1-(2,2-difluoro-3-hydroxypropyl)-5-(4-fluorophenyl)-3-methyl-1H-pyrazol-4-yl]-2H-1,4-benzoxazin-3(4H)-one (0.30 g) in pyridine (6 ml) and acetonitrile (3 ml) was added ethyl chlorocarbonate (0.10 ml) at room temperature and the reaction mixture was stirred for 2 days at room temperature. Ethyl chlorocarbonate (1.03 ml) was added to the mixture and the mixture was stirred for 2 days at 60° C. The mixture was treated with saturated aqueous NaHCO₃ and extracted with ethyl acetate. The organic layer was washed with brine, dried over anhydrous magnesium sulfate and concentrated in vacuo. The residue was purified by chromatography on silica gel using n-hexane/ethyl acetate as an eluent and recrystallized from diisopropyl ether to give the title compound as white crystals (0.18 g).

¹H-NMR (300 MHz, DMSO-d₆) δ: 1.22 (3H, t, J=7.2 Hz), 2.20 (3H, s), 4.14 (2H, q, J=7.2 Hz), 4.37-4.51 (4H, m), 4.54 (2H, s), 6.59 (1H, dd, J=8.3, 2.3 Hz), 6.66 (1H, d, J=1.9 Hz), 6.82 (1H, d, J=8.0 Hz), 7.23-7.34 (4H, m), 10.60 (1H, s).

Mp 142° C.

LCMS (ESI⁺) M+H⁺: 490.

### Example 188

### 2,2-Difluoro-3-[5-(4-fluorophenyl)-3-methyl-4-(3-oxo-3,4-dihydro-2H-1,4-benzoxazin-6-yl)-1H-pyrazol-1-yl]propyl L-alaninate hydrochloride

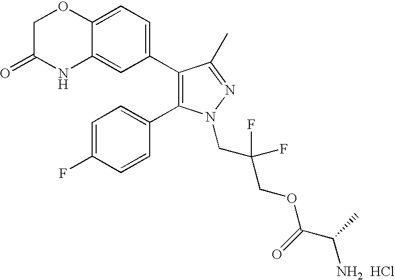

To a mixture of 6-[1-(2,2-difluoro-3-hydroxypropyl)-5-(4-fluorophenyl)-3-methyl-1H-pyrazol-4-yl]-2H-1,4-benzoxazin-3(4H)-one (0.40 g) and N-(tert-butoxycarbonyl)-L-alanine (0.45 g) in pyridine (8 ml) was added WSC (0.46 g) at room temperature and the reaction mixture was stirred for 7 h at room temperature. N-(tert-butoxycarbonyl)-L-alanine (0.90 g) and WSC (0.92 g) were added to the mixture and the mixture was stirred overnight at 60° C. The mixture was treated with saturated aqueous NaHCO₃ and extracted with ethyl acetate. The organic layer was washed with brine, dried over anhydrous magnesium sulfate and concentrated in vacuo. The residue was purified by chromatography on silica gel using n-hexane/ethyl acetate as an eluent. The residue was treated with 4N HCl in ethyl acetate (2 ml) for 1 h, concentrated in vacuo and recrystallized from ethyl acetate and diisopropyl ether to give the title compound as white crystals (0.14 g).

¹H-NMR (300 MHz, DMSO-d₆) δ: 1.37 (3H, d, J=7.16 Hz). 2.21 (3H, s), 4.10-4.22 (1H, m), 4.45-4.74 (6H, m), 6.58 (1H, dd, J=8.3, 1.9 Hz), 6.67 (1H, d, J=1.9 Hz), 6.83 (1H, d, J=8.3 Hz), 7.21-7.39 (4H, m), 8.43 (3H, br. s.), 10.63 (1H, s).

### Example 189

### 2,2-Difluoro-3-[5-(4-fluorophenyl)-3-methyl-4-(3-oxo-3,4-dihydro-2H-1,4-benzoxazin-6-yl)-1H-pyrazol-1-yl]propyl 1-methylethyl carbonate

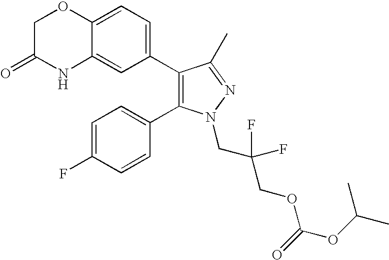

To a solution of 6-[1-(2,2-difluoro-3-hydroxypropyl)-5-(4-fluorophenyl)-3-methyl-1H-pyrazol-4-yl]-2H-1,4-benzoxazin-3(4H)-one (0.30 g) in pyridine (3 ml) and acetonitrile (3 ml) was added dropwise 1-methylethyl chlorocarbonate (1.76 ml) at 60° C. and the reaction mixture was stirred for 4 days at 60° C. The mixture was treated with saturated aqueous NaHCO₃ and extracted with ethyl acetate. The organic layer was separated, washed with brine, dried over anhydrous magnesium sulfate and concentrated in vacuo. The residue was recrystallized from ethyl acetate/n-hexane to give the title compound as white crystals (0.10 g).

¹H-NMR (300 MHz, DMSO-d₆) δ: 1.23 (6H, d, J=6.4 Hz), 2.20 (3H, s), 4.35-4.53 (4H, m), 4.54 (2H, s), 4.76 (1H, quin, J=6.4 Hz), 6.59 (1H, dd, J=8.1, 2.1 Hz), 6.66 (1H, d, J=8.1 Hz), 6.82 (1H, d, J=8.1 Hz), 7.22-7.35 (4H, m), 10.61 (1H, s).

### Example 190

### 2,2-Difluoro-3-[5-(4-fluorophenyl)-3-methyl-4-(3-oxo-3,4-dihydro-2H-1,4-benzoxazin-6-yl)-1H-pyrazol-1-yl]propyl L-valinate hydrochloride

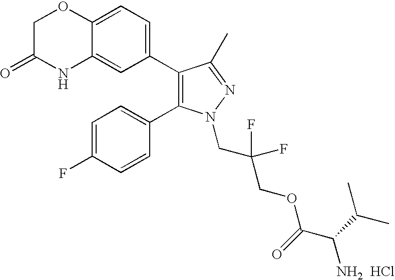

To a mixture of 6-[1-(2,2-difluoro-3-hydroxypropyl)-5-(4-fluorophenyl)-3-methyl-1H-pyrazol-4-yl]-2H-1,4-benzoxazin-3(4H)-one (0.35 g) and N-(tert-butoxycarbonyl)-L-valine (0.45 g) in pyridine (8 ml) was added WSC (0.46 g) at room temperature and the reaction mixture was stirred for 7 h at room temperature. N-(tert-butoxycarbonyl)-L-valine (1.46 g) and WSC (1.29 g) were added to the mixture and the mixture was further stirred overnight at 60° C. N-(tert-butoxycarbonyl)-L-valine (0.91 g) and WSC (0.81 g) were added to the mixture and the mixture was stirred overnight at 60° C. The mixture was treated with saturated aqueous NaHCO₃ and extracted with ethyl acetate. The organic layer was washed with brine, dried over anhydrous magnesium sulfate and concentrated in vacuo. The residue was purified by chromatography on silica gel using n-hexane/ethyl acetate as an eluent. The residue was treated with 4N HCl in ethyl acetate (2 ml) for 1 h, concentrated in vacuo and recrystallized from ethyl acetate and ethanol to give the title compound as white crystals (0.16 g).

¹H-NMR (300 MHz, DMSO-d₆) δ: 0.81-1.02 (6H, m), 1.05-1.10 (1H, m), 2.21 (3H, s) 3.44 (1H, d, J=7.19 Hz), 4.49-4.81 (6H, m), 6.58 (1H, dd, J=8.2, 2.1 Hz), 6.68 (1H, t, J=2.1 Hz), 6.82 (1H, d, J=8.2 Hz), 7.20-7.36 (4H, m), 8.59 (3H, br. s.), 10.65 (1H, br. s.).

### Example 191

### Sodium 5-{2,2-Difluoro-3-[5-(4-fluorophenyl)-3-methyl-4-(3-oxo-3,4-dihydro-2H-1,4-benzoxazin-6-yl)-1H-pyrazol-1-yl]propoxy}-5-oxopentanoate

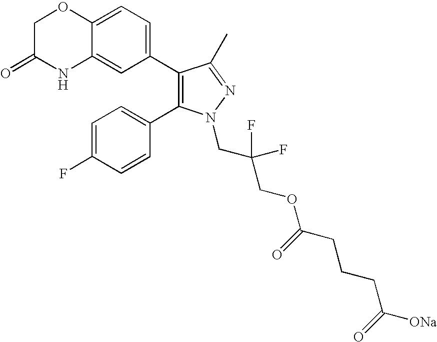

To a solution of 6-[1-(2,2-difluoro-3-hydroxypropyl)-5-(4-fluorophenyl)-3-methyl-1H-pyrazol-4-yl]-2H-1,4-benzoxazin-3(4H)-one (0.30 g) in pyridine (3 ml) was added glutaric anhydride (0.13 g) and the reaction mixture was stirred for 24 h at room temperature. Glutaric anhydride (0.52 g) was added to the mixture and the mixture was stirred for 2 days 60° C. The mixture was treated with 1N HCl and extracted with ethyl acetate. The organic layer was washed with brine, dried over anhydrous magnesium sulfate and concentrated in vacuo. The residue was purified by reverse-phase HPLC. The residue (0.30 g) was dissolved in THF (6 ml) and ethanol (3 ml), and 1N NaOH was added dropwise below 10° C. The mixture was stirred for 1 h at room temperature and concentrated in vacuo. The residue was recrystallized from ethyl acetate/ethanol to give the title compound as white crystals (0.21 g).

¹H-NMR (300 MHz, DMSO-d₆) δ: 1.47-1.68 (2H, m), 1.86 (2H, m), 2.12-2.23 (5H, m), 4.26 (2H, t, J=12.6 Hz), 4.43-4.67 (4H, m), 6.37 (1H, d, J=8.3 Hz), 6.74 (1H, d, J=8.3 Hz), 7.00 (1H, br. s.), 7.19-7.37 (4H, m), 12.04 (1H, br. s.).

LCMS (ESI⁺) M+H⁺: 532.

### Example 192

### 2-({2,2-Difluoro-3-[5-(4-fluorophenyl)-3-methyl-4-(3-oxo-3,4-dihydro-2H-1,4-benzoxazin-6-yl)-1H-pyrazol-1-yl]propoxy}carbonyl)benzoic acid

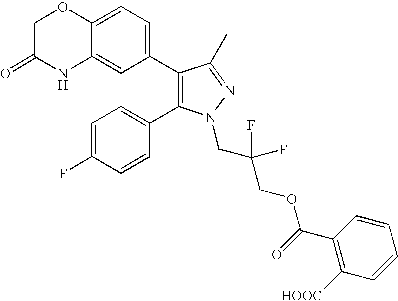

To a solution of 6-[1-(2,2-difluoro-3-hydroxypropyl)-5-(4-fluorophenyl)-3-methyl-1H-pyrazol-4-yl]-2H-1,4-benzoxazin-3(4H)-one (0.30 g) in pyridine (3 ml) was added phthalic anhydride (0.16 g) and the reaction mixture was stirred for 4 h at 60° C. Phthalic anhydride (0.48 g) was added to the mixture and the mixture was stirred for 20 h at 60° C. The mixture was treated with 1N HCl and extracted with ethyl acetate. The organic layer was washed with brine, dried over anhydrous magnesium sulfate and concentrated in vacuo. The residue was purified by reverse-phase HPLC and recrystalized from ethyl acetate/n-hexane to give the title compound as white crystals (0.25 g).

¹H-NMR (300 MHz, DMSO-d₆) δ: 2.21 (3H, s), 4.48-4.68 (6H, m), 6.58 (1H, d, J=8.3 Hz), 6.65 (1H, s), 6.82 (1H, d, J=8.33 Hz), 7.09-7.33 (4H, m), 7.51-7.60 (1H, m), 7.60-7.74 (2H, m), 7.74-7.84 (1H, m), 10.60 (1H, br. s.). LCMS (ESI⁺) M+H⁺: 566.

### Example 193

### 2,2-Difluoro-3-[5-(4-fluorophenyl)-3-methyl-4-(3-oxo-3,4-dihydro-2H-1,4-benzoxazin-6-yl)-1H-pyrazol-1-yl]propyl L-isoleucinate hydrochloride

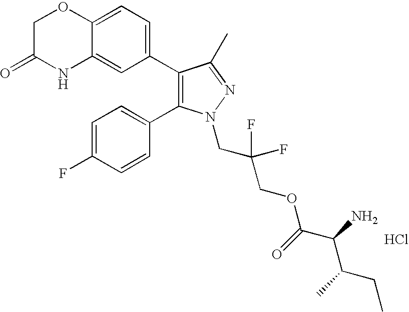

To a mixture of 6-[1-(2,2-difluoro-3-hydroxypropyl)-5-(4-fluorophenyl)-3-methyl-1H-pyrazol-4-yl]-2H-1,4-benzoxazin-3(4H)-one (0.35 g) and N-(tert-butoxycarbonyl)-L-isoleucine (0.81 g) in pyridine (7 ml) was added WSC (0.64 g) at 60° C. and the reaction mixture was stirred for 7 h at 60° C. N-(tert-butoxycarbonyl)-L-isoleucine (2.00 g) and WSC (1.61 g) were added to the mixture and the mixture was stirred overnight at 70° C. The mixture was treated with saturated aqueous NaHCO₃ and extracted with ethyl acetate. The organic layer was washed with brine, dried over anhydrous magnesium sulfate and concentrated in vacuo. The residue was purified by chromatography on silica gel using n-hexane/ethyl acetate as an eluent. The residue was treated with 4N HCl in ethyl acetate (2 ml) for 1 h, concentrated in vacuo and recrystalized from ethyl acetate and ethanol to give the title compound as white crystals (0.17 g).

¹H-NMR (300 MHz, DMSO-d₆) δ: 0.71-0.97 (6H, m), 1.22-1.55 (2H, m), 1.82 (1H, br. s.), 2.21 (3H, s), 4.08 (1H, br. s.), 4.43-4.92 (6H, m), 6.48-6.76 (2H, m), 6.83 (1H, d, J=8.3 Hz), 7.30 (4H, d, J=8.3 Hz), 8.35 (3H, br. s.), 10.61 (1H, s).

LCMS (ESI⁺) M+H⁺: 531.5

### Example 194

### 2,2-Difluoro-3-[5-(4-fluorophenyl)-3-methyl-4-(3-oxo-3,4-dihydro-2H-1,4-benzoxazin-6-yl)-1H-pyrazol-1-yl]propyl-L-leucinate hydrochloride

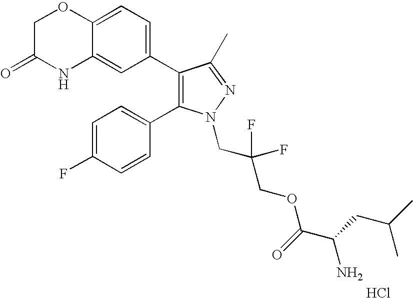

To a mixture of 6-[1-(2,2-difluoro-3-hydroxypropyl)-5-(4-fluorophenyl)-3-methyl-1H-pyrazol-4-yl]-2H-1,4-benzoxazin-3(4H)-one (0.35 g) and N-(tert-butoxycarbonyl)-L-leucine (1.55 g) in pyridine (7 ml) was added WSC (1.29 g) at 60° C. and the reaction mixture was stirred for 14 h at 60° C. N-(tert-butoxycarbonyl)-L-leucine (3.10 g) and WSC (2.58 g) were added to the mixture and the mixture was further stirred overnight at 70° C. The mixture was treated with saturated aqueous NaHCO₃ and extracted with ethyl acetate. The organic layer was washed with brine, dried over anhydrous magnesium sulfate and concentrated in vacuo. The residue was purified by chromatography on silica gel using n-hexane/ethyl acetate as an eluent. The residue was treated with 4N HCl in ethyl acetate (2 ml) for 1 h, concentrated in vacuo and recrystallized from diisopropyl ether and ethanol to give the title compound as white crystals (0.31 g).

¹H-NMR (300 MHz, DMSO-d₆) δ: 0.88 (6H, d, J=5.7 Hz), 1.55-1.67 (2H, m), 1.67-1.82 (1H, m), 2.21 (3H, s), 4.03 (1H, s), 4.37-4.75 (6H, m), 6.58 (1H, dd, J=8.3, 1.9 Hz), 6.66 (1H, s), 6.83 (1H, d, J=8.3 Hz), 7.22-7.38 (4H, m), 8.48 (3H, br. s.), 10.64 (1H, s).

Mp 215° C. (decomposition)

LCMS (ESI⁺) M+H⁺: 530.

### Example 195

### 2,2-Difluoro-3-[5-(4-fluorophenyl)-3-methyl-4-(3-oxo-3,4-dihydro-2H-1,4-benzoxazin-6-yl)-1H-pyrazol-1-yl]propyl L-prolinate hydrochloride

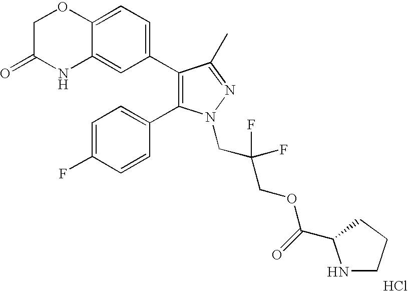

To a mixture of 6-[1-(2,2-difluoro-3-hydroxypropyl)-5-(4-fluorophenyl)-3-methyl-1H-pyrazol-4-yl]-2H-1,4-benzoxazin-3(4H)-one (0.35 g) and N-(tert-butoxycarbonyl)-L-proline (1.81 g) in pyridine (7 ml) was added WSC (1.61 g) at 60° C. and the reaction mixture was stirred for 6 h at 60° C. The mixture was treated with saturated aqueous NaHCO₃ and extracted with ethyl acetate. The organic layer was washed with brine, dried over anhydrous magnesium sulfate and concentrated in vacuo. The residue was purified by chromatography on silica gel using n-hexane/ethyl acetate as an eluent. The residue was treated with 4N HCl in ethyl acetate (2 ml) for 1 h, concentrated in vacuo and recrystallized from diisopropyl ether and ethanol to give the title compound as white crystals (0.18 g).

¹H-NMR (300 MHz, DMSO-d₆) δ: 1.86-2.02 (3H, m), 2.18-2.31 (4H, m), 3.15-3.27 (2H, m), 4.39-4.69 (7H, m), 6.58 (1H, dd, J=8.1, 2.1 Hz), 6.66 (1H, s), 6.83 (1H, d, J=8.1 Hz), 7.30 (4H, d, J=7.2 Hz), 9.30 (2H, br. s.), 10.63 (1H, br. s.).

Mp 165° C. (decomposition)

### Preparation 236

### 4-Benzyl 1-{2,2-difluoro-3-[5-(4-fluorophenyl)-3-methyl-4-(3-oxo-3,4-dihydro-2H-1,4-benzoxazin-6-yl)-1H-pyrazol-1-yl]propyl} N-(tert-butoxycarbonyl)-L-aspartate

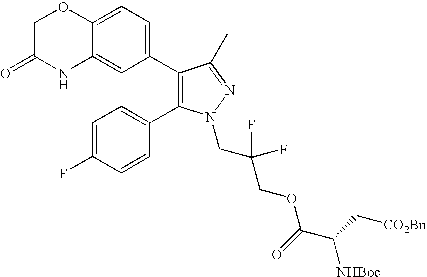

To a mixture of 6-[1-(2,2-difluoro-3-hydroxypropyl)-5-(4-fluorophenyl)-3-methyl-1H-pyrazol-4-yl]-2H-1,4-benzoxazin-3(4H)-one (0.50 g), (2S)-4-(benzyloxy)-2-[(tert-butoxycarbonyl)amino]-4-oxobutanoic acid (1.94 g) in pyridine (15 ml) was added WSC (1.15 g) at room temperature and the reaction mixture was stirred for 24 h at 60° C. The mixture was treated with saturated aqueous NaHCO₃ and extracted with ethyl acetate. The organic layer was washed with brine, dried over anhydrous magnesium sulfate and concentrated in vacuo. The residue was purified by chromatography on silica gel using n-hexane/ethyl acetate as an eluent to give the title compound (0.53 g).

¹H-NMR (300 MHz, DMSO-d₆) δ: 1.36 (9H, s), 2.20 (3H, s), 2.69-2.90 (3H, m), 4.32-4.60 (7H, m), 5.10 (2H, s), 6.54-6.63 (1H, m), 6.65 (1H, d, J=1.9 Hz), 6.82 (1H, d, J=8.0 Hz), 7.24-7.31 (4H, m), 7.35 (5H, s), 10.61 (1H, s).

LCMS (ESI⁺) M+H⁺: 723.

### Example 196

### 1-{2,2-Difluoro-3-[5-(4-fluorophenyl)-3-methyl-4-(3-oxo-3,4-dihydro-2H-1,4-benzoxazin-6-yl)-1H-pyrazol-1-yl]propyl}4-ethyl L-aspartate hydrochloride

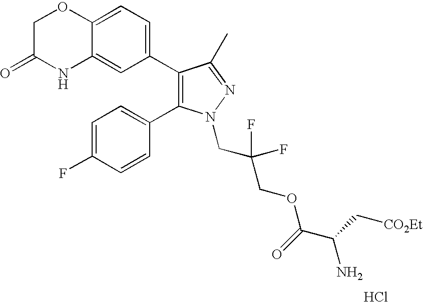

To a mixture of 4-benzyl 1-{2,2-difluoro-3-[5-(4-fluorophenyl)-3-methyl-4-(3-oxo-3,4-dihydro-2H-1,4-benzoxazin-6-yl)-1H-pyrazol-1-yl]propyl}N-(tert-butoxycarbonyl)-L-aspartate (0.45 g) and palladium hydroxide 20% on carbon (wetted with ca. 50% water) (1.94 g) in ethanol (5 ml) and ethyl acetate (1 ml) was added ammonium formate (0.15 g) at 80° C. and the reaction mixture was stirred for 20 min at 80° C. under argon atmosphere. The mixture was treated with saturated aqueous NaHCO₃ and filtered. The filtrate was extracted with ethyl acetate. The organic layer was washed with brine, dried over anhydrous magnesium sulfate and concentrated in vacuo. The residue was purified by reverse-phase HPLC to give a brown solid (0.24 g). The solid was treated with 4N HCl-ethyl acetate (2 ml) and ethanol (0.5 ml). The mixture was stirred for 1 h at room temperature and concentrated in vacuo. The residue was recrystallized from ethyl acetate/n-hexane to give the title compound as white crystals (0.18 g).

¹H-NMR (300 MHz, DMSO-d₆) δ: 0.98-1.10 (2H, m), 1.18 (3H, t, J=6.9 Hz), 2.19 (3H, s), 4.10 (2H, q, J=6.9 Hz), 4.39-4.65 (7H, m), 6.58 (1H, dd, J=8.3, 1.9 Hz), 6.65 (1H, s), 6.83 (1H, d, J=8.3 Hz), 7.20-7.35 (4H, m), 8.54 (3H, br. s.), 10.62 (1H, s).

LCMS (ESI⁺) M+H⁺: 561.

### Example 197

### 2,2-Difluoro-3-[5-(4-fluorophenyl)-3-methyl-4-(3-oxo-3,4-dihydro-2H-1,4-benzoxazin-6-yl)-1H-pyrazol-1-yl]propyl dimethylcarbamate

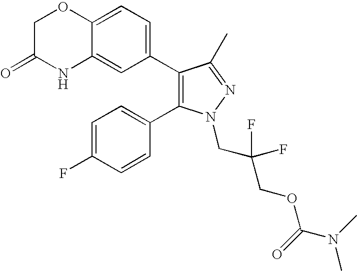

To a solution of 6-[1-(2,2-difluoro-3-hydroxypropyl)-5-(4-fluorophenyl)-3-methyl-1H-pyrazol-4-yl]-2H-1,4-benzoxazin-3(4H)-one (0.30 g) in pyridine (15 ml) was added dropwise dimethylcarbamic chloride (6.5 ml) at 0° C. and the reaction mixture was stirred for 1 h at 0° C. and stirred for 32 h at room temperature. The mixture was treated with saturated aqueous NaNCO₃ and extracted with ethyl acetate. The organic layer was washed with brine, dried over anhydrous magnesium sulfate and concentrated in vacuo. The residue was purified by silica-gel column chromatography eluted with ethyl acetate/n-hexane and recrystallized from ethyl acetate/n-hexane to give the title compound as white crystals (0.17 g).

¹H-NMR (300 MHz, DMSO-d₆) δ: 2.20 (3H, s), 2.70 (3H, s), 2.80 (3H, s), 4.28 (2H, t, J=13.6 Hz), 4.43-4.59 (4H, m), 6.58 (1H, dd, J=8.3, 1.9 Hz), 6.66 (1H, d, J=2.3 Hz), 6.82 (1H, d, J=8.3 Hz), 7.21-7.38 (4H, m), 10.61 (1H, s).

Mp 146° C.

### Example 198

### 2,2-Difluoro-3-[5-(4-fluorophenyl)-3-methyl-4-(3-oxo-3,4-dihydro-2H-1,4-benzoxazin-6-yl)-1H-pyrazol-1-yl]propyl methylcarbamate

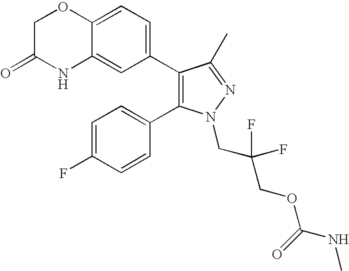

To a solution of triethyl amine (1.10 ml) in acetonitrile was added dropwise triphosgene (2.35 g) and the mixture was stirred for 5 min. 6-[1-(2,2-difluoro-3-hydroxypropyl)-5-(4-fluorophenyl)-3-methyl-1H-pyrazol-4-yl]-2H-1,4-benzoxazin-3(4H)-one (0.33 g) was added to the mixture and stirred for 1 h at 0° C. The mixture was treated with saturated aqueous NaHCO₃ and extracted with ethyl acetate. The organic layer was washed with brine, dried over anhydrous magnesium sulfate and concentrated in vacuo. The residue was purified with silica-gel column chromatography eluted with ethyl acetate/n-hexane and recrystallized from ethyl acetate/n-hexane to give the title compound as white crystals (0.21 g).

¹H-NMR (300 MHz, DMSO-d₆) δ: 2.20 (3H, s), 2.57 (3H, d, J=4.5 Hz), 4.17-4.65 (6H, m), 6.58 (1H, dd, J=8.1, 2.1 Hz), 6.66 (1H, s), 6.82 (1H, d, J=8.1 Hz), 7.28 (4H, d, J=7.6 Hz), 10.61 (1H, br. s.).

Mp 112° C.

LCMS (ESI⁺) M+H⁺: 475.

### Experimental Example 1

The following procedures described in this Example were carried out according to the methods described in Molecular Cloning—Cold Spring Harbor Laboratory (1989) or protocols specified by manufacturers.

(1) Cloning of Human Mineralocorticoid Receptor (hMR) cDNA

hMR cDNA was amplified by polymerase chain reaction (PCR) from human kidney cDNA library. Full-length cDNA was constructed from two fragments of hMR cDNA amplified separately. The primers were designed referring to the nucleotide sequence of hMR cDNA reported by Arriza et. al (Science 1987; 237: 268-275).

hMR-U:

(SEQ ID No. 1)

5′-GGGGCTCGAGGCAGGGATGGAGACCAAAGGCTAC-3′
hMR-1911L:

(SEQ ID No. 2)

5′-GGATACCCATCACTTCTTCTAGACGACAGG-3′
hMR-1686U:

(SEQ ID No. 3)

5′-AGTGGGTATTAAACAAGAACCAGATGACGG-3′
hMR-L:

(SEQ ID No. 4)

5′-GGGAGGTACCTTCTGGGCAGCGGGCAGTCACTTC-3′

The PCR reactions were carried out using PyrobestR® DNA polymerase (Takara). The PCR products were electrophoresed in agarose gel, and 1.7 kb (region (i)) and 1.5 kb (region (ii)) DNA fragments were recovered. Each DNA fragment was inserted into pCR®4Blunt-TOPO® vector (Invitrogen). The resulting plasmids thus obtained were designated as pB-hMR (i) and pB-hMR (ii). To obtain the full-length hMR cDNA, pB-hMR (i) was digested with XhoI and PvuI, and pB-hMR (ii) was digested with PvuI and KpnI, respectively, and the two cDNA fragments were ligated into pBlueScript®IISK+ vector (Stratagene). The resulting plasmid thus obtained was designated as pB-hMR.

### (2) Construction of hMR Expression Plasmid

pMCMVneo (described in WO03/099793) was digested with XhoI and KpnI, and 5.6 kb fragment was ligated with 2.9 kb hMR cDNA fragment obtained by digestion of pB-hMR (described in above (1)) with XhoI and KpnI. The plasmid thus obtained was designated as pMCMVneo-hMR.

### (3) Expression of hMR in FreeStyle 293 Cells and Preparation of Cell Lysate

FreeStyle 293 cells were inoculated at 3×10⁸ cells in 270 ml FreeStyle™ 293 Expression Medium (Invitrogen) in a 1000 ml Erlenmeyer flask. The cells were treated with 30 ml of the transfection mixture containing 300 μg of pMCMVneo-hMR obtained in above (2) and 400 μl of 293fectin™ Reagent (Invitrogen). The transfected cells were cultivated for 48 hr at 37° C. in 8% CO₂ atmosphere at 125 rpm. The cultivated cells were centrifuged and washed with TEG buffer (10 mM Tris-HCl (pH 7.2), 50 mM EDTA, 10% glycerol), and resuspended in 10 ml TEGM buffer (10 mM Tris-HCl (pH 7.2), 1 mM EDTA, 10% glycerol, 1 mM β-mercaptoethanol, 10 mM sodium molybdate, 1 mM dithiothreitol, 2 tablets/100 ml of protease inhibitor cocktail tablets (Roche)). The cell suspension was frozen with liquid nitrogen and thawed on ice, and ultra-centrifuged at 186,000×g for 20 min at 4° C. The supernatant fraction including hMR (hMR lysate) was collected and stored at −80° C.

### (4) Measurement of Inhibition Activity Against Binding of hMR and Aldosterone

[³H]-Aldosterone (Amersham Biosciences) as ligand was added at 10 nM to the reaction mixture including test compound at various concentration and hMR lysate (lot.19: 0.675 mg/mL, lot.20: 0.675 mg/mL, lot.21: 0.765 mg/mL, lot.22: 0.900 mg/mi, lot.23: 0.750 mg/mL) obtained in above (3) and mixture was filled up to 50.5 μl with TEGM buffer. The reaction mixture was incubated for 16 hr at 4° C. and 35 μl of dextran/gelatin coated charcoal suspension (5% charcoal, 0.5% dextran T-70 (Amersham Biosciences), 0.1% gelatin (SIGMA), 10 mM Tris HCl (pH 7.2), 1 mM EDTA) was added thereto to separate bound and free radioactive aldosterone. The mixture containing charcoal was incubated for 10 min at 4° C. and centrifuged at 910×g for 10 min at 4° C.

Radioactivity in 30 μl of the supernatant was measured by TopCount™ (Packard).

For the determination of nonspecific binding, cold Aldosterone instead of drug was added to reaction mixture at 100 μM. Specific binding was determined by subtracting nonspecific binding from total binding.

### (5) Experimental Results

Table 1 shows inhibition rate of compounds at 10⁻⁵ M. From the results of Table 1, it is clear that compound (I) to and a salt thereof of the present invention have superior MR antagonistic activity.

TABLE 1

| Example Compound | Inhibition rate (at 10⁻⁵ M) |
|---|---|
| Example 1 | +++ |
| Example 6 | +++ |
| Example 25 | +++ |
| Example 26 | +++ |
| Example 83 | +++ |
| Example 87 | +++ |
| Example 109 | +++ |
| Example 125 | +++ |
| Example 128 | +++ |
| Example 135 | +++ |
| Example 146 | +++ |
| Example 159 | +++ |

+++ ≧ 90%

The mineralocorticoid receptor antagonist of the present invention (e.g., hypertension therapeutic agent etc.) can be produced, for example, according to the following formulations.

In the following formulations, as the components (additive) other than the active ingredient, those recited in the Japan Pharmacopoeia, the Japan Pharmacopoeia Japanese Pharmaceutical Codex or Japanese Pharmaceutical Excipients and the like can be used.

### 1. Capsule

| | | |
|---|---|---|
| (1) compound obtained in Example 1 | 40 | mg |
| (2) lactose | 70 | mg |
| (3) microcrystalline cellulose | 9 | mg |
| (4) magnesium stearate | 1 | mg |
| 1 capsule | 120 | mg |

(1), (2), (3) and ½ of (4) are admixed and granulated. The remaining (4) is added and the whole is sealed in a gelatin capsule.

### 2. Tablet

| | | |
|---|---|---|
| (1) compound obtained in Example 1 | 40 | mg |
| (2) lactose | 58 | mg |
| (3) cornstarch | 18 | mg |
| (4) microcrystalline cellulose | 3.5 | mg |
| (5) magnesium stearate | 0.5 | mg |
| 1 tablet | 120 | mg |

(1), (2), (3), ⅔ of (4) and ½ of (5) are admixed and granulated. The remaining (4) and (5) are added to the granules and the mixture is compression-molded into a tablet.

### 3. Capsule

| | | |
|---|---|---|
| (1) compound obtained in Example 55 | 40 | mg |
| (2) lactose | 70 | mg |
| (3) microcrystalline cellulose | 9 | mg |
| (4) magnesium stearate | 1 | mg |
| 1 capsule | 120 | mg |

(1), (2), (3) and ½ of (4) are admixed and granulated. The rest of (4) is added and the whole is sealed in a gelatin capsule.

### 4. Tablet

| | | |
|---|---|---|
| (1) compound obtained in Example 55 | 40 | mg |
| (2) lactose | 58 | mg |
| (3) cornstarch | 18 | mg |
| (4) microcrystalline cellulose | 3.5 | mg |
| (5) magnesium stearate | 0.5 | mg |
| 1 tablet | 120 | mg |

(1), (2), (3), ⅔ of (4) and ½ of (5) are admixed and granulated. The remaining (4) and (5) are added to the granules and the mixture is compression-molded into a tablet.

### INDUSTRIAL APPLICABILITY

The compound of the present invention has a superior mineralocorticoid receptor antagonistic action and is useful as an agent for the prophylaxis or treatment of a disease or condition mediated by the mineralocorticoid receptor activation such as hypertension, cardiac failure and the like.

1. A compound represented by the formula (I): 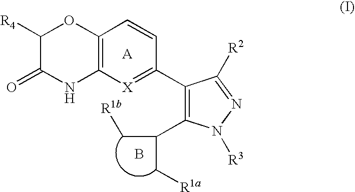
wherein
ring A is a benzene ring or a pyridine ring, each of which is optionally substituted;
ring B is a benzene ring or a 5-or 6-membered aromatic heterocycle, each of which is optionally substituted (wherein two substituents of said benzene ring or said 5- or 6-membered aromatic heterocycle can be bound and form a ring);
X is CX¹ or N;
X¹ is a hydrogen atom, a halogen atom or an optionally substituted C₁₋₆ alkyl;
each of R^{1a} and R^{1b} is independently a hydrogen atom, a halogen atom, a hydroxy, an optionally substituted C₁₋₆ alkyl or an optionally substituted C₁₋₆ alkoxy;
R² is a hydrogen atom, a halogen atom, an optionally substituted C₁₋₆ alkyl, an optionally substituted C₃₋₆ cycloalkyl, an optionally substituted C₁₋₆ alkoxy or an optionally substituted amino group;
R³ is a hydrogen atom or an optionally substituted hydrocarbon group; and
R⁴ is a hydrogen atom, a halogen atom, or an optionally substituted C₁₋₆ alkyl or an optionally substituted hydroxyl,
provided that, R³ is not a hydrogen atom when R² is a hydrogen atom, and R³ is not methyl when at least one of R^{1a} and R^{1b} is methyl.

2. The compound of claim 1, wherein
each of R^{1a} and R^{1b} is independently a hydrogen atom or a halogen atom; and
R³ is an optionally substituted C₁₋₆ alkyl, an optionally substituted C₃₋₆ cycloalkyl, an optionally substituted C₃₋₆ alkenyl or an optionally substituted C₃₋₆ alkynyl.

3. The compound of claim 1, wherein R² is an optionally substituted C₁₋₆ alkyl.

4. The compound of claim 1, wherein R³ is a hydrocarbon group optionally substituted by halogen, cyano, nitro, optionally substituted hydroxy, optionally substituted alkylthio, acyl, optionally substituted carboxy, optionally substituted amino, optionally substituted cycloalkyl, optionally substituted aromatic hydrocarbon ring, optionally substituted aromatic heterocycle, optionally substituted non-aromatic heterocycle, or optionally substituted sulfonamido.

5. The compound of claim 1, wherein R³ is a C₁₋₆ alkyl optionally substituted by halogen, cyano, nitro, optionally substituted hydroxy, optionally substituted alkylthio, acyl, optionally substituted carboxy, optionally substituted amino, optionally substituted cycloalkyl, optionally substituted aromatic hydrocarbon ring, optionally substituted aromatic heterocycle, optionally substituted non-aromatic heterocycle, or optionally substituted sulfonamido.

6. The compound of claim 1, wherein R⁴ is a hydrogen atom.

7. The compound of claim 1, wherein ring B is an optionally substituted benzene ring.

8. The compound of claim 1,
wherein
each of R^{1a} and R^{1b} is independently a hydrogen atom or a halogen atom;
R² is an optionally substituted C₁₋₆ alkyl;
R³ is an optionally substituted C₁₋₆ alkyl;
R⁴ is a hydrogen atom; and
ring B is an optionally substituted benzene ring.

9. A prodrug of the compound of claim 1.

10. A pharmaceutical composition comprising the compound of claim 1 or a prodrug thereof.

11. The pharmaceutical composition of claim 10, wherein the composition is an aldosterone receptor antagonist.

12. The pharmaceutical composition of claim 10, wherein the composition is an agent for preventing or treating hypertension.

13. The pharmaceutical composition of claim 10, wherein the composition is an agent for preventing or treating organ damage caused by hypertension.
